(19) United States
(12) Patent Application Publication
(10) Pub. No.: US 2010/0279302 A1
(43) Pub. Date: Nov. 4, 2010

(54) METHODS OF PREDICTING PAIRABILITY AND SECONDARY STRUCTURES OF RNA MOLECULES

(75) Inventors: Eran Segal, Rehovot (IL); Michael Kertesz, Rehovot (IL); Howard Y. Chang, Stanford, CA (US); John Rinn, Boston, MA (US); Adam Adler, Emerald Hills, CA (US); Yue Wan, Stanford, CA (US)

Correspondence Address: MARTIN D. MOYNIHAN d/b/a PRTSI, INC., P.O. BOX 16446, ARLINGTON, VA, 22215, US

(73) Assignees: Yeda Research And Development Co., Ltd., Rehovot (IL); The Board of Trustees of the Leland Stanford Junior University, Palo Alto, CA (US)

(21) Appl. No.: 12/773,977

(22) Filed: May 5, 2010

Related U.S. Application Data
(63) Continuation-in-part of application No. PCT/IL2010/000246, filed on Mar. 24, 2010.
(60) Provisional application No. 61/202,665, filed on Mar. 24, 2009.

(51) Int. Cl. C12Q 1/68; G01N 33/50

(52) U.S. Cl. 435/6; 436/94

(57) ABSTRACT

Provided are methods of predicting a pairability of nucleotides of a plurality of RNA polynucleotides by (a) simultaneously determining a paired state or an unpaired state of nucleotides of the plurality of RNA polynucleotides; and (b) corresponding the paired state or the unpaired state of the nucleotides to a database of nucleic acid sequences, the database comprises nucleic acid sequences representing the plurality of RNA polynucleotides, thereby determining the pairability of nucleotides of the plurality of RNA polynucleotides. Also provided are methods of determining a secondary structure of a plurality of RNA molecules; methods of determining if a molecule is capable of modulating a secondary structure of at least one RNA polynucleotide of a plurality of RNA polynucleotides; and methods of screening for a marker associated with a pathology.

### RELATED APPLICATION/S

This application is a continuation-in-part (CIP) of PCT Patent Application No. PCT/IL2010/000246 filed Mar. 24, 2010, which claims the benefit of priority under 35 USC 119(e) of U.S. Provisional Patent Application No. 61/202,665 filed Mar. 24, 2009. The contents of all of the above applications are incorporated by reference as if fully set forth herein.

### FIELD AND BACKGROUND OF THE INVENTION

The present invention, in some embodiments thereof, relates to methods of predicting pairability of nucleotides comprised in RNA polynucleotides and, more particularly, but not exclusively, to methods of determining secondary structures of RNA polynucleotides.

RNA structure is important for the function and regulation of RNA, it plays a key role in many biological processes, and largely determines the activity of several classes of non-coding genes (e.g., transfer RNAs and ribosomal RNAs). In addition, substantial regulation of genes that code for proteins occurs post-transcriptionally, in RNA transport, localization, translation, and degradation. This regulation often occurs through structural elements that affect recognition by specific RNA binding proteins. In addition to specific RNA structures, the accessibility of different regions of the RNA was recently shown to be important in several processes such as the ability of microRNAs to bind their targets, control of translation speed and control of translation initiation (Kertesz, M., et al., 2007; Ingolia, N. T., et al., 2009; Ameres, S. L., et al., 2007; Watts, J. M. et al. 2009). Thus, the identification of the structure and accessibility of RNAs is a key to understanding their activity and regulation.

Experimentally, advanced methods for measuring RNA structure such as X-ray crystallography, Nuclear magnetic resonance (NMR) and cryo-electron microscopy, provide detailed three-dimensional descriptions of the probed RNA. However, these methods can only probe a single RNA structure per experiment, and are limited in the length of the probed RNA. Indeed, only ˜750 structures from various organisms were collectively solved by these methods in the past three decades, the vast majority of which being relatively short RNAs (<50 nucleotides).

As they are easier to implement, chemical and enzymatic probing methods have become widely used for RNA secondary structure analysis [Brenowitz, M., et al., 2002; Alkemar, G. & Nygard, O. 2006; Romaniuk, P. J., et al., 1988]. For example, the analyzed RNA can be radiolabelled at one end and digested with an RNase that preferentially cuts double-stranded nucleotides. The length distribution of the resulting RNA fragments is then used to infer which nucleotides of the original RNA molecule were in a double-stranded conformation. Enzymatic probing, however, is also limited to the measurement of one RNA structure per experiment, and depending on whether the enzymatic activity is assayed using standard gel or capillary electrophoresis, only ˜100-600 nucleotides can be analyzed at a time [Deigan, K. E., 2009; Das, R. et al. 2008; US 2010/0035761]. Although there has been considerable success in probing RNA structures of increasing lengths [Watts, J. M. et al. 2009; Mitra, S., 2008; Wilkinson, K. A. et al. 2008] these methods require the extension of multiple sequence-specific primers (derived from the RNA-of-interest) for the analysis of each RNA molecule, and thus cannot be implemented on more than one RNA molecule at a time. Thus, to date, no genome-scale collection of RNA structures currently exists.

Given the experimental difficulties in measuring RNA structure, algorithms for predicting RNA structure from primary sequence have been developed and applied in many settings [Kertesz, M., 2007; Rabani, M., 2008; Zuker, M. 2003; Hofacker, I. L., 2002; Do, C. B., 2006; Mathews, D. H., 1999; Mathews, D. H. 2006]. Although prediction algorithms achieve accuracies of ˜40-70% [Dowell, R. D. & Eddy, S. R. 2004; Doshi, K. J., 2004], their predictive power is limited by the complexity of modeling important factors such as long-distance intramolecular connections or pseuodoknots. More importantly, since there is little experimental data regarding how environmental factors such as changes in pH, temperature, or interactions with metabolites and RNA binding proteins affect RNA structure, these effects cannot be predicted reliably with existing algorithms.

Additional background art include Hofacker L I., et al. Fast folding and comparison of RNA secondary structures. Monatshefte Fr. Chemie. 125:167-188, 1994; Do C B., Woods D A., et al. CONTRAfold: RNA seconday structure prediction without physics-based models. Bioinfomatics 22:90-98, 2006.

### SUMMARY OF THE INVENTION

According to an aspect of some embodiments of the present invention there is provided a method of predicting a pairability of nucleotides of a plurality of RNA polynucleotides, the method comprising: (a) simultaneously determining a paired state or an unpaired state of nucleotides of the plurality of RNA polynucleotides; and (b) corresponding the paired state or the unpaired state of the nucleotides to a database of nucleic acid sequences, the database comprises nucleic acid sequences representing the plurality of RNA polynucleotides, thereby determining the pairability of nucleotides of the plurality of RNA polynucleotides.

According to an aspect of some embodiments of the present invention there is provided a method of determining a secondary structure of a plurality of RNA polynucleotides, the method comprising: (a) predicting the pairability of nucleotides of the plurality of RNA polynucleotides according to the method of the invention; and (b) determining the secondary structure of the plurality of RNA polynucleotides based on the predicted pairability of the nucleotides, thereby determining the secondary structure of the plurality of the RNA polynucleotides.

According to an aspect of some embodiments of the present invention there is provided a method of determining if a molecule is capable of modulating a secondary structure of at least one RNA polynucleotide of a plurality of RNA polynucleotides, the method comprising: (a) contacting the plurality of RNA polynucleotides with the molecule; and (b) comparing a secondary structure of the plurality of RNA polynucleotides following the contacting to a secondary structure of the plurality of RNA polynucleotides prior to the contacting, wherein an alteration above a predetermined threshold in the secondary structure of an RNA polynucleotide following the contacting indicates that the molecule modulates the secondary structure of the RNA polynucleotide, thereby determining if the molecule is capable of modulating the secondary structure of the at least one RNA polynucleotide of the plurality of molecules.

According to an aspect of some embodiments of the present invention there is provided a method of determining if a molecule is capable of modulating a secondary structure of a plurality of RNA polynucleotides, the method comprising (a) contacting the plurality of RNA polynucleotides with the molecule; and (b) determining a secondary structure of the plurality of RNA polynucleotides according to the method of the invention following the contacting and comparing the secondary structure to a secondary structure of the same plurality of RNA polynucleotides prior to the contacting, wherein an alteration above a predetermined threshold of the secondary structure following the contacting indicates that the molecule modulates the secondary structure of the RNA polynucleotides, thereby determining if the molecule is capable of modulating the secondary structure of the plurality of RNA polynucleotides.

According to an aspect of some embodiments of the present invention there is provided a method of determining if a molecule is capable of modulating a secondary structure of at least one RNA polynucleotide of a plurality of RNA polynucleotides, the method comprising (a) contacting the plurality of RNA polynucleotides with the molecule; and (b) determining a secondary structure of the plurality of RNA polynucleotides according to the method of the invention following the contacting and comparing the secondary structure to a secondary structure of the same plurality of RNA polynucleotides prior to the contacting, wherein an alteration above a predetermined threshold of the secondary structure of at least one RNA polynucleotide of the plurality of the RNA molecules following the contacting indicates that the molecule modulates the secondary structure of the at least one RNA polynucleotide, thereby determining if the molecule is capable of modulating the secondary structure of the at least one RNA polynucleotide of a plurality of RNA polynucleotides.

According to an aspect of some embodiments of the present invention there is provided a method of screening for a marker associated with a pathology, the method comprising identifying at least one RNA polynucleotide having an altered secondary structure between cells associated with the pathology and cells devoid of the pathology, wherein an alteration above a predetermined threshold between the secondary structure of the at least one RNA polynucleotide in the cells associated with the pathology and the secondary structure of the at least one RNA polynucleotide in the cells devoid of the pathology indicates that the at least one RNA polynucleotide is associated with the pathology, thereby screening for a marker associated with the pathology.

According to an aspect of some embodiments of the invention, there is provided a method of predicting a pairability of nucleotides of a plurality of RNA polynucleotides, comprising: (a) digesting a sample comprising the RNA polynucleotide with an RNase selected from the group consisting of: (i) an RNase which specifically cleaves a phosphodiester bond of a paired RNA, and (ii) an RNase which specifically cleaves a phosphodiester bond of an unpaired RNA, to thereby obtain digested RNA polynucleotides, to thereby obtain digested RNA polynucleotides; (b) determining a nucleic acid sequence of the digested RNA polynucleotides, and (c) computing an occurrence of a nucleotide of each of the plurality of RNA polynucleotides within the nucleic acid sequence of the digested RNA polynucleotides, thereby predicting the pairability of the nucleotides of the plurality of the RNA polynucleotides.

According to an aspect of some embodiments of the invention, there is provided a method of predicting a pairability of a nucleotide of an RNA polynucleotide, comprising: (a) digesting a sample comprising the RNA polynucleotide with an RNase selected from the group consisting of: (i) an RNase which specifically cleaves a phosphodiester bond of a paired RNA, and (ii) an RNase which specifically cleaves a phosphodiester bond of an unpaired RNA, to thereby obtain digested RNA polynucleotides, to thereby obtain digested RNA polynucleotides; and (b) determining a nucleic acid sequence of the digested RNA polynucleotides using a sequencing apparatus selected from the group consisting of SOLEXA™ (Illumina), PYROSEQUENCING™ 454 (Roche Diagnostics Corporation) and SOLiD™ (Life Technologies), and Helicos (Helicos BioSciences Corporation); (c) computing an occurrence of a nucleotide of the RNA polynucleotide within the nucleic acid sequence of the digested RNA polynucleotides, thereby predicting the pairability of the nucleotide of the RNA polynucleotide.

According to some embodiments of the invention, determining the paired state or the unpaired state is effected using an RNA structure—dependent agent.

According to some embodiments of the invention, the RNA structure—dependent agent is an RNase selected from the group consisting of: (i) an RNase which specifically cleaves a phosphodiester bond of a paired RNA, and (ii) an RNase which specifically cleaves a phosphodiester bond of an unpaired RNA.

According to some embodiments of the invention, the RNase is an endonuclease.

According to some embodiments of the invention, the RNA structure—dependent agent is a chemical selected from the group consisting of: (i) a chemical which specifically binds to an unpaired RNA, and; (ii) a chemical which specifically binds to a paired RNA.

According to some embodiments of the invention, the RNA structure—dependent agent is a chemical selected from the group consisting of: (i) a chemical which specifically modifies an unpaired RNA, and; (ii) a chemical which specifically modifies to a paired RNA.

According to some embodiments of the invention, the RNA structure—dependent agent is a chemical which specifically binds to an unpaired RNA.

According to some embodiments of the invention, binding of the chemical to the RNA is effected covalently.

According to some embodiments of the invention, modification of the RNA by the chemical effected covalently.

According to some embodiments of the invention, the determining the paired state or the unpaired state of the nucleotides is effected by digesting the plurality of RNA polynucleotides with the RNase to thereby obtain digested RNA polynucleotides.

According to some embodiments of the invention, the method further comprising subjecting the digested RNA polynucleotide to reverse transcription to thereby obtain complementary DNA polynucleotides.

According to some embodiments of the invention, determining the paired state or the unpaired state of the nucleotides is effected by reverse transcription of the plurality of RNA polynucleotides following binding of the plurality of RNA polynucleotides with the chemical, to thereby obtain complementary DNA polynucleotides.

According to some embodiments of the invention, corresponding the paired state or the unpaired state of the nucleotides to the data base nucleic acid sequences is effected by comparing a nucleic acid sequence of the complementary DNA polynucleotides with the data base nucleic acid sequences.

According to some embodiments of the invention, the method further comprising computing an occurrence of a nucleotide of each of the plurality of RNA polynucleotides within the nucleic acid sequence of the complementary DNA polynucleotides.

According to some embodiments of the invention, the nucleic acid sequence of the complementary DNA polynucleotides is determined using a sequencing apparatus selected from the group consisting SOLEXA™ (Illumina), PYROSEQUENCING™ 454 (Roche Diagnostics Corporation), SOLiD™ (Life Technologies), and Helicos (Helicos BioSciences Corporation).

According to some embodiments of the invention, determination of the nucleic acid sequence of the complementary DNA polynucleotides is effected for each of the complementary DNA polynucleotides.

According to some embodiments of the invention, computing the occurrence is performed on a nucleotide corresponding to a first nucleotide and/or a last nucleotide of each of the complementary DNA polynucleotides.

According to some embodiments of the invention, a higher occurrence of the nucleotide within the complementary DNA polynucleotides obtained using the RNA structure—dependent agent which is specific to the paired RNA as compared to an expected occurrence of the nucleotide indicates that the nucleotide is in the paired state in the RNA polynucleotide prior to being treated with the RNA structure—dependent agent.

According to some embodiments of the invention, a higher occurrence of the nucleotide within the complementary DNA polynucleotides obtained using the RNA structure—dependent agent which is specific to the unpaired RNA as compared to an expected occurrence of the nucleotide indicates that the nucleotide is in the unpaired state in the RNA polynucleotide prior to being treated with the RNA structure—dependent agent.

According to some embodiments of the invention, a higher occurrence of the nucleotide within the complementary DNA polynucleotides obtained using the RNA structure—dependent agent which is specific to the paired RNA as compared to an occurrence of the nucleotide in the complementary DNA polynucleotides obtained using the RNA structure—dependent agent which is specific to the unpaired RNA indicates that the nucleotide is in the paired state in the RNA polynucleotide prior to being treaed with the RNA structure—dependent agent, and vice versa.

According to some embodiments of the invention, a higher occurrence of the nucleotide within the complementary DNA polynucleotides obtained using the RNA structure—dependent agent which is specific to the unpaired RNA as compared to an occurrence of the nucleotide in the complementary DNA polynucleotides obtained using the RNA structure—dependent agent which is specific to the paired RNA indicates that the nucleotide is in the unpaired state in the RNA polynucleotide prior to the being treated with the RNA structure—dependent agent, and vice versa.

According to some embodiments of the invention, the method further comprising removing proteins from the plurality of the RNA polynucleotides prior to the determining the paired state or the unpaired state of the nucleotides of the plurality of RNA polynucleotides.

According to some embodiments of the invention, the method further comprising denaturing the plurality of the RNA polynucleotides prior to the determining the paired state or the unpaired state of the nucleotides of the plurality of RNA polynucleotides.

According to some embodiments of the invention, the method further comprising subjecting the plurality of the RNA polynucleotides to conditions which allow folding of the RNA polynucleotides following the denaturing.

According to some embodiments of the invention, the RNase which specifically cleaves the phosphodiester bond of the paired RNA is selected from the group consisting of RNase V1 (EC 3.1.27.8) and RNase R.

According to some embodiments of the invention, the RNase which specifically which specifically cleaves the phosphodiester bond of the unpaired RNA is selected from the group consisting of RNase S1 (EC 3.1.30.1), RNase T1 (EC 3.1.27.3) and RNase A (EC 3.1.27.5).

According to some embodiments of the invention, the plurality of RNA polynucleotides are obtained from a cell of an organism.

According to some embodiments of the invention, the secondary structure of the plurality of RNA polynucleotides is determined according to the method of claim 2.

According to some embodiments of the invention, the pairability is determined for each of the nucleotides of at least two of the plurality of RNA polynucleotides.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-F depict a method of measuring structural properties of an RNA transcript by deep sequencing according to some embodiments of the invention. FIG. 1A—RNA molecule is cleaved by RNase V1 at two positions (red triangles, marked ‘1’ and ‘2’); FIG. 1B—The resulting fragments are size-fractionated; FIG. 1C—The RNA fragments are converted to DNA (by reverse transcriptase) and subjected to DNA sequencing; FIG. 1D—The sequenced fragments are aligned to the reference genome (from which the RNA is derived). Each aligned sequence provides structural evidence about two bases. In this example, the cytosine right before the fragment (marked by underlined “C” and ‘1’ in FIG. 1D) and the last uracil of the fragment (marked by underlined “U” and ‘2’ in FIG. 1D) are each likely to be paired in the original structure of the RNA molecule (before the RNA molecule was subjected to digestion with RNase). FIG. 1E—Per-base pairability is obtained by summing the evidence provided by multiple sequences. FIG. 1F—The secondary structure of the RNA molecule can be reconstructed from the pairability estimation.

FIGS. 2A-D depict a method of measuring structural properties of RNA by deep sequencing according to some embodiments of the invention. FIG. 2A—An mRNA molecule which includes a CAP at the 5′ end and a poly A at the 3′ end is subjected to in vitro folding. FIG. 2B—The RNA molecules are cleaved by RNase V1, which cuts 3′ of double-stranded RNA, leaving a 5′ phosphate at a base which immediately follows a base-paired nucleotide in the RNA nucleic acid sequence. One such cut is illustrated by a red arrow. Following random fragmentation, V1-generated fragments are specifically captured and subjected to deep sequencing. Each aligned sequence provides structural evidence about a single base. A large number of reads aligned to the same base indicates that the base is cleaved multiple times by RNase V1 and is thus more likely to be in double stranded conformation. The marked red square illustrates the evidence obtained from one mapped sequence (red), which in this case suggests that the cytosine was paired in the original RNA structure (since the 5′-phosphate fragmented RNA sequence mapped to the uracil which follows that cytosine). Additional evidence (gray boxes) is collected by mapping more sequences (gray horizontal bars). FIG. 2C—Same as in FIG. 2B, but when the RNA sample is treated with RNase S1, which cuts 3′ of single-stranded RNA. Collected reads in this case suggest that the base was unpaired in the original RNA structure. FIG. 2D—Using the binomial test to combine the data extracted from the two complementary experiments (FIG. 2B and FIG. 2C), a nucleotide-resolution score is obtained, representing the likelihood that the inspected base was in a double- or single-stranded conformation.

FIGS. 3A-D demonstrate that PARS correctly recapitulates results of RNA footprinting. FIG. 3A—The PARS signal obtained for bases 50-110 of the yeast gene CCW12 (YLR110C; SEQ ID NO:1) using the double-stranded cutter RNase V1 (red bars, top) or single-stranded cutter RNase S1 (green bars, bottom) accurately matches the signals obtained by traditional footprinting of that same transcript domain (black lines). The PARS signal is shown as the number of sequence reads which mapped to each nucleotide of the inspected domain; footprinting results are obtained by automated quantification of the RNase lanes shown in FIG. 3B. The red arrows indicate RNase V1 cleavages and the green arrows indicate RNase S1 cleavages as shown in the gel (FIG. 3B). FIG. 3B—8% acrylamide/7M urea gel analysis of RNase V1 (lanes 5, 6) and S1 (lanes 3, 4) probing of CCW12. Additionally, RNase T1 ladder (lanes 2, 8), alkaline hydrolysis (lanes 1, 9), and no RNase treatment (lane 7) are shown. The red arrows indicate RNase V1 cleavages and the green arrows indicate RNase S1 cleavages. FIG. 3C—The PARS signal obtained from bases 50-120 of the yeast gene RPL41A (YDL184C; SEQ ID NO:2) matches the signals obtained by traditional footprinting (FIG. 3D). Red bars (top)=the double-stranded cutter RNase V1; Green bars (bottom)=single-stranded cutter RNase S1. FIG. 3D—8% acrylamide/7M urea gel analysis of RNase V1 (lanes 5, 6) and S1 (lanes 7, 8) probing of RPL41A. RNase T1 ladder (lane 2), alkaline hydrolysis (lanes 1, 9), and no RNase treatment (lane 4) are shown. The red arrows indicate RNase V1 cleavages and the green arrows indicate RNase S1 cleavages.

FIGS. 4A-B demonstrate that PARS correctly recapitulates results of RNA footprinting. Raw number of reads obtained using RNase V1 (red bars) or RNase S1 (green bars) and the resulting PARS score (blue bars) along the inspected domains of ASH1 (SEQ ID NO: 3218; FIG. 4A) and URE2 (SEQ ID NO: 3219; FIG. 4B). Also shown are the known structures of the inspected domains. Nucleotides are color-coded according to their computed PARS score (double-stranded in green, single-stranded in red).

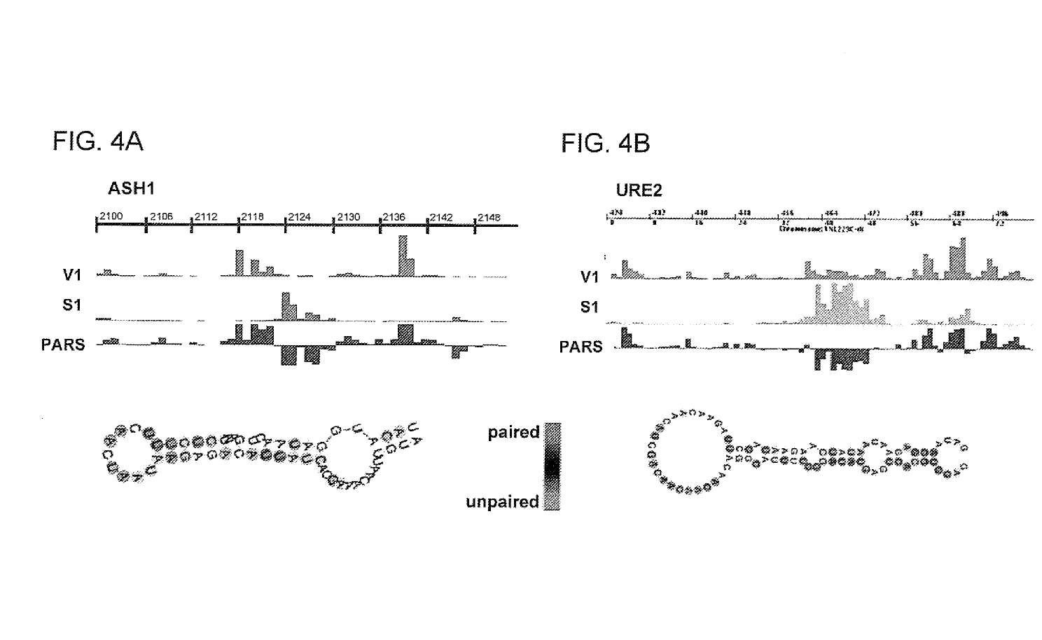

FIGS. 5A-D demonstrate that functional units of the transcript are demarcated by distinct properties of RNA structure. FIG. 5A—Significant correspondence between PARS and computational predictions of RNA structure. The Vienna package (Hofacker, I. L. et al., 2002) was used in order to fold the 3000 yeast mRNAs used in the analysis, and the predicted double-stranded probability of each nucleotide was extracted. Shown is the average predicted double-stranded probability of each nucleotide (y-axis), where nucleotides were sorted by their PARS score (x-axis). Higher PARS scores denote bases that are more likely to be double stranded. Average and standard deviation from 1000 shuffle experiments in which a random prediction score was assigned to each probed base are shown in gray. FIG. 5B—Discrete Fourier transform of average PARS score across the coding region (blue line), 3′ UTR (red line) and 5′ UTR (green line). The high amplitude for a cycle of n=3 bases can clearly be seen for the coding region, whereas the untranslated regions show no structural periodicity. FIG. 5C—a histogram showing the PARS score obtained for each of the three positions of every codon, averaged across all codons (blue bars). FIG. 5D—Shown is the PARS score across the 5′ untranslated region, the coding region (CDS), and the 3′ untranslated region, averaged across all transcripts used in the analysis as a function of position along the transcript. Transcripts were aligned by their translational start and stop sites for the left and right panel, respectively; start and stop codons are indicated by gray bars; horizontal bars denote the average PARS score per region (5′ UTR, coding sequence, 3′ UTR).

FIGS. 6A-D demonstrate that the structure around start codons correlates with low translational efficiency. FIG. 6A—Sliding window analysis of local PARS score and ribosome density as reported by Ingolia, N. T., et al., 2009. Shown is the significance (p-value) of the anti-correlation between average PARS score along a 40 bp-wide window and the reported ribosome density. FIG. 6B—k-means clustergram of PARS scores across the 80 by window surrounding the translation start site of all transcripts for which enough coverage was obtained. Red represents highly structured areas, green areas that are less structured. The average structural profile and number of member genes is shown to the right of each cluster. FIG. 6C—Cumulative distribution plot of ribosome occupancy for each cluster and the associated Kolmogorov-Smirnoff test p-value between the distribution of cluster 1 and 3. FIG. 6D—Tendency for less RNA structure in the first 30 bases of open reading frame (ORFs) encoding predicted secretory proteins. While structure typically builds up immediately upon entry to the coding sequence (CDS), genes predicted to code for secretory proteins retain low structure in the first ˜30 bases of the CDS, consistent with the dual function SSCR having structural features of UTR rather than CDS (Palazzo, A. F. et al. 2007). Shown are the average relative PARS scores (methods) across a 30 by sliding window for the 499 genes coding for secretory proteins (blue), the remaining 2501 genes (green) and the mean and standard deviation obtained from 1000 shuffle experiments in which sets of 499 genes were randomly selected (gray).

FIGS. 7A-D demonstrate that the enzyme concentration used in PARS cuts RNA with a single hit kinetics and occurs at regions resulting from intra-molecular interactions. FIG. 7A—Shown are traces indicating footprinting intensities of P³²-labeled in vitro transcribed YDL184C (SEQ ID NO:14) that were quantified using SAFA [Semi-automate footprinting analysis—more info at Hypertext Transfer Protocol://rnajournal (dot) cshlp (dot) org/content/11/3/344 (dot) full]. The footprint of YDR184C obtained with the RNase V1 concentration used in PARS matches very well with the footprint obtained with a 5 fold dilution of RNase V1 (Pearson's correlation coefficient=0.95). FIG. 7B—The footprint of YDR184C obtained with the RNase S1 concentration used in PARS matches well with the footprint obtained with a 5 fold dilution of RNase S1 (Pearson's correlation coefficient=0.75). FIG. 7C—P³²-labeled RNA is folded and cleaved either by itself or is folded and cleaved in a population of mRNAs. YDR184C folds into a similar structure when it is alone in solution or when it is in the presence of other RNAs (Pearson's correlation coefficient=0.97); FIG. 7D—P³² RNA mixed with 1 μs of yeast total RNA is either folded at 10 μl or 100 μl of a buffer containing 10 mM Tris pH 7, 10 mM MgCl₂, 100 mM KCl) before being cleaved by RNase V1. YDR184C folds into a similar conformation with or without 10× dilution, indicating that most of the folding is driven by intra-molecular interactions (Pearson's correlation coefficient=0.9).

FIGS. 8A-B demonstrate that the protocol according to some embodiments of the invention captures fragments generated from V1 cleavages and not random fragmentation products from alkaline hydolysis. FIG. 8A—A gel image which shows RNA libraries ran on 5% native polyacrylamide gel electrophoresis (PAGE) and stained using ethidium bromide. Fragments above 120 bases indicate yeast RNA fragments that were ligated to adaptors and cloned into a library. The RNAs are either treated (“V1”) or not treated (“Fragment”) before they are fragmented at 95° C. for 3.5 minutes and further ligated to 5′ and 3′ adaptors. Lanes 1, 2, 3 and 4 refer to the amount of library that is amplified with 15, 21, 26, and 31, cycles of PCR, respectively. Typically, the native PAGE is excised between 150 bases to 250 bases for high throughput sequencing. “MW” molecular weight marker in bases. FIG. 8B—Quantitative PCR (qPCR) quantification of the library after 18 cycles of PCR amplification and size selection between 150-250 bases using native PAGE. The “Y” axis represents arbitrary units).

FIGS. 9A-C demonstrate the sampling of cleaved RNA fragments in proportion to their abundance according to the protocol of some embodiments of the invention. FIG. 9A—Histogram showing for the number of transcripts as a function of load obtained by merging the readout of all seven replicates of the PARS experiment. Load is defined as the number of fragments that mapped to a given mRNA divided by the mRNA length. Applying a threshold of load>1, a structural information for 3196 transcripts (solid black line) is obtained. A threshold of load>1 was chosen as a means to ensure that the analyzed transcripts have sufficient coverage. By performing more sequencing runs, better coverage can be obtained, allowing PARS to obtain structural information for many more transcripts. For example, it is likely that structures of ˜1100 additional transcripts will be obtained by doubling the number of sequencing runs (dashed line). FIG. 9B—Comparison of mRNA abundance levels per transcript between three biological replicates of the samples treated by the double-stranded cutter RNase V 1. The abundance level of each transcript is computed as the total number of reads mapped to the transcript divided by the transcript length; The units on the “X”, “Y” and “Z” axes are loads. These results show that the method is not biased towards sampling specific transcripts. FIG. 9C—Same as FIG. 9B, but when comparing the abundance levels and those of the ribosomal profiling method of Ingolia, N. T., et al., 2009 and RNA-Seq method of Nagalakshmi, U. et al. 2008.

FIGS. 10A-D compare sequence-dependent bias using various protocols. FIG. 10A—Shown is the sequence specificity across all sequence reads that was uniquely mapped to the genome from the V1 libraries generated according to the present teachings. The specificity was derived from an alignment of the 20 nucleotides in the genome that surround the first mapped base of each sequence read and are shown as a standard position specific scoring matrix (PSSM), which displays the information content of the nucleotide distribution at each position of the alignment. FIG. 10B—Same as FIG. 10A, for the data obtained from S1 libraries generated according to the present teachings. FIGS. 10C-D—Same as FIG. 10A, for the RNA-Seq data obtained in the study of Nagalakshmi, U. et al. 2008 and the ribosomal profiling data obtained in Ingolia, N. T., 2009. The sequence composition at these bases did not show a strong sequence bias at the first base or around it, suggesting that RNase cleavage, adaptor ligation, and cDNA conversion do not introduce significant sequence biases.

FIG. 11 is a graph demonstrating that the protocol according to some embodiments of the invention has minimal bias towards particular regions of the transcript. Shown is the number of sequence reads along each nucleotide of the annotated coding region of each transcript, averaged across all transcripts. The number of sequence reads are shown after normalizing for the abundance of each transcript, by dividing the number of sequence reads at each nucleotide with the total number of reads for its embedding transcript. Since transcripts vary in length, the position of each normalized read is then projected onto a 0-1 range denoting the 5′ to 3′ end of the coding region of each transcript. Data is shown for the double-stranded (red) and single-stranded (green) cutters, and for the RNA-Seq data (blue) of Nagalakshmi, U. et al. 2008 and ribosomal profiling data (pink) of Ingolia, N. T., 2009.

FIGS. 12A-D demonstrate PARS's ability to solve long RNA structures. FIG. 12A-B—Single-stranded and double-stranded signal of PARS obtained using the RNase S1 (green bars, FIG. 12A) and RNase V1 (red bars, FIG. 12B) across the 2.2 kb HOTAIR (SEQ ID NO:5) Rinn, J. L. et al. 2007) transcript which was analyzed according to the method of some embodiments of the invention, and which structure was previously unknown. FIG. 12C-D—Detailed view of the PARS V1 signal from FIG. 12B across two domains from the full transcript. For each domain, shown is the signal obtained when subjecting this domain to traditional footprinting (black line). The correlations between PARS and traditional footprinting are indicated.

FIGS. 13A-E demonstrate that PARS correctly recapitulates results of RNA footprinting. FIG. 13A—RNase V1 cleaves the folded p4p6 domain (SEQ ID NO:7) of Tetrahymena ribozyme at four distinct sites, which are accurately captured by PARS. Shown is the double-stranded signal of PARS obtained using the double-stranded cutter RNase V1 (red bars), for the p4p6 domain of the Tetrahymena ribozyme, one of the control fragments added to the samples. The signal is shown as the number of sequence reads mapped along each nucleotide of the p4p6 domain. Also shown is the signal obtained on the p4p6 domain using traditional footprinting (black line) and automated quantification of the RNase V1 lane shown in FIG. 13B. FIG. 13B—The gel resulting from RNase V1 (Lanes 7, 8) enzymatic probing of the p4p6 domain. Alkaline hydrolysis (Lanes 1, 2), RNase T1 ladder (Lanes 3, 4) and no RNase treatment (Lane 6) are also shown; FIG. 13C—Single-stranded signal of PARS obtained using the single-stranded cutter RNase S1 (green bars), compared to the signal obtained using traditional footprinting (black line). Green arrows indicate cleavages that are seen in gel (FIG. 13D). FIG. 13D—The gel resulting from RNase V1 (Lane 2) and RNase S1 (Lane 3) enzymatic probing of the p4p6 domain. Alkaline hydrolysis (Lanes 6, 7), RNase T1 ladder (Lane 5) and no RNase treatment (Lane 4) are also shown. FIG. 13E—Known secondary structure of the p4p6 domain43. Arrows mark nucleotides that were identified by both PARS and enzymatic probing as double-stranded (red arrows) or single-stranded (green arrow).

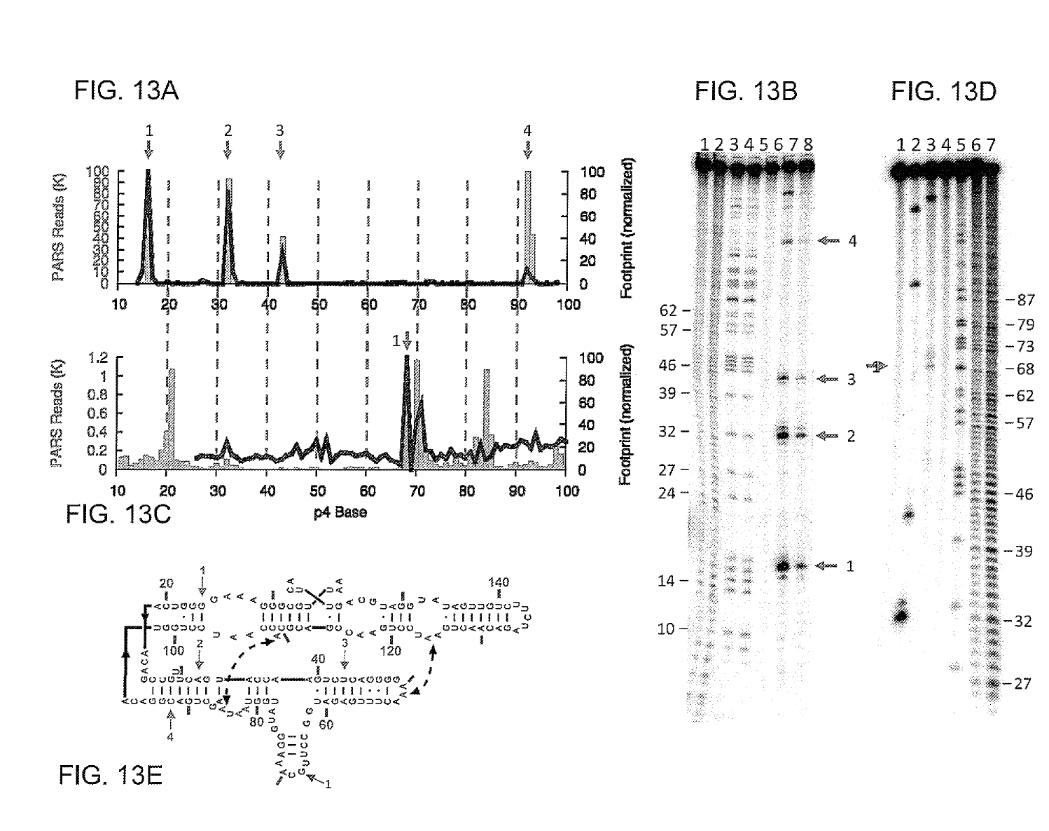

FIGS. 14A-D demonstrate that PARS correctly recapitulates results of RNA footprinting for the p9-9.2 domain of the Tetrahymena ribozyme. FIG. 14A—RNase V1 cleaves the folded p9-9.2 domain of the Tetrahymena ribozyme at two distinct sites, which are accurately captured by PARS. The double-stranded signal of PARS obtained using the double-stranded cutter RNase V1 (red bars) is shown as the number of sequence reads mapped along each nucleotide of the p4p6 domain. Also shown is the signal obtained on the p4p6 domain using traditional footprinting (black line) and automated quantification of the RNase V1 lane shown in FIG. 14C. Red arrows indicate cleavages that are seen in gel (FIG. 14C). FIG. 14B—Single-stranded signal of PARS obtained using the single-stranded cutter RNase S1 (green bars), compared to the signal obtained using traditional footprinting (black line). Green arrows indicate cleavages that are seen in gel (FIG. 14C). FIG. 14C—The gel resulting from RNase V1 (Lane 9) and RNase S1 (Lanes 7, 8, 9 at pH 7 and Lanes 5, 6 at pH 4.5). Alkaline hydrolysis (Lanes 1, 2), RNase T1 ladder (Lane 3) and no RNase treatment (Lane 10) are also shown. FIG. 14D—Known secondary structure of the p9-9.2 domain (Cech, T. R., et al., 1994). Arrows mark nucleotides that were identified by both PARS and enzymatic probing as double-stranded (red arrows) or single-stranded (green arrows).

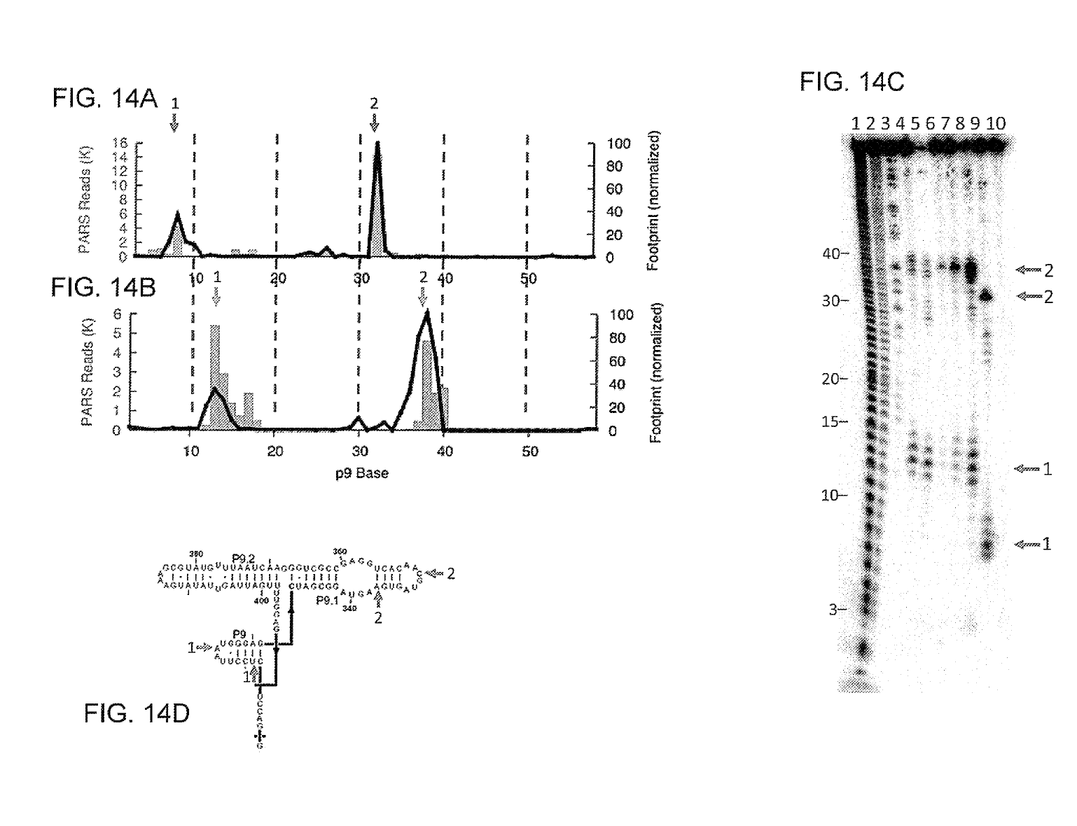

FIGS. 15A-C demonstrate that PARS correctly recapitulates known RNA structures. FIGS. 15A-B—Raw number of reads obtained using RNase V1 (red bars) or RNase S1 (green bars) and the resulting PARS score (blue bars) along the inspected domain of ASH1-E2 (FIG. 15A) and ASH1-E3 (FIG. 15B). FIG. 15C—Shown is the known structure of the inspected domains. Nucleotides are color-coded according to their computed PARS score (paired nucleotides are marked in red, unpaired nucleotides are marked in green).

FIG. 16 is a histogram depicting the effect of folding window size in computational predictions of RNA structure on correspondence to PARS. Shown is the z-score obtained by comparing the average prediction score for bases, which obtained a high PARS score (≧4.5) to random shuffle. Providing the folding algorithm information regarding more than 40 bases around the probed nucleotide does not improve its predictive power.

FIG. 17 is a schematic illustration demonstrating that distinct patterns of secondary structures in mRNA are associated with cytotopic localization and protein function. For each gene, the average PARS score was separately computed for the 5′ UTR (5′-untranslated region), CDS (coding sequence), and 3′ UTR (3′-untranslated region). For each of these three regions, the Wilcoxon rank sum test was used to compute a p-value for whether genes with similar Gene Ontology (GO) annotations have PARS scores that are higher or lower than expected. Multiple-hypothesis correction was done by FDR with a cutoff of 0.05. The Wilcoxon rank sum test results for each GO category are listed in Table 3. As can be seen, mRNAs encoding proteins with specific sub-cellular localizations (blue) or function in several metabolic pathways (yellow) tend to have excess secondary structure in the coding regions, while mRNAs encoding ribosomal proteins (dark red) tend to have less secondary structure than expected in their 5′UTR and CDS.

FIGS. 18A-B depicts PARS scores (FIG. 18B) and the predicted secondary structure (FIG. 18A) of the YAL038W RNA polynucleotide (SEQ ID NO:9).

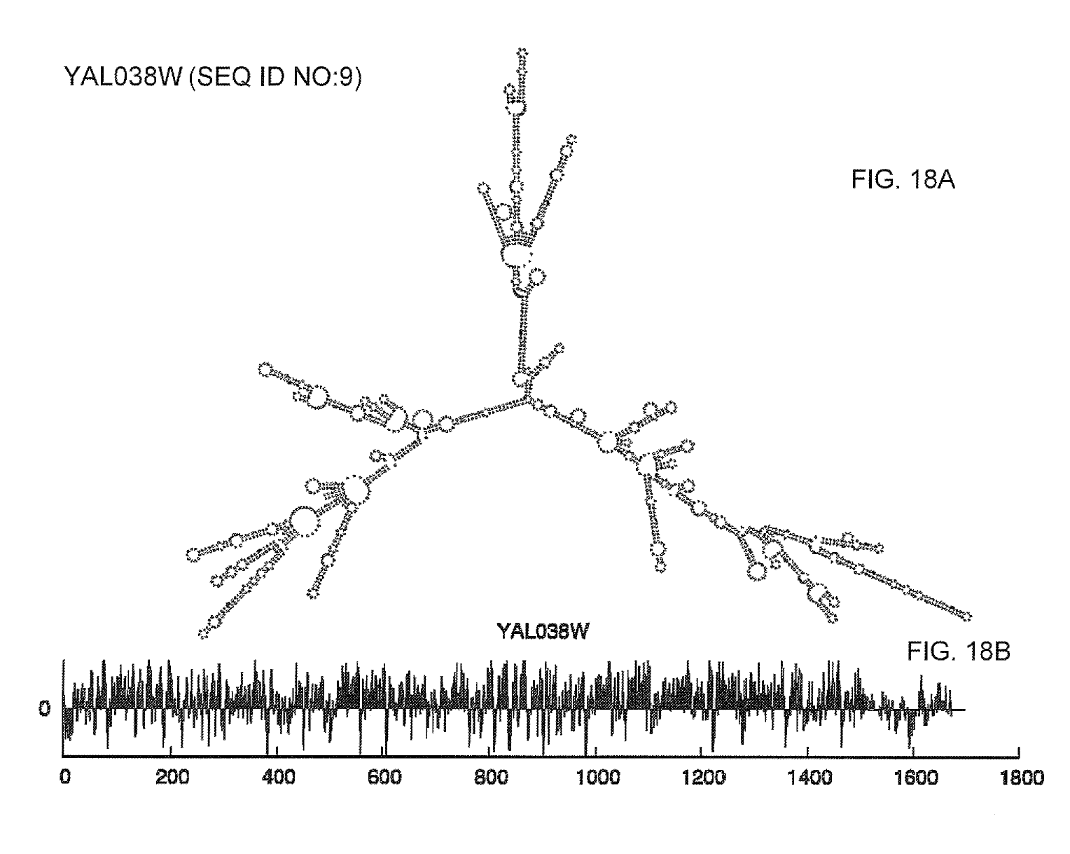

FIGS. 19A-B depicts PARS scores (FIG. 19B) and the predicted secondary structure (FIG. 19A) of the YCR012W RNA polynucleotide (SEQ ID NO:10).

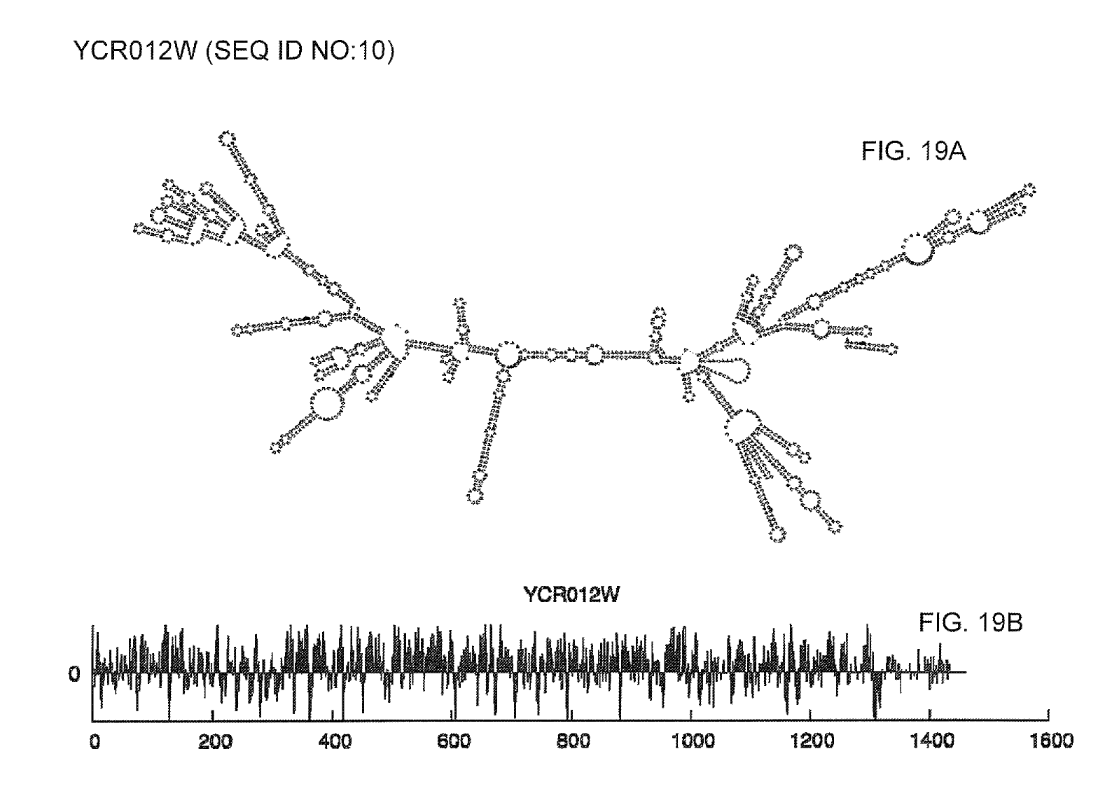

FIGS. 20A-B depicts PARS scores (FIG. 20B) and the predicted secondary structure (FIG. 20A) of the YCR031C RNA polynucleotide (SEQ ID NO:11).

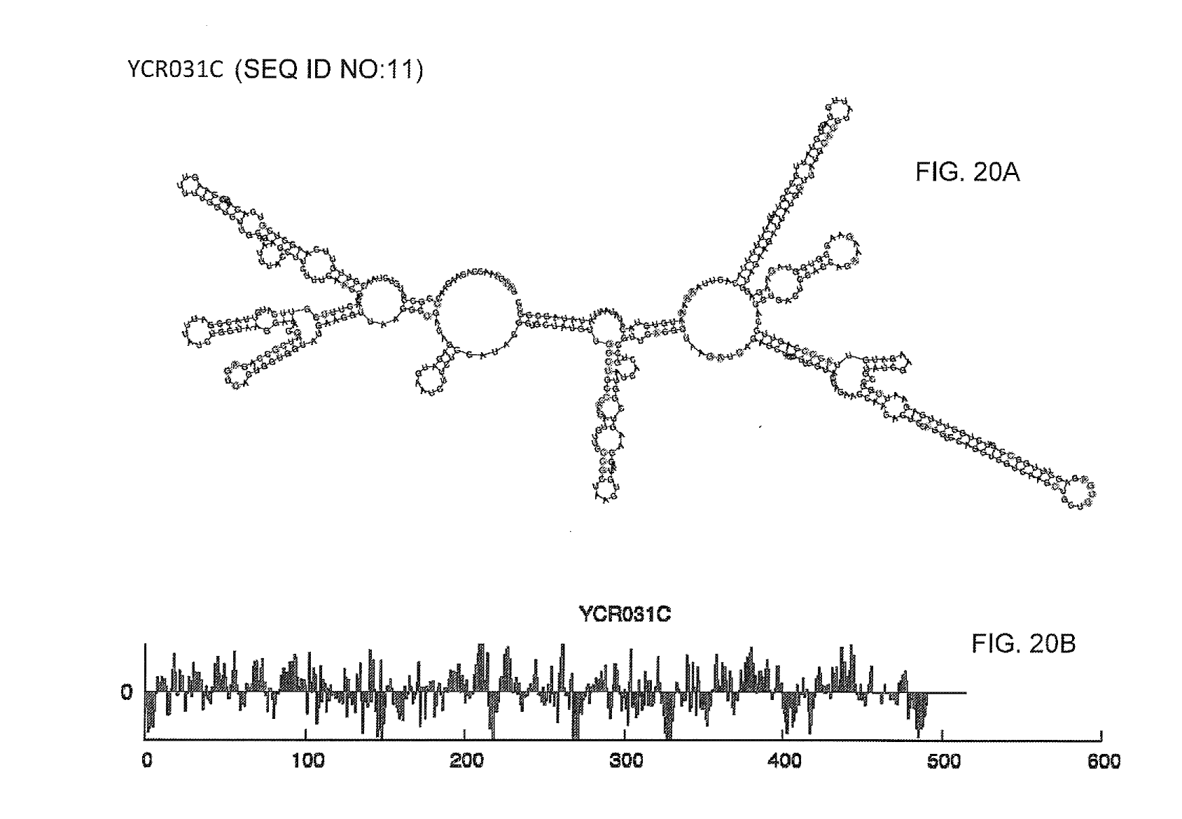

FIGS. 21A-B depicts PARS scores (FIG. 21B) and the predicted secondary structure (FIG. 21A) of the YDL081C RNA polynucleotide (SEQ ID NO:12).

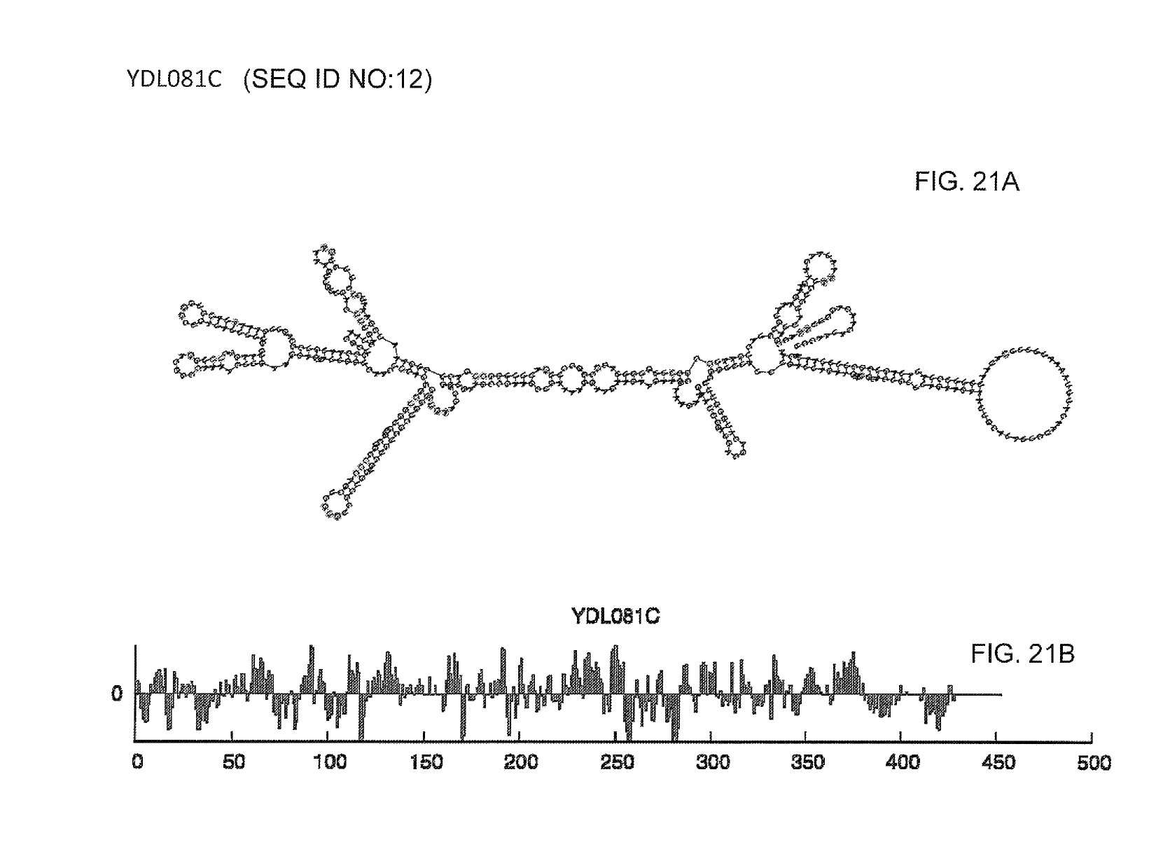

FIGS. 22A-B depicts PARS scores (FIG. 22B) and the predicted secondary structure (FIG. 22A) of the YDL133C-A RNA polynucleotide (SEQ ID NO:13).

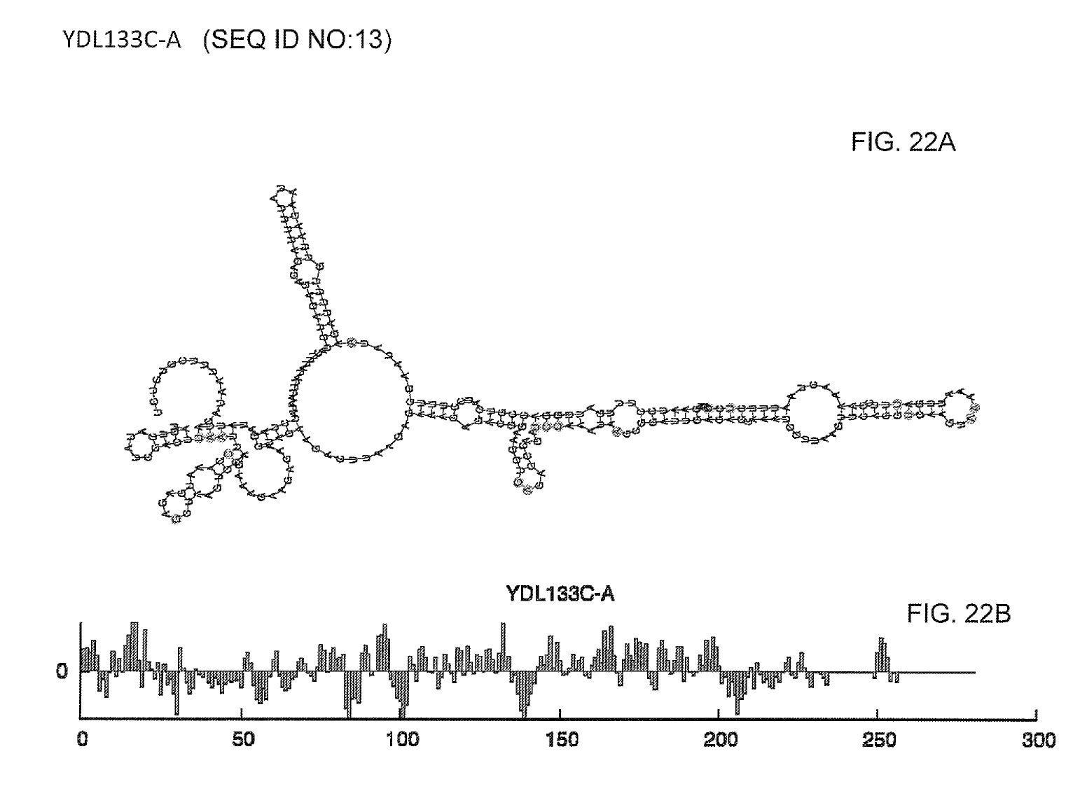

FIGS. 23A-B depicts PARS scores (FIG. 23B) and the predicted secondary structure (FIG. 23A) of the YDL184C RNA polynucleotide (SEQ ID NO:14).

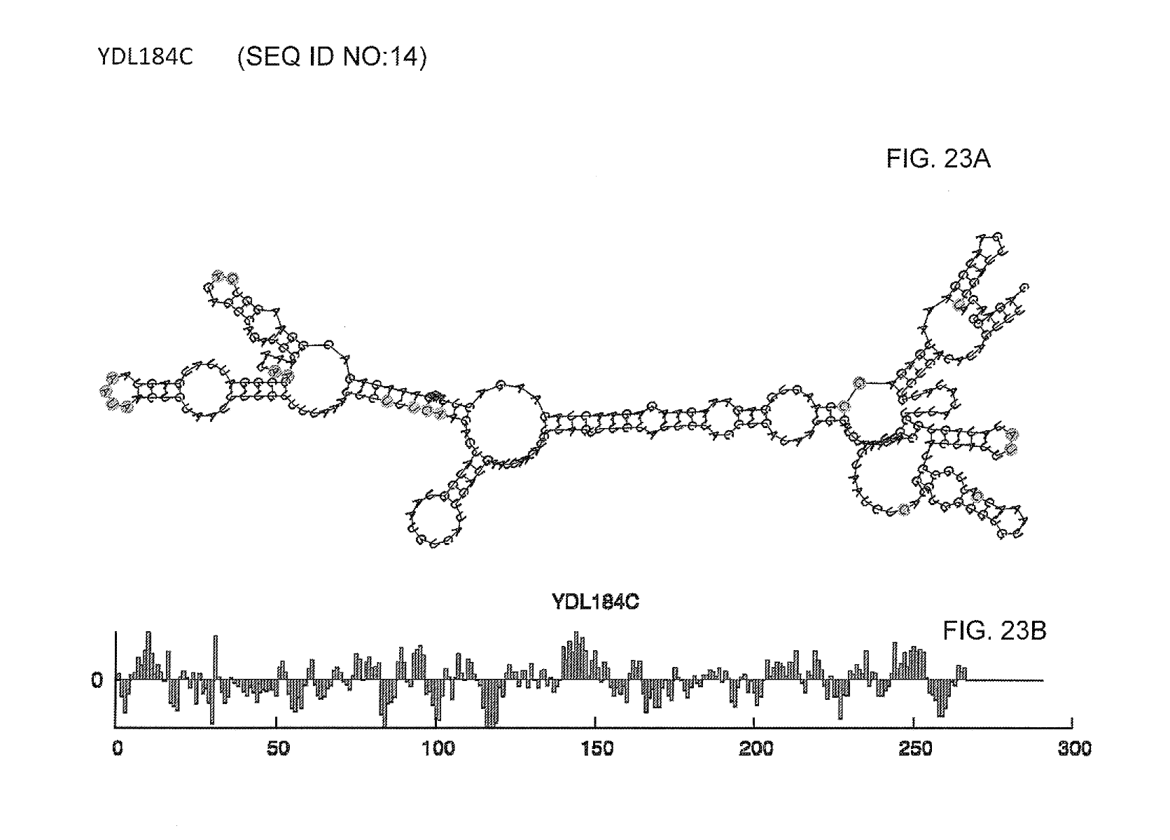

FIGS. 24A-B depicts PARS scores (FIG. 24B) and the predicted secondary structure (FIG. 24A) of the YDR050C RNA polynucleotide (SEQ ID NO:15).

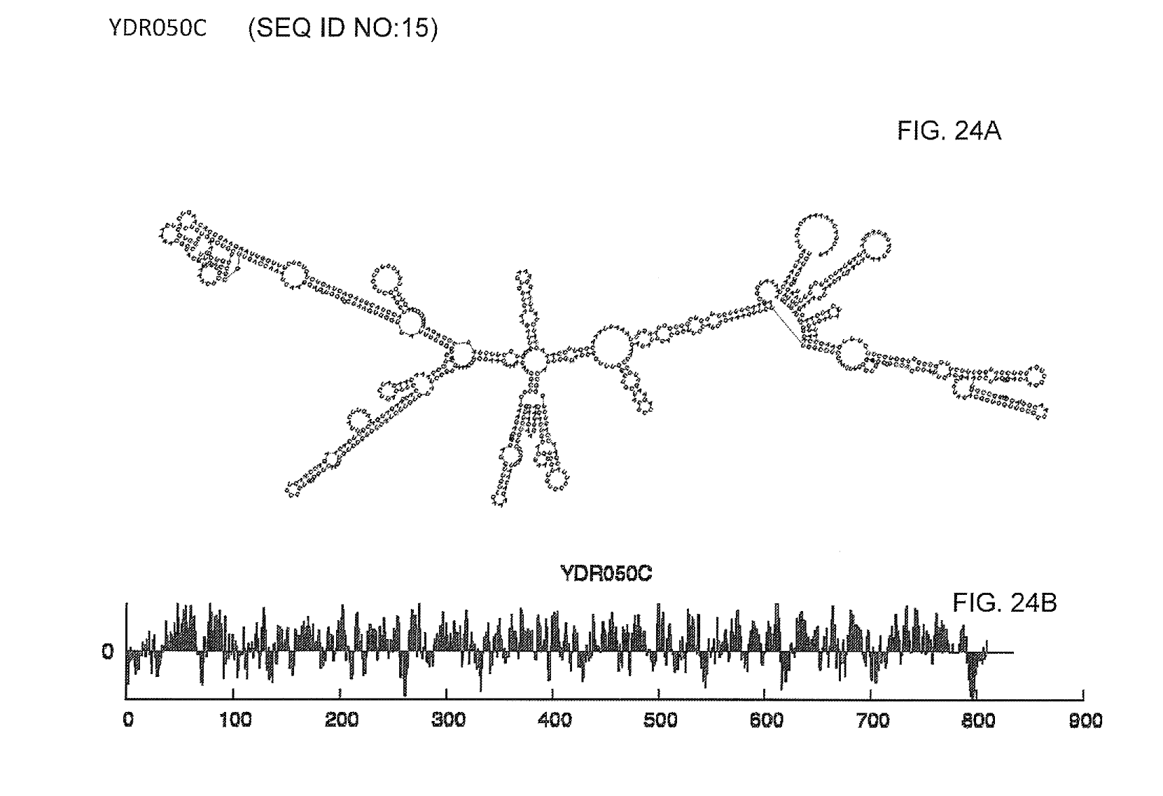

FIGS. 25A-B depicts PARS scores (FIG. 25B) and the predicted secondary structure (FIG. 25A) of the YDR064W RNA polynucleotide (SEQ ID NO:16).

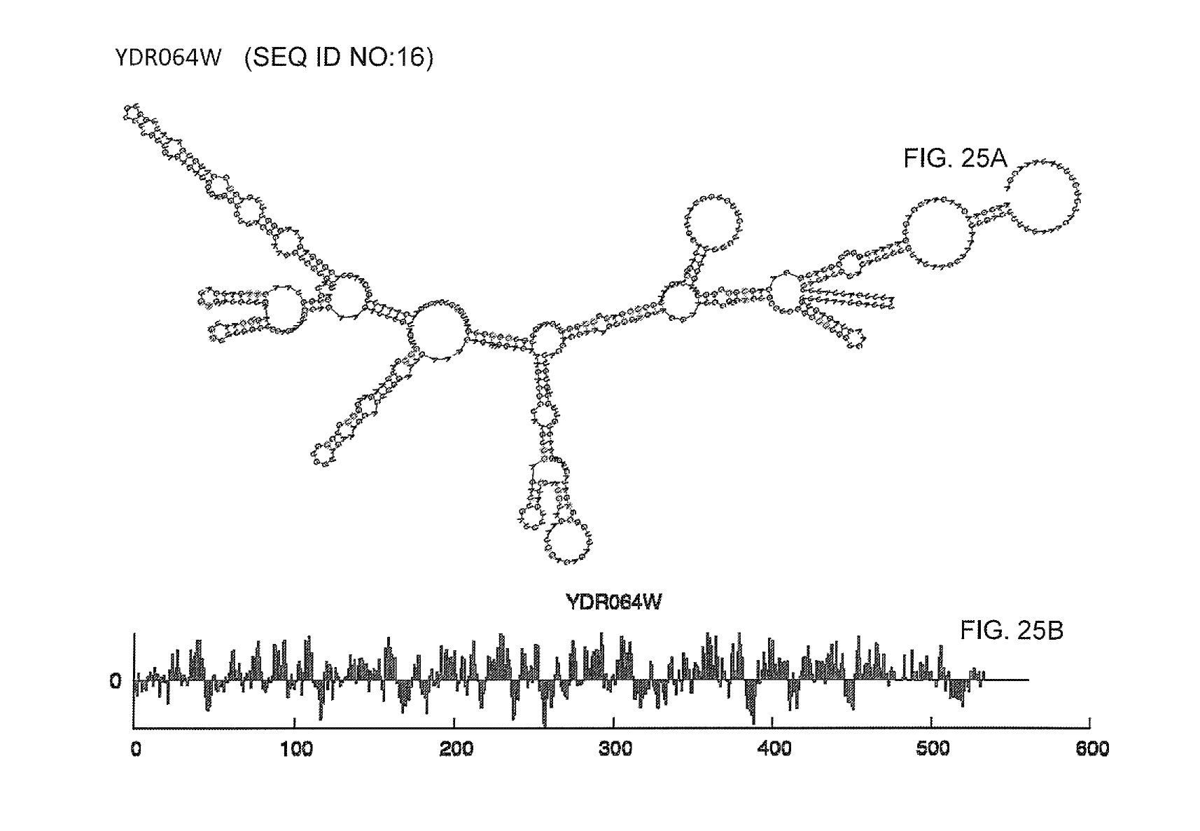

FIGS. 26A-B depicts PARS scores (FIG. 26B) and the predicted secondary structure (FIG. 26A) of the YDR155C RNA polynucleotide (SEQ ID NO:17).

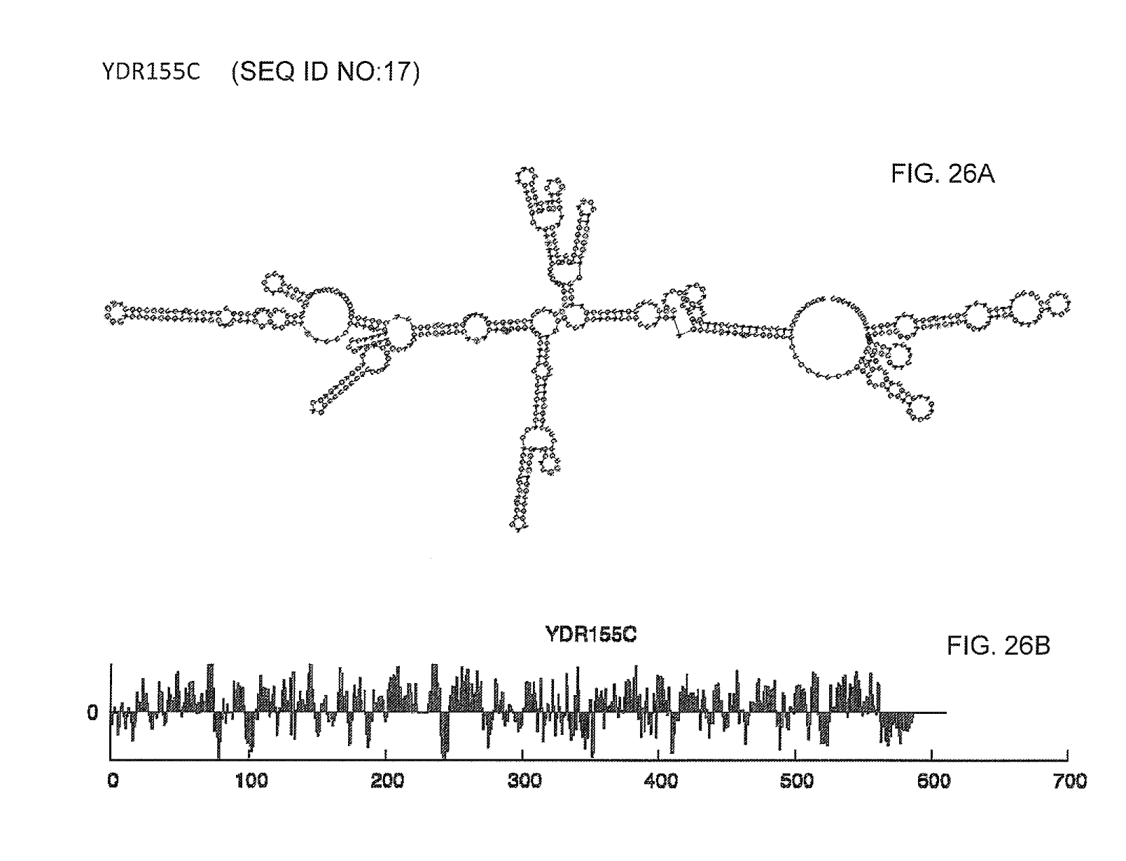

FIGS. 27A-B depicts PARS scores (FIG. 27B) and the predicted secondary structure (FIG. 27A) of the YDR382W RNA polynucleotide (SEQ ID NO:18).

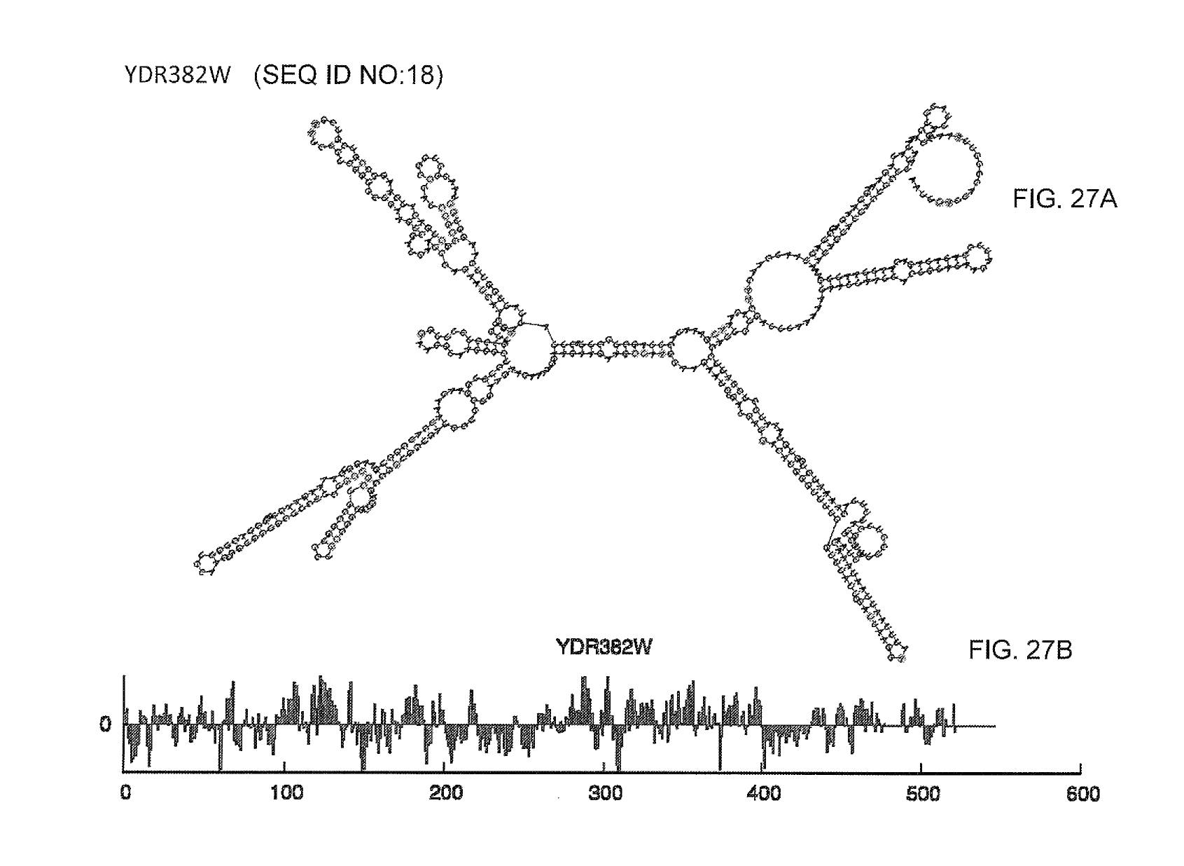

FIGS. 28A-B depicts PARS scores (FIG. 28B) and the predicted secondary structure (FIG. 28A) of the YDR524C-B RNA polynucleotide (SEQ ID NO:19).

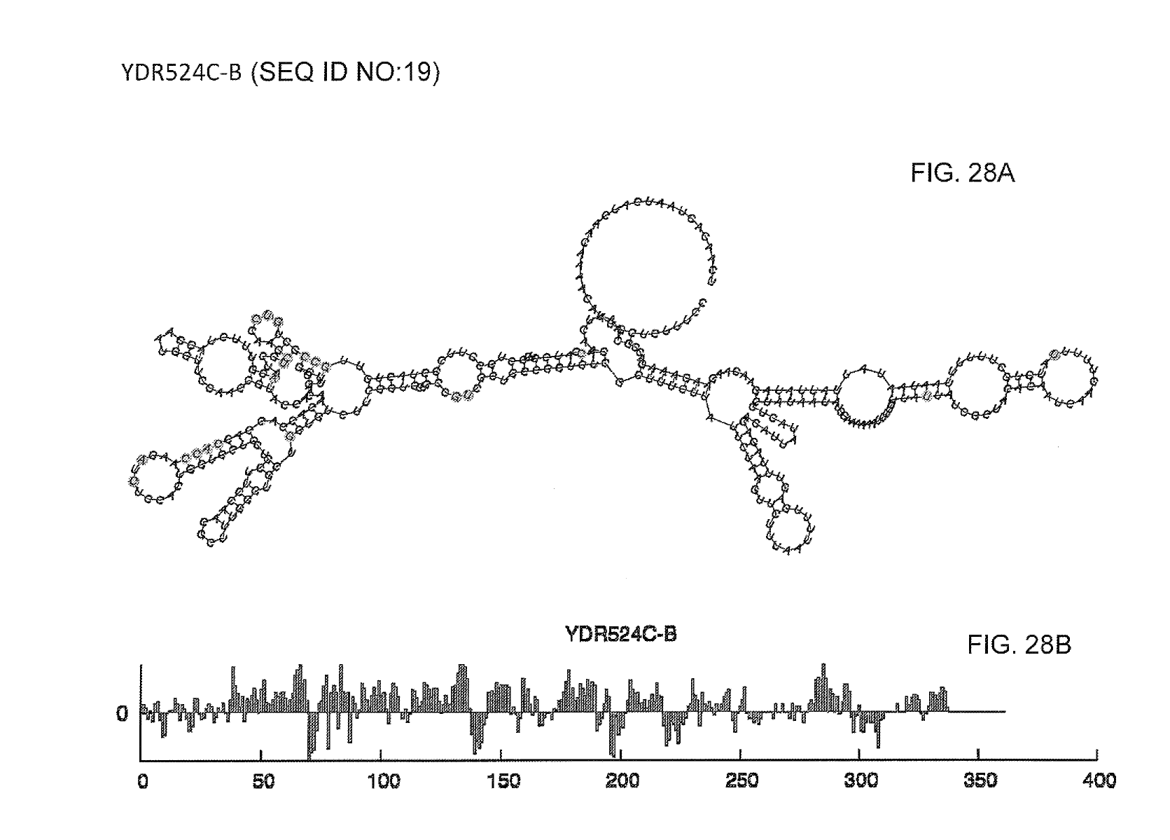

FIGS. 29A-B depicts PARS scores (FIG. 29B) and the predicted secondary structure (FIG. 29A) of the YFR032C-A RNA polynucleotide (SEQ ID NO:20).

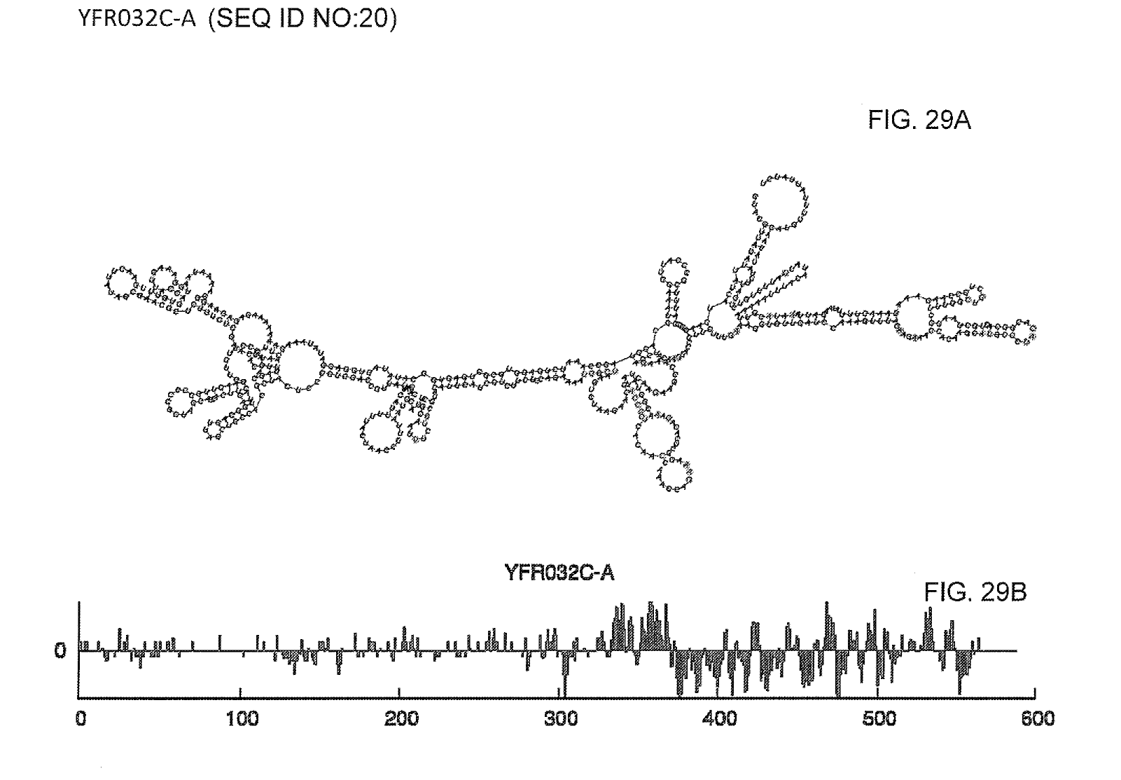

FIGS. 30A-B depicts PARS scores (FIG. 30B) and the predicted secondary structure (FIG. 30A) of the YGL030W RNA polynucleotide (SEQ ID NO:21).

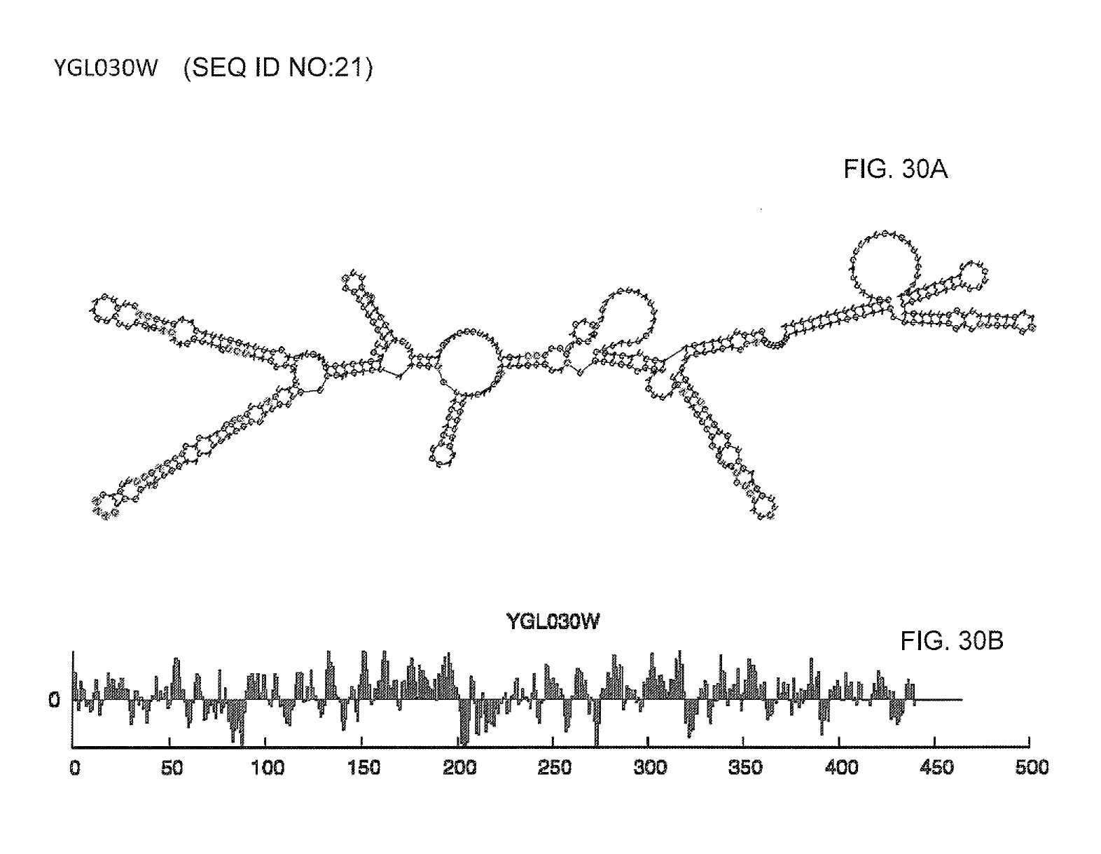

FIGS. 31A-B depicts PARS scores (FIG. 31B) and the predicted secondary structure (FIG. 31A) of the YGL103W RNA polynucleotide (SEQ ID NO:22).

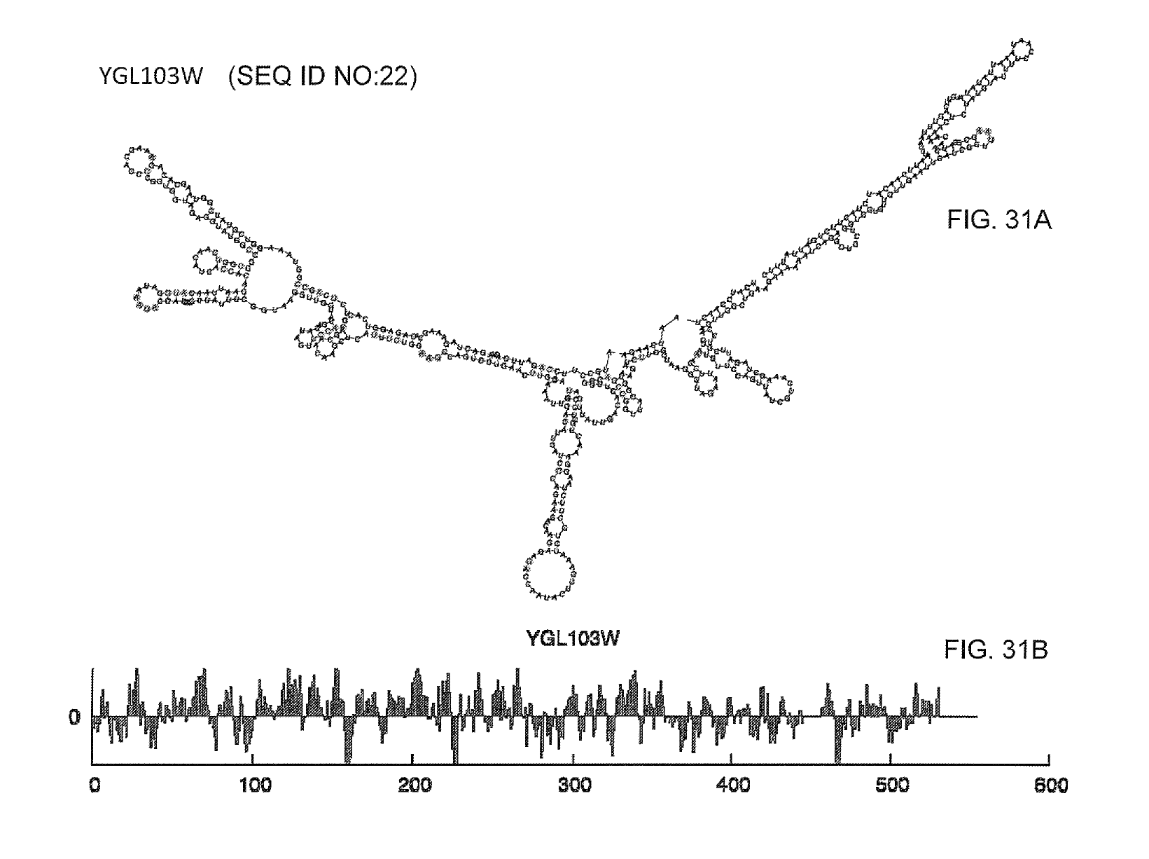

FIGS. 32A-B depicts PARS scores (FIG. 32B) and the predicted secondary structure (FIG. 32A) of the YGL123W RNA polynucleotide (SEQ ID NO:23).

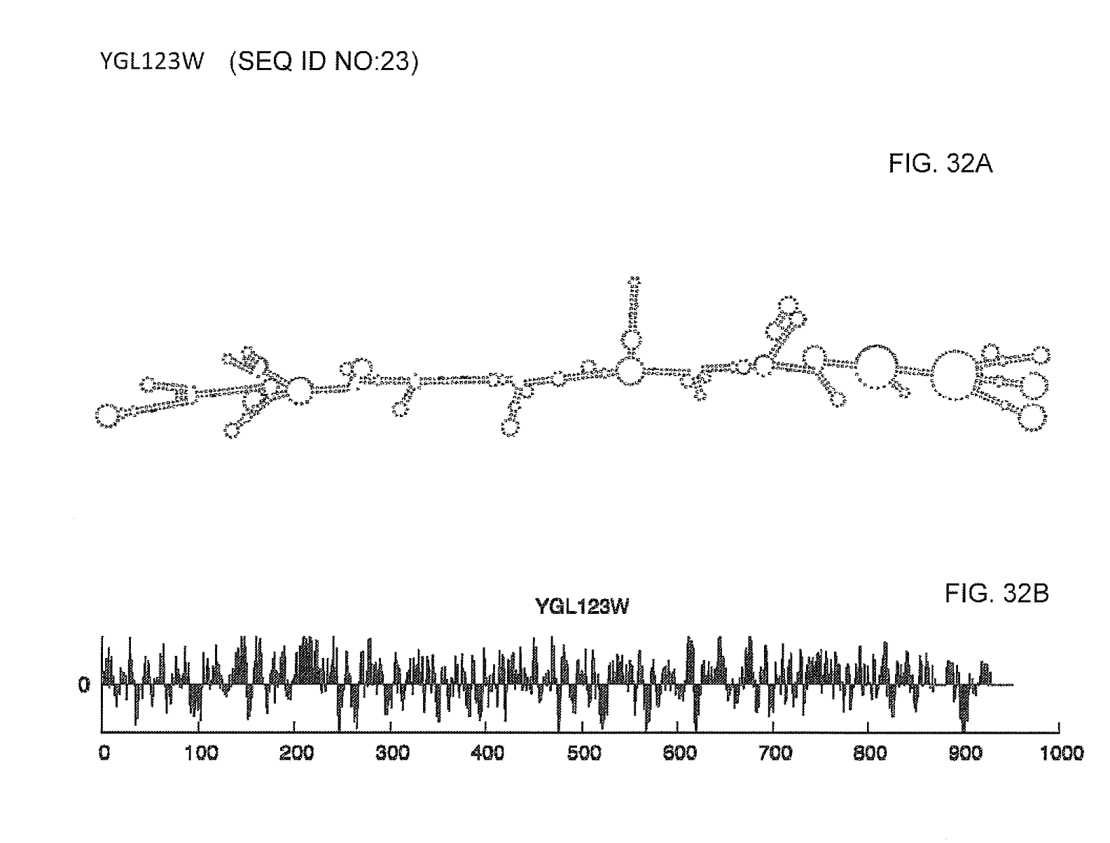

FIGS. 33A-B depicts PARS scores (FIG. 33B) and the predicted secondary structure (FIG. 33A) of the YGL147C RNA polynucleotide (SEQ ID NO:24).

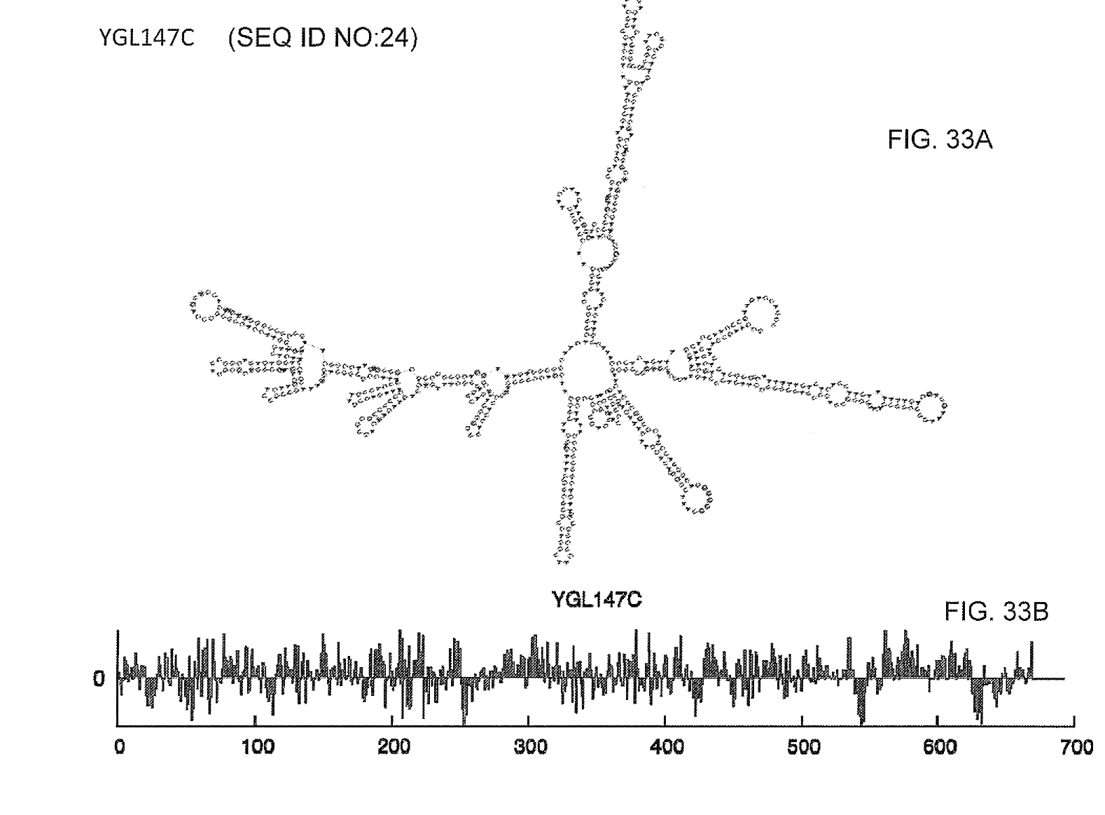

FIGS. 34A-B depicts PARS scores (FIG. 34B) and the predicted secondary structure (FIG. 34A) of the YGR192C RNA polynucleotide (SEQ ID NO:25).

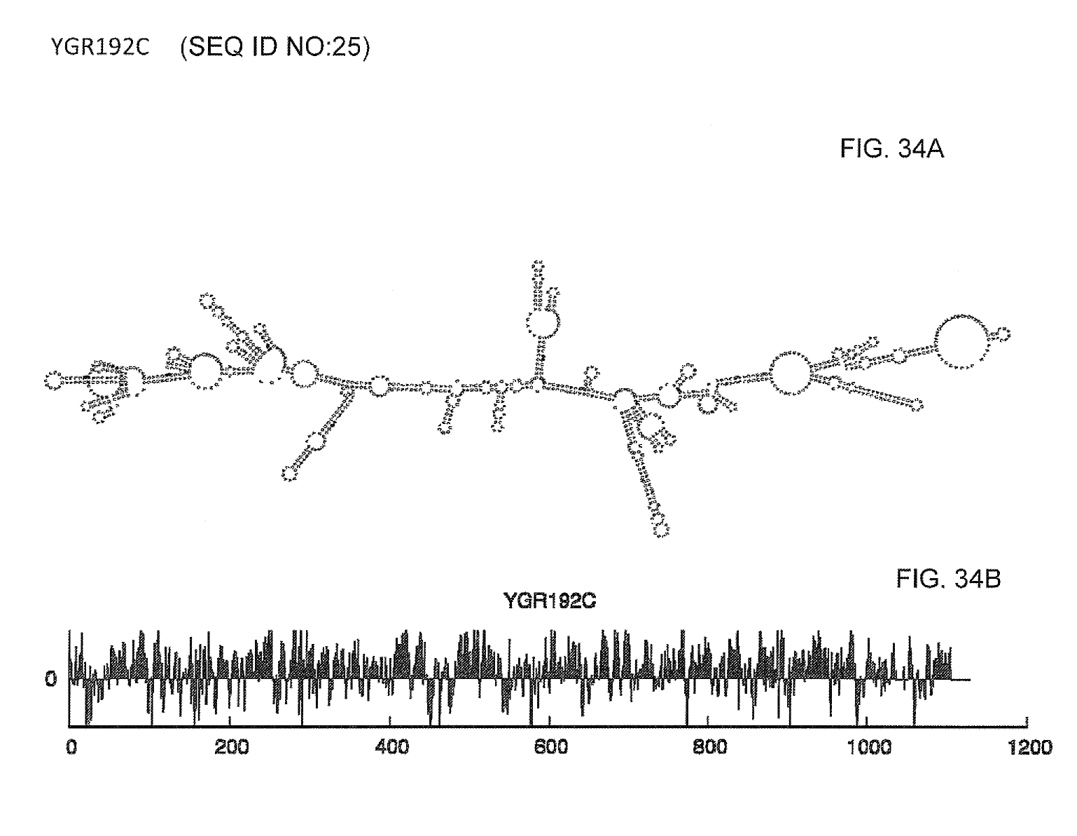

FIGS. 35A-B depicts PARS scores (FIG. 35B) and the predicted secondary structure (FIG. 35A) of the YHL015W RNA polynucleotide (SEQ ID NO:26).

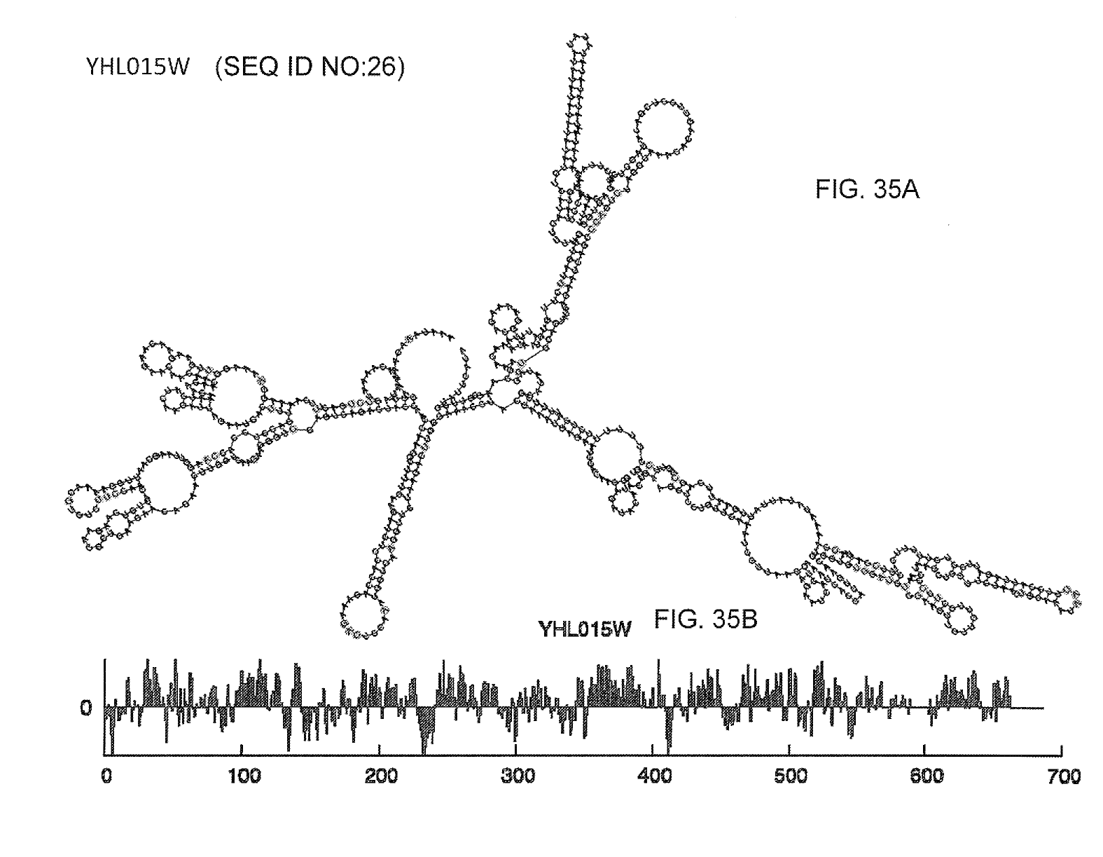

FIGS. 36A-B depicts PARS scores (FIG. 36B) and the predicted secondary structure (FIG. 36A) of the YHR021C RNA polynucleotide (SEQ ID NO:27).

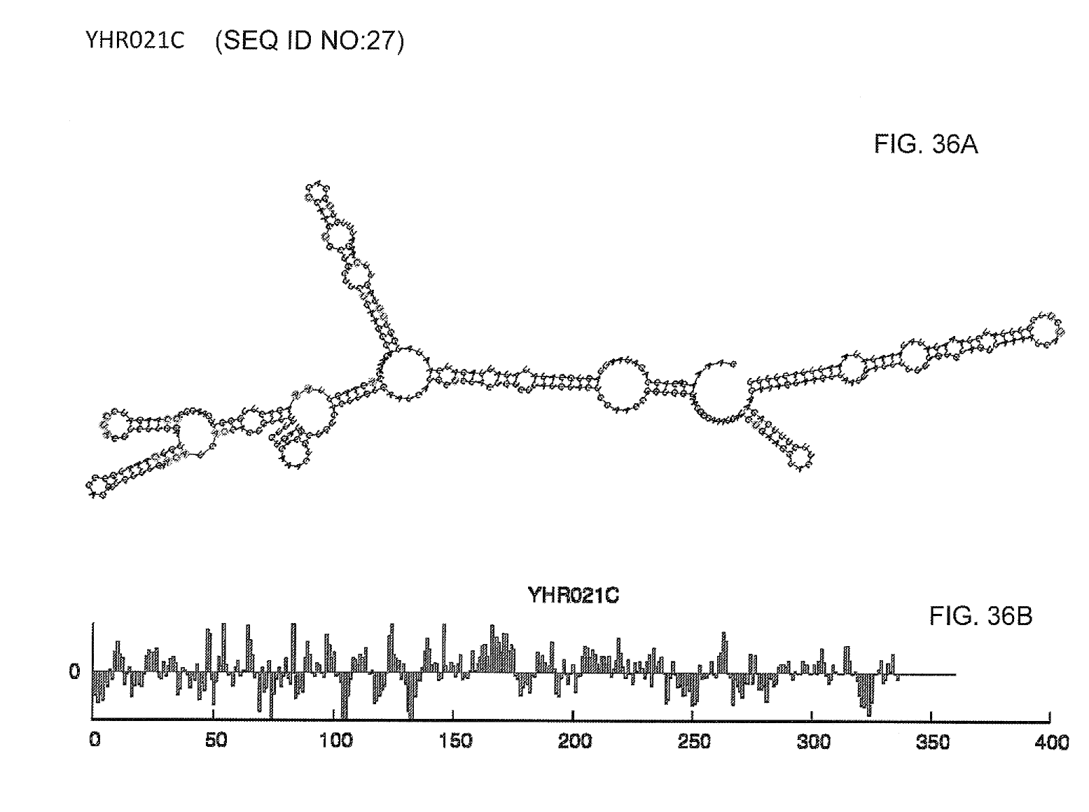

FIGS. 37A-B depicts PARS scores (FIG. 37B) and the predicted secondary structure (FIG. 37A) of the YHR141C RNA polynucleotide (SEQ ID NO:28).

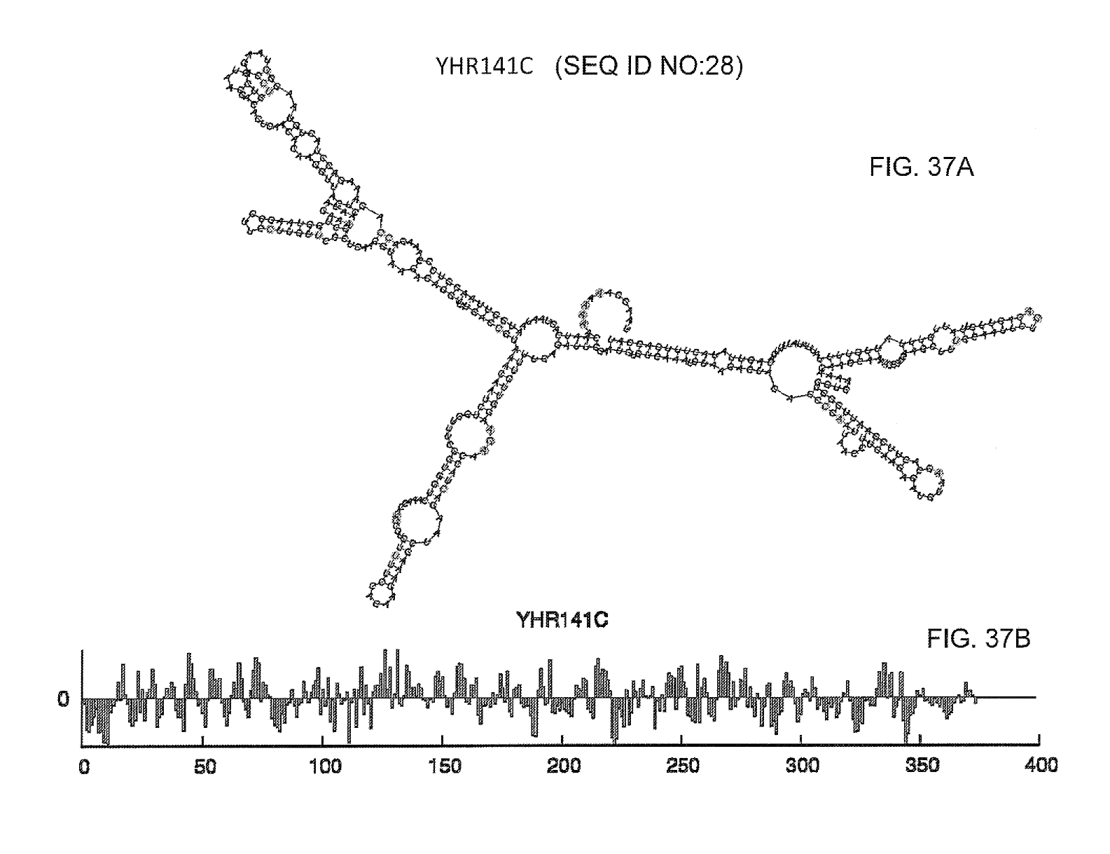

FIGS. 38A-B depicts PARS scores (FIG. 38B) and the predicted secondary structure (FIG. 38A) of the YHR174W RNA polynucleotide (SEQ ID NO:29).

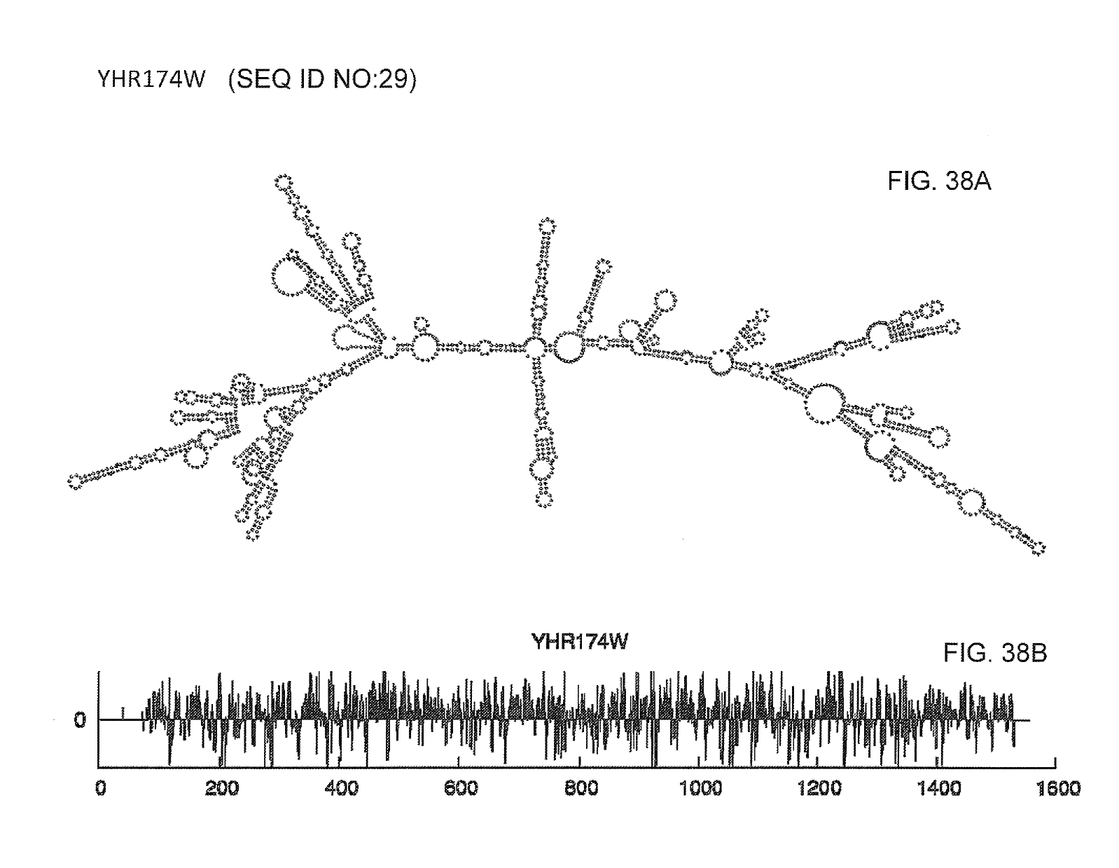

FIGS. 39A-B depicts PARS scores (FIG. 39B) and the predicted secondary structure (FIG. 39A) of the YJL189W RNA polynucleotide (SEQ ID NO:30).

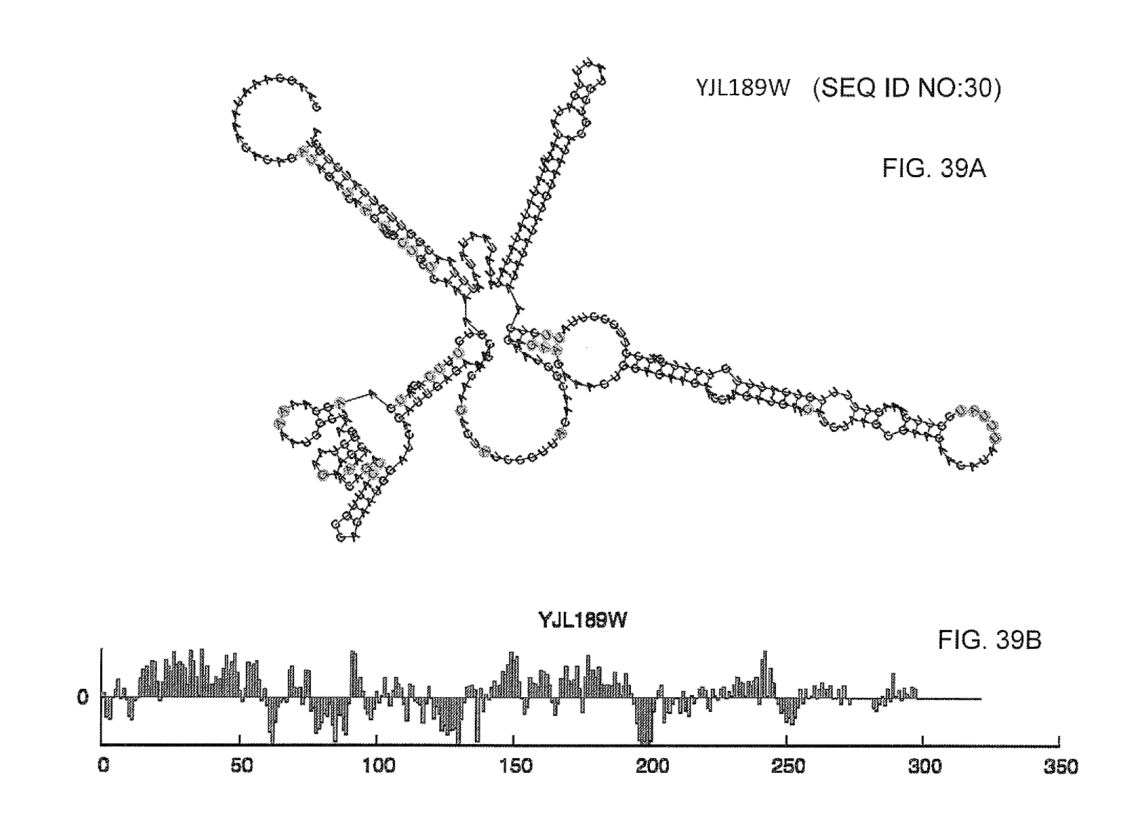

FIGS. 40A-B depicts PARS scores (FIG. 40B) and the predicted secondary structure (FIG. 40A) of the YJL190C RNA polynucleotide (SEQ ID NO:31).

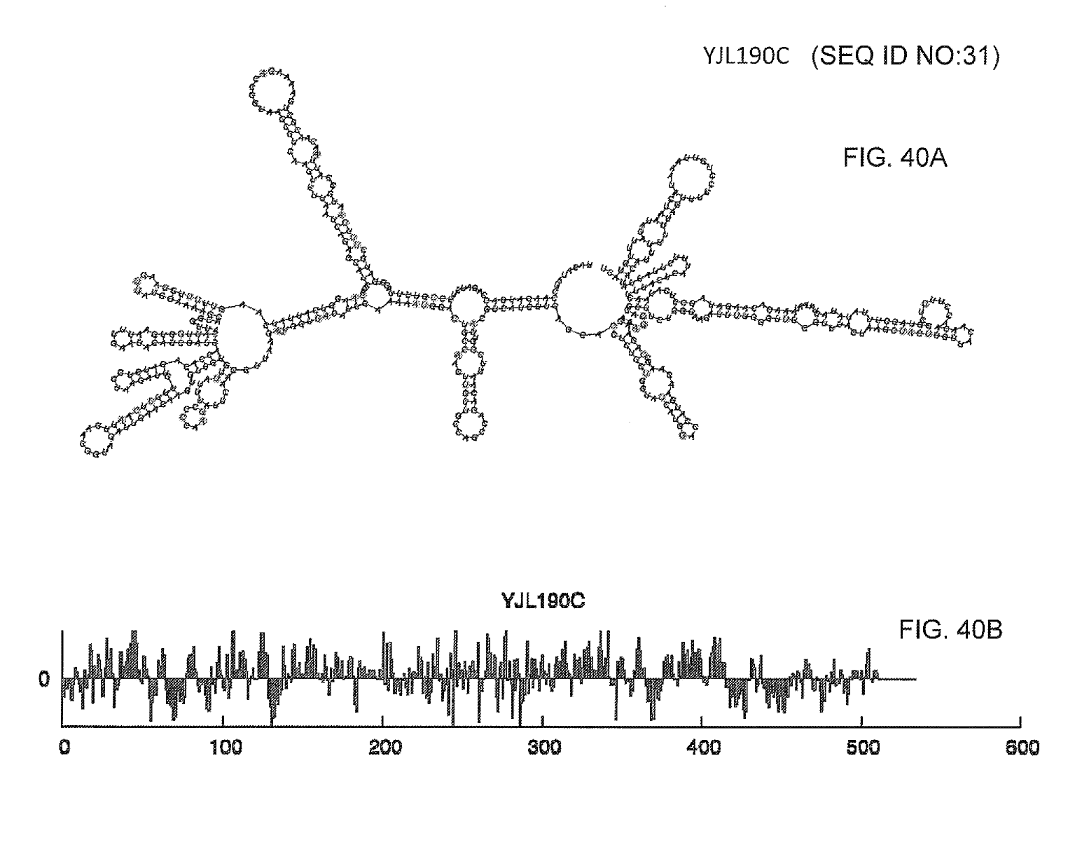

FIGS. 41A-B depicts PARS scores (FIG. 41B) and the predicted secondary structure (FIG. 41A) of the YJR123W RNA polynucleotide (SEQ ID NO:32).

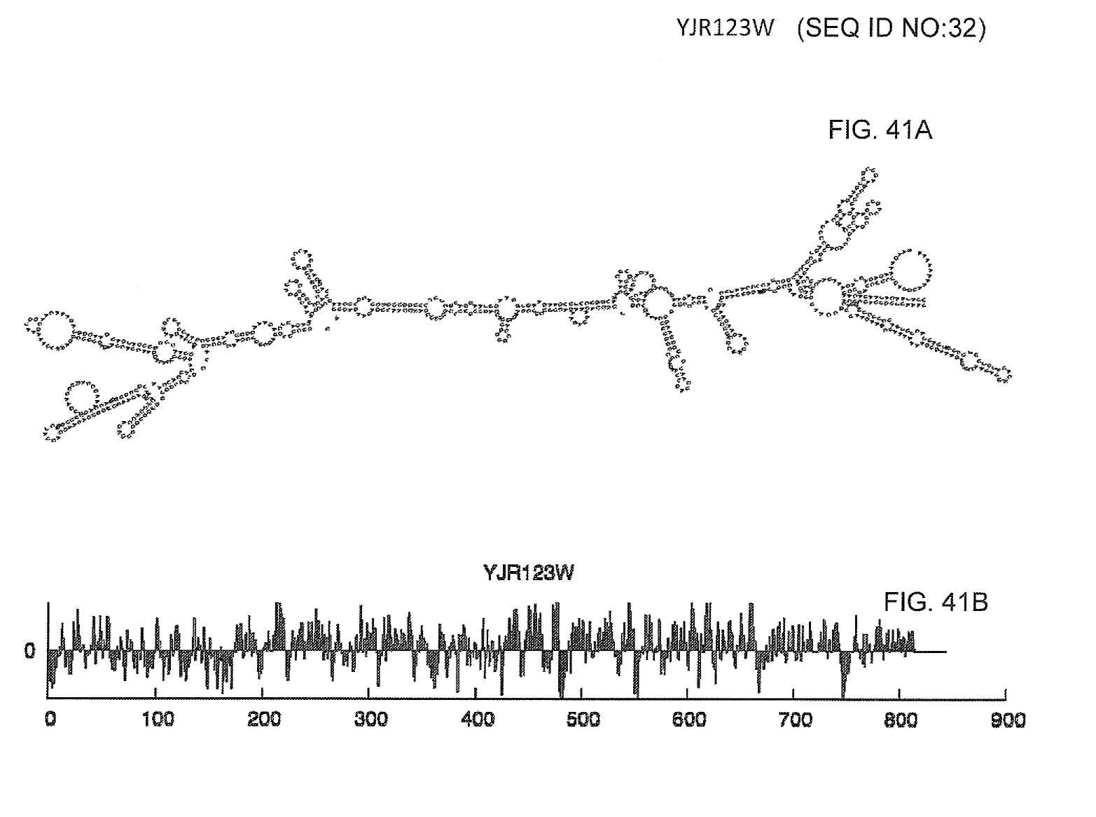

FIGS. 42A-B depicts PARS scores (FIG. 42B) and the predicted secondary structure (FIG. 42A) of the YDL081C RNA polynucleotide (SEQ ID NO:33).

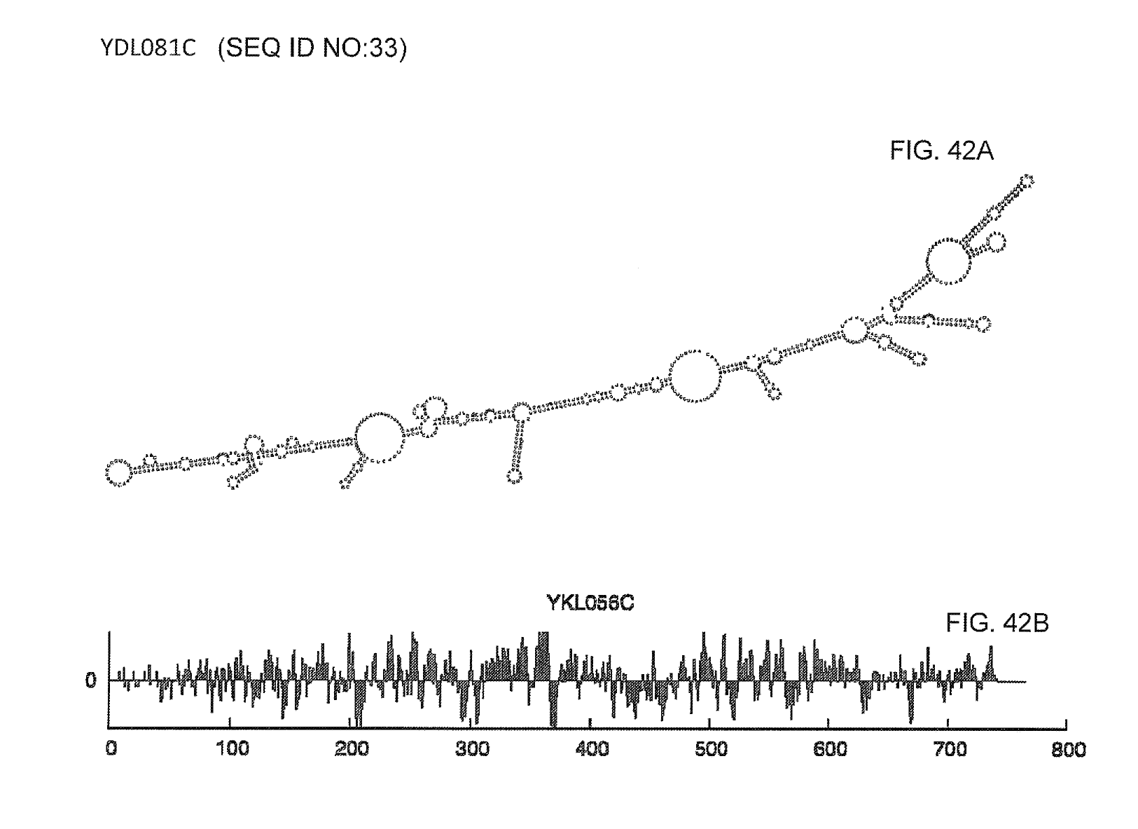

FIGS. 43A-B depicts PARS scores (FIG. 43B) and the predicted secondary structure (FIG. 43A) of the YKL060C RNA polynucleotide (SEQ ID NO:34).

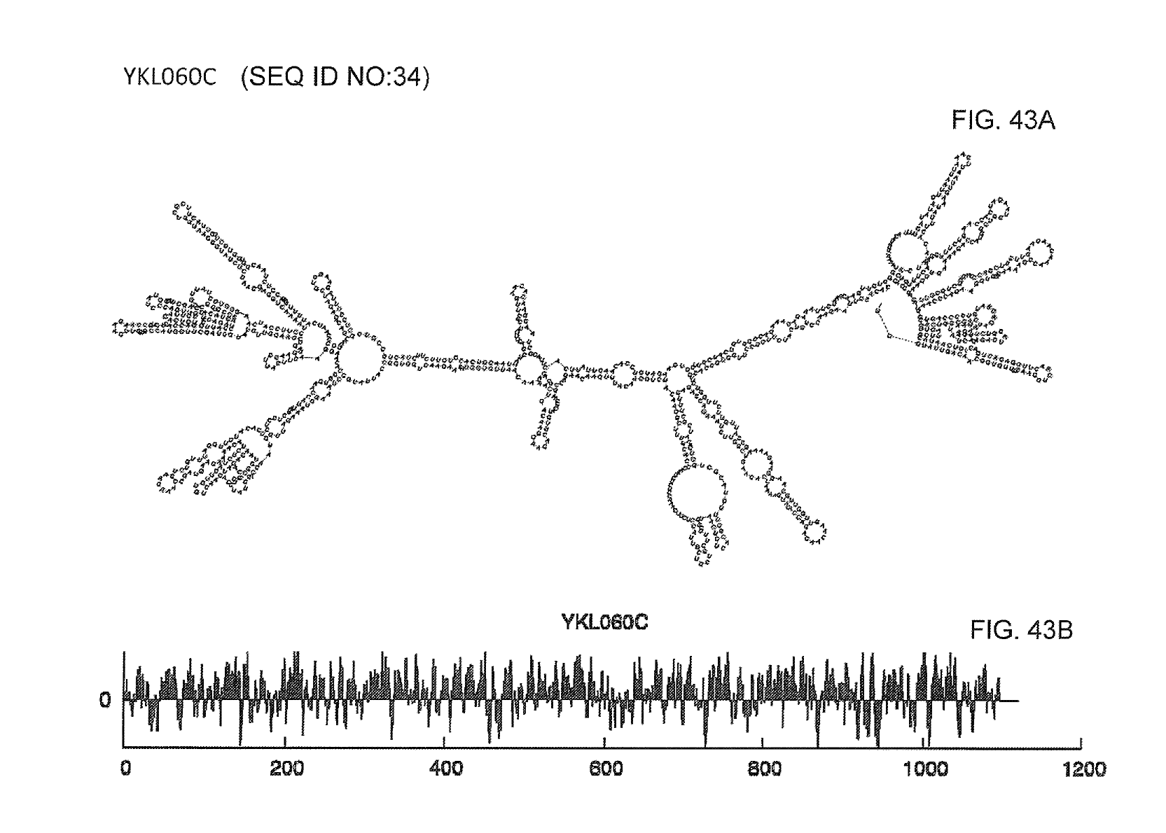

FIGS. 44A-B depicts PARS scores (FIG. 44B) and the predicted secondary structure (FIG. 44A) of the YKL152C RNA polynucleotide (SEQ ID NO:35).

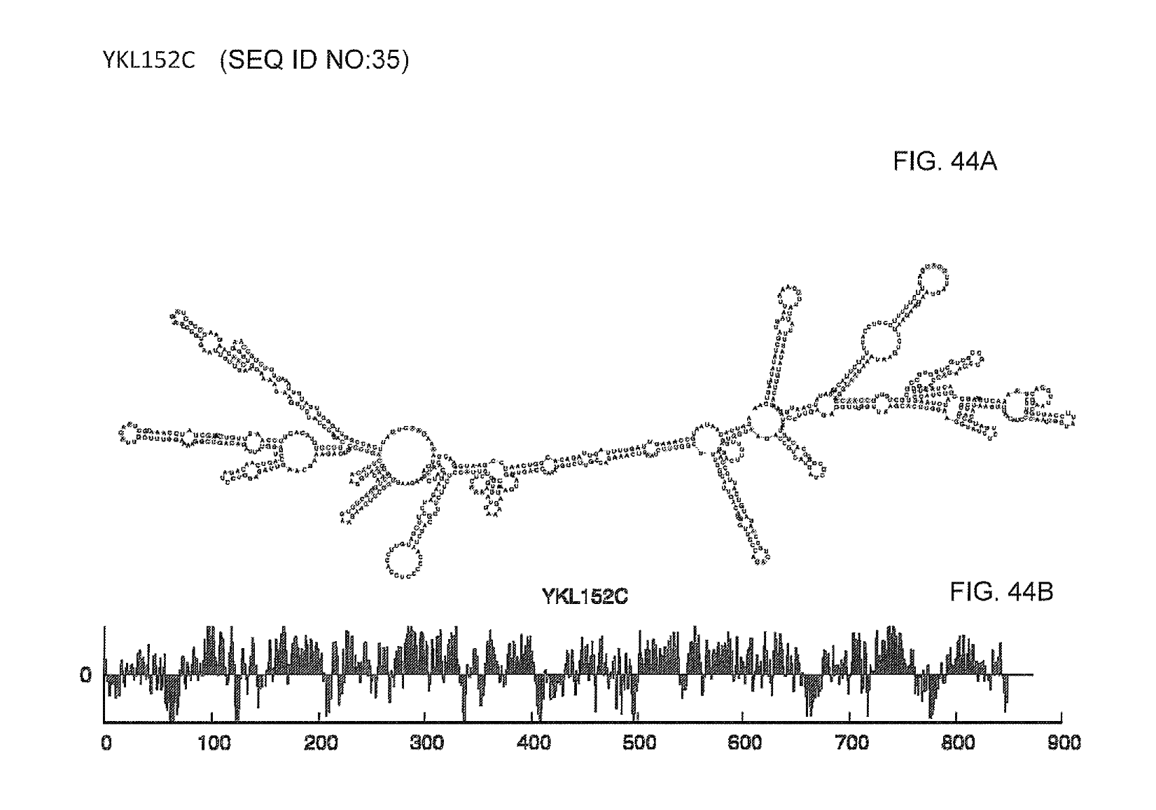

FIGS. 45A-B depicts PARS scores (FIG. 45B) and the predicted secondary structure (FIG. 45A) of the YKR057W RNA polynucleotide (SEQ ID NO:36).

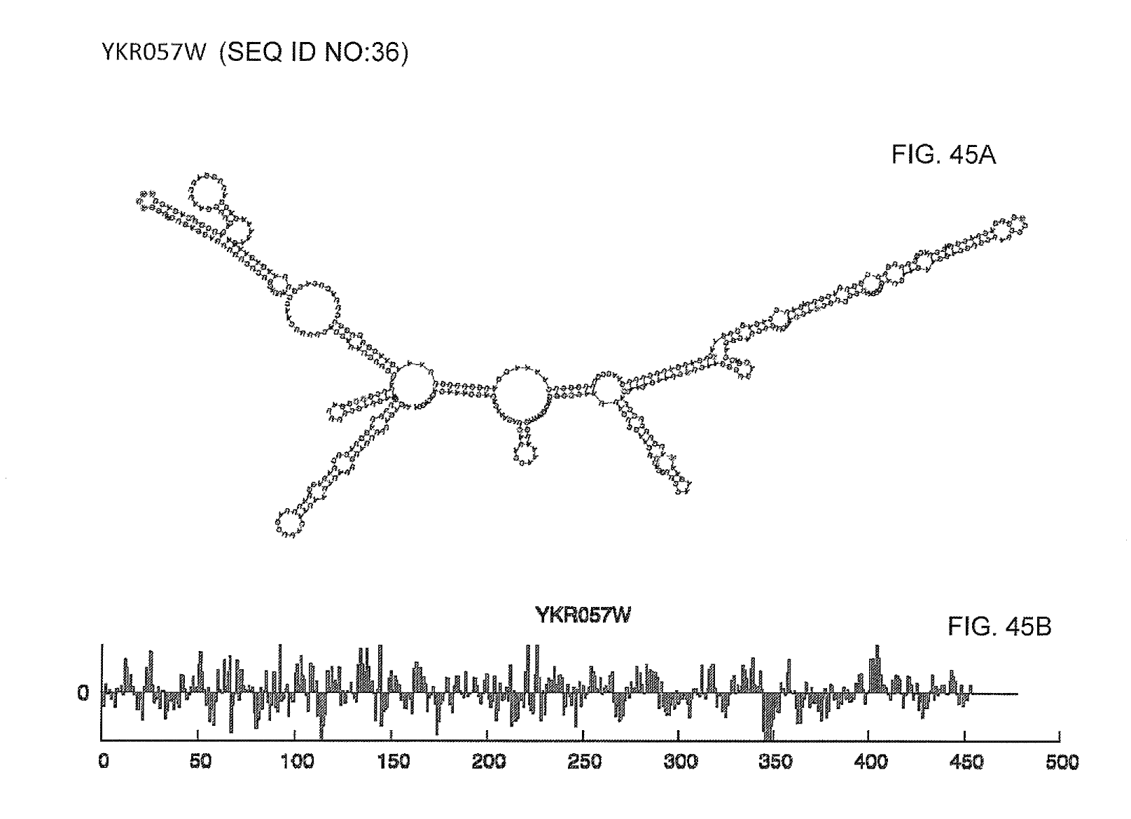

FIGS. 46A-B depicts PARS scores (FIG. 46B) and the predicted secondary structure (FIG. 46A) of the YLR043C RNA polynucleotide (SEQ ID NO:37).

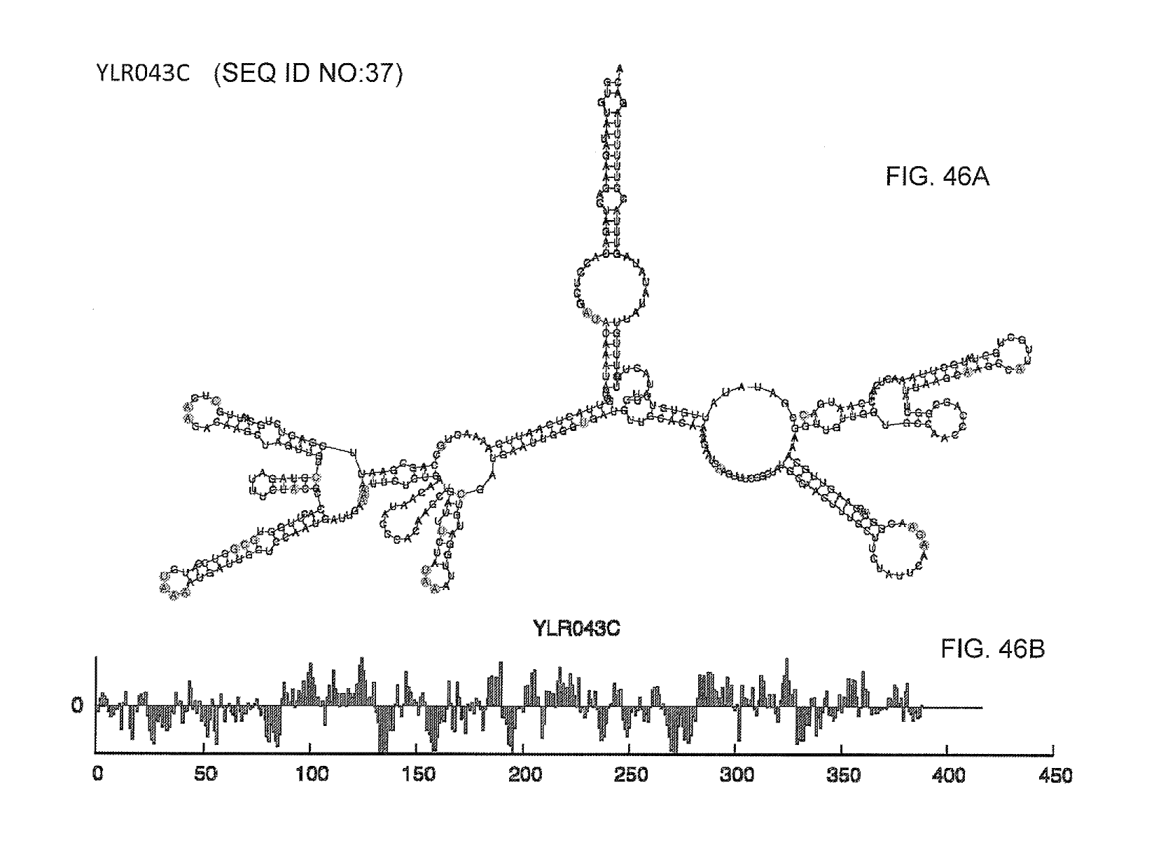

FIGS. 47A-B depicts PARS scores (FIG. 47B) and the predicted secondary structure (FIG. 47A) of the YLR044C RNA polynucleotide (SEQ ID NO:38).

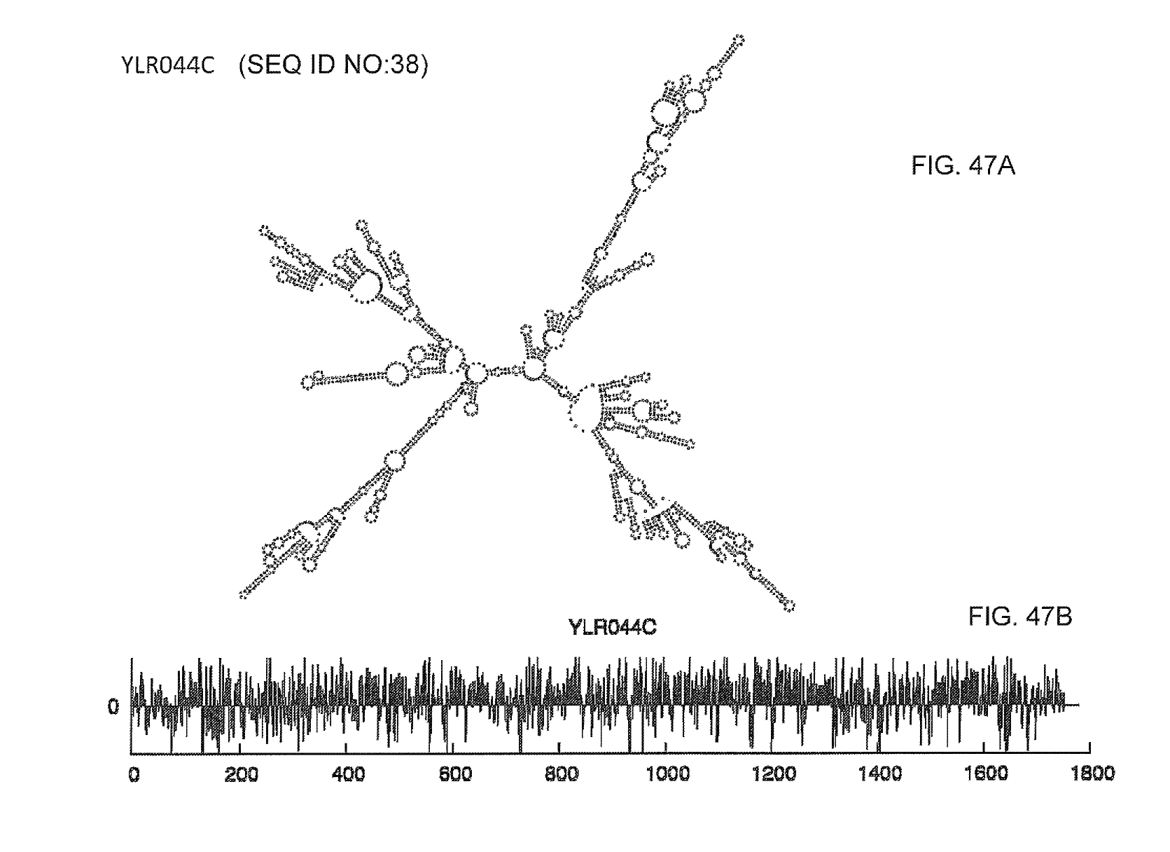

FIGS. 48A-B depicts PARS scores (FIG. 48B) and the predicted secondary structure (FIG. 48A) of the YLR061W RNA polynucleotide (SEQ ID NO:39).

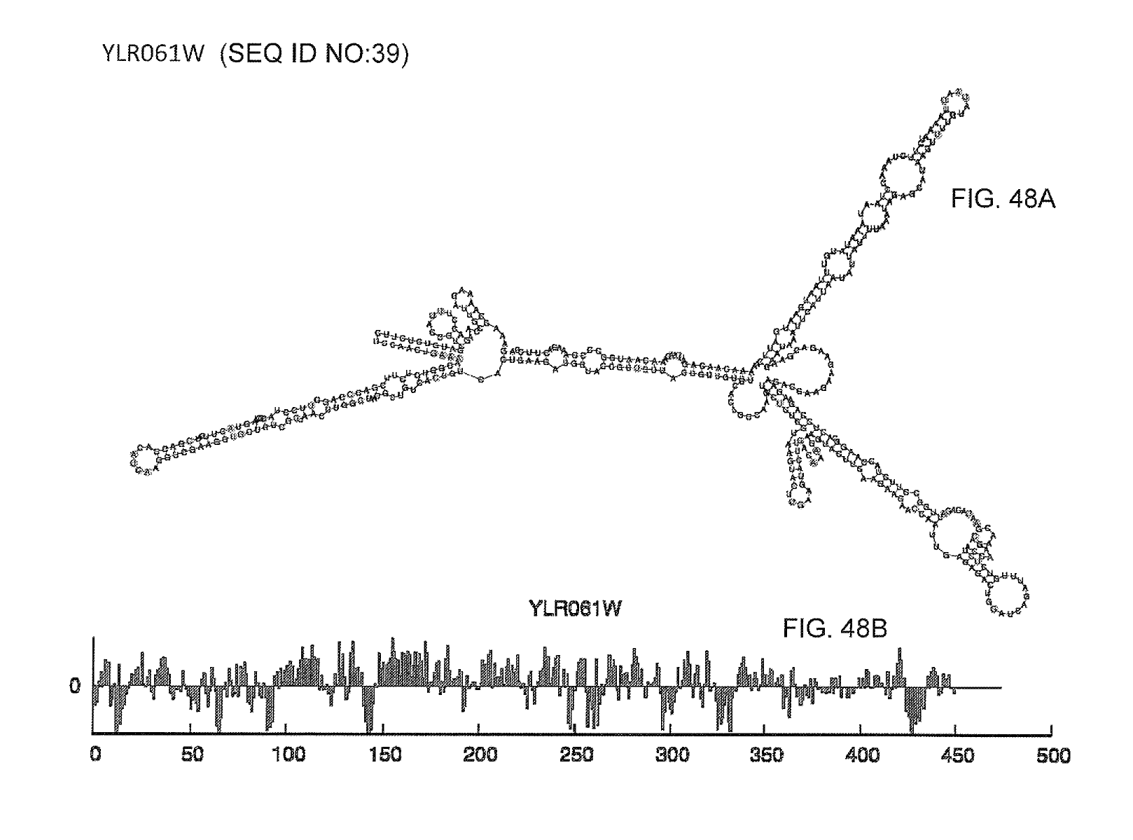

FIGS. 49A-B depicts PARS scores (FIG. 49B) and the predicted secondary structure (FIG. 49A) of the YLR075W RNA polynucleotide (SEQ ID NO:40).

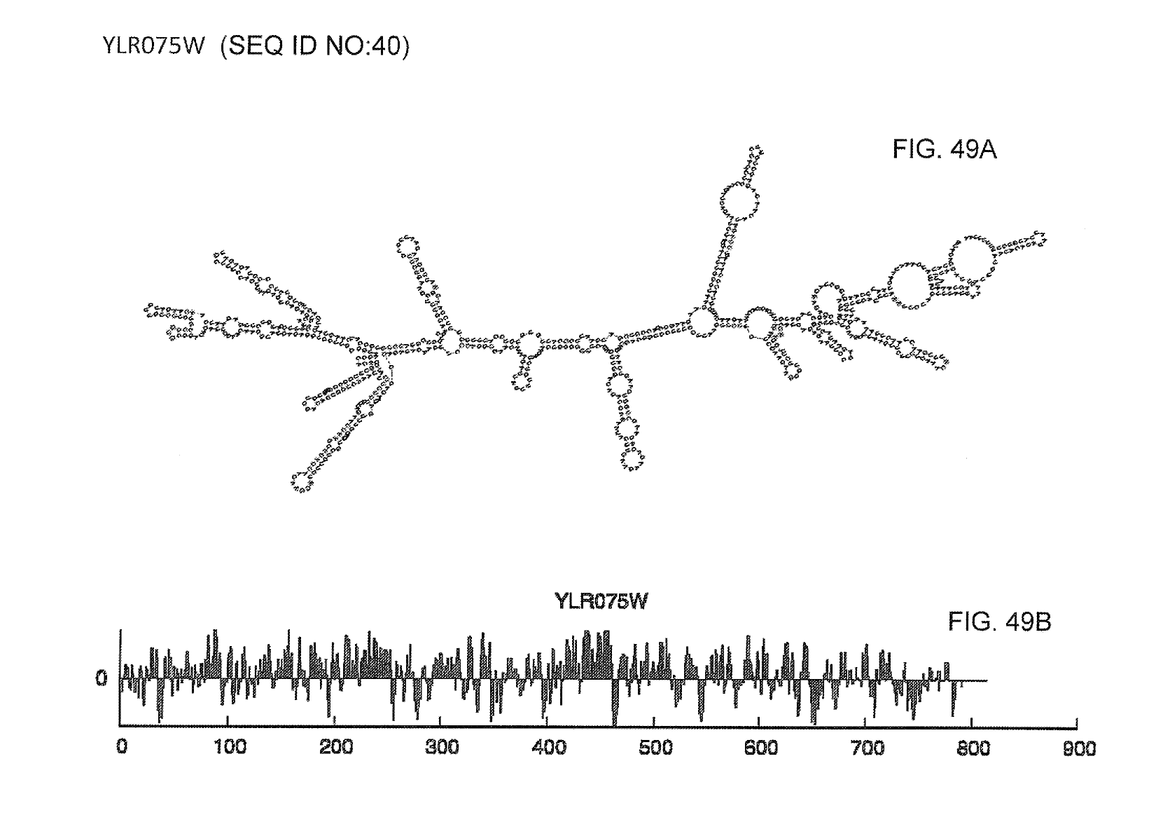

FIGS. 50A-B depicts PARS scores (FIG. 50B) and the predicted secondary structure (FIG. 50A) of the YLR110C RNA polynucleotide (SEQ ID NO:1).

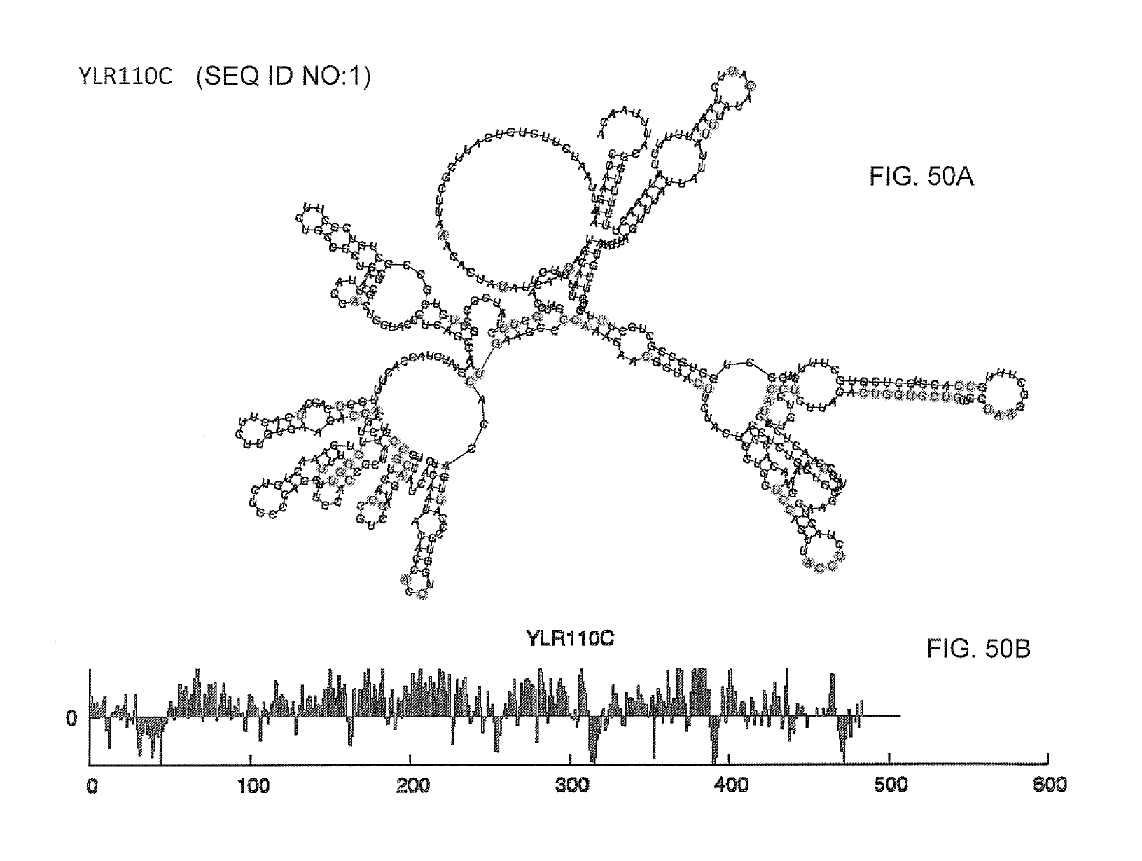

FIGS. 51A-B depicts PARS scores (FIG. 51B) and the predicted secondary structure (FIG. 51A) of the YLR167W RNA polynucleotide (SEQ ID NO:41).

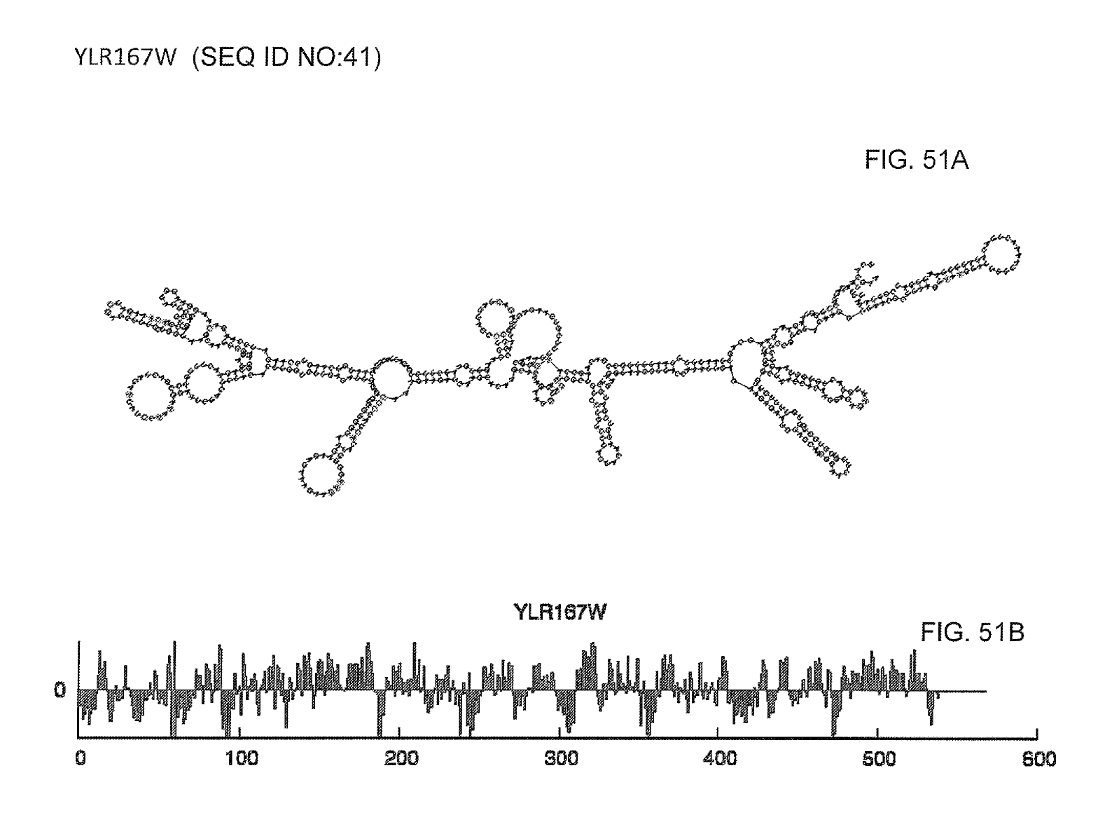

FIGS. 52A-B depicts PARS scores (FIG. 52B) and the predicted secondary structure (FIG. 52A) of the YLR249W RNA polynucleotide (SEQ ID NO:42).

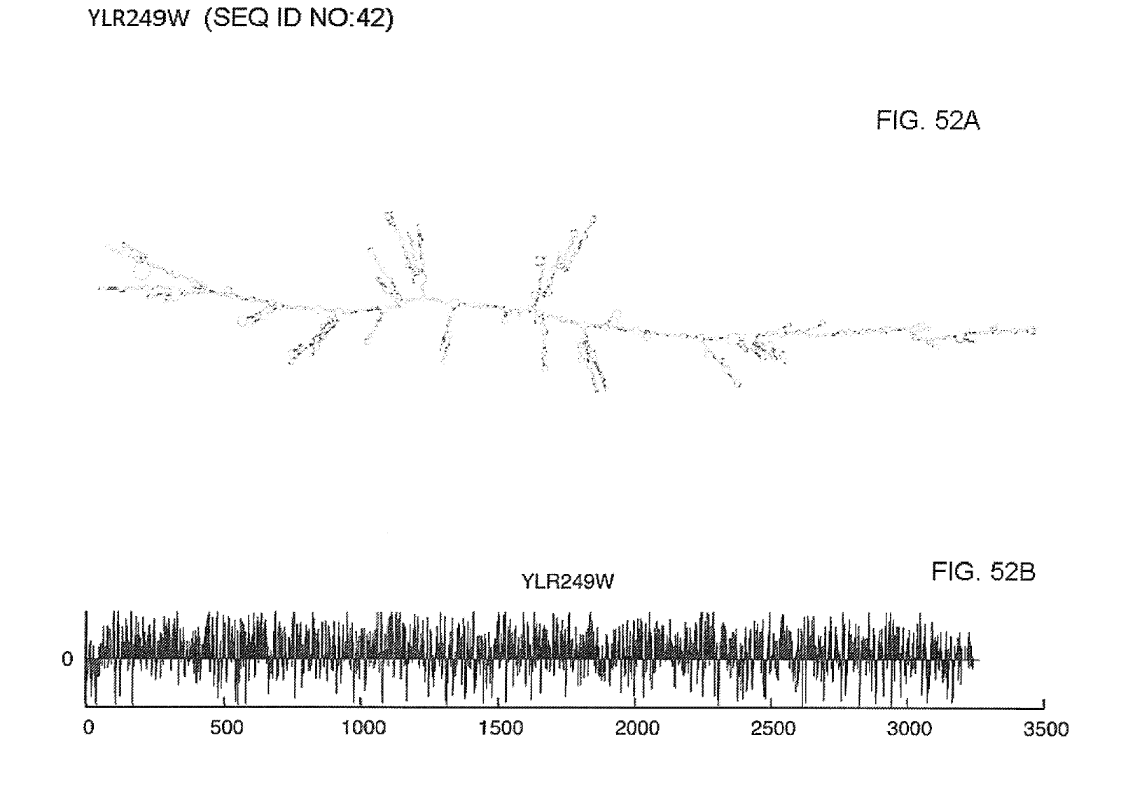

### DESCRIPTION OF SPECIFIC EMBODIMENTS OF THE INVENTION

The present invention, in some embodiments thereof, relates to methods of predicting the pairability of ribonucleotides in a plurality of RNA polynucleotides, and, more particularly, but not exclusively, to methods of determining secondary and/or tertiary structures of RNA polynucleotides.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

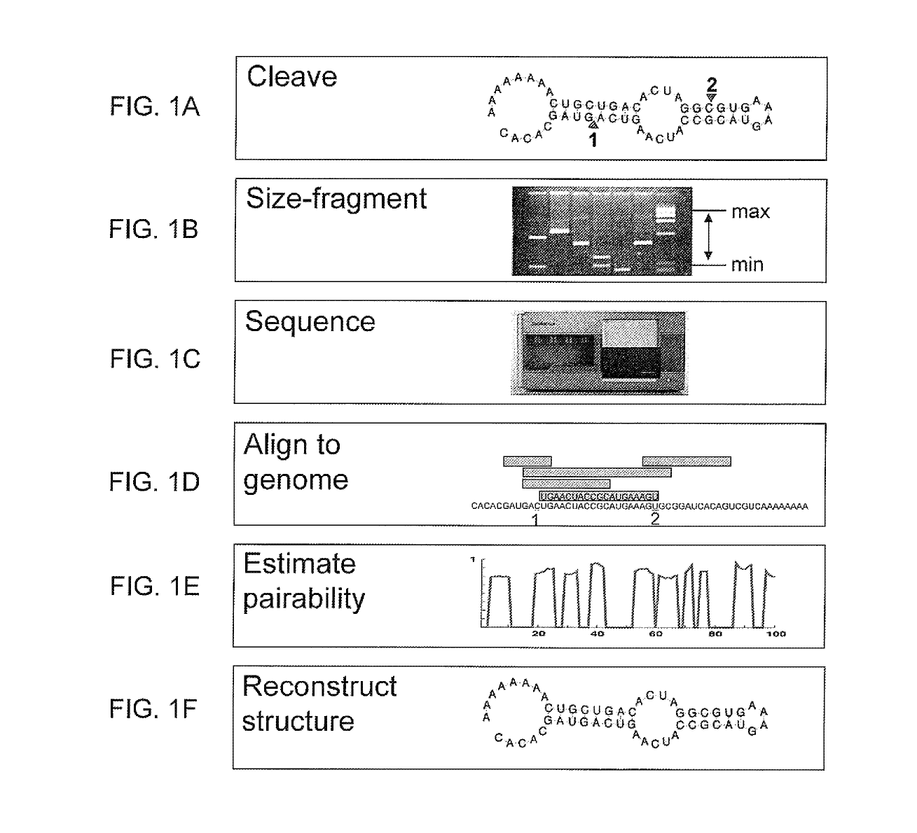

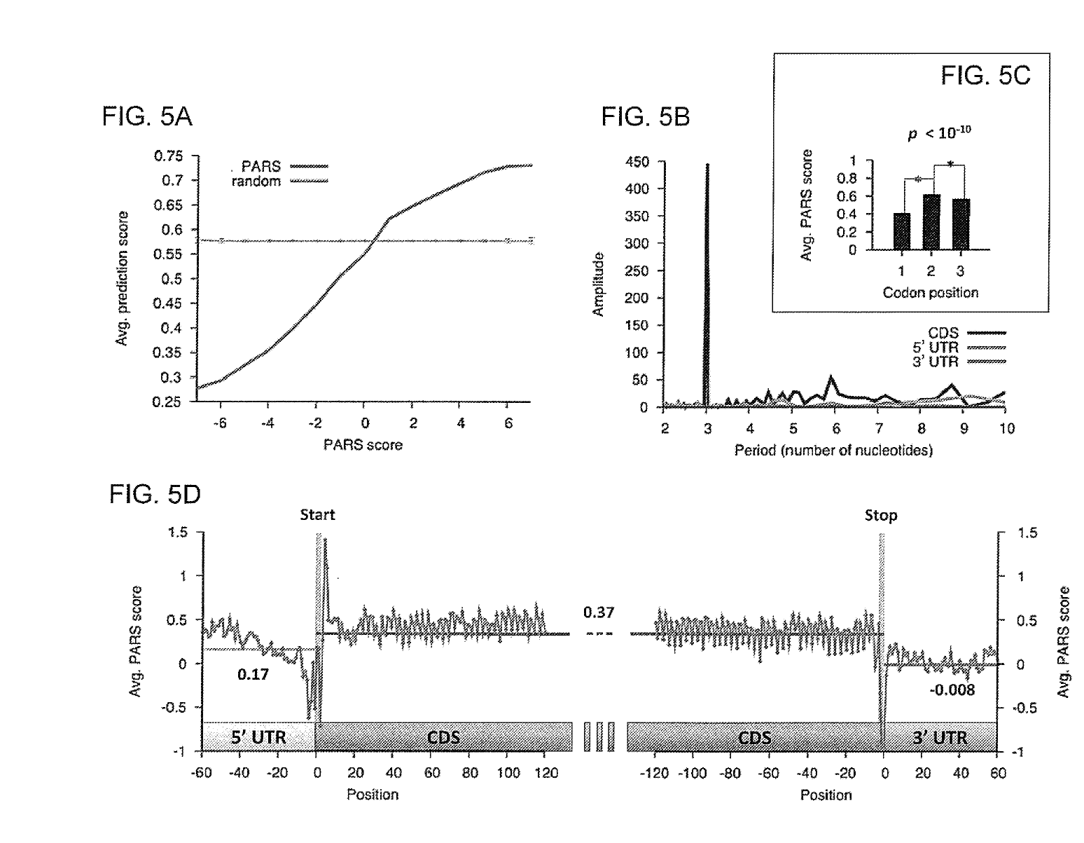

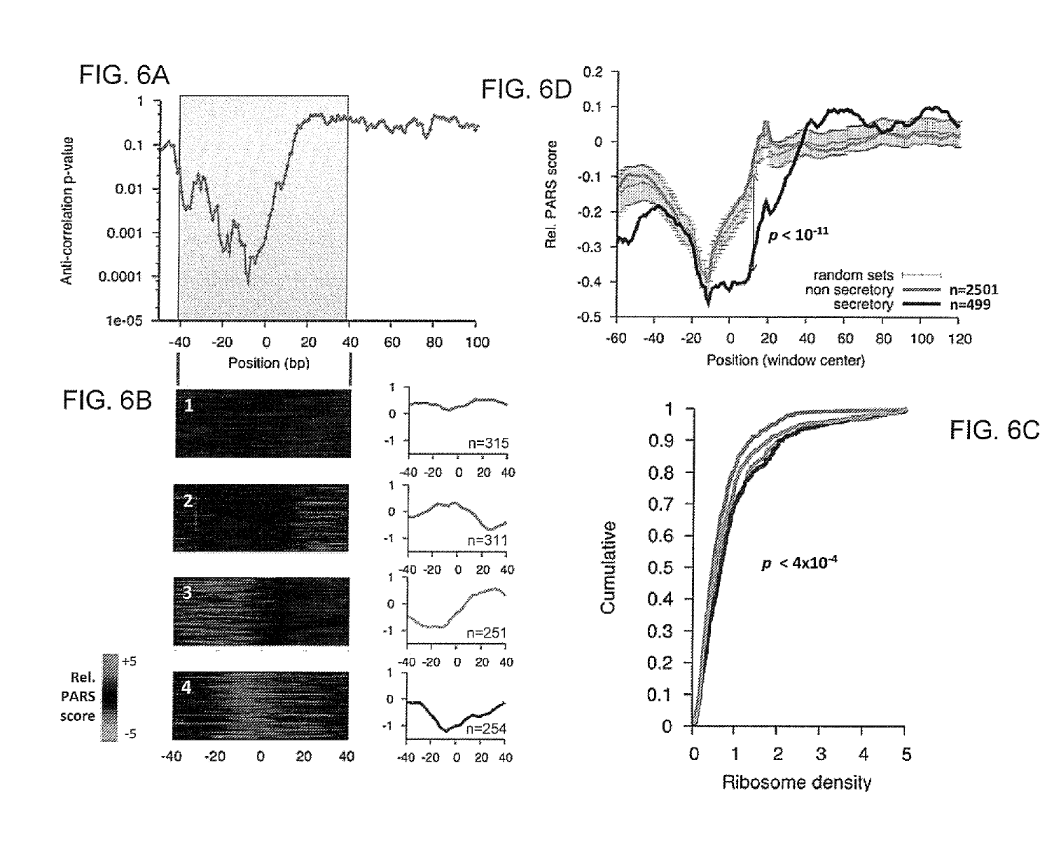

The present inventors have uncovered a novel method of predicting the pairability and secondary structure of multiple RNA polynucleotides simultaneously. Thus, as shown in the Examples section which follows, the novel strategy employs deep sequencing fragments of RNAs that were treated with structure-specific enzymes or chemicals, and mapping the resulting cleavage sites at a single nucleotide resolution, allowing to simultaneously profile thousands of RNAs of various lengths (FIGS. 1A-F, 2A-D and Examples 1 and 2). The novel method termed “Parallel Analysis of RNA Structure (PARS)” was applied to profile the secondary structure of the mRNAs of the budding yeast S. cerevisiae. The analysis revealed several RNA structural properties of yeast transcripts, including the existence of more secondary structure over coding regions compared to untranslated regions (FIG. 5D), a three-nucleotide periodicity of secondary structure across coding regions (FIGS. 5B and C), and a relationship between the efficiency with which an mRNA is translated and the lack of structure over its translation start site (FIGS. 6A-C). Thus, using the teachings described herein the present inventors were capable of determining the structural profiles of over 3000 distinct transcripts of the entire yeast transcriptome (Table 5, Example 2; and Supplementary Data). The novel method described herein is readily applicable to other organisms and to profiling RNA structure in diverse conditions, thus enabling studies of the dynamics of secondary structure at a genomic scale. The results presented herein demonstrate the feasibility of the novel method of the invention as a high-throughput method for probing the structure of multiple RNAs both in vitro and in vivo.

According to an aspect of some embodiments of the invention, there is provided a method of predicting a pairability of nucleotides of a plurality of RNA polynucleotides, the method comprising: (a) simultaneously determining a paired state or an unpaired state of nucleotides of the plurality of RNA polynucleotides; and (b) corresponding the paired state or the unpaired state of the nucleotides to a database of nucleic acid sequences, the database comprises nucleic acid sequences representing the plurality of RNA polynucleotides, thereby determining the pairability of nucleotides of the plurality of RNA polynucleotides.

As used herein the term “pairability” refers to the paired or the unpaired state of a nucleotide in a given RNA polynucleotide.

Base-pairing of nucleotides occur between nucleotide strands via hydrogen bonds. Within a DNA molecule, base-pairs are formed between adenine (A) and thymine (T); as well as between guanine (G) and cytosine (C). In RNA polynucleotides, base pairing is formed between uracil (U) (instead of thymine) and adenine; as well as between guanine and cytosine.

As used herein the phrase “predicting a pairability of a nucleotide of an RNA polynucleotide” refers to the likelihood that a specific nucleotide of an RNA polynucleotide is in a paired state, or in an unpaired state.

According to some embodiments of the invention, the pairability of a nucleotide-of-interest is determined with respect to other nucleotide(s) of the same RNA polynucleotide (intra molecule base pairs).

According to some embodiments of the invention, the pairability of a nucleotide-of-interest is determined with respect to nucleotide(s) of another RNA polynucleotide, e.g., inter molecules base pairs.

The RNA polynucleotide can be a synthetic, recombinant or naturally occurring RNA. For example, the RNA polynucleotide can be obtained from an in vitro transcription of a nucleic acid coding sequence. Additionally or alternatively, the RNA polynucleotide can be isolated from a cell (e.g., a prokaryotic or eukaryotic cell) or from a virus (e.g., viral RNA which infects human or animal cells).

According to some embodiments of the invention, the RNA is purified from a cytoplasm of a cell.

It should be noted that an RNA polynucleotide of a cell or a virus can be in a purified form or in an unpurified (e.g., crude) form.

As used herein the phrase “purified form” with respect to RNA refers to being substantially free of non-RNA molecules such as proteins, DNA, and the like.

The sample comprising the RNA polynucleotides can be purified to remove proteins or DNA therefrom. For example, purification of RNA can be performed using hot (65° C.) acid phenol followed by chloroform, which thereby separates the RNA from proteins and DNA. While phenol and chloroform denatures proteins, the low pH of acid phenol (e.g., pH about 4) causes the DNA to be in included in the phenol phase and hence the aqueous phase comprises mostly RNA.

According to some embodiments of the invention, the RNA polynucleotide is in a native form. As used herein the phrase “native form” refers to the secondary and/or a tertiary structure of the RNA in vivo (e.g., within a living cell, tissue or organism) where it may associate with other molecules (e.g., DNA, proteins).

It should be noted that those of skills in the art are capable of identifying conditions imitating those present in vivo so as to enable an RNA polynucleotide to acquire in vitro the native form.

According to some embodiments of the invention, the sample comprising the RNA polynucleotide can be any in vitro or in vivo sample.

According to some embodiments of the invention, each of the RNA polynucleotides can be of any length such as from a few nucleotides to tens of nucleotides [e.g., from about 10-200 nucleotides, e.g., from about 50 nucleotides to about 200 nucleotides]; hundreds of nucleotides [e.g., from about 100 nucleotides to about 1000 nucleotides] or thousands of nucleotides [e.g., from about 1000 nucleotides to about 50,000 nucleotides or more).

According to some embodiments of the invention, each of the RNA polynucleotides comprises more than about 500 nucleotides, e.g., more than about 550 nucleotides, e.g., more than about 600 nucleotides, e.g., more than about 650 nucleotides, e.g., more than about 700 nucleotides, e.g., more than about 750 nucleotides, e.g., more than about 800 nucleotides, e.g., more than about 850 nucleotides, e.g., more than about 900 nucleotides, e.g., more than about 950 nucleotides, e.g. more than about 1000 nucleotides, e.g., more than about 1050 nucleotides, e.g. more than about 1100 nucleotides, e.g., more than about 1150 nucleotides, e.g. more than about 1200 nucleotides, e.g., more than about 1250 nucleotides, e.g. more than about 1300 nucleotides, e.g., more than about 1400 nucleotides, e.g. more than about 1450 nucleotides, e.g., more than about 1500 nucleotides, e.g. more than about 1550 nucleotides, e.g., more than about 1600 nucleotides, e.g. more than about 1650 nucleotides, e.g., more than about 1700 nucleotides, e.g. more than about 1750 nucleotides, e.g., more than about 1800 nucleotides, e.g. more than about 1900 nucleotides, e.g., more than about 2000 nucleotides, e.g. more than about 2500 nucleotides, e.g., more than about 3000 nucleotides, e.g. more than about 3500 nucleotides, e.g., more than about 4000 nucleotides, e.g. more than about 4500 nucleotides, e.g., more than about 5000 nucleotides, e.g. more than about 5500 nucleotides, e.g., more than about 6000 nucleotides, e.g., more than about 6500 nucleotides, e.g., more than about 7000 nucleotides, e.g., more than about 7500 nucleotides, e.g., more than about 8000 nucleotides, e.g., more than about 9000 nucleotides, e.g., more than about 10000 nucleotides, e.g., more than about 11000 nucleotides, e.g., more than about 12000 nucleotides, e.g., more than about 13000 nucleotides, e.g., more than about 14000 nucleotides, e.g., more than about 15000 nucleotides, e.g., between about 15000 to about 50000 nucleotides, or more.

A non-limiting example of a long RNA polynucleotide which secondary structure can be determined by the method of some embodiments of the invention is the homo sapiens HECT, UBA and WWE domain containing 1 (HUWE1)(GenBank Accession No. NM_{—}031407) which consists of 14734 nucleotides (including untranslated region) of which 13125 nucleotides of coding region.

According to some embodiments of the invention, the RNA polynucleotide is an in vitro transcribed RNA (e.g., from a nucleic acid construct which comprises a coding sequence encoding the RNA transcript and a promoter for directing transcription of the RNA). In vitro transcription of RNA is well known in the art.

As described, the method predicts the pairability of nucleotides in a plurality of RNA polynucleotides.

As used herein the phrase “plurality of RNA polynucleotides” refers to two or more distinct RNA molecules. It should be noted that two RNA polynucleotides are considered distinct from each other if their nucleic acid sequence is different in at least one nucleotide.

According to some embodiments of the invention each of the plurality of RNA molecules comprises a distinct coding sequence. It should be noted that two coding sequences are considered distinct from each other if their nucleic acid sequence is different in at least one nucleotide.

As described, determining the paired state or the unpaired state of nucleotides of the plurality of RNA polynucleotides is performed simultaneously.

According to some embodiments of the invention, the pairability of the nucleotides is performed simultaneously for all the RNA polynucleotides of the plurality of RNA polynucleotides.

As used herein the term “simultaneously” refers to performed in a single reaction mixture (e.g., a single tube), without needing to repeat the reaction for each RNA of the plurality of RNA polynucleotides, and/or for each portion of a single long RNA polynucleotide.

According to some embodiments of the invention, each of the plurality of the RNA polynucleotides is encoded by a different coding sequence, e.g., alternative splicing variants, RNA transcripts of different genes, RNA transcripts of different species.

According to some embodiments of the invention, the sample comprising the plurality of RNA polynucleotides is obtained from a cell of an organism.

According to some embodiments of the invention, the plurality of RNA polynucleotides are obtained from a biological sample which comprises cells or components thereof (e.g., cell exertion) such as body fluids, e.g., as whole blood, serum, plasma, cerebrospinal fluid, urine, lymph fluids, and various external secretions of the respiratory, intestinal and genitourinary tracts, tears, saliva, milk as well as white blood cells, tissue biopsy, malignant tissues, amniotic fluid and chorionic villi.

According to some embodiments of the invention, the pairability is determined for each of the nucleotides of at least two of the plurality of RNA polynucleotides.

According to some embodiments of the invention, determining the paired state or the unpaired state of nucleotides of the plurality of RNA polynucleotides is performed simultaneously for at least two RNA polynucleotides, e.g., for at least 3 RNA polynucleotides, e.g., for at least 4 RNA polynucleotides, e.g., for at least 5 RNA polynucleotides, e.g., for at least 6 RNA polynucleotides, e.g., for at least 7 RNA polynucleotides, e.g., for at least 8 RNA polynucleotides, e.g., for at least 9 RNA polynucleotides, e.g., for at least about 10, at least about 20, at least about 50, at least about 100, at least about 200, at least about 300, at least about 400, at least about 500, at least about 1000, at least about 2000, at least about 3000, at least about 5000, at least about 10,000, at least about 20,000, at least about 30,000, at least about 40,000, at least about 50,000, at least about 100,000, at least about 200,000 RNA polynucleotides, and more, e.g., of a whole transcriptom of a cell of an organism (e.g., human, animal, plant, bacteria, yeast).

According to some embodiments of the invention, determining the paired state or the unpaired state is effected using an RNA structure—dependent agent.

As used herein the phrase “RNA structure—dependent agent” refers to an agent which activity on an RNA molecule (e.g., cleavage or modification) or which binding to an RNA molecule is dependent on the secondary structure of the RNA, e.g., the pairability of the RNA nucleotides comprising the polynucleotide.

According to some embodiments of the invention, the RNA structure—dependent agent is an RNase selected from the group consisting of: (i) an RNase which specifically cleaves a phosphodiester bond of a paired RNA, and (ii) an RNase which specifically cleaves a phosphodiester bond of an unpaired RNA.

According to some embodiments of the invention the RNase cleaves a phosphodiester bond 3′ of a paired nucleotide.

According to some embodiments of the invention the RNase cleaves a phosphodiester bond 3′ of an unpaired nucleotide.

Following is a non-limiting list of RNases which cut single stranded RNA and which can be used along with the method of some embodiments of the invention: RNAse I [cleaves 3′-end of all 4 residues (A, G, C, U) with no base preference (Hypertext Transfer Protocol://World Wide Web (dot) epibio (dot) com/item (dot) asp?ID=347; e.g., Cat. No. N6901K, Epicentre® Biotechnologies, Madison, Wis.; Ambion® Cat. No. AM2294, AM2295 Hypertext Transfer Protocol://World Wide Web (dot) ambion (dot) com/index (dot) html)]; RNAse A REC3.1.27.5, cleaves 3′-end of unpaired C and U residues, leaving a 3′-phosphorylated product; e.g., Ambion® Cat. Nos. AM2270, AM2271, AM2272, AM2274]; RNase T1 [EC 3.1.27.3, it is sequence specific for single stranded RNAs, it cleaves 3′-end of unpaired G residues; e.g., Ambion® Cat. No. AM2280, AM2283]; RNase T2 (is sequence specific for single stranded RNAs; it cleaves 3′-end of all 4 residues, but preferentially 3′-end of “A”); RNase U2 (is sequence specific for single stranded RNAs; it cleaves 3′-end of unpaired A residues); RNase PhyM (is sequence specific for single stranded RNAs; it cleaves 3′-end of unpaired A and U residues).

According to some embodiments of the invention the RNase leaves a 3′-OH and a 5′-phosphate after cleavage of the phosphodiester bond.

In specific embodiments, e.g., when using RNAse A, the RNase leaves a 3′-phosphate and a 5′-OH after cleavage of the phosphodiester bond. In such case, prior to ligation the digested RNA molecules are first phosphorylated in order to obtain a 5′-phosphate at the 5′-end of each of the digested RNA molecules.

According to some embodiments of the invention, the RNase is an endonuclease. According to some embodiments of the invention, the RNase is devoid of an exonuclease activity. According to some embodiments of the invention, the Nase has no processivity. According to some embodiments of the invention, RNase cuts only one phosphodiester bond once it recognizes the specific structure of RNA (i.e., a paired or an unpaired).

According to some embodiments of the invention the RNase which specifically cuts single stranded RNA (cleaves a phosphodiester bond of an unpaired RNA) is RNase S1 (EC 3.1.30.1), RNase T1 (EC 3.1.27.3) and/or RNase A (EC 3.1.27.5).

Following is a non-limiting list of RNases which cut double stranded RNA and which can be used along with the method of some embodiments of the invention: RNase V1 (is non-sequence specific for double stranded RNAs, it cleaves base-paired nucleotide residues, e.g., Ambion® Cat. No. AM2275) and RNase R (which is able to degrade RNA with secondary structures without help of accessory factors).

According to some embodiments of the invention the RNase causes nicks in the double stranded RNA (cleavage of only one phosphodiester bond between paired nucleotides).

According to some embodiments of the invention the RNase which specifically cuts double stranded RNA (cleaves a phosphodiester bond of a paired RNA) is RNase V1 (EC 3.1.27.8).

The RNases can be obtained from various commercial suppliers such as Applied Biosystems and Ambion®. Additionally or alternatively, the RNases can be recombinantly synthesized by transforming a host cell with a nucleic acid construct which comprises the coding region of RNase under the control of a promoter (e.g., a constitutive promoter).

According to some embodiments of the invention, the RNA structure—dependent agent is a chemical selected from the group consisting of: (i) a chemical which specifically binds to or modifies an unpaired RNA, and; (ii) a chemical which specifically binds to or modifies a paired RNA.

As used herein the term “modifies” refers to covalent modification of a nucleotide. Examples include, but are not limited to, acetylation, phosphorylation, methylation and the like.

According to some embodiments of the invention, the RNA structure—dependent chemical directly modifies the nucleotide.

According to some embodiments of the invention, the RNA structure—dependent chemical accelerates the covalent modification of a nucleotide. For example, 1M7 is a chemical which accelerates the addition of an acetyl group to a flexible base in an RNA polynucleotide because these bases (the flexible bases) undergo the reaction better. The more flexible bases tend to be single stranded regions.

According to some embodiments of the invention, the specific binding of the chemical to the unpaired RNA or the modification of the unpaired RNA by the chemical is at least one order of magnitude higher than to a paired RNA, e.g., at least two orders of magnitude higher, e.g., at least three orders of magnitude higher, e.g., at least four orders of magnitude higher, e.g., at least five orders of magnitude higher, e.g., at least six orders of magnitude higher than to a paired RNA, or more.

According to some embodiments of the invention, the binding of the chemical to the RNA is effected covalently. For example, the chemical can modify the RNA molecule by covalently attaching to the RNA.

Non-limiting examples of a chemical which specifically binds to or modifies an unpaired RNA include 1-cyclohexyl-3(2-morpholinoethyl)carbodiimide metho-p-toluenesulfate (CMCT), dimethyl sulfate (DMS), and 1-methyl-7-niro-isatoic anhydride (1M7; Mortimer S A, 2007, J. Am. Chem. Soc. 129: 4144-4145).

It should be noted that the conditions under which the RNA structure—dependent agent binds to/modifies (in the case of a structure—dependent chemical) or digests (in the case of a structure—dependent RNase) the plurality of RNA polynucleotides are selected such that following such binding (or modification) or digestion the plurality of RNA polynucleotides are sufficiently represented for each of the sensitive regions in the RNA, namely, there is at least one polynucleotide which is specifically cut (by RNase), bound to the chemical or modified by the chemical in each of the sensitive regions in the RNA, i.e., the paired or unpaired nucleotides.

These conditions include the concentration of active agent (i.e., the RNase or the structure—dependent chemical), reaction temperature, reaction time, salt concentration and type, ions concentration and type, and other reagents as described in the Examples section which follows.

According to some embodiments of the invention, the conditions enable obtaining complementary DNA polynucleotides with an average length of about 50-500 nucleotides.

According to some embodiments of the invention the RNA structure—dependent agent cleaves (with respect to RNase) or binds/modifies (with respect to the chemical) at least once each RNA polynucleotide.

According to some embodiments of the invention the RNA structure—dependent agent cleaves (with respect to RNase) or binds/modifies (with respect to the chemical) at a single phosphodiester bond of each RNA polynucleotide.

According to some embodiments of the invention determining the paired state or the unpaired state of the nucleotides is performed by digesting the plurality of RNA polynucleotides with the RNase to thereby obtain digested RNA polynucleotides.

According to some embodiments of the invention, prior to subjecting the sample to treatment with the RNA structure-dependent agent, the proteins and/or other cellular components such as DNA, polysaccharides, membranes are removed from the sample.

According to some embodiments of the invention, the method further comprising denaturing the plurality of the RNA polynucleotides prior to determining the paired state or the unpaired state of the nucleotides of the plurality of RNA polynucleotides.

According to some embodiments of the invention, the method further comprising subjecting the plurality of the RNA polynucleotides to conditions which allow the folding of the RNA polynucleotides following the denaturing [e.g., heat to 90° C., cool on ice, and slowly bring to room temperature (10 mM Tris pH 7, 10 mM MgCl₂, 100 mM KCl)].

According to some embodiments of the invention, prior to being subjected to sequencing (determination of the nucleic acid sequence) the digested RNA polynucleotides are converted to DNA molecules. Such a conversion can be using an enzyme such as reverse transcriptase (e.g., EC 2.7.7.49).

Prior to reverse transcription, the digested RNA polynucleotides are ligated to universal adapters [(i.e., adapters (primers) which are not specific to a certain sequence of the RNA polynucleotide of interest, but rather are the same for all the plurality of RNA polynucleotides].

According to some embodiments of the invention, the adaptors preferentially ligate to 5′-phosphate.

Ligation can be done using any RNA ligase. Examples include T4 RNA ligase-2 and RNA ligase-1.

According to some embodiments of the invention, the ligation is performed with RNA ligase-2 which ligates only 5′-phosphate to 3′-OH of RNA.

According to some embodiments of the invention, the method does not involve design of sequence specific primers for each RNA polynucleotide-of-interest.

According to some embodiments of the invention, the method does not involve extension of sequence specific primers which are derived from the RNA polynucleotide-of-interest but rather use of sequencing primers which attach to the universal adapters.

According to some embodiments of the invention, the reverse transcription of the digested RNA polynucleotides is performed on 5′-phosphate-containing digested RNA molecules.

Additionally or alternatively, when the RNA is treated with chemical(s) which specifically bind to or modifies a single stranded or a double stranded RNA, determining the paired state or the unpaired state of the nucleotides can be performed by reverse transcription of the plurality of RNA polynucleotides following binding/modification by the chemical, to thereby obtain complementary DNA polynucleotides.

Once obtained, the complementary DNA polynucleotides are subjected to determination of nucleic acid sequence.

Various sequencing technologies which are known in the art can be used along with the method of the invention. For example, SOLEXA™ (Illumina), PYROSEQUENCING™ 454 (Roche Diagnostics Corporation) and SOLiD™ (Life Technologies), and Helicos (Helicos BioSciences Corporation).

Universal primers (adapters) for ligation and reverse transcription are usually provided along with the kits for deep sequencing. For example, when using the SOLID™ sequencing, the following SOLiD 2.0 Oligos can be used: The P1 adapter (SEQ ID NOs:44 and 45, which form a double strand DNA with an overhang), the P2 Adapter (SEQ ID NOs:46 and 47, which form a double strand DNA with an overhang) and the library PCR Primers 1 (SEQ ID NO:48) and 2 (SEQ ID NO:49). When the SOLEXA™ sequencing is used the following oligos can be used: 5′ RNA adapter (SEQ ID NO:50), 3′ RNA adapter (SEQ ID NO:51), RT primer (SEQ ID NO:52), small RNA PCR primers 1 (SEQ ID NO:53) and 2 (SEQ ID NO:54).

According to some embodiments of the invention, determination of the nucleic acid sequence is performed on each of the digested RNA polynucleotides.

According to some embodiments of the invention, sequence determination is performed simultaneously on a plurality of digested RNA polynucleotides.

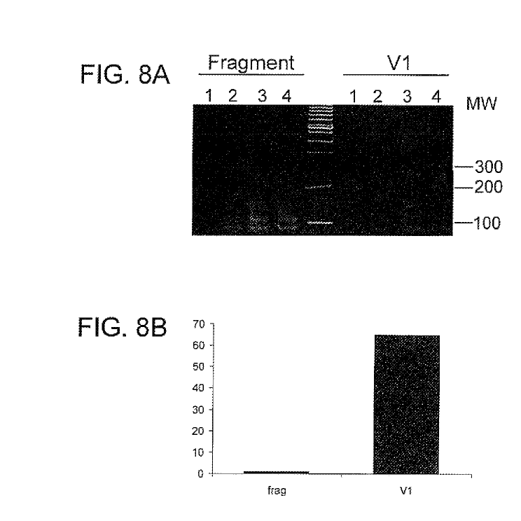

For example, as shown in Example 2 (FIGS. 8A-B) of the Examples section which follows, the digested RNA polynucleotides which comprise the 5′-phosphate (as opposed to randomly fragmented RNA polynucleotides which comprise 5′-OH) are ligated to adaptors so as to conjugate the adaptor which is used for reverse transcription and subsequently for sequence determination (sequencing).

According to some embodiments of the invention, corresponding the paired state or the unpaired state of the nucleotides to the data base nucleic acid sequences is performed by comparing a nucleic acid sequence of the complementary DNA polynucleotides with the database comprises nucleic acid sequences representing the plurality of RNA polynucleotides.

The nucleic acid sequences which represent the plurality of RNA polynucleotides and which are comprised in the database can be DNA, RNA, complementary DNA (cDNA), complementary RNA (cRNA), sense RNA, antisense RNA, genomic DNA, a transcriptome derived from a genome (bioinformatically deduced transcriptome), a transcriptome derived from transcripts extracted from a cell [e.g., from a pathological cell or a healthy cell (devoid of the pathology); from a cell before treatment with a drug/agent or a cell after treatment with the drug/agent; from a cell in an undifferentiated state or a differentiated cell; from cells at various differentiation stages; from an embryonic cell or a mature cell; from a stem cell or a differentiated cell and the like], and/or any combination thereof. The database can be experimentally determined (e.g., by sequencing of nucleic acid sequences obtained from a cell or using recombinant tools in vitro), can be obtained using bioinformatics tools or by a combination of both. For example, the database can include a sequence which is obtained by sequencing of cDNA encoding the RNA. For example, the database can be a transcriptome of a whole genome obtained by bioinformatics tools; the database can be a transcriptome obtained by sequencing of a whole genome RNA; the transcriptome can be of a specific cell, cell line, tissue and the like. Additionally or alternatively, database can be obtained from various bioinformatics tools available online such as through the National Center for Biotechnology Information or other well know databases.

Sequence comparison methods (also referred to as sequence alignment) can be performed computationally using various DNA analysis bioinformatics tools, which are freely available through the web (see e.g., the Hypertext Transfer Protocol://blast (dot) ncbi (dot) nlm (dot) nih (dot) gov/). Non-limiting examples of sequence comparisons methods include BLAST, ALIGN, Bioconductor Biostrings::pairwise Alignment, BioPerl dpAlign (Hypertext Transfer Protocol://World Wide Web (dot) bioperl (dot) org/wiki/Main_Page), BLASTZ, LASTZ, DOTLET, JAligner, LALIGN, malign, matcher, MCALIGN2, MUMmer, needle, HMMER, Ngila, PatternHunter, ProbA (also propA), REPuter, SEQALN, SIM, GAP, NAP, LAP, SIM, SLIM Search, Sequences Studio, SWIFT suit, stretcher, tranalign, water and wordmatch [for additional info see Hypertext Transfer Protocol://en (dot) wikipedia (dot) org/wiki/Sequence_alignment_software]. It should be noted that many sequence alignments can be also performed automatically.

According to some embodiments of the invention the method of some embodiments of the invention further comprising computing an occurrence of a nucleotide of each of the plurality of RNA polynucleotides within the nucleic acid sequence of the complementary DNA polynucleotides.

As used herein the phrase “occurrence of a nucleotide . . . within the nucleic acid sequence of the complementary DNA polynucleotides” refers to the frequency (e.g., in absolute numbers or in percentages) in which a certain nucleotide of an RNA polynucleotide (prior to being treated with the RNA structure—dependent agent) appears in the complementary DNA polynucleotides.

According to some embodiments of the invention the occurrence is computed for each nucleotide of the complementary DNA polynucleotide(s).

According to some embodiments of the invention the occurrence is computed for each nucleotide of each of the complementary DNA polynucleotide(s).

According to some embodiments of the invention the occurrence is computed (calculated) for a nucleotide which appears first (i.e., at the 5′ end) of the complementary DNA polynucleotide(s), e.g., on each of the complementary DNA polynucleotides.

According to some embodiments of the invention the occurrence is computed for a nucleotide which appears last (i.e., at the 3′ end) of the complementary DNA polynucleotide(s), e.g., on each of the complementary DNA polynucleotides.

According to some embodiments of the invention the occurrence is computed for both nucleotides which appear first (i.e., at the 5′ end) and last (i.e., at the 3′ end) of the complementary DNA polynucleotide(s), (e.g., on each of the complementary DNA polynucleotides.

According to some embodiments of the invention two complementary DNA sequences are considered distinct if their nucleic acid sequence is different in at least one nucleotide.

According to some embodiments of the invention a complementary DNA sequence is considered unique if it maps to a single location (sequence) in the genome (from which the RNA polynucleotide is derived).

According to some embodiments of the invention a higher occurrence of the nucleotide within the complementary DNA polynucleotides obtained using the RNA structure—dependent agent which specifically cleaves or binds/modifies the paired RNA as compared to an expected occurrence of the nucleotide indicates that the nucleotide is in the pair state in the RNA polynucleotide prior to being treated with the digested with RNA structure—dependent agent.

As used herein the phrase “expected occurrence” refers to the occurrence of a nucleotide within the complementary DNA polynucleotides which would have been obtained if the RNA was randomly digested without any preference to a sequence or a structure (i.e., to a paired or unpaired nucleotide).

According to some embodiments of the invention a higher occurrence of a certain nucleotide within the complementary DNA polynucleotides obtained using the RNase which specifically cleaves a phosphodiester bond of a paired RNA as compared to an expected occurrence of the nucleotide indicates that the nucleotide forms a base-pair in the RNA polynucleotide prior to being digested with the RNase.

According to some embodiments of the invention a higher occurrence of the nucleotide within the complementary DNA polynucleotides obtained using the RNA structure—dependent agent which specifically cleaves or binds/modifies the unpaired RNA as compared to an expected occurrence of the nucleotide indicates that the nucleotide is in the unpair state in the RNA polynucleotide prior to being treated with the digested with RNA structure—dependent agent.

According to some embodiments of the invention a higher occurrence of the nucleotide within the complementary DNA polynucleotides obtained using the RNase which specifically cleaves a phosphodiester bond of an unpaired RNA as compared to an expected occurrence of the nucleotide in the nucleic acid sequence indicates that the nucleotide does not form a base-pair (i.e., is in an unpair state) in the RNA polynucleotide prior to being digested with the RNase.

According to some embodiments of the invention a higher occurrence of the nucleotide within the complementary DNA polynucleotides obtained using the RNA structure—dependent agent which specifically cleaves or binds/modifies the paired RNA as compared to an occurrence of the nucleotide in the complementary DNA polynucleotides obtained using the RNA structure—dependent agent which specifically cleaves or binds/modifies the unpaired RNA indicates that the nucleotide is in the pair state in the RNA polynucleotide prior to being treated with the RNA structure—dependent agent, and vice versa, namely, a lower occurrence of the nucleotide within the complementary DNA polynucleotides obtained using the RNA structure—dependent agent which specifically cleaves or binds/modifies the paired RNA as compared to an occurrence of the nucleotide in the complementary DNA polynucleotides obtained using the RNA structure—dependent agent which specifically cleaves or binds/modifies the unpaired RNA indicates that the nucleotide is in the unpair state in the RNA polynucleotide prior to being treated with the RNA structure—dependent agent.

According to some embodiments of the invention a higher occurrence of the nucleotide within the complementary DNA polynucleotides obtained using the RNase which specifically cleaves a phosphodiester bond of a paired RNA as compared to an occurrence of the nucleotide in the complementary DNA polynucleotides obtained using the RNase which specifically cleaves a phosphodiester bond of an unpaired RNA indicates that the nucleotide forms a base-pair in the RNA polynucleotide prior to being digested with the RNase, and vice versa, namely, a lower occurrence of the nucleotide within the complementary DNA polynucleotides obtained using the RNase which specifically cleaves a phosphodiester bond of a paired RNA as compared to an occurrence of the nucleotide in the complementary DNA polynucleotides obtained using the RNase which specifically cleaves a phosphodiester bond of an unpaired RNA indicates that the nucleotide does not form a base-pair (i.e., is unpaired) in the RNA polynucleotide prior to being digested with the RNase.

According to some embodiments of the invention a higher occurrence of the nucleotide within the complementary DNA polynucleotides obtained using the RNA structure—dependent agent which specifically cleaves or binds/modifies the unpaired RNA as compared to an occurrence of the nucleotide in the complementary DNA polynucleotides obtained using the RNA structure—dependent agent which specifically cleaves or binds/modifies the paired RNA indicates that the nucleotide is in the unpair state in the RNA polynucleotide prior to the being treated with the RNA structure—dependent agent, and vice versa, namely, a lower occurrence of the nucleotide within the complementary DNA polynucleotides obtained using the RNA structure—dependent agent which specifically cleaves or binds/modifies the unpaired RNA as compared to an occurrence of the nucleotide in the complementary DNA polynucleotides obtained using the RNA structure—dependent agent which specifically cleaves or binds/modifies the paired RNA indicates that the nucleotide is in the pair state in the RNA polynucleotide prior to the being treated with the RNA structure—dependent agent.

According to some embodiments of the invention a higher occurrence of the nucleotide within the complementary DNA polynucleotides obtained using the RNase which specifically cleaves a phosphodiester bond of an unpaired RNA as compared to an occurrence of the nucleotide in the complementary DNA polynucleotides obtained using the RNase which specifically cleaves a phosphodiester bond of a paired RNA indicates that the nucleotide does not form a base-pair (i.e., is unpaired) in the RNA polynucleotide prior to the being digested with the RNase, and vice versa, namely, a lower occurrence of the nucleotide within the complementary DNA polynucleotides obtained using the RNase which specifically cleaves a phosphodiester bond of an unpaired RNA as compared to an occurrence of the nucleotide in the complementary DNA polynucleotides obtained using the RNase which specifically cleaves a phosphodiester bond of a paired RNA indicates that the nucleotide forms a base-pair in the RNA polynucleotide prior to the being digested with the RNase.

Thus, the teachings of the invention can be used to determine the pairability of nucleotides in a single RNA polynucleotide in a single “run” (e.g., of any length, including large transcripts which cannot be subjected to conventional footprinting, e.g., due to the gel-size limitation) as well as to determine the pairability of nucleotides of a plurality of RNA polynucleotides (e.g., simultaneously, in a “single run”).

For example, when a plurality of RNA polynucleotides are included in a sample, the RNase(s) digests the mixture of RNA polynucleotides, and the digested RNA polynucleotides (which include a mixture of fragments deriving from the plurality of RNA polynucleotides) are subjected to sequence determination. The identified nucleic acid sequences are compared to the sequences of the original RNA polynucleotides (e.g., as determined prior to digesting the RNA polynucleotides with RNases, or as known from the database), and the occurrence of a nucleotide of each of the original RNA polynucleotide (of the plurality of the RNA polynucleotides) is determined within the sequences of the digested RNA polynucleotides. Since the sequences of the digested RNA polynucleotides align to the original sequences of the RNA polynucleotides (before digestion) one can calculate the frequency of fragments beginning or ending at a certain nucleotide of the original RNA polynucleotide. For example, if a high frequency of the RNase V1—digested RNA polynucleotides begin with a certain nucleotide (e.g., a nucleotide at position 500 of the RNA polynucleotide), then such a high frequency indicates that the nucleotide preceding this nucleotide, i.e., the nucleotide at position 499 of the RNA polynucleotide, forms a base-pair in the original RNA polynucleotide. Additionally or alternatively, if a high frequency of the RNase S1—digested RNA polynucleotides begin with a nucleotide at position 520 of the RNA polynucleotide, then such a high frequency indicates that the nucleotide preceding this nucleotide, i.e., the nucleotide at position 519 of the RNA polynucleotide does not form a base-pair (i.e., is unpaired) in the original RNA polynucleotide.

Given that the pairability of each of the nucleotides of an RNA polynucleotide or a plurality of RNA polynucleotides can be determined with high reliability, the teachings of the invention can be used to determine the secondary structure of an RNA polynucleotide or a plurality of RNA polynucleotides.

Thus, according to an aspect of some embodiments of the invention there is provided a method of determining a secondary structure of an RNA polynucleotide. The method is effected by (a) predicting the pairability of nucleotides of the plurality of RNA polynucleotides according to the method of the invention; and (b) determining the secondary structure of the RNA polynucleotide based on the predicted pairability of the nucleotides, thereby determining the secondary structure of the RNA polynucleotide.

As used herein the phrase “secondary structure of an RNA polynucleotide” refers to the folding state of the RNA polynucleotide by forming hydrogen bonds between complementary nucleotides (e.g., adenine and uracil; and cytosine and guanine).

It should be noted that various methods and algorithms are known in the art for determining a secondary structure of an RNA based on the pairability of the nucleotides comprising the RNA polynucleotide. Examples of suitable algorithms which can be used along with the method of some embodiments of the invention include, but are not limited to, Mfold [Zuker, M. Mfold web server for nucleic acid folding and hybridization prediction. Nucleic Acids Res 31, 3406-15 (2003)], Vienna [Hofacker, I. L., Fekete, M. & Stadler, P. F. Secondary structure prediction for aligned RNA sequences. J Mol Biol 319, 1059-66 (2002)] and CONTRAfold [Do, C. B., Woods, D. A. & Batzoglou, S. CONTRAfold: RNA secondary structure prediction without physics-based models. Bioinformatics 22, e90-8 (2006)].

The teachings of the invention can be also used to predict the tertiary structure of an RNA polynucleotide. Examples of suitable algorithms which can be used along with the method of some embodiments of the invention include, but are not limited to the algorithm which models the prediction of tertiary structure as constraint satisfactory problem (CSP) [described in Major F, Turcotte M, Gautheret D, Lapalme G, Fillion E, Cedergren R. The combination of symbolic and numerical computation for three-dimensional modeling of RNA. Science. 1991 Sep. 13; 253(5025):1255-60; which is fully incorporated herein by reference in its entirety]; the MC-SYM algorithm for which the CSP approach is used [described in Major F, Gautheret D, Cedergren R. Reproducing the three-dimensional structure of a tRNA molecule from structural constraints. Proc Natl Acad Sci USA. 1993 Oct. 15; 90(20):9408-12; which is fully incorporated herein by reference in its entirety]; the MANIP algorithm which uses as an input database of known fragments and secondary structure and provides as an output a complex 3D architecture; the NAB algorithm which uses as an input secondary structure and distance constraints and provides as an output the 3D structure; the ERNA-3D algorithm which uses as an input the Secondary structure and provides as an output 3D structures; the MC-Sym algorithm which uses as an input a secondary structure, distance, torsion and other structural constraints, database of known fragments and which provides as an output series of 3D structures; the RNA2D3D algorithm which uses as an input secondary structure, can also use known fragments, and provides as an output a 3D structure; the YAMMP (YUP) algorithm which uses as an input a reduced model representations and secondary structure and provides as an output a 3D structure. For additional details see Bruce A Shapiro et al. Bridging the gap in RNA structure prediction. Current Opinion in Structural Biology, 17:157-165, 2007, which is fully incorporated by reference herein in its entirety.

Thus, as mentioned above, using the teachings of the invention the present inventors were capable of determining the structural profiles of over 3000 distinct transcripts of the entire yeast transcriptome (Table 5, Example 2 of the Examples section which follows and in the Supplementary Data file. This includes the structures of the RNA polynucleotides comprising the nucleic acid sequence selected from the group consisting of SEQ ID NOs:1, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, 41, 42, and 55-3219.

The secondary structure of an RNA molecule can be used to understand biological processes which involve the RNA molecule and/or which are regulated by the RNA molecule. Additionally or alternatively, the secondary structure of an RNA can be used to identify RNA molecules having a similar secondary and optionally also tertiary structure, which can be referred to as “structural homologues”.

As used herein the phrase “structural homologues” refers to molecules having a common secondary structure.

For example, a common versus different secondary structure of an RNA molecule can be defined using RNAdistance [Hofacker I. L. Vienna RNA secondary structure server. Nucleic Acids Res. 2003;31:3429-3431, which is fully incorporated by reference in its entirety].

According to some embodiments of the invention, the structural homologues exhibit also sequence homology (homology in the primary nucleic acid sequence).

Sequence homology can be determined using any homology comparison software, including for example, the BlastN software of the National Center of Biotechnology Information (NCBI) such as by using default parameters.

According to some embodiments of the invention the sequence homology is at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 93%, at least about 94%, at least about 95%, at least about 96%, at least about 97%, at least about 98%, at least about 99%, e.g., 100% between the two structural homologues.

According to some embodiments of the invention, the structural homologues do not exhibit sequence homology. For example, two RNA molecules can share a similar secondary structure yet can belong to different gene families with different primary nucleic acid sequence. For example the RNA motives which are recognized by RNA binding proteins may appear in many distinct RNA molecules.

It should be noted that determination of a secondary structure of an RNA with an unknown function can be used to predict the function of the RNA based on the function of another RNA(s) which exhibits a structural homology to the RNA with the unknown function.

Once obtained, the secondary structures of the RNA polynucleotides can be used to identify molecules which can modulate (e.g., disrupt) the secondary (and subsequently also the tertiary) structure of an RNA polynucleotide.

Thus, according to an aspect of some embodiments of the invention there is provided a method of determining if a molecule is capable of modulating a secondary structure of an RNA polynucleotide. The method is effected by (a) contacting the plurality of RNA polynucleotides with the molecule and; (b) comparing a secondary structure of the plurality of RNA polynucleotides following the contacting to a secondary structure of the plurality of RNA polynucleotides prior to the contacting, wherein an alteration above a predetermined threshold of the secondary structure of an RNA polynucleotide following the contacting indicates that the molecule modulates the secondary structure of the RNA polynucleotide, thereby determining if the molecule is capable of modulating the secondary structure of the at least one RNA polynucleotide of the plurality of molecules.

According to some embodiments of the invention, the secondary structure of the RNA polynucleotide prior to and/or following the contacting is determined according to the method of the invention.

According to an aspect of some embodiments of the invention there is provided a method of determining if a molecule is capable of modulating a secondary structure of a plurality of RNA polynucleotides, the method is effected by: (a) contacting the plurality of RNA polynucleotides with the molecule; and (b) determining a secondary structure of the plurality of RNA polynucleotides according to the method of the invention following the contacting and comparing the secondary structure to a secondary structure of the same RNA polynucleotides prior to the contacting, wherein an alteration above a predetermined threshold of the secondary structure following the contacting indicates that the molecule modulates the secondary structure of the RNA polynucleotides, thereby determining if the molecule is capable of modulating the secondary structure of the plurality of RNA polynucleotides.

The molecule which is contacted with the plurality of RNA polynucleotides can be any small molecule, DNA, RNA (e.g., an RNA silencing agent), a peptide, an amino acid, a sugar, a carbohydrate, a fat molecule, an antibody, an antibiotic, a drug (e.g., chemotherapeutic drug) and a toxin.

As used herein, the term “RNA silencing agent” refers to an RNA which is capable of inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression.

According to some embodiments of the invention, contacting is effected by adding the molecule to a sample comprising the plurality of RNA polynucleotides. The sample can be an in vitro sample (e.g., isolated cells, isolated RNA molecules), an ex vivo sample (e.g., a sample obtained from a living organism, e.g., human, e.g., blood, tissue biopsy, body fluids, which can optionally be further cultured outside the body, e.g., under in vitro conditions), or an in vivo sample (within a living organism).

It should be noted that contacting can be effected for a time period sufficient for binding of the molecule to at least one of the plurality of RNA polynucleotides and optionally modulating the RNA secondary structure thereof, and those of skills in the art are capable of adjusting the conditions needed for such an effect to occur.

As used herein the phrase “above a predetermined threshold” refers to the increase or decrease in the number or percentage of nucleotides of RNA polynucleotide which change their pairness state (i.e., being in a paired or unpaired state) following the contact with the molecule.

According to some embodiments of the invention, the predetermined threshold is a change in the pairness of at least one nucleotide, at least two nucleotides, at least three nucleotides, at least four nucleotides, at least 5 nucleotides, at least 6nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 25 nucleotides, at least 30 nucleotides, at least 35 nucleotides, at least 40 nucleotides, at least 45 nucleotides, at least 50 nucleotides, at least 55 nucleotides, at least 60 nucleotides, at least 65 nucleotides, at least 70 nucleotides, at least 75 nucleotides, at least 80 nucleotides, at least 85 nucleotides, at least 90 nucleotides, at least 95 nucleotides, at least 100 nucleotides, at least 110 nucleotides, at least 120 nucleotides, at least 130 nucleotides, at least 140 nucleotides, at least 150 nucleotides, at least 200 nucleotides, or more of the nucleotides comprising the RNA polynucleotide.

According to some embodiments of the invention, the predetermined threshold is a change in the pairness of at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, or more of the nucleotides comprising the RNA polynucleotide.

The teachings of the invention can be used to identify molecules which modulate the secondary structure of at least one molecule of a plurality of molecules (e.g., a plurality of RNA molecules which are comprised in a biological sample, such as in a single cell, in body fluids or in a tissue biopsy).

According to some embodiments of the invention, the molecule(s) modulates the secondary structure of at least 2, at least 3, at least 4, at least 5, at least 6, at least 7, at least 10, at least 20, at least 30, at least 40, at least 50 or more RNA polynucleotides of a plurality RNA polynucleotides comprised in a sample.

Since the RNA structure affects the function of the RNA and since alterations in RNA's structure and/or activity are involved in the pathogenesis of many pathologies (disease, disorder or condition), the teachings of the invention can be used to screen for pathology associated markers.

Thus, according to an aspect of some embodiments of the invention there is provided a method of screening for a marker associated with a pathology. The method is effected by identifying at least one RNA polynucleotide having an altered secondary structure between cells associated with the pathology and cells devoid of the pathology (from a control subject), wherein an alteration above a predetermined threshold between the secondary structure of the RNA polynucleotide in the cells associated with the pathology and the secondary structure of the RNA polynucleotide in the cells devoid of the pathology indicates that the at least one RNA polynucleotide is associated with the pathology, thereby screening for a marker associated with the pathology.

According to some embodiments of the invention, the cells associated with the pathology can be derived from the pathology (e.g., a tissue exhibiting histological markers of the pathology).

According to some embodiments of the invention, the cells devoid of the pathology can be obtained from a control subject or from a healthy, non-affected cell of a subject who is affected by the pathology (e.g., in case of a solid tumor, the cells devoid of the pathology can be obtained from a healthy tissue, or blood).

Screening for diagnostic or therapeutic targets can be effected under in vitro, ex vivo or in vivo conditions are described above.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### Examples

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Methods

Media and growth conditions—Yeast strain S288C was grown at 30° C. to exponential phase (4×10⁷ cells/nil) in yeast peptone dextrose (YPD) medium.

RNA preparation—Total RNA was extracted from cells using a using hot, acid phenol (Sigma) essentially as described in A. Lee, K. D. Hansen, J. Bullard, S. Dudoit, G. Sherlock, PLoS Genet 4, e1000299 (December 2008), which is fully incorporated herein by reference. Poly(A) RNA was obtained by purifying twice using the Poly(A) purist Kit according to manufacturer's instructions (Ambion).

Preparation of RNA transcripts in vitro—RNA transcripts of P4P6 (SEQ ID NO:7), P9-9.2 (SEQ ID NO:8), HOTAIR [GenBank Accession No. DQ926657.1); SEQ ID NO:5)], fragments of HOTAIR, are obtained by PCR followed by in vitro transcription using RiboMAX Large Scale RNA production Systems Kit according to the manufacturer's instructions (Promega). The RNA was purified using 8% denaturing polyacrylamide gel electrophoresis (PAGE) prepared with 19:1 acrylamide:bisacrylamide, 7 M urea and 90 mM Tris-borate, 2 mM EDTA). The RNA bands were visualized by UV shadowing and excised out of the gel. The RNA was recovered by passive diffusion into water overnight at 4° C., followed by ethanol precipitation (0.3 M Sodium Acetate, 1% glycogen and 3 volumes of 100% ethanol) and resuspended in water.

Full length YKL185W (Ash1) [GenBank Accession No. NC_{—}001143.8 (94504.96270); SEQ ID NO:3), GenBank Accession No. NC_{—}001143.8 (94172.96368); SEQ ID NO: 3218], a fragment of YNL229C (GenBank Accession No. NC_{—}001146: (219138.220202, complement); SEQ ID NO:43; the fragment of YNL229C includes nucleotides 3-368 of SEQ ID NO:43), YLR110C [GenBank Accession No. NC_{—}001144.4 (370144 . . . 369638, complement); SEQ ID NO:1], YDL184C [GenBank Accession No. NC_{—}001136.9 (130408.130485, complement); SEQ ID NO:2] were obtained by PCR using primers against the yeast genome followed by in vitro transcription using RiboMAX Large Scale RNA production Systems Kit according to the manufacturer's instructions (Promega). The RNAs were purified using RNeasy Mini kit (Qiagen) following manufacturer's instructions.

Enzymatic Structure Probing—In vitro transcribed RNA was treated with 5 units of Antarctic Phosphatase (NEB) at 37° C. for 30 minutes, followed by heat inactivation at 65° C. for 7 minutes. T4 polynucleotide kinase (PNK) was then used to add [γ-³²P]ATP to the 5′-end of RNA by incubating at 37° C. for 30 minutes. An equal volume of RNA loading dye (95% Formamide, 18 mM EDTA, 0.025% SDS, 0.025% Xylene Cyanol, 0.025% Bromophenol Blue) was added before the RNA was run on a 8%, 7 M urea, denaturing PAGE gel. Bands corresponding to the right size were excised out of the gel. The gel slices were freezed on dry ice and thawed at room temperature for three times, and RNA was recovered by immersing the gel slice in 100 μl of water, at 4° C., overnight. The amount of radioactivity present was measured by scintillation spectroscopy.

Prior to structure mapping, the labeled RNA was added to 1 μg of total yeast RNA and was renatured by heating to 90° C., cooled on ice, and slowly brought to room temperature in structure buffer (10 mM Tris pH 7, 100 mM KCl, 10 mM MgCl₂). Structure determination was obtained by digesting with dilutions of RNase V1 (EC 3.1.27.8; Ambion) and RNase S1 (EC 3.1.30.1; Fermentas) at room temperature for 15 minutes. The reaction was stopped by using inactivation and precipitation buffer (Ambion), the RNA was recovered using ethanol precipitation and was dissolved in RNA loading dye. The RNA was resolved by running a 8% denaturing PAGE gel.

Additional structure depended RNases which were used include RNase T1 (EC 3.1.27.3) and RNase A (EC3.1.27.5).

T1 urea sequencing ladder was obtained by incubating labeled RNA, mixed with 1 μg of total RNA, in sequencing buffer (20 mM sodium citrate pH 5, 1 mM EDTA, 7 M urea) at 50° C. for 5 minutes. The samples were cooled to room temperature and cleaved using 10-100 fold dilutions of RNase T1 for 15 minutes. The reaction was stopped by adding inactivation and precipitation buffer (Ambion), and the RNA was recovered using ethanol precipitation and dissolved in RNA loading dye. The RNA was resolved by running a 8% denaturing PAGE gel.

Alkaline hydrolysis ladder was obtained by incubating labeled RNA in alkaline hydrolysis buffer (50 mM Sodium Carbonate [NaHCO₃/Na₂Co₃] pH 9.2, 1 mM EDTA) at 95° C. for 5-10 minutes. An equal volume of the RNA loading dye was added to the fragmented RNA and resolved using 8% denaturing PAGE gel.

Quantification of band intensities—Band intensities on the sequencing gel are quantified using SAFA.

SOLiD™ Applied Biosystems Library construction—P4P6, P9-9.2, HOTAIR, YKL185W and fragment of YNL229C were doped into poly(A)+ mRNA as controls. The RNA pool was then folded and probed for structure using 0.01 Units of RNase V1 (Ambion), or 1000 Units of S1 nuclease (Fermentas), in a 100 μl reaction volume, as described above. To capture the cleaved fragments and convert them into a library for Solid sequencing, the present inventors used the SOLiD™ Small RNA Expression Kit (Ambion) and modified the manufacturer's instructions as follows.

Briefly: RNase V1 and S1 nuclease cleaved RNA pool was further fragmented using alkaline hydrolysis buffer at 95° C. for 3 minutes. The fragments were resolved on a 6% denaturing PAGE gel and a band corresponding to 75-200 bases of RNA size was excised out of the PAGE gel. The gel slice was frozen and thawed three times and crushed. RNA was recovered by passive diffusion into water at 4° C., overnight, followed by ethanol precipitation. The RNAs were ligated to 5′ adaptors by adding T4 RNA ligase-2 (EC6.5.1.3) and adaptor mixA (SOLiD™ Small RNA Expression Kit) and incubating at 16° C., overnight. The RNA was then treated with Antarctic Phosphatase (NEB), 37° C. for 1 hour, and heat inactivated at 65° C. for 7 minutes. Adaptor mixA was re-added to the RNA to maximize ligation to the 3′ end of the RNA and incubated at 16° C. for 6 hours. Reverse transcription was carried out using ArrayScript reverse transcriptase (Ambion) (EC 2.7.7.49) and a primer which binds to the adaptor and the RNA was removed using RNase H. 18-20 rounds of PCR using the Taq polymerase (EC2.7.7.7) were carried out using SOLiD PCR primers (of the universal adapters) provided in the kit.

SOLiD™ Sequencing—cDNA libraries were amplified onto beads by subjected to emulsion PCR, enrichment and the resulting beads were deposited onto the surface of a glass slide according to the standard protocol described in the SOLiD Library Preparation Guide (Applied Biosystems). 35-50 by sequences were generated on a SOLiD™ System sequencing platform according to the standard protocol described in the SOLiD Instrument Operation Guide (Applied Biosystems). The sequences generated were further analyzed.

Sequence mapping—Obtained sequences were truncated to 35 by before mapping, and required to map uniquely to either the yeast genome or transcriptome, allowing up to one mismatch and no insertions or deletions. Exemplary mapping results are provided in Tables 1 and 2 below.

TABLE 1

| lane | input reads | mapped to genome | % | mapped to transcriptome | % | uniquely mapped | % |
|---|---|---|---|---|---|---|---|
| V1 rep#1 | 11863204 | 9279439 | 78.22 | 9011318 | 75.96 | 6629996 | 55.89 |
| V1 rep#2 | 7160815 | 4600352 | 64.24 | 4473520 | 62.47 | 3341250 | 46.66 |
| V1 rep#3 | 6528001 | 4356121 | 66.73 | 4250412 | 65.11 | 3097273 | 47.45 |
| V1 rep#4 | 29180169 | 20033105 | 68.65 | 19508366 | 66.85 | 12976611 | 44.47 |
| S1 rep#1 | 23115630 | 16076977 | 69.55 | 15783615 | 68.28 | 11086282 | 47.96 |
| S1 rep#2 | 23023964 | 15713013 | 68.25 | 15426287 | 67.00 | 10668210 | 46.34 |
| S1 rep#3 | 23749516 | 16537687 | 69.63 | 16223036 | 68.31 | 10896861 | 45.88 |
| TOTAL | 124621299 | 86596694 | 69.49 | 84676554 | 67.95 | 58696483 | 47.10 |

Table 1. Columns show, for each replicate (“lane”), the number of raw sequences obtained (“input reads”), the number of sequences, which mapped to the yeast genome or transcriptome (“mapped to genome”, “mapped to transcriptome” respectively) and the number of reads which mapped uniquely.
“V1” - RNase V1;
“S1” - RNase S1;
“rep#” - repetition No.

TABLE 2
Table 2. Continuation of Table 1. Columns show, for each
replicate (“lane”), the number of raw sequences which
mapped uniquely, non uniquely, or not annotated/not mapped.

| | unique | non unique | not annotated | not mapped |
|---|---|---|---|---|
| V1 rep#1 | 6629996 | 2381322 | 268121 | 2583765 |
| V1 rep#2 | 3341250 | 1132270 | 126832 | 2560463 |
| V1 rep#3 | 3097273 | 1153139 | 105709 | 2171880 |
| V1 rep#4 | 12976611 | 6531755 | 524739 | 9147064 |
| S1 rep#1 | 11086282 | 4697333 | 293362 | 7038653 |
| S1 rep#2 | 10668210 | 4758077 | 286726 | 7310951 |
| S1 rep#3 | 10896861 | 5326175 | 314651 | 7211829 |

Mapping of the short reads to the yeast transcriptome was done using version 1.1.0 of SHRiMP (2) downloaded from Hypertext Transfer Protocol://compbio (dot) cs (dot) toronto (dot) edu/shrimp/. The alignment started from the first base of the read, as PARS relies on the first base to recover a valid enzyme cleavage point. Reads that were not uniquely mapped were discarded and all genomic locations to which those reads mapped were marked as ‘unmappable’ due to ambiguity. In addition, genomic locations from which no reads were obtained in any of the replicates were also marked ‘unmappable’.

Genome and transcriptome assembly—The yeast genome was downloaded from The Saccharomyces Genome Database (SGD, Hypertext Transfer Protocol://World Wide Web (dot) yeastgenome (dot) org/) on June 2008. The yeast transcriptome was assembled by SGD annotations (downloaded June 2008). Untranslated regions (UTR) lengths were taken from Nagalkshmi et al (U. Nagalakshmi et al., in Science. (2008), vol. 320, pp. 1344-9). The set of genes predicted to encode secretory proteins is based on Emanuelsson et al (O. Emanuelsson, S. Brunak, G. von Heijne, H. Nielsen, Nat Protoc 2, 953 (2007).

Quantifying cleavage data—For each nucleotide along a transcript, the number of reads whose first mapped base was one base 3′ of the inspected nucleotide were counted.

The load of a transcript is defined as the total number of reads that mapped to the transcript, divided by the effective transcript length, which is the annotated transcript length minus the number of unmappable locations (see “sequence mapping” above). This measure is a proxy to the transcript's abundance in the sample. The ratio score of a nucleotide is defined as the ratio between the number of reads obtained for that nucleotide and the load of that transcript.

Computing the PARS Score—For each nucleotide, the logarithm of the ratio between the number of reads obtained for that nucleotide in the V1-treated sample and that obtained in the S1-treated sample was computed.

Specifically, the PARS Score is defined as the log₂ of the ratio between the number of times the nucleotide immediately downstream to the inspected nucleotide was observed as the first base when treated with RNase V1 and the number of times it was observed in the RNase S1 treated sample. The score of base i is thus defined as:

$${Score}_{i} = {log}_{2} ( \frac{\vert V {1}_{i + 1} \vert + 1}{\vert S {1}_{i + 1} \vert + 1} ) \qquad Formula I$$

where |V1ᵢ₊₁| and |S1ᵢ₊₁| are the number of times the nucleotide immediately downstream to the inspected nucleotide was observed as the first base of a sequence read in the V1- and S1-treated samples, respectively.

To account for differences in overall sequencing depth between the V1- and S1-treated samples, the number of reads for each nucleotide is normalized prior to the computation of the ratio:

|V1ᵢ|=k_{V}·RawV1ᵢ|

|S1ᵢ|=k_{S}·RawS1ᵢ| Formula II:

Where RawS1ᵢ and RawV1ᵢ are the raw number of reads observed for nucleotide i in the V1 and S1 treated samples, respectively, and the normalizing constants kᵥ and kₛ are computed as follows:

$${k}_{v} = \frac{( \vert V 1 \vert + \vert S 1 \vert ) / 2}{\vert V 1 \vert} \\ {k}_{s} = \frac{( \vert V 1 \vert + \vert S 1 \vert ) / 2}{\vert S 1 \vert} \qquad Formula III$$

Higher PARS (and positive) scores indicate higher double stranded propensity and lower (and negative) scores indicate that the base was less likely to be in a double-stranded conformation. The PARS score was capped to ±7, i.e., values, which were above +7 or below −7, were set to +7 or −7, respectively. Nucleotides with zero evidence counts on both lanes have a zero PARS score and were excluded from all subsequent analysis.

Enrichment of Gene Ontology annotations in over- and under-structured genes—For each gene, the average PARS score of its 5′ UTR, CDS, and 3′ UTR were computed separately, and the Wilcoxon rank sum test was used to ask whether genes with similar Gene Ontology (GO) annotations tend to have similar average PARS scores in any of the inspected regions. Multiple-hypothesis correction was done by FDR with a cutoff of 0.05. The Wilcoxon rank sum test results obtained for each gene are listed in Table 3 below.

TABLE 3

| 3UTR_under | p-value cut off for FDR < 0.05 is: 0 |
|---|---|
| 0.000750043 | hydrolase activity, acting on ester bonds |
| 0.001319102 | glutamine family amino acid metabolic process |
| 0.002189252 | endoribonuclease activity |
| 0.003019397 | protein processing |
| 0.004916578 | ribonuclease activity |
| 0.00499214 | pyrimidine nucleotide metabolic process |
| 0.006528625 | peptide pheromone maturation |
| 0.006640651 | tRNA processing |
| 0.006742024 | pyrimidine nucleotide biosynthetic process |
| 0.007395877 | heterocycle metabolic process |
| 0.007733319 | serine-type peptidase activity |
| 0.008096726 | ribosomal large subunit export from nucleus |
| 0.008278377 | carbamoyl-phosphate synthase activity |
| 0.008394098 | aromatic compound metabolic process |
| 0.008498933 | serine-type endopeptidase activity |
| 0.008694451 | endonuclease activity, active with either ribo- |
| | or deoxyribonucleic acids and producing |
| | 5-phosphomonoesters |
| 0.008694451 | endoribonuclease activity, producing |
| | 5-phosphomonoesters |
| 0.010131165 | response to DNA damage stimulus |
| 0.010152929 | glutamine family amino acid biosynthetic process |
| 0.010335072 | peptide binding |
| 0.010897536 | tRNA-specific ribonuclease activity |
| 0.012570831 | nucleotidase activity |
| 0.01429522 | response to acid |
| 0.014393705 | regulation of cell organization and biogenesis |
| 0.014522823 | S-acyltransferase activity |
| 0.01482766 | phosphoric ester hydrolase activity |
| 0.017919989 | tRNA metabolic process |
| 0.018360699 | Golgi trans face |
| 0.018642169 | phosphoprotein phosphatase activity |
| 0.020012565 | nucleotide-excision repair |
| 0.021266951 | methionine salvage |
| 0.022306714 | tRNA splicing |
| 0.022689331 | pyrimidine nucleoside triphosphate |
| | metabolic process |
| 0.022898849 | ligase activity, forming carbon-nitrogen bonds |
| 0.023122353 | amine metabolic process |
| 0.024498463 | glutamine family amino acid catabolic process |
| 0.024681545 | endonuclease activity |
| 0.024818508 | nuclease activity |
| 0.026346734 | GTPase binding |
| 0.026837208 | arginine metabolic process |
| 0.029170098 | malonyltransferase activity |
| 0.029695255 | oxidoreductase activity, acting on the CH—NH |
| | group of donors |
| 0.030601005 | purine base metabolic process |
| 0.030955322 | regulation of actin filament length |
| 0.03224123 | enzyme binding |
| 0.032960492 | intramolecular oxidoreductase activity, |
| | interconverting aldoses and ketoses |
| 0.033338633 | CTP synthase activity |
| 0.033543885 | arginine biosynthetic process |
| 0.034237777 | condensed chromosome, pericentric region |
| 0.034478999 | Rab GTPase binding |
| 5UTR_under | p-value cut off for FDR < 0.05 is: 0.000089723 |
| 2.03E−11 | structural constituent of ribosome |
| 1.51E−07 | cytosolic part |
| 2.48E−07 | ribosome |
| 7.99E−07 | large ribosomal subunit |
| 2.02E−06 | eukaryotic 48S initiation complex |
| 2.20E−06 | translation |
| 4.09E−06 | cytosolic large ribosomal subunit |
| | (sensu Eukaryota) |
| 7.20E−06 | small ribosomal subunit |
| 8.97E−05 | ribonucleoprotein complex |
| 0.000367609 | non-membrane-bound organelle |
| 0.000389687 | cytosol |
| 0.003542231 | glucose transporter activity |
| 0.003542231 | sugar transporter activity |
| 0.003622177 | organelle envelope lumen |
| 0.007340989 | transferase activity, transferring |
| | amino-acyl groups |
| 0.007997786 | macromolecule biosynthetic process |
| 0.008214458 | nucleosome assembly |
| 0.00996107 | nucleotide salvage |
| 0.012316766 | vacuole fusion, non-autophagic |
| 0.012538545 | transferase activity, transferring |
| | pentosyl groups |
| 0.012639188 | endonuclease activity |
| 0.012889267 | ubiquinone metabolic process |
| 0.014278032 | incipient bud site |
| 0.01562772 | cation: cation antiporter activity |
| 0.01562772 | monovalent cation:proton antiporter activity |
| 0.01580949 | GTP binding |
| 0.016047657 | H3/H4 histone acetyltransferase activity |
| 0.016284862 | macromolecule metabolic process |
| 0.016529259 | mitochondrial electron transport chain |
| 0.017350402 | serine hydrolase activity |
| 0.018444916 | Noc complex |
| 0.019652579 | mitochondrial membrane organization |
| | and biogenesis |
| 0.019700762 | extrinsic to organelle membrane |
| 0.01975247 | mitochondrial intermembrane space protein |
| | transporter complex |
| 0.020330901 | hexose transport |
| 0.022236721 | potassium ion transporter activity |
| 0.022236721 | pyruvate kinase activity |
| 0.022518483 | ribonucleoprotein binding |
| 0.022518483 | signal recognition particle binding |
| 0.023712751 | electron transport |
| 0.029939285 | histone acetyltransferase activity |
| 0.031186126 | inner mitochondrial membrane organization and |
| | biogenesis |
| 0.031186126 | protein import into mitochondrial inner membrane |
| 0.032096015 | purine nucleotide salvage |
| 0.032326688 | regulation of proteolysis |
| 0.032382639 | potassium ion homeostasis |
| 0.032598121 | organellar ribosome |
| 0.033881374 | peptidyltransferase activity |
| 0.033947186 | propionate metabolic process |
| 0.035160959 | glycogen biosynthetic process |
| CDS_under | p-value cut off for FDR < 0.05 is: 0.000059318 |
| 7.36E−09 | organellar ribosome |
| 2.10E−06 | organellar large ribosomal subunit |
| 5.93E−05 | DNA-dependent DNA replication |
| 0.000279759 | endonuclease activity |
| 0.000475526 | N-acyltransferase activity |
| 0.000500567 | mitochondrial part |
| 0.000622771 | DNA replication |
| 0.000683981 | mitochondrion |
| 0.000725939 | nuclease activity |
| 0.000811079 | histone acetyltransferase activity |
| 0.000930727 | nucleoplasm |
| 0.000984124 | organellar small ribosomal subunit |
| 0.001081724 | acetyltransferase activity |
| 0.001318414 | transcription factor complex |
| 0.001679761 | organelle inner membrane |
| 0.002721625 | protein tyrosine phosphatase activity |
| 0.003106832 | DNA repair |
| 0.003937244 | DNA strand elongation |
| 0.004379707 | endoribonuclease activity |
| 0.004700336 | replication fork |
| 0.004783396 | extrinsic to organelle membrane |
| 0.00518612 | thiol-disulfide exchange intermediate activity |
| 0.005603358 | GINS complex |
| 0.005712685 | Smc5-Smc6 complex |
| 0.00578419 | coenzyme biosynthetic process |
| 0.006017568 | protein amino acid acetylation |
| 0.006726906 | transcription factor TFIIH complex |
| 0.006742436 | sulfur utilization |
| 0.007800927 | transferase activity, transferring |
| | amino-acyl groups |
| 0.008018034 | ribonuclease activity |
| 0.008074945 | mitochondrial inner membrane protein insertion |
| | complex |
| 0.008140021 | mitochondrial lumen |
| 0.008344143 | protein methyltransferase activity |
| 0.008454898 | condensed chromosome |
| 0.008583497 | protein modification |
| 0.008590118 | protein amino acid acylation |
| 0.009611525 | mitochondrial inner membrane peptidase complex |
| 0.009692241 | endonuclease activity, active with either ribo- |
| | or deoxyribonucleic acids and producing |
| | 3-phosphomonoesters |
| 0.009692241 | tRNA-intron endonuclease complex |
| 0.010731481 | DNA metabolic process |
| 0.01087757 | H3/H4 histone acetyltransferase activity |
| 0.011090259 | lagging strand elongation |
| 0.011562966 | tRNA-specific ribonuclease activity |
| 0.011639807 | protein acetyltransferase complex |
| 0.012067095 | post-translational protein modification |
| 0.012087132 | mitochondrial protein processing |
| 0.012446309 | coenzyme A metabolic process |
| 0.0126325 | chromosome |
| 0.012787202 | replisome |
| 0.012952384 | vitamin biosynthetic process |

Predicted structure data—The Vienna package [I. L. Hofacker, M. Fekete, P. F. Stadler, J Mol Biol 319, 1059 (Jun. 21, 2002)] was used to fold transcripts, calculate the partition function of the structures ensemble and base pairing probabilities. Global and local (folding in selected short sliding windows) folding schemes were examined. To compute the pairing probability of a nucleotide the transcript was re-folded for every window, the window was moved a single base-pair at a time, and the average pairability reported for that nucleotide was taken across all windows that cover it.

Periodicity and codon signature—Periodicity analysis was done by a straightforward application of Discrete Fourier Transform to the average PARS score collected from the following genomic features: last 100 bases of the 5′ UTR, first 200 bases of the coding sequence, 100 first bases of the 3′ UTR.

The codon signature shown in the inset of FIG. 5C was computed by separately averaging the PARS score reported for each codon position, collected from the entire coding sequence of each of the 3000 mRNAs that went into our analysis. The reported p-values are computed by applying a t-test on the distribution of PARS scores of the different codon positions.

Clustering structure profiles—The present inventors applied k-means clustering to the structural profiles of all genes whose 5′ UTR is at least 50 bases long. To bring all profiles to the same baseline the present inventors used a relative PARS score, which is obtained by subtracting the average PARS score of the gene from each nucleotide. To account for missing values in the clustering, the present inventors first smoothed the profile by interpolating neighboring data (±10 window average) to assign a PARS score to bases that were unmappable. No missing values are required for further analysis.

Nucleotide-resolution raw reads and PARS scores for the 3000 genes included in our analysis can be visualized and downloaded at Hypertext Transfer Protocol://genie (dot) weizmann (dot) ac (dot) il/pubs/PARS010.

### Example I

### Parallel Analysis of RNA Structure

The following example describes a method of predicting the pairability (pairness, i.e., being in a base-pair or not) of each nucleotide of an RNA molecule (RNA polynucleotide) according to some embodiments of the invention which can be used to determine the secondary structure of an RNA molecule.

Determination of pairability of RNA molecules using a single enzyme—In the first step, a pool of different RNA species whose structural properties is to be measured is treated with one of several enzymes that cleaves specific RNA structures (e.g., enzymes that cleave at paired nucleotides). Next, the digested RNA pool is size-fractionated on a gel to select bands of a specified size range, followed by conversion of the RNA molecules to DNA, and subjecting the DNA to deep-sequencing to read millions of digested fragments. Finally, the millions of sequence reads are map to the reference genome, and these mapped sequences are used to estimate the pairability of every nucleotide in each of the original RNAs, based on the number of times that the sequences mapped to every nucleotide. For example, a nucleotide that appeared as the first base in a large number of the read sequences upon treatment with an enzyme that specifically cleaves paired bases, is likely to be paired to some other nucleotide in the original RNA structure.

FIGS. 1A-F schematically illustrate the basic method steps according to some embodiments of the invention. The method consists of two main stages. The first stage is experimental, where an RNA pool is treated with a structure-specific ribonuclease which cleave the RNA at specific double stranded or single stranded sites (FIG. 1A), is subjected to size-fractionation (FIG. 1B), and conversion to DNA followed by deep-sequencing to read millions of the resulting DNA fragments (FIG. 1C). The second stage is computational, where these millions of read sequences are taken as input, mapped to the reference genome (FIG. 1D) and the positioning and abundance of the mapped sequence is computed using an the algorithm to extract the structural evidence of the RNA. This evidence is then converted to a per-base score representing the pairability profile of the original RNA transcripts (FIG. 1E). For applications that require the secondary structure of the RNAs, rather than their accessibility, the extracted pairability profiles can then be used in conjunction with an RNA folding algorithm (e.g. Hofacker L I., et al. Fast folding and comparison of RNA secondary structures. Monatshefte Fr. Chemie. 125:167-188, 1994; Do C B., Woods D A., et al. CONTRAfold: RNA seconday structure prediction without physics-based models. Bioinfomatics 22:90-98, 2006) to construct a pairability-constrained secondary structure of the original RNA transcript (FIG. 1F).

### Example 2

### High-Throughput Measurement of RNA Structure Using One or Two Specific RNases

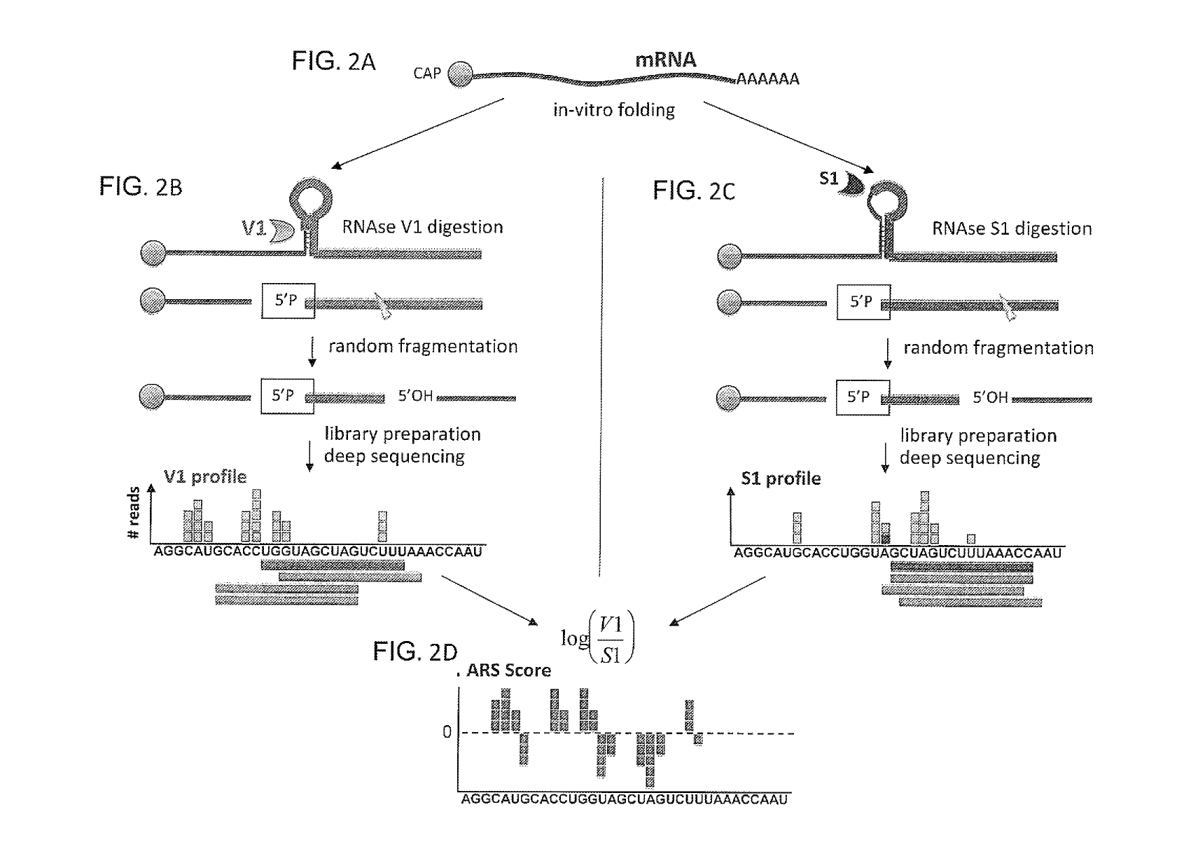

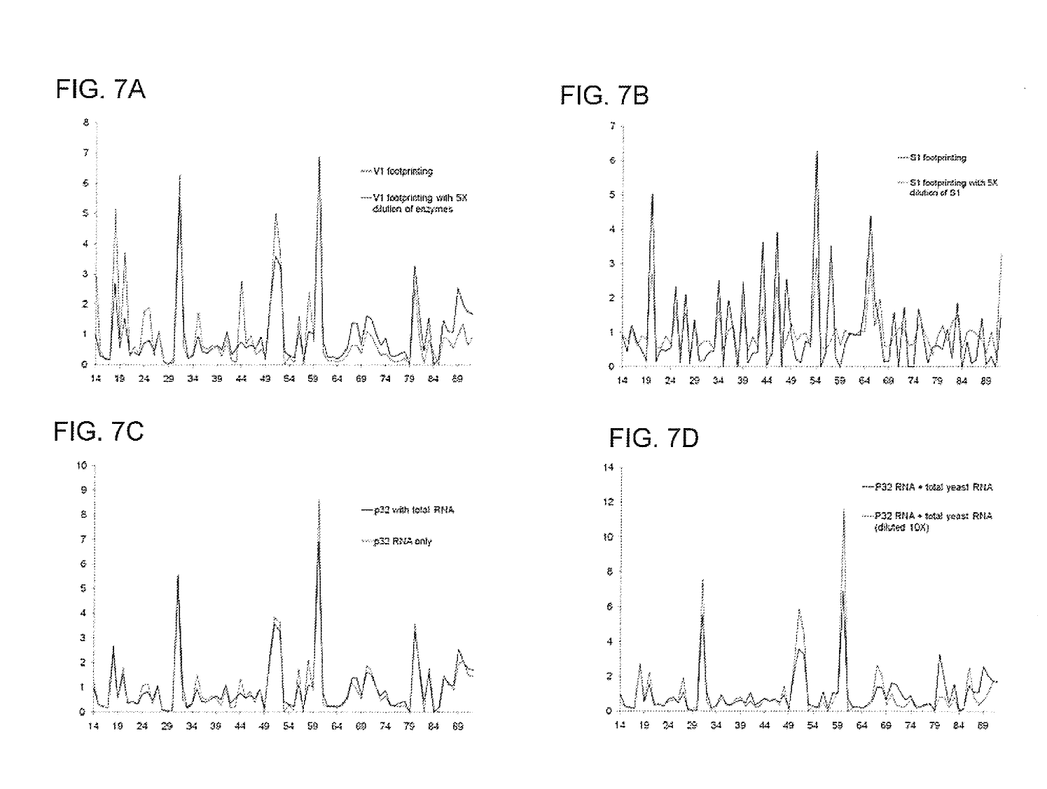

RNA structure in vivo is influenced by many factors. As a starting point for high throughput measurement of RNA structure, the present inventors have focused on RNA structures that may be strongly specified by the primary sequence of RNA itself. To simultaneously measure structural properties of many different RNAs from yeast, the present inventors extracted poly-adenylated transcripts from log-phase growing yeast, renatured the transcripts in vitro by standard methods in the presence of 10 mM Mg²⁺, and treated the resulting pool with RNase V1 and separately, with RNase S1. RNase V1 preferentially cleaves phosphodiester bonds 3′ of double-stranded RNA, while RNase S1 preferentially cleaves 3′ of single-stranded RNA. Obtaining data from these two independent and complementary enzymes allows the measurement of the degree to which each nucleotide is in a single- or double-stranded conformation (FIGS. 2A-D). Renaturation and enzymatic cleavage conditions were such that the cleavage reactions occur with single-hit kinetics (FIGS. 7A-B and where intramolecular RNA-RNA interactions are observed without heterotypic intermolecular interactions in the complex RNA pool (FIGS. 7C-D). For control, the present inventors used two additional RNA molecules, one, with a known secondary structure (Tetrahymena group I intron ribozyme) and the other with an unknown secondary structure (HOTAIR).

A splinted ligation method was used to specifically ligate V1 and S1 cleaved RNA to adaptors. The ligation was performed using T4 RNA Ligase 2 [also known as T4 Rn12 (gp24.1)], which exhibits both intermolecular and intramolecular RNA strand joining activity and which requires an adjacent 5′ phosphate and 3′ OH for ligation [Hypertext Transfer Protocol://World Wide Web (dot) neb (dot) com/nebecomm/products/productM0239 (dot) asp)]. The ligated RNA fragments were converted into cDNA libraries suitable for deep sequencing. As both enzymes leave a 5′ phosphate at the cleavage point and since only 5′ phosphoryl-terminated RNA are capable of ligating to adaptors, V1- and S1-cleaved fragments were enriched and selected against random fragmentation and degradation products that typically have 5′ hydroxyl (FIGS. 8A-B). Thus, each observed cleavage site provides evidence that the nucleotide which precedes the cleavage site (i.e., which is located 5′ of the cleavage site) on the uncut RNA molecule was in a double-stranded (for V1-treated samples) or single-stranded (for S1-treated samples) conformation. By combining many such evidence points a quantitative measure at nucleotide resolution was obtained that represents the degree to which a nucleotide was in a double- or single-stranded conformation.

Next, a scoring scheme was sought to allow the merge the results of the complementary RNase V1 and RNase S1 experiments into a single score describing the probability that each nucleotide was in a double- or single-stranded conformation. Ideally, such a scoring scheme should cancel non-specific cleavage present in both experiments and be invariant to transcript abundance. The scoring scheme is based on the ratio between the number of reads obtained for each nucleotide in the two experiments. For each nucleotide, the log of this ratio was used to define its PARS score, such that positive and higher PARS scores denote higher probabilities for nucleotides to be in double-stranded conformation while negative PARS scores suggest that the nucleotide was in a single-stranded conformation.

Four independent V1 experiments and three independent Si experiments were performed, resulting in a total of ˜85 million sequence reads that map to the yeast genome, of which ˜97% mapped to annotated transcripts (Tables 1 and 2 above).

The degree to which each base is cleaved by V1 or S1 was reproducible across the biological replicates (correlation=0.60−0.93, Table 4).

TABLE 4
Reproducibility of PARS signal at single nucleotide resolution

| | | Correlation |
|---|---|---|
| RNase S1 rep. 1 | RNase S1 rep. 2 | 0.93 |
| RNase S1 rep. 1 | RNase S1 rep. 3 | 0.76 |
| RNase S1 rep. 2 | RNase S1 rep. 3 | 0.60 |
| RNase V1 rep. 1 | RNase V1 rep. 2 | 0.75 |
| RNase V1 rep. 1 | RNase V1 rep. 3 | 0.73 |
| RNase V1 rep. 1 | RNase V1 rep. 4 | 0.62 |
| RNase V1 rep. 2 | RNase V1 rep. 3 | 0.91 |
| RNase V1 rep. 2 | RNase V1 rep. 4 | 0.61 |
| RNase V1 rep. 3 | RNase V1 rep. 4 | 0.64 |

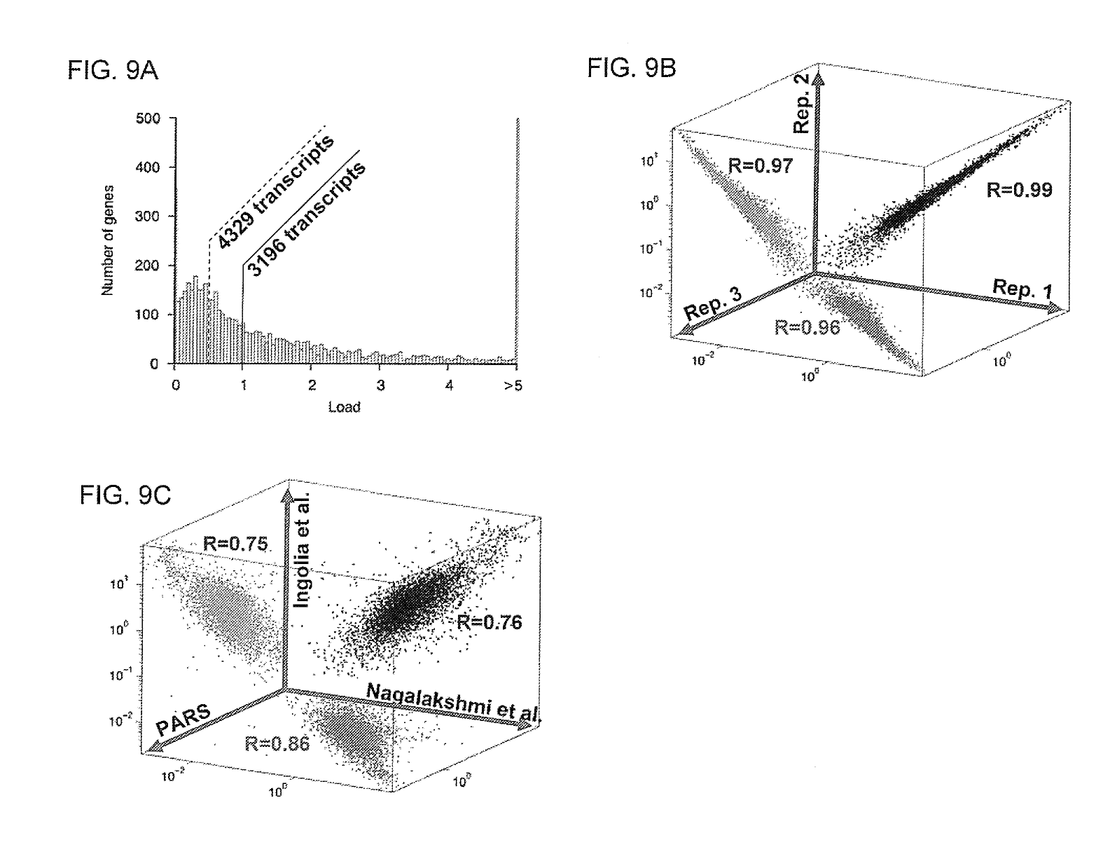

By combining the reads obtained across all replicates, PARS is able to provide per-nucleotide structural measurements for transcripts whose average nucleotide coverage is above 1.0 (Table 5, FIG. 9A).

TABLE 5
List of genes for which the RNA secondary structure was resolved by the method of the invention

| | SEQ | | | | | SEQ | | | |
|---|---|---|---|---|---|---|---|---|---|
| | ID | | | | | ID | | | |
| Gene | NO: | Length | Reads | Load | Gene | NO: | Length | Reads | Load |
| YAL003W | 79 | 844 | 111,557 | 132.18 | YJL143W | 1728 | 834 | 6,493 | 7.79 |
| YAL005C | 76 | 2,120 | 26,722 | 33.59 | YJL145W | 1729 | 991 | 3,500 | 3.53 |
| YAL007C | 75 | 842 | 5,414 | 6.43 | YJL148W | 1730 | 807 | 1,911 | 2.37 |
| YAL009W | 80 | 921 | 1,527 | 1.66 | YJL151C | 1648 | 471 | 16,613 | 35.27 |
| YAL012W | 81 | 1,352 | 63,777 | 47.17 | YJL154C | 1647 | 2,918 | 4,022 | 1.38 |
| YAL013W | 82 | 1,338 | 1,364 | 1.02 | YJL157C | 1646 | 2,628 | 6,839 | 2.60 |
| YAL014C | 74 | 950 | 2,960 | 3.12 | YJL158C | 1645 | 876 | 51,412 | 61.58 |
| YAL015C | 73 | 1,315 | 1,353 | 1.03 | YJL159W | 1731 | 1,601 | 66,741 | 53.74 |
| YAL016W | 83 | 1,978 | 7,226 | 3.65 | YJL166W | 1732 | 550 | 4,932 | 8.97 |
| YAL020C | 72 | 1,053 | 2,292 | 2.18 | YJL167W | 1733 | 1,211 | 14,097 | 11.64 |
| YAL021C | 71 | 2,783 | 8,059 | 2.93 | YJL171C | 1644 | 1,408 | 5,610 | 3.98 |
| YAL022C | 70 | 1,627 | 11,859 | 7.29 | YJL172W | 1734 | 1,898 | 20,735 | 10.98 |
| YAL023C | 69 | 2,475 | 33,893 | 13.70 | YJL173C | 1643 | 428 | 3,160 | 7.38 |
| YAL025C | 68 | 1,110 | 1,141 | 1.03 | YJL174W | 1735 | 1,118 | 10,997 | 9.84 |
| YAL026C | 67 | 4,282 | 4,954 | 1.16 | YJL177W | 1736 | 690 | 28,003 | 49.80 |
| YAL029C | 66 | 4,499 | 6,673 | 1.48 | YJL178C | 1642 | 937 | 5,828 | 6.22 |
| YAL030W | 84 | 482 | 1,097 | 2.28 | YJL179W | 1737 | 508 | 1,043 | 2.05 |
| YAL032C | 65 | 1,209 | 1,513 | 1.25 | YJL183W | 1738 | 1,410 | 13,233 | 9.38 |
| YAL033W | 85 | 613 | 1,367 | 2.23 | YJL184W | 1739 | 476 | 868 | 1.82 |
| YAL035W | 86 | 3,134 | 8,548 | 2.73 | YJL186W | 1740 | 1,875 | 14,869 | 7.93 |
| YAL036C | 64 | 1,297 | 8,401 | 6.48 | YJL189W | 30 | 321 | 121,564 | 385.47 |
| YAL038W | 9 | 1,698 | 686,228 | 404.26 | YJL190C | 31 | 534 | 97,239 | 211.24 |
| YAL039C | 63 | 897 | 1,593 | 1.78 | YJL191W | 1741 | 540 | 12,108 | 30.62 |
| YAL040C | 62 | 2,073 | 2,468 | 1.19 | YJL192C | 1641 | 800 | 10,364 | 12.95 |
| YAL041W | 87 | 2,711 | 3,616 | 1.33 | YJL193W | 1742 | 1,440 | 1,930 | 1.34 |
| YAL042W | 88 | 1,332 | 31,968 | 24.00 | YJL196C | 1640 | 1,088 | 11,542 | 10.61 |
| YAL043C | 61 | 2,466 | 4,850 | 1.97 | YJL198W | 1743 | 2,889 | 9,452 | 3.28 |
| YAL044C | 60 | 581 | 15,522 | 26.72 | YJL200C | 1639 | 2,542 | 8,410 | 3.31 |
| YAL044W-A | 89 | 446 | 2,645 | 5.93 | YJL205C | 1638 | 309 | 620 | 2.01 |
| YAL046C | 59 | 524 | 2,177 | 4.15 | YJL210W | 1744 | 881 | 3,270 | 3.71 |
| YAL049C | 58 | 890 | 4,429 | 4.98 | YJL212C | 1637 | 2,532 | 8,389 | 3.31 |
| YAL053W | 90 | 2,514 | 6,080 | 2.42 | YJL217W | 1745 | 792 | 3,606 | 4.55 |
| YAL058W | 91 | 1,614 | 9,361 | 5.80 | YJR001W | 1746 | 1,951 | 15,028 | 7.70 |
| YAL059W | 92 | 740 | 1,891 | 2.55 | YJR002W | 1747 | 2,160 | 3,654 | 1.69 |
| YAL060W | 93 | 1,353 | 9,178 | 6.78 | YJR004C | 1636 | 2,091 | 59,114 | 28.27 |
| YAR002C-A | 57 | 763 | 21,418 | 28.07 | YJR005W | 1748 | 2,441 | 2,635 | 1.08 |
| YAR002W | 94 | 1,746 | 4,086 | 2.34 | YJR006W | 1749 | 1,527 | 1,554 | 1.02 |
| YAR003W | 95 | 1,806 | 5,369 | 2.97 | YJR007W | 1750 | 1,170 | 7,939 | 6.79 |
| YAR007C | 56 | 1,983 | 6,647 | 3.35 | YJR009C | 1635 | 1,129 | 43,752 | 127.93 |
| YAR014C | 55 | 2,443 | 2,893 | 1.18 | YJR010C-A | 1634 | 498 | 2,578 | 5.22 |
| YAR015W | 96 | 1,052 | 8,539 | 8.12 | YJR010W | 1751 | 1,777 | 2,269 | 1.28 |
| YAR027W | 97 | 895 | 2,050 | 2.33 | YJR013W | 1752 | 1,301 | 2,646 | 2.03 |
| YAR028W | 98 | 950 | 2,574 | 2.72 | YJR014W | 1753 | 736 | 2,904 | 3.95 |
| YAR068W | 99 | 486 | 1,138 | 2.58 | YJR015W | 1754 | 1,666 | 17,588 | 10.56 |
| YAR071W | 100 | 1,404 | 1,118 | 3.34 | YJR016C | 1633 | 1,910 | 25,953 | 13.59 |
| YBL001C | 225 | 423 | 3,985 | 9.42 | YJR017C | 1632 | 754 | 6,566 | 8.71 |
| YBL002W | 237 | 635 | 24,082 | 38.73 | YJR019C | 1631 | 1,121 | 2,046 | 1.82 |
| YBL003C | 224 | 611 | 17,413 | 35.41 | YJR021C | 1630 | 1,148 | 1,157 | 1.01 |
| YBL006C | 223 | 716 | 4,495 | 6.28 | YJR024C | 1629 | 849 | 8,258 | 9.81 |
| YBL007C | 222 | 3,794 | 18,494 | 4.89 | YJR025C | 1628 | 729 | 1,434 | 1.97 |
| YBL008W-A | 238 | 240 | 296 | 1.23 | YJR040W | 1755 | 2,443 | 3,702 | 1.51 |
| YBL009W | 239 | 2,187 | 2,629 | 1.20 | YJR041C | 1627 | 3,786 | 3,866 | 1.02 |
| YBL011W | 240 | 2,486 | 6,116 | 2.47 | YJR042W | 1756 | 2,391 | 3,834 | 1.60 |
| YBL015W | 241 | 1,901 | 2,836 | 1.49 | YJR044C | 1626 | 644 | 3,644 | 5.66 |
| YBL016W | 242 | 1,418 | 3,514 | 2.48 | YJR045C | 1625 | 2,302 | 48,822 | 21.71 |
| YBL017C | 221 | 4,971 | 13,594 | 2.75 | YJR046W | 1757 | 1,894 | 4,104 | 2.17 |
| YBL018C | 220 | 468 | 1,342 | 2.87 | YJR047C | 1624 | 604 | 3,705 | 7.46 |
| YBL020W | 243 | 1,961 | 4,046 | 2.06 | YJR048W | 1758 | 1,272 | 3,591 | 2.83 |
| YBL022C | 219 | 3,572 | 8,467 | 2.37 | YJR049C | 1623 | 1,774 | 1,885 | 1.06 |
| YBL024W | 244 | 2,255 | 5,340 | 2.37 | YJR051W | 1759 | 1,578 | 2,745 | 1.74 |
| YBL025W | 245 | 637 | 803 | 1.26 | YJR057W | 1760 | 741 | 1,897 | 2.56 |
| YBL026W | 246 | 481 | 998 | 2.07 | YJR058C | 1622 | 537 | 1,337 | 2.49 |
| YBL027W | 247 | 684 | 46,917 | 96.71 | YJR059W | 1761 | 2,643 | 4,009 | 1.52 |
| YBL028C | 218 | 549 | 1,402 | 2.55 | YJR060W | 1762 | 1,769 | 3,342 | 1.89 |
| YBL030C | 217 | 1,323 | 12,048 | 9.11 | YJR062C | 1621 | 1,450 | 1,823 | 1.26 |
| YBL032W | 248 | 1,395 | 13,060 | 9.36 | YJR063W | 1763 | 551 | 3,897 | 7.07 |
| YBL033C | 216 | 1,130 | 2,129 | 1.88 | YJR064W | 1764 | 1,782 | 29,691 | 16.66 |
| YBL035C | 215 | 2,364 | 2,855 | 1.21 | YJR065C | 1620 | 1,490 | 16,777 | 11.26 |
| YBL036C | 214 | 924 | 1,773 | 1.92 | YJR067C | 1619 | 525 | 538 | 1.02 |
| YBL038W | 249 | 812 | 1,561 | 1.92 | YJR068W | 1765 | 1,326 | 2,572 | 1.94 |
| YBL039C | 213 | 1,875 | 25,229 | 13.46 | YJR069C | 1618 | 755 | 2,223 | 2.94 |
| YBL040C | 212 | 761 | 12,537 | 16.47 | YJR070C | 1617 | 1,086 | 15,179 | 13.98 |
| YBL041W | 250 | 816 | 6,338 | 7.77 | YJR072C | 1616 | 1,221 | 5,525 | 4.52 |
| YBL042C | 211 | 2,138 | 5,298 | 2.48 | YJR073C | 1615 | 711 | 5,373 | 7.56 |
| YBL045C | 210 | 1,733 | 9,414 | 5.43 | YJR074W | 1766 | 762 | 1,768 | 2.32 |
| YBL047C | 209 | 4,513 | 13,645 | 3.02 | YJR075W | 1767 | 1,438 | 13,303 | 9.25 |
| YBL050W | 251 | 1,017 | 3,739 | 3.68 | YJR076C | 1614 | 1,410 | 2,537 | 1.80 |
| YBL054W | 252 | 1,874 | 3,002 | 1.60 | YJR077C | 1613 | 1,211 | 16,128 | 13.32 |
| YBL055C | 208 | 1,421 | 2,508 | 1.76 | YJR080C | 1612 | 1,328 | 2,003 | 1.51 |
| YBL056W | 253 | 1,882 | 9,388 | 5.00 | YJR085C | 1611 | 386 | 6,297 | 16.31 |
| YBL057C | 207 | 702 | 4,740 | 6.75 | YJR086W | 1768 | 401 | 467 | 1.20 |
| YBL058W | 254 | 1,419 | 12,088 | 8.52 | YJR088C | 1610 | 1,015 | 1,884 | 1.86 |
| YBL059C-A | 206 | 563 | 1,260 | 2.24 | YJR091C | 1609 | 3,457 | 4,397 | 1.27 |
| YBL061C | 205 | 2,271 | 2,900 | 1.28 | YJR094W-A | 1769 | 450 | 50,650 | 122.30 |
| YBL064C | 204 | 868 | 3,360 | 3.87 | YJR096W | 1770 | 1,052 | 1,593 | 1.51 |
| YBL068W | 255 | 1,122 | 4,617 | 4.12 | YJR100C | 1608 | 1,093 | 1,110 | 1.01 |
| YBL069W | 256 | 1,429 | 2,752 | 1.93 | YJR101W | 1771 | 1,027 | 2,768 | 2.70 |
| YBL071W-A | 257 | 533 | 2,257 | 4.23 | YJR103W | 1772 | 1,896 | 6,236 | 3.29 |
| YBL072C | 203 | 1,003 | 29,468 | 56.82 | YJR104C | 1607 | 542 | 79,233 | 146.19 |
| YBL076C | 202 | 3,301 | 44,561 | 13.50 | YJR105W | 1773 | 1,103 | 81,270 | 73.68 |
| YBL079W | 258 | 4,816 | 4,859 | 1.01 | YJR109C | 1606 | 3,526 | 4,263 | 1.21 |
| YBL082C | 201 | 1,377 | 4,958 | 3.60 | YJR112W-A | 1774 | 462 | 1,697 | 3.67 |
| YBL087C | 200 | 504 | 38,789 | 119.98 | YJR113C | 1605 | 858 | 3,884 | 4.53 |
| YBL089W | 259 | 1,475 | 2,001 | 1.36 | YJR116W | 1775 | 1,189 | 2,130 | 1.83 |
| YBL091C | 199 | 1,367 | 7,109 | 5.20 | YJR117W | 1776 | 1,494 | 10,531 | 7.05 |
| YBL092W | 260 | 797 | 109,788 | 137.82 | YJR118C | 1604 | 760 | 2,320 | 3.05 |
| YBL093C | 198 | 905 | 2,149 | 2.38 | YJR120W | 1777 | 351 | 863 | 2.46 |
| YBL095W | 261 | 933 | 1,182 | 1.27 | YJR121W | 1778 | 1,607 | 56,586 | 35.22 |
| YBL098W | 262 | 1,529 | 2,317 | 1.52 | YJR123W | 32 | 844 | 341,441 | 406.40 |
| YBL099W | 263 | 2,203 | 16,989 | 7.71 | YJR124C | 1603 | 1,628 | 5,713 | 3.51 |
| YBL102W | 264 | 887 | 5,631 | 6.35 | YJR125C | 1602 | 1,352 | 5,034 | 3.72 |
| YBR002C | 197 | 1,000 | 1,135 | 1.14 | YJR126C | 1601 | 2,436 | 4,160 | 1.71 |
| YBR003W | 265 | 1,575 | 2,783 | 1.77 | YJR131W | 1779 | 1,706 | 2,426 | 1.42 |
| YBR004C | 196 | 1,447 | 1,770 | 1.22 | YJR132W | 1780 | 3,388 | 6,091 | 1.80 |
| YBR005W | 266 | 823 | 1,055 | 1.28 | YJR133W | 1781 | 827 | 3,363 | 4.07 |
| YBR009C | 195 | 484 | 9,431 | 29.11 | YJR135W-A | 1782 | 386 | 1,433 | 3.71 |
| YBR010W | 267 | 582 | 20,844 | 45.89 | YJR137C | 1600 | 4,436 | 6,256 | 1.41 |
| YBR011C | 194 | 965 | 46,247 | 47.92 | YJR139C | 1599 | 1,208 | 57,459 | 48.69 |
| YBR014C | 193 | 760 | 2,525 | 3.32 | YJR141W | 1783 | 1,189 | 1,242 | 1.04 |
| YBR015C | 192 | 2,031 | 11,419 | 5.62 | YJR142W | 1784 | 1,143 | 1,440 | 1.26 |
| YBR016W | 268 | 633 | 2,457 | 3.88 | YJR143C | 1598 | 2,463 | 30,081 | 12.21 |
| YBR017C | 191 | 2,883 | 5,637 | 1.96 | YJR144W | 1785 | 901 | 4,198 | 4.66 |
| YBR022W | 269 | 769 | 1,260 | 1.64 | YJR145C | 1597 | 876 | 30,916 | 104.25 |
| YBR023C | 190 | 3,668 | 12,932 | 3.53 | YJR147W | 1786 | 1,425 | 3,274 | 2.30 |
| YBR025C | 189 | 1,408 | 26,229 | 18.63 | YJR148W | 1787 | 1,240 | 26,603 | 21.46 |
| YBR026C | 188 | 1,235 | 2,922 | 2.37 | YKL001C | 1867 | 690 | 2,793 | 4.05 |
| YBR028C | 187 | 1,578 | 2,344 | 1.48 | YKL002W | 1879 | 855 | 1,789 | 2.09 |
| YBR029C | 186 | 2,180 | 6,534 | 3.00 | YKL003C | 1866 | 485 | 909 | 1.87 |
| YBR030W | 270 | 1,838 | 2,673 | 1.47 | YKL004W | 1880 | 1,698 | 17,427 | 10.26 |
| YBR031W | 271 | 1,353 | 31,080 | 77.78 | YKL006C-A | 1865 | 366 | 1,243 | 3.40 |
| YBR034C | 185 | 1,223 | 8,612 | 7.04 | YKL006W | 1881 | 556 | 33,582 | 105.55 |
| YBR035C | 184 | 803 | 6,038 | 7.52 | YKL007W | 1882 | 967 | 2,915 | 3.01 |
| YBR036C | 183 | 1,339 | 18,960 | 14.16 | YKL008C | 1864 | 1,464 | 14,995 | 10.24 |
| YBR037C | 182 | 960 | 1,756 | 1.83 | YKL009W | 1883 | 847 | 4,365 | 5.15 |
| YBR038W | 272 | 3,158 | 5,765 | 1.83 | YKL013C | 1863 | 697 | 9,426 | 13.52 |
| YBR039W | 273 | 1,208 | 6,780 | 5.61 | YKL016C | 1862 | 663 | 5,919 | 8.93 |
| YBR041W | 274 | 2,240 | 8,469 | 3.78 | YKL018W | 1884 | 1,162 | 3,462 | 2.98 |
| YBR042C | 181 | 1,285 | 4,717 | 3.67 | YKL019W | 1885 | 1,103 | 5,597 | 5.07 |
| YBR043C | 180 | 2,345 | 4,837 | 2.06 | YKL021C | 1861 | 1,473 | 1,892 | 1.28 |
| YBR046C | 179 | 1,082 | 1,228 | 1.13 | YKL024C | 1860 | 923 | 7,323 | 9.19 |
| YBR048W | 275 | 709 | 30,820 | 59.95 | YKL025C | 1859 | 2,082 | 2,249 | 1.08 |
| YBR052C | 178 | 709 | 7,026 | 9.91 | YKL027W | 1886 | 1,453 | 2,450 | 1.69 |
| YBR053C | 177 | 1,171 | 3,416 | 2.92 | YKL028W | 1887 | 1,789 | 1,828 | 1.03 |
| YBR054W | 276 | 1,713 | 3,653 | 2.14 | YKL029C | 1858 | 2,055 | 8,655 | 4.21 |
| YBR056W | 277 | 1,582 | 4,828 | 3.05 | YKL032C | 1857 | 2,111 | 2,193 | 1.08 |
| YBR058C | 176 | 2,393 | 3,951 | 1.65 | YKL033W-A | 1888 | 870 | 11,977 | 13.77 |
| YBR058C-A | 175 | 396 | 2,076 | 5.24 | YKL034W | 1889 | 2,394 | 3,240 | 1.35 |
| YBR061C | 174 | 1,144 | 2,193 | 1.92 | YKL035W | 1890 | 1,742 | 38,209 | 21.93 |
| YBR062C | 173 | 637 | 1,103 | 1.73 | YKL039W | 1891 | 1,966 | 6,145 | 3.12 |
| YBR067C | 172 | 773 | 16,386 | 21.44 | YKL040C | 1856 | 1,428 | 3,179 | 2.23 |
| YBR068C | 171 | 2,082 | 12,769 | 6.13 | YKL043W | 1892 | 1,259 | 3,288 | 2.61 |
| YBR069C | 170 | 2,068 | 28,207 | 13.65 | YKL045W | 1893 | 1,783 | 1,959 | 1.10 |
| YBR070C | 169 | 797 | 2,059 | 2.58 | YKL046C | 1855 | 1,609 | 9,714 | 6.04 |
| YBR071W | 278 | 957 | 1,666 | 1.74 | YKL047W | 1894 | 1,748 | 3,050 | 1.74 |
| YBR072W | 279 | 870 | 1,392 | 1.60 | YKL051W | 1895 | 1,616 | 2,892 | 1.79 |
| YBR073W | 280 | 2,914 | 3,674 | 1.26 | YKL052C | 1854 | 929 | 1,199 | 1.29 |
| YBR074W | 281 | 2,962 | 8,492 | 2.87 | YKL053C-A | 1853 | 424 | 1,788 | 4.22 |
| YBR077C | 168 | 814 | 17,955 | 22.06 | YKL054C | 1852 | 2,490 | 3,264 | 1.35 |
| YBR078W | 282 | 1,611 | 71,735 | 44.53 | YKL056C | 1851 | 765 | 380,923 | 498.20 |
| YBR079C | 167 | 2,985 | 20,931 | 7.01 | YKL057C | 1850 | 3,186 | 4,441 | 1.39 |
| YBR080C | 166 | 2,420 | 5,725 | 2.36 | YKL058W | 1896 | 747 | 3,153 | 4.22 |
| YBR082C | 165 | 778 | 8,425 | 10.83 | YKL060C | 34 | 1,119 | 1,132,099 | 1011.71 |
| YBR084C-A | 164 | 796 | 34,075 | 57.33 | YKL063C | 1849 | 814 | 977 | 1.20 |
| YBR084W | 283 | 3,160 | 22,587 | 7.15 | YKL065C | 1848 | 681 | 5,018 | 7.37 |
| YBR085C-A | 163 | 649 | 1,586 | 2.44 | YKL067W | 1897 | 714 | 6,952 | 9.74 |
| YBR085W | 284 | 1,182 | 1,203 | 1.02 | YKL068W | 1898 | 3,117 | 4,758 | 1.53 |
| YBR086C | 162 | 3,183 | 16,272 | 5.11 | YKL068W-A | 1899 | 615 | 626 | 1.02 |
| YBR087W | 285 | 1,130 | 3,881 | 3.43 | YKL069W | 1900 | 608 | 1,817 | 2.99 |
| YBR088C | 161 | 805 | 5,687 | 7.06 | YKL073W | 1901 | 2,833 | 5,644 | 1.99 |
| YBR089C-A | 160 | 873 | 2,351 | 2.98 | YKL077W | 1902 | 1,490 | 16,833 | 11.30 |
| YBR091C | 159 | 444 | 1,118 | 2.52 | YKL080W | 1903 | 1,301 | 21,840 | 16.79 |
| YBR092C | 158 | 1,440 | 21,746 | 18.70 | YKL081W | 1904 | 1,474 | 109,763 | 75.30 |
| YBR093C | 157 | 1,488 | 9,946 | 8.77 | YKL084W | 1905 | 516 | 1,590 | 3.11 |
| YBR094W | 286 | 2,409 | 2,729 | 1.13 | YKL085W | 1906 | 1,511 | 11,190 | 7.42 |
| YBR096W | 287 | 847 | 7,305 | 8.62 | YKL087C | 1847 | 949 | 1,349 | 1.42 |
| YBR101C | 156 | 922 | 2,063 | 2.24 | YKL094W | 1907 | 1,057 | 5,041 | 4.77 |
| YBR103W | 288 | 1,608 | 2,013 | 1.25 | YKL096W | 1908 | 866 | 7,857 | 9.07 |
| YBR104W | 289 | 1,137 | 2,656 | 2.34 | YKL096W-A | 1909 | 500 | 50,871 | 103.45 |
| YBR106W | 290 | 753 | 84,557 | 112.29 | YKL098W | 1910 | 1,195 | 1,503 | 1.26 |
| YBR109C | 155 | 644 | 9,243 | 14.35 | YKL099C | 1846 | 906 | 1,137 | 1.25 |
| YBR110W | 291 | 1,451 | 2,914 | 2.01 | YKL100C | 1845 | 1,875 | 6,622 | 3.53 |
| YBR111C | 154 | 779 | 9,472 | 12.16 | YKL103C | 1844 | 1,763 | 3,507 | 1.99 |
| YBR111W-A | 292 | 475 | 592 | 1.25 | YKL104C | 1843 | 2,549 | 14,516 | 5.69 |
| YBR112C | 153 | 3,188 | 4,248 | 1.40 | YKL106W | 1911 | 1,604 | 5,058 | 3.15 |
| YBR115C | 152 | 4,322 | 7,008 | 1.62 | YKL110C | 1842 | 1,237 | 3,331 | 2.69 |
| YBR118W | 293 | 1,479 | 8,363 | 58.90 | YKL112W | 1912 | 3,571 | 3,650 | 1.02 |
| YBR121C | 151 | 2,166 | 27,078 | 12.50 | YKL113C | 1841 | 1,403 | 1,597 | 1.14 |
| YBR122C | 150 | 740 | 2,171 | 2.93 | YKL116C | 1840 | 1,983 | 3,830 | 1.93 |
| YBR125C | 149 | 1,528 | 2,569 | 1.68 | YKL117W | 1913 | 827 | 13,999 | 17.03 |
| YBR126C | 148 | 1,653 | 6,507 | 3.94 | YKL119C | 1839 | 753 | 1,599 | 2.12 |
| YBR127C | 147 | 1,748 | 26,509 | 15.16 | YKL120W | 1914 | 1,249 | 1,527 | 1.22 |
| YBR129C | 146 | 1,136 | 1,514 | 1.33 | YKL122C | 1838 | 593 | 1,147 | 1.93 |
| YBR133C | 145 | 2,549 | 7,447 | 2.92 | YKL126W | 1915 | 2,293 | 13,135 | 5.73 |
| YBR135W | 294 | 658 | 2,608 | 4.10 | YKL127W | 1916 | 1,877 | 10,679 | 5.70 |
| YBR137W | 295 | 895 | 1,654 | 1.85 | YKL128C | 1837 | 1,043 | 5,078 | 4.87 |
| YBR139W | 296 | 1,724 | 5,021 | 2.91 | YKL130C | 1836 | 903 | 2,791 | 3.09 |
| YBR142W | 297 | 2,510 | 3,025 | 1.20 | YKL135C | 1835 | 2,334 | 4,588 | 1.97 |
| YBR143C | 144 | 1,452 | 12,300 | 8.47 | YKL137W | 1917 | 540 | 1,259 | 2.33 |
| YBR145W | 298 | 1,219 | 1,321 | 1.09 | YKL138C | 1834 | 546 | 792 | 1.45 |
| YBR146W | 299 | 1,103 | 3,644 | 3.30 | YKL138C-A | 1833 | 291 | 329 | 1.13 |
| YBR149W | 300 | 1,166 | 12,941 | 11.10 | YKL140W | 1918 | 1,972 | 3,220 | 1.63 |
| YBR151W | 301 | 1,232 | 5,814 | 4.72 | YKL141W | 1919 | 719 | 7,488 | 10.41 |
| YBR154C | 143 | 832 | 2,700 | 3.25 | YKL142W | 1920 | 849 | 5,132 | 6.05 |
| YBR158W | 302 | 2,062 | 13,665 | 6.63 | YKL144C | 1832 | 1,366 | 2,158 | 1.58 |
| YBR159W | 303 | 1,272 | 12,769 | 10.07 | YKL145W | 1921 | 1,618 | 13,819 | 8.54 |
| YBR160W | 304 | 1,054 | 3,142 | 2.98 | YKL146W | 1922 | 2,183 | 5,354 | 2.45 |
| YBR162C | 142 | 1,759 | 41,584 | 23.75 | YKL148C | 1831 | 2,281 | 5,256 | 2.30 |
| YBR162W-A | 305 | 363 | 1,564 | 4.31 | YKL150W | 1923 | 1,163 | 5,186 | 4.46 |
| YBR164C | 141 | 766 | 2,679 | 3.50 | YKL151C | 1830 | 1,353 | 4,482 | 3.33 |
| YBR165W | 306 | 954 | 1,389 | 1.46 | YKL152C | 35 | 872 | 988,496 | 1133.60 |
| YBR166C | 140 | 1,490 | 2,110 | 1.42 | YKL154W | 1924 | 866 | 1,474 | 1.70 |
| YBR170C | 139 | 1,913 | 3,055 | 1.60 | YKL156W | 1925 | 468 | 46,608 | 102.92 |
| YBR171W | 307 | 776 | 1,751 | 2.26 | YKL157W | 1926 | 3,034 | 14,363 | 4.74 |
| YBR172C | 138 | 2,430 | 3,246 | 1.33 | YKL160W | 1927 | 527 | 1,912 | 3.63 |
| YBR173C | 137 | 510 | 2,605 | 5.11 | YKL163W | 1928 | 1,600 | 1,611 | 1.12 |
| YBR175W | 308 | 1,008 | 4,416 | 4.38 | YKL164C | 1829 | 1,336 | 20,454 | 21.36 |
| YBR177C | 136 | 1,473 | 8,000 | 5.43 | YKL165C | 1828 | 2,858 | 7,838 | 2.74 |
| YBR181C | 135 | 810 | 11,269 | 59.27 | YKL166C | 1827 | 1,386 | 2,541 | 1.83 |
| YBR183W | 309 | 1,268 | 1,275 | 1.01 | YKL167C | 1826 | 553 | 671 | 1.21 |
| YBR185C | 134 | 1,004 | 2,046 | 2.04 | YKL170W | 1929 | 630 | 1,158 | 1.84 |
| YBR187W | 310 | 1,047 | 21,173 | 20.22 | YKL172W | 1930 | 1,400 | 1,519 | 1.09 |
| YBR188C | 133 | 539 | 611 | 1.13 | YKL174C | 1825 | 1,982 | 3,084 | 1.56 |
| YBR189W | 311 | 682 | 96,130 | 181.52 | YKL175W | 1931 | 1,633 | 8,633 | 5.29 |
| YBR191W | 312 | 510 | 27,158 | 92.52 | YKL176C | 1824 | 2,870 | 9,995 | 3.48 |
| YBR193C | 132 | 963 | 1,402 | 1.46 | YKL178C | 1823 | 1,413 | 26,412 | 18.69 |
| YBR195C | 131 | 1,359 | 2,482 | 1.83 | YKL180W | 1932 | 691 | 43,083 | 76.45 |
| YBR196C | 130 | 1,846 | 198,210 | 107.37 | YKL181W | 1933 | 1,553 | 30,359 | 19.55 |
| YBR197C | 129 | 820 | 1,383 | 1.69 | YKL182W | 1934 | 6,564 | 96,019 | 14.63 |
| YBR199W | 313 | 1,599 | 8,999 | 5.63 | YKL183W | 1935 | 1,031 | 3,157 | 3.06 |
| YBR200W | 314 | 1,926 | 2,533 | 1.31 | YKL184W | 1936 | 1,625 | 6,923 | 4.26 |
| YBR202W | 315 | 2,778 | 4,267 | 1.54 | YKL186C | 1822 | 999 | 2,811 | 2.81 |
| YBR205W | 316 | 1,215 | 13,342 | 10.98 | YKL190W | 1937 | 731 | 2,297 | 3.14 |
| YBR207W | 317 | 1,550 | 4,175 | 2.69 | YKL191W | 1938 | 1,708 | 11,475 | 6.72 |
| YBR210W | 318 | 672 | 1,584 | 2.39 | YKL192C | 1821 | 522 | 8,634 | 16.56 |
| YBR211C | 128 | 1,166 | 1,193 | 1.03 | YKL194C | 1820 | 1,796 | 2,135 | 1.19 |
| YBR212W | 319 | 2,392 | 2,939 | 1.24 | YKL195W | 1939 | 1,364 | 2,064 | 1.51 |
| YBR214W | 320 | 1,848 | 3,838 | 2.08 | YKL196C | 1819 | 777 | 8,637 | 11.12 |
| YBR218C | 127 | 3,778 | 11,277 | 3.17 | YKL205W | 1940 | 3,536 | 3,557 | 1.01 |
| YBR220C | 126 | 1,958 | 7,869 | 4.36 | YKL206C | 1818 | 887 | 2,353 | 2.65 |
| YBR221C | 125 | 1,518 | 44,619 | 29.40 | YKL207W | 1941 | 837 | 11,416 | 13.64 |
| YBR222C | 124 | 1,846 | 19,866 | 10.76 | YKL210W | 1942 | 3,212 | 26,820 | 8.35 |
| YBR229C | 123 | 2,991 | 6,520 | 2.18 | YKL211C | 1817 | 1,635 | 14,324 | 8.78 |
| YBR230C | 122 | 430 | 2,612 | 6.07 | YKL212W | 1943 | 2,304 | 14,136 | 6.15 |
| YBR230W-A | 321 | 383 | 1,039 | 2.71 | YKL213C | 1816 | 2,237 | 3,143 | 1.40 |
| YBR231C | 121 | 1,011 | 1,085 | 1.07 | YKL214C | 1815 | 708 | 1,733 | 2.45 |
| YBR233W-A | 322 | 560 | 635 | 1.13 | YKL216W | 1944 | 1,216 | 8,056 | 6.62 |
| YBR234C | 120 | 1,247 | 9,436 | 7.57 | YKL219W | 1945 | 1,224 | 1,909 | 1.67 |
| YBR235W | 323 | 3,525 | 5,961 | 1.69 | YKR001C | 1814 | 2,269 | 10,043 | 4.43 |
| YBR236C | 119 | 1,485 | 2,473 | 1.66 | YKR003W | 1946 | 1,475 | 2,005 | 1.36 |
| YBR241C | 118 | 1,599 | 2,399 | 1.50 | YKR006C | 1813 | 864 | 2,481 | 2.87 |
| YBR242W | 324 | 932 | 2,428 | 2.60 | YKR007W | 1947 | 646 | 1,219 | 1.89 |
| YBR243C | 117 | 1,347 | 4,294 | 3.19 | YKR013W | 1948 | 1,229 | 29,669 | 24.16 |
| YBR244W | 325 | 649 | 1,275 | 1.96 | YKR014C | 1812 | 933 | 1,784 | 1.91 |
| YBR246W | 326 | 1,295 | 4,651 | 3.59 | YKR018C | 1811 | 2,357 | 7,218 | 3.06 |
| YBR247C | 116 | 1,550 | 1,942 | 1.25 | YKR025W | 1949 | 989 | 1,930 | 1.95 |
| YBR248C | 115 | 1,790 | 4,899 | 2.74 | YKR026C | 1810 | 1,147 | 2,970 | 2.59 |
| YBR249C | 114 | 1,338 | 41,908 | 31.32 | YKR028W | 1950 | 3,428 | 4,788 | 1.40 |
| YBR251W | 327 | 1,044 | 1,502 | 1.44 | YKR030W | 1951 | 962 | 1,219 | 1.27 |
| YBR252W | 328 | 522 | 8,405 | 16.10 | YKR035W-A | 1952 | 756 | 810 | 1.07 |
| YBR253W | 329 | 506 | 812 | 1.60 | YKR037C | 1809 | 1,012 | 1,395 | 1.38 |
| YBR254C | 113 | 672 | 947 | 1.41 | YKR038C | 1808 | 1,574 | 5,467 | 3.54 |
| YBR256C | 112 | 769 | 3,838 | 4.99 | YKR042W | 1953 | 1,528 | 48,179 | 31.56 |
| YBR260C | 111 | 2,123 | 2,133 | 1.00 | YKR043C | 1807 | 980 | 11,353 | 11.58 |
| YBR261C | 110 | 834 | 4,709 | 5.65 | YKR044W | 1954 | 1,459 | 2,750 | 1.89 |
| YBR262C | 109 | 420 | 1,634 | 3.89 | YKR045C | 1806 | 621 | 1,000 | 1.62 |
| YBR263W | 330 | 1,473 | 11,112 | 7.55 | YKR046C | 1805 | 1,040 | 1,510 | 1.47 |
| YBR264C | 108 | 735 | 764 | 1.04 | YKR048C | 1804 | 1,377 | 15,872 | 11.53 |
| YBR265W | 331 | 1,143 | 5,770 | 5.05 | YKR049C | 1803 | 452 | 1,705 | 3.77 |
| YBR267W | 332 | 1,309 | 2,958 | 2.26 | YKR051W | 1955 | 1,706 | 1,912 | 1.12 |
| YBR268W | 333 | 420 | 1,841 | 4.38 | YKR052C | 1802 | 1,313 | 1,867 | 1.42 |
| YBR269C | 107 | 549 | 1,822 | 3.32 | YKR056W | 1956 | 2,228 | 3,485 | 1.59 |
| YBR272C | 106 | 1,611 | 1,860 | 1.15 | YKR057W | 36 | 477 | 100,574 | 247.73 |
| YBR273C | 105 | 1,438 | 2,089 | 1.45 | YKR059W | 1957 | 1,319 | 8,734 | 28.85 |
| YBR274W | 334 | 1,823 | 2,194 | 1.20 | YKR062W | 1958 | 1,166 | 2,291 | 1.96 |
| YBR276C | 104 | 2,551 | 4,403 | 1.73 | YKR065C | 1801 | 702 | 4,206 | 5.99 |
| YBR278W | 335 | 699 | 835 | 1.19 | YKR066C | 1800 | 1,210 | 10,191 | 8.46 |
| YBR283C | 103 | 1,760 | 43,703 | 24.83 | YKR068C | 1799 | 769 | 1,750 | 2.27 |
| YBR286W | 336 | 1,697 | 88,041 | 51.91 | YKR070W | 1959 | 1,250 | 4,002 | 3.20 |
| YBR287W | 337 | 1,373 | 4,801 | 3.50 | YKR071C | 1798 | 1,341 | 2,349 | 1.75 |
| YBR288C | 102 | 1,560 | 3,343 | 2.14 | YKR074W | 1960 | 625 | 3,853 | 6.17 |
| YBR291C | 101 | 1,091 | 2,217 | 2.03 | YKR079C | 1797 | 2,658 | 3,628 | 1.37 |
| YBR293W | 338 | 2,038 | 2,529 | 1.24 | YKR080W | 1961 | 1,265 | 3,750 | 2.96 |
| YCL001W | 403 | 673 | 3,308 | 4.91 | YKR081C | 1796 | 1,139 | 1,945 | 1.71 |
| YCL002C | 396 | 898 | 3,001 | 3.34 | YKR082W | 1962 | 3,576 | 3,918 | 1.10 |
| YCL005W | 404 | 929 | 4,430 | 4.77 | YKR084C | 1795 | 1,959 | 3,889 | 1.99 |
| YCL005W-A | 405 | 326 | 3,580 | 10.98 | YKR085C | 1794 | 631 | 3,765 | 5.97 |
| YCL008C | 395 | 1,274 | 2,753 | 2.16 | YKR087C | 1793 | 1,156 | 1,861 | 1.61 |
| YCL009C | 394 | 1,003 | 27,839 | 27.75 | YKR088C | 1792 | 1,078 | 4,880 | 4.53 |
| YCL010C | 393 | 873 | 2,023 | 2.32 | YKR089C | 1791 | 2,851 | 3,324 | 1.17 |
| YCL011C | 392 | 1,385 | 10,091 | 7.29 | YKR092C | 1790 | 1,369 | 5,236 | 3.94 |
| YCL012C | 391 | 585 | 647 | 1.11 | YKR093W | 1963 | 2,082 | 7,083 | 3.40 |
| YCL016C | 390 | 1,312 | 1,849 | 1.41 | YKR094C | 1789 | 557 | 31,154 | 91.38 |
| YCL017C | 389 | 1,713 | 7,931 | 4.63 | YKR095W-A | 1964 | 428 | 540 | 1.26 |
| YCL018W | 406 | 1,095 | 3,990 | 3.64 | YKR100C | 1788 | 1,359 | 1,649 | 1.21 |
| YCL025C | 388 | 2,164 | 3,169 | 1.47 | YLL001W | 2126 | 2,479 | 3,306 | 1.33 |
| YCL027W | 407 | 1,628 | 1,859 | 1.14 | YLL006W | 2127 | 1,356 | 1,596 | 1.18 |
| YCL028W | 408 | 1,292 | 6,857 | 5.31 | YLL008W | 2128 | 2,396 | 3,347 | 1.40 |
| YCL030C | 387 | 2,503 | 18,546 | 7.41 | YLL009C | 2094 | 335 | 889 | 2.65 |
| YCL031C | 386 | 1,015 | 1,095 | 1.08 | YLL010C | 2093 | 1,759 | 2,627 | 1.50 |
| YCL033C | 385 | 585 | 3,682 | 6.30 | YLL012W | 2129 | 1,907 | 3,150 | 1.65 |
| YCL034W | 409 | 1,269 | 4,504 | 3.56 | YLL013C | 2092 | 3,240 | 3,443 | 1.07 |
| YCL035C | 384 | 469 | 2,702 | 5.76 | YLL014W | 2130 | 515 | 4,713 | 9.15 |
| YCL036W | 410 | 1,790 | 2,963 | 1.66 | YLL018C | 2091 | 1,831 | 22,136 | 12.09 |
| YCL037C | 383 | 1,522 | 2,074 | 1.37 | YLL018C-A | 2090 | 415 | 1,374 | 3.31 |
| YCL038C | 382 | 1,587 | 1,838 | 1.16 | YLL019C | 2089 | 2,793 | 3,109 | 1.11 |
| YCL040W | 411 | 1,714 | 14,056 | 13.87 | YLL022C | 2088 | 1,288 | 1,554 | 1.21 |
| YCL043C | 381 | 1,682 | 96,962 | 57.67 | YLL023C | 2087 | 976 | 5,492 | 5.63 |
| YCL044C | 380 | 1,428 | 2,359 | 1.65 | YLL024C | 2086 | 2,057 | 53,059 | 71.99 |
| YCL045C | 379 | 2,359 | 16,054 | 6.80 | YLL026W | 2131 | 2,859 | 14,417 | 5.04 |
| YCL047C | 378 | 868 | 948 | 1.09 | YLL027W | 2132 | 948 | 1,626 | 1.71 |
| YCL049C | 377 | 1,190 | 1,376 | 1.16 | YLL028W | 2133 | 2,062 | 4,275 | 2.08 |
| YCL050C | 376 | 1,115 | 5,336 | 4.79 | YLL029W | 2134 | 2,475 | 4,872 | 1.97 |
| YCL052C | 375 | 1,437 | 1,900 | 1.32 | YLL031C | 2085 | 3,259 | 7,747 | 2.38 |
| YCL055W | 412 | 1,316 | 1,360 | 1.03 | YLL033W | 2135 | 693 | 785 | 1.13 |
| YCL057C-A | 374 | 420 | 3,948 | 9.40 | YLL035W | 2136 | 2,189 | 3,363 | 1.54 |
| YCL057W | 413 | 2,283 | 10,889 | 4.77 | YLL036C | 2084 | 1,594 | 2,025 | 1.27 |
| YCL059C | 373 | 1,070 | 1,845 | 1.72 | YLL038C | 2083 | 1,016 | 1,933 | 1.90 |
| YCL064C | 372 | 1,180 | 12,884 | 10.92 | YLL039C | 2082 | 1,353 | 7,510 | 7.78 |
| YCR002C | 371 | 1,042 | 3,911 | 3.75 | YLL041C | 2081 | 1,113 | 4,069 | 3.66 |
| YCR004C | 370 | 1,080 | 19,992 | 18.51 | YLL043W | 2137 | 2,103 | 5,317 | 2.54 |
| YCR005C | 369 | 1,555 | 3,421 | 2.20 | YLL045C | 2080 | 849 | 42,536 | 83.79 |
| YCR008W | 414 | 2,201 | 3,802 | 1.73 | YLL048C | 2079 | 5,243 | 14,241 | 2.72 |
| YCR009C | 368 | 922 | 6,224 | 6.75 | YLL049W | 2138 | 657 | 1,448 | 2.24 |
| YCR011C | 367 | 3,340 | 9,078 | 2.72 | YLL050C | 2078 | 547 | 45,655 | 83.53 |
| YCR012W | 10 | 1,460 | 641,453 | 440.20 | YLL051C | 2077 | 2,373 | 5,170 | 2.18 |
| YCR017C | 366 | 3,077 | 10,990 | 3.57 | YLL053C | 2076 | 460 | 463 | 1.02 |
| YCR018C | 365 | 806 | 918 | 1.14 | YLL058W | 2139 | 1,848 | 1,859 | 1.01 |
| YCR020C | 364 | 686 | 693 | 1.01 | YLL061W | 2140 | 1,814 | 3,249 | 1.79 |
| YCR020C-A | 363 | 421 | 1,406 | 3.34 | YLR002C | 2075 | 2,092 | 2,442 | 1.17 |
| YCR023C | 362 | 2,157 | 6,093 | 2.82 | YLR003C | 2074 | 978 | 993 | 1.01 |
| YCR024C-A | 361 | 123 | 4,160 | 38.62 | YLR005W | 2141 | 1,658 | 2,095 | 1.26 |
| YCR024C-B | 360 | 439 | 4,631 | 10.55 | YLR007W | 2142 | 1,077 | 2,549 | 2.37 |
| YCR026C | 359 | 2,507 | 4,219 | 1.68 | YLR008C | 2073 | 770 | 4,618 | 6.00 |
| YCR027C | 358 | 998 | 1,517 | 1.52 | YLR009W | 2143 | 912 | 2,046 | 2.24 |
| YCR028C | 357 | 1,768 | 6,106 | 3.45 | YLR016C | 2072 | 790 | 1,550 | 1.96 |
| YCR028C-A | 356 | 632 | 7,394 | 11.70 | YLR017W | 2144 | 1,130 | 10,688 | 9.46 |
| YCR030C | 355 | 2,828 | 5,135 | 1.81 | YLR018C | 2071 | 1,058 | 2,504 | 2.37 |
| YCR031C | 11 | 514 | 92,411 | 248.42 | YLR019W | 2145 | 1,422 | 3,435 | 2.41 |
| YCR034W | 415 | 1,240 | 18,764 | 15.13 | YLR020C | 2070 | 1,814 | 3,005 | 1.66 |
| YCR036W | 416 | 1,122 | 2,231 | 1.99 | YLR021W | 2146 | 615 | 1,202 | 1.95 |
| YCR037C | 354 | 2,772 | 4,426 | 1.60 | YLR022C | 2069 | 802 | 2,842 | 3.54 |
| YCR039C | 353 | 633 | 1,292 | 2.04 | YLR023C | 2068 | 1,886 | 2,531 | 1.34 |
| YCR040W | 417 | 528 | 2,146 | 4.06 | YLR027C | 2067 | 1,304 | 43,966 | 33.72 |
| YCR043C | 352 | 575 | 2,752 | 4.79 | YLR028C | 2066 | 2,036 | 26,655 | 13.09 |
| YCR044C | 351 | 1,194 | 6,256 | 5.24 | YLR029C | 2065 | 709 | 62,620 | 130.97 |
| YCR046C | 350 | 615 | 2,560 | 4.16 | YLR034C | 2064 | 1,707 | 4,714 | 2.76 |
| YCR047C | 349 | 1,063 | 2,095 | 1.97 | YLR038C | 2063 | 622 | 1,942 | 3.12 |
| YCR048W | 418 | 2,016 | 7,636 | 3.79 | YLR040C | 2062 | 798 | 10,939 | 13.71 |
| YCR051W | 419 | 809 | 3,862 | 4.77 | YLR043C | 37 | 416 | 80,872 | 195.04 |
| YCR052W | 420 | 1,683 | 5,179 | 3.08 | YLR044C | 38 | 1,778 | 650,942 | 457.33 |
| YCR053W | 421 | 1,633 | 48,458 | 29.69 | YLR046C | 2061 | 1,257 | 1,660 | 1.32 |
| YCR057C | 348 | 2,999 | 7,475 | 2.50 | YLR048W | 2147 | 1,176 | 70,470 | 68.95 |
| YCR059C | 347 | 953 | 10,295 | 10.89 | YLR049C | 2060 | 1,319 | 2,202 | 1.67 |
| YCR060W | 422 | 475 | 2,202 | 4.65 | YLR050C | 2059 | 548 | 4,029 | 7.35 |
| YCR061W | 423 | 2,314 | 4,107 | 1.77 | YLR052W | 2148 | 906 | 1,002 | 1.11 |
| YCR065W | 424 | 1,819 | 4,311 | 2.37 | YLR056W | 2149 | 1,282 | 50,139 | 39.11 |
| YCR067C | 346 | 3,399 | 6,895 | 2.04 | YLR058C | 2058 | 1,566 | 31,070 | 19.85 |
| YCR068W | 425 | 1,563 | 1,949 | 1.25 | YLR059C | 2057 | 980 | 3,948 | 4.03 |
| YCR069W | 426 | 1,287 | 10,322 | 8.02 | YLR060W | 2150 | 2,082 | 38,127 | 18.31 |
| YCR072C | 345 | 1,661 | 4,533 | 2.73 | YLR061W | 39 | 473 | 102,779 | 221.24 |
| YCR073W-A | 427 | 1,077 | 3,224 | 3.02 | YLR064W | 2151 | 950 | 3,440 | 3.62 |
| YCR075C | 344 | 899 | 3,409 | 3.82 | YLR065C | 2056 | 654 | 4,873 | 7.45 |
| YCR075W-A | 428 | 343 | 576 | 1.68 | YLR066W | 2152 | 702 | 3,802 | 5.42 |
| YCR076C | 343 | 856 | 1,104 | 1.29 | YLR069C | 2055 | 2,447 | 2,569 | 1.05 |
| YCR077C | 342 | 2,571 | 8,106 | 3.15 | YLR073C | 2054 | 713 | 1,775 | 2.50 |
| YCR082W | 429 | 516 | 4,468 | 8.67 | YLR074C | 2053 | 568 | 3,365 | 5.92 |
| YCR083W | 430 | 526 | 1,906 | 3.63 | YLR075W | 40 | 814 | 342,918 | 421.27 |
| YCR084C | 341 | 2,668 | 13,302 | 5.01 | YLR077W | 2153 | 1,925 | 2,769 | 1.44 |
| YCR087C-A | 340 | 648 | 1,312 | 2.02 | YLR078C | 2052 | 861 | 2,882 | 3.35 |
| YCR088W | 431 | 1,900 | 5,997 | 3.16 | YLR083C | 2051 | 2,168 | 15,233 | 7.03 |
| YCR090C | 339 | 624 | 4,335 | 6.95 | YLR084C | 2050 | 3,753 | 4,416 | 1.18 |
| YCR094W | 432 | 1,311 | 1,861 | 1.42 | YLR085C | 2049 | 1,412 | 1,499 | 1.06 |
| YDL004W | 627 | 768 | 4,326 | 5.63 | YLR088W | 2154 | 2,058 | 4,968 | 2.41 |
| YDL006W | 628 | 1,296 | 2,120 | 1.64 | YLR089C | 2048 | 2,144 | 5,791 | 2.70 |
| YDL007W | 629 | 1,434 | 4,929 | 3.44 | YLR090W | 2155 | 1,561 | 1,649 | 1.06 |
| YDL008W | 630 | 623 | 1,302 | 2.09 | YLR091W | 2156 | 1,224 | 1,425 | 1.16 |
| YDL012C | 616 | 467 | 1,245 | 2.67 | YLR093C | 2047 | 851 | 2,992 | 3.52 |
| YDL014W | 631 | 1,328 | 31,658 | 24.86 | YLR097C | 2046 | 1,035 | 1,041 | 1.01 |
| YDL015C | 615 | 1,173 | 18,030 | 15.37 | YLR099C | 2045 | 1,369 | 6,664 | 4.87 |
| YDL018C | 614 | 824 | 2,290 | 2.78 | YLR099W-A | 2157 | 345 | 2,995 | 8.68 |
| YDL019C | 613 | 4,158 | 4,202 | 1.01 | YLR100W | 2158 | 1,157 | 15,768 | 13.63 |
| YDL020C | 612 | 1,958 | 2,714 | 1.39 | YLR104W | 2159 | 500 | 1,066 | 2.17 |
| YDL022W | 632 | 1,380 | 16,892 | 12.24 | YLR105C | 2044 | 1,134 | 1,154 | 1.02 |
| YDL029W | 633 | 1,313 | 15,662 | 11.93 | YLR109W | 2160 | 630 | 86,884 | 137.91 |
| YDL040C | 611 | 2,680 | 8,581 | 3.20 | YLR110C | 1 | 507 | 339,952 | 673.06 |
| YDL045C | 610 | 1,090 | 1,221 | 1.12 | YLR113W | 2161 | 1,761 | 22,410 | 12.73 |
| YDL045W-A | 634 | 440 | 1,410 | 3.20 | YLR118C | 2043 | 800 | 3,652 | 4.56 |
| YDL046W | 635 | 859 | 9,394 | 10.94 | YLR120C | 2042 | 2,181 | 5,642 | 2.59 |
| YDL047W | 636 | 1,315 | 3,082 | 2.34 | YLR121C | 2041 | 1,829 | 2,253 | 1.23 |
| YDL051W | 637 | 961 | 10,972 | 11.42 | YLR126C | 2040 | 880 | 1,009 | 1.15 |
| YDL052C | 609 | 1,011 | 6,553 | 6.48 | YLR129W | 2162 | 3,042 | 5,766 | 1.89 |
| YDL053C | 608 | 898 | 1,411 | 1.58 | YLR130C | 2039 | 1,457 | 11,791 | 8.09 |
| YDL054C | 607 | 1,618 | 2,233 | 1.38 | YLR134W | 2163 | 1,929 | 12,574 | 7.90 |
| YDL055C | 606 | 1,458 | 208,464 | 142.98 | YLR137W | 2164 | 1,141 | 1,856 | 1.63 |
| YDL060W | 638 | 2,507 | 4,388 | 1.76 | YLR138W | 2165 | 3,174 | 3,808 | 1.20 |
| YDL061C | 605 | 760 | 31,236 | 42.76 | YLR141W | 2166 | 1,491 | 1,485 | 1.05 |
| YDL063C | 604 | 2,144 | 3,514 | 1.64 | YLR144C | 2038 | 2,400 | 3,213 | 1.34 |
| YDL064W | 639 | 594 | 3,209 | 5.40 | YLR146C | 2037 | 996 | 4,471 | 4.49 |
| YDL066W | 640 | 1,450 | 19,385 | 13.37 | YLR146W-A | 2167 | 286 | 1,023 | 3.58 |
| YDL067C | 603 | 449 | 5,551 | 12.36 | YLR147C | 2036 | 417 | 928 | 2.23 |
| YDL070W | 641 | 2,072 | 3,657 | 1.76 | YLR150W | 2168 | 1,052 | 120,912 | 114.93 |
| YDL072C | 602 | 732 | 8,898 | 12.16 | YLR153C | 2035 | 2,344 | 30,071 | 12.83 |
| YDL075W | 642 | 433 | 49,191 | 133.27 | YLR154C | 2034 | 450 | 1,648 | 3.66 |
| YDL076C | 601 | 1,178 | 1,530 | 1.30 | YLR163C | 2033 | 1,569 | 2,416 | 1.54 |
| YDL078C | 600 | 1,145 | 7,434 | 6.49 | YLR164W | 2169 | 749 | 885 | 1.18 |
| YDL081C | 12 | 452 | 218,001 | 508.52 | YLR167W | 41 | 568 | 311,636 | 686.85 |
| YDL082W | 643 | 769 | 53,423 | 87.18 | YLR170C | 2032 | 708 | 832 | 1.17 |
| YDL083C | 599 | 550 | 52,806 | 125.61 | YLR172C | 2031 | 975 | 6,758 | 6.93 |
| YDL084W | 644 | 1,608 | 55,652 | 34.61 | YLR175W | 2170 | 1,594 | 16,096 | 10.15 |
| YDL085C-A | 598 | 490 | 982 | 2.02 | YLR177W | 2171 | 1,993 | 2,686 | 1.36 |
| YDL086W | 645 | 908 | 5,398 | 5.94 | YLR178C | 2030 | 792 | 1,738 | 2.19 |
| YDL087C | 597 | 786 | 856 | 1.09 | YLR179C | 2029 | 705 | 17,967 | 25.48 |
| YDL088C | 596 | 1,877 | 2,283 | 1.22 | YLR180W | 2172 | 1,453 | 57,943 | 41.75 |
| YDL090C | 595 | 1,327 | 1,698 | 1.28 | YLR183C | 2028 | 1,684 | 2,292 | 1.36 |
| YDL092W | 646 | 588 | 1,931 | 3.28 | YLR185W | 2173 | 385 | 22,229 | 68.06 |
| YDL093W | 647 | 2,500 | 4,162 | 1.66 | YLR186W | 2174 | 883 | 6,334 | 7.17 |
| YDL095W | 648 | 2,632 | 30,849 | 11.72 | YLR188W | 2175 | 2,297 | 4,767 | 2.07 |
| YDL097C | 594 | 1,387 | 10,911 | 7.87 | YLR190W | 2176 | 1,906 | 6,908 | 3.62 |
| YDL100C | 593 | 1,147 | 22,266 | 19.41 | YLR192C | 2027 | 1,074 | 6,878 | 6.40 |
| YDL103C | 592 | 1,583 | 4,727 | 2.99 | YLR193C | 2026 | 747 | 1,430 | 1.91 |
| YDL108W | 649 | 1,046 | 1,664 | 1.59 | YLR194C | 2025 | 949 | 3,086 | 3.27 |
| YDL110C | 591 | 670 | 705 | 1.05 | YLR195C | 2024 | 1,666 | 6,253 | 3.77 |
| YDL111C | 590 | 798 | 4,160 | 5.21 | YLR196W | 2177 | 1,936 | 2,916 | 1.51 |
| YDL112W | 650 | 4,464 | 5,007 | 1.12 | YLR197W | 2178 | 1,741 | 17,699 | 10.18 |
| YDL116W | 651 | 2,289 | 3,310 | 1.45 | YLR199C | 2023 | 1,032 | 2,368 | 2.29 |
| YDL120W | 652 | 776 | 1,815 | 2.34 | YLR200W | 2179 | 507 | 559 | 1.10 |
| YDL121C | 589 | 641 | 3,560 | 5.55 | YLR201C | 2022 | 910 | 1,182 | 1.30 |
| YDL123W | 653 | 485 | 3,116 | 6.42 | YLR203C | 2021 | 1,473 | 5,980 | 4.06 |
| YDL124W | 654 | 1,068 | 5,573 | 5.22 | YLR204W | 2180 | 493 | 746 | 1.51 |
| YDL125C | 588 | 801 | 18,616 | 23.24 | YLR206W | 2181 | 1,989 | 3,697 | 1.88 |
| YDL126C | 587 | 2,607 | 48,211 | 18.50 | YLR207W | 2182 | 2,601 | 2,821 | 1.08 |
| YDL128W | 655 | 1,500 | 12,006 | 8.00 | YLR208W | 2183 | 1,256 | 13,918 | 11.08 |
| YDL130W | 656 | 433 | 53,427 | 138.87 | YLR209C | 2020 | 1,019 | 4,118 | 4.04 |
| YDL131W | 657 | 1,479 | 4,468 | 4.31 | YLR212C | 2019 | 1,723 | 3,170 | 1.84 |
| YDL132W | 658 | 2,817 | 4,394 | 1.56 | YLR214W | 2184 | 2,325 | 8,914 | 3.83 |
| YDL133C-A | 13 | 280 | 191,688 | 883.35 | YLR216C | 2018 | 1,180 | 13,045 | 11.05 |
| YDL134C | 586 | 1,625 | 6,681 | 4.63 | YLR219W | 2185 | 2,519 | 3,119 | 1.24 |
| YDL135C | 585 | 841 | 1,489 | 1.77 | YLR220W | 2186 | 1,134 | 4,365 | 3.85 |
| YDL136W | 659 | 528 | 3,405 | 15.48 | YLR221C | 2017 | 913 | 1,559 | 1.71 |
| YDL137W | 660 | 1,072 | 15,079 | 15.16 | YLR222C | 2016 | 2,553 | 4,356 | 1.71 |
| YDL140C | 584 | 5,350 | 14,504 | 2.72 | YLR224W | 2187 | 1,329 | 2,816 | 2.12 |
| YDL141W | 661 | 2,522 | 5,919 | 2.35 | YLR229C | 2015 | 642 | 8,237 | 12.83 |
| YDL143W | 662 | 1,728 | 15,402 | 8.92 | YLR231C | 2014 | 1,472 | 8,129 | 5.52 |
| YDL144C | 583 | 1,184 | 3,368 | 2.84 | YLR237W | 2188 | 1,797 | 2,222 | 1.28 |
| YDL145C | 582 | 3,983 | 20,628 | 5.18 | YLR241W | 2189 | 2,349 | 4,769 | 2.03 |
| YDL147W | 663 | 1,525 | 4,825 | 3.16 | YLR242C | 2013 | 966 | 1,535 | 1.59 |
| YDL155W | 664 | 1,545 | 3,815 | 2.47 | YLR243W | 2190 | 974 | 2,145 | 2.20 |
| YDL157C | 581 | 528 | 1,583 | 3.00 | YLR244C | 2012 | 1,323 | 18,589 | 14.05 |
| YDL160C | 580 | 1,852 | 5,494 | 2.97 | YLR245C | 2011 | 429 | 586 | 1.37 |
| YDL160C-A | 579 | 433 | 697 | 1.61 | YLR248W | 2191 | 2,110 | 10,095 | 4.78 |
| YDL164C | 578 | 2,452 | 4,018 | 1.64 | YLR249W | 42 | 3,263 | 638,371 | 198.69 |
| YDL165W | 665 | 662 | 2,251 | 3.40 | YLR250W | 2192 | 839 | 4,912 | 5.85 |
| YDL166C | 577 | 685 | 1,315 | 1.92 | YLR253W | 2193 | 1,899 | 2,059 | 1.08 |
| YDL167C | 576 | 2,355 | 2,887 | 1.23 | YLR256W | 2194 | 4,509 | 31,037 | 6.89 |
| YDL168W | 666 | 1,277 | 5,224 | 4.09 | YLR257W | 2195 | 1,245 | 11,840 | 9.51 |
| YDL171C | 575 | 6,585 | 18,155 | 2.76 | YLR258W | 2196 | 2,546 | 5,338 | 2.10 |
| YDL173W | 667 | 1,060 | 1,320 | 1.25 | YLR259C | 2010 | 1,826 | 29,028 | 15.90 |
| YDL174C | 574 | 1,892 | 5,191 | 2.74 | YLR262C | 2009 | 648 | 999 | 1.54 |
| YDL178W | 668 | 1,675 | 3,116 | 1.86 | YLR262C-A | 2008 | 290 | 5,198 | 17.92 |
| YDL180W | 669 | 2,063 | 2,954 | 1.43 | YLR264W | 2197 | 860 | 66,243 | 80.67 |
| YDL181W | 670 | 433 | 1,052 | 2.43 | YLR265C | 2007 | 1,149 | 1,521 | 1.32 |
| YDL182W | 671 | 1,401 | 6,626 | 6.93 | YLR266C | 2006 | 2,106 | 2,462 | 1.17 |
| YDL184C | 14 | 290 | 148,521 | 654.28 | YLR268W | 2198 | 800 | 4,154 | 5.19 |
| YDL185W | 672 | 3,346 | 61,574 | 18.40 | YLR270W | 2199 | 1,255 | 2,105 | 1.68 |
| YDL188C | 573 | 1,380 | 4,357 | 3.63 | YLR274W | 2200 | 2,472 | 3,515 | 1.42 |
| YDL190C | 572 | 3,197 | 5,524 | 1.73 | YLR276C | 2005 | 1,978 | 2,170 | 1.10 |
| YDL191W | 673 | 513 | 2,865 | 14.17 | YLR285W | 2201 | 1,026 | 1,769 | 1.72 |
| YDL192W | 674 | 641 | 22,378 | 39.72 | YLR286C | 2004 | 2,067 | 65,921 | 31.90 |
| YDL193W | 675 | 1,334 | 3,473 | 2.60 | YLR287C | 2003 | 1,370 | 2,741 | 2.00 |
| YDL195W | 676 | 4,069 | 14,368 | 3.53 | YLR287C-A | 2002 | 397 | 49,129 | 153.53 |
| YDL198C | 571 | 1,140 | 4,463 | 3.91 | YLR290C | 2001 | 931 | 934 | 1.00 |
| YDL201W | 677 | 1,003 | 1,955 | 1.95 | YLR291C | 2000 | 1,286 | 7,735 | 6.01 |
| YDL202W | 678 | 887 | 1,209 | 1.36 | YLR292C | 1999 | 735 | 3,916 | 5.33 |
| YDL203C | 570 | 2,192 | 2,676 | 1.22 | YLR293C | 1998 | 780 | 35,159 | 45.53 |
| YDL205C | 569 | 1,274 | 2,969 | 2.35 | YLR295C | 1997 | 650 | 3,138 | 4.83 |
| YDL208W | 679 | 625 | 6,852 | 10.96 | YLR300W | 2202 | 1,669 | 52,523 | 31.47 |
| YDL212W | 680 | 824 | 14,162 | 17.19 | YLR301W | 2203 | 897 | 8,293 | 9.24 |
| YDL213C | 568 | 745 | 1,057 | 1.42 | YLR303W | 2204 | 1,335 | 35,423 | 26.53 |
| YDL215C | 567 | 3,635 | 3,649 | 1.00 | YLR304C | 1996 | 2,651 | 47,811 | 18.04 |
| YDL217C | 566 | 925 | 1,543 | 1.67 | YLR305C | 1995 | 5,754 | 10,136 | 1.76 |
| YDL219W | 681 | 558 | 1,409 | 2.52 | YLR310C | 1994 | 4,967 | 5,243 | 1.06 |
| YDL224C | 565 | 2,454 | 3,977 | 1.62 | YLR312W-A | 2205 | 904 | 1,343 | 1.49 |
| YDL226C | 564 | 1,156 | 4,247 | 3.67 | YLR314C | 1993 | 1,716 | 3,070 | 1.79 |
| YDL227C | 563 | 1,761 | 2,517 | 1.43 | YLR315W | 2206 | 462 | 685 | 1.48 |
| YDL229W | 682 | 1,982 | 56,974 | 83.24 | YLR324W | 2207 | 1,859 | 2,117 | 1.14 |
| YDL230W | 683 | 1,122 | 1,379 | 1.23 | YLR325C | 1992 | 478 | 90,046 | 188.59 |
| YDL231C | 562 | 3,378 | 5,648 | 1.67 | YLR328W | 2208 | 1,560 | 3,946 | 2.53 |
| YDL232W | 684 | 419 | 4,811 | 11.48 | YLR330W | 2209 | 2,175 | 5,051 | 2.37 |
| YDL235C | 561 | 730 | 2,107 | 2.89 | YLR332W | 2210 | 1,745 | 2,801 | 1.60 |
| YDL236W | 685 | 1,148 | 6,418 | 5.61 | YLR333C | 1991 | 887 | 17,070 | 21.03 |
| YDL237W | 686 | 1,271 | 6,468 | 5.09 | YLR335W | 2211 | 2,275 | 4,098 | 1.80 |
| YDR001C | 560 | 2,542 | 3,756 | 1.48 | YLR340W | 2212 | 1,087 | 594,533 | 560.32 |
| YDR002W | 687 | 852 | 20,887 | 24.54 | YLR342W | 2213 | 6,138 | 75,364 | 12.39 |
| YDR003W | 688 | 747 | 1,132 | 1.52 | YLR344W | 2214 | 527 | 21,350 | 52.69 |
| YDR003W-A | 689 | 317 | 366 | 1.16 | YLR347C | 1990 | 2,981 | 13,149 | 4.41 |
| YDR007W | 690 | 902 | 3,302 | 3.66 | YLR348C | 1989 | 935 | 3,242 | 3.47 |
| YDR011W | 691 | 4,987 | 22,406 | 4.49 | YLR350W | 2215 | 783 | 4,618 | 5.93 |
| YDR012W | 692 | 1,204 | 11,497 | 43.13 | YLR351C | 1988 | 994 | 6,903 | 6.99 |
| YDR013W | 693 | 746 | 949 | 1.27 | YLR353W | 2216 | 2,017 | 2,436 | 1.21 |
| YDR016C | 559 | 387 | 1,816 | 4.69 | YLR354C | 1987 | 1,147 | 68,994 | 60.15 |
| YDR019C | 558 | 1,392 | 3,294 | 2.37 | YLR355C | 1986 | 1,445 | 73,439 | 50.82 |
| YDR020C | 557 | 1,173 | 1,906 | 1.63 | YLR356W | 2217 | 750 | 1,269 | 1.69 |
| YDR023W | 694 | 1,568 | 17,921 | 11.43 | YLR359W | 2218 | 1,643 | 20,143 | 12.26 |
| YDR025W | 695 | 577 | 24,991 | 65.33 | YLR361C | 1985 | 2,085 | 2,142 | 1.03 |
| YDR028C | 556 | 3,620 | 4,155 | 1.15 | YLR363C | 1984 | 831 | 896 | 1.08 |
| YDR032C | 555 | 784 | 32,062 | 40.90 | YLR363W-A | 2219 | 460 | 837 | 1.82 |
| YDR033W | 696 | 1,643 | 83,809 | 51.26 | YLR367W | 2220 | 1,017 | 19,783 | 20.97 |
| YDR034C-A | 554 | 207 | 335 | 1.65 | YLR370C | 1983 | 706 | 4,809 | 6.81 |
| YDR035W | 697 | 1,424 | 5,091 | 3.57 | YLR372W | 2221 | 1,285 | 21,483 | 16.72 |
| YDR036C | 553 | 1,681 | 3,256 | 1.94 | YLR375W | 2222 | 1,124 | 10,471 | 9.32 |
| YDR037W | 698 | 1,915 | 41,221 | 21.53 | YLR378C | 1982 | 1,793 | 21,924 | 12.23 |
| YDR041W | 699 | 778 | 1,279 | 1.64 | YLR380W | 2223 | 1,440 | 6,064 | 4.21 |
| YDR044W | 700 | 1,248 | 8,931 | 7.16 | YLR384C | 1981 | 4,176 | 8,012 | 1.92 |
| YDR045C | 552 | 642 | 1,686 | 2.63 | YLR387C | 1980 | 1,421 | 3,155 | 2.22 |
| YDR046C | 551 | 1,955 | 10,201 | 5.22 | YLR388W | 2224 | 740 | 33,396 | 46.93 |
| YDR047W | 701 | 1,362 | 5,193 | 3.81 | YLR389C | 1979 | 3,274 | 6,605 | 2.02 |
| YDR050C | 15 | 834 | 274,791 | 329.67 | YLR390W | 2225 | 615 | 1,031 | 1.68 |
| YDR051C | 550 | 1,078 | 1,157 | 1.07 | YLR390W-A | 2226 | 978 | 11,234 | 11.67 |
| YDR054C | 549 | 1,400 | 1,772 | 1.27 | YLR395C | 1978 | 563 | 2,854 | 5.07 |
| YDR055W | 702 | 1,545 | 8,109 | 5.25 | YLR397C | 1977 | 2,343 | 4,972 | 2.12 |
| YDR056C | 548 | 764 | 3,097 | 4.05 | YLR399C | 1976 | 2,271 | 4,948 | 2.18 |
| YDR059C | 547 | 590 | 1,255 | 2.13 | YLR401C | 1975 | 2,073 | 3,529 | 1.70 |
| YDR060W | 703 | 3,218 | 4,391 | 1.37 | YLR403W | 2227 | 2,437 | 4,700 | 1.94 |
| YDR061W | 704 | 1,788 | 2,069 | 1.16 | YLR404W | 2228 | 1,002 | 1,341 | 1.34 |
| YDR062W | 705 | 2,049 | 8,602 | 4.20 | YLR405W | 2229 | 1,253 | 1,784 | 1.42 |
| YDR063W | 706 | 633 | 1,703 | 2.69 | YLR406C | 1974 | 534 | 22,766 | 48.39 |
| YDR064W | 16 | 561 | 121,002 | 215.69 | YLR407W | 2230 | 1,151 | 1,217 | 1.06 |
| YDR071C | 546 | 714 | 7,377 | 10.33 | YLR409C | 1973 | 2,878 | 6,658 | 2.31 |
| YDR072C | 545 | 2,326 | 6,470 | 2.78 | YLR410W | 2231 | 3,567 | 6,284 | 1.76 |
| YDR073W | 707 | 1,157 | 1,464 | 1.29 | YLR412W | 2232 | 891 | 2,204 | 2.47 |
| YDR074W | 708 | 3,006 | 5,872 | 1.95 | YLR413W | 2233 | 2,225 | 34,138 | 15.34 |
| YDR077W | 709 | 1,417 | 32,564 | 27.83 | YLR414C | 1972 | 997 | 874 | 1.32 |
| YDR079C-A | 544 | 444 | 1,095 | 2.47 | YLR418C | 1971 | 1,532 | 2,334 | 1.52 |
| YDR084C | 543 | 764 | 4,466 | 5.84 | YLR420W | 2234 | 1,198 | 5,881 | 4.91 |
| YDR086C | 542 | 522 | 7,711 | 14.77 | YLR421C | 1970 | 713 | 3,408 | 4.78 |
| YDR087C | 541 | 905 | 2,591 | 2.86 | YLR426W | 2235 | 1,102 | 1,451 | 1.32 |
| YDR090C | 540 | 1,380 | 2,423 | 1.76 | YLR427W | 2236 | 2,272 | 2,558 | 1.13 |
| YDR091C | 539 | 2,214 | 20,517 | 9.27 | YLR429W | 2237 | 2,040 | 10,786 | 5.29 |
| YDR092W | 710 | 621 | 6,576 | 10.59 | YLR432W | 2238 | 1,745 | 21,787 | 16.07 |
| YDR093W | 711 | 4,839 | 8,475 | 1.75 | YLR437C | 1969 | 498 | 2,297 | 4.83 |
| YDR098C | 538 | 858 | 4,720 | 5.50 | YLR438C-A | 1968 | 410 | 892 | 2.18 |
| YDR099W | 712 | 1,872 | 11,531 | 6.36 | YLR438W | 2239 | 1,385 | 11,745 | 8.48 |
| YDR100W | 713 | 588 | 3,593 | 6.11 | YLR439W | 2240 | 1,150 | 1,242 | 1.08 |
| YDR101C | 537 | 1,884 | 9,448 | 5.01 | YLR441C | 1967 | 974 | 64,036 | 83.30 |
| YDR105C | 536 | 1,738 | 5,820 | 3.35 | YLR443W | 2241 | 1,347 | 3,336 | 2.48 |
| YDR107C | 535 | 2,096 | 2,558 | 1.22 | YLR447C | 1966 | 1,216 | 18,824 | 15.48 |
| YDR111C | 534 | 1,751 | 2,287 | 1.31 | YLR448W | 2242 | 844 | 24,969 | 32.89 |
| YDR115W | 714 | 439 | 1,315 | 2.99 | YLR449W | 2243 | 1,296 | 4,228 | 3.28 |
| YDR116C | 533 | 980 | 1,722 | 1.76 | YLR450W | 2244 | 3,375 | 6,721 | 1.99 |
| YDR119W | 715 | 2,440 | 16,732 | 6.86 | YLR452C | 1965 | 2,357 | 8,256 | 3.50 |
| YDR120C | 532 | 1,911 | 3,282 | 1.72 | YLR459W | 2245 | 1,263 | 4,258 | 3.37 |
| YDR121W | 716 | 794 | 1,925 | 2.42 | YML001W | 2373 | 766 | 12,530 | 16.36 |
| YDR127W | 717 | 4,993 | 14,527 | 2.91 | YML004C | 2355 | 1,169 | 10,762 | 9.21 |
| YDR129C | 531 | 2,108 | 16,845 | 7.99 | YML008C | 2354 | 1,252 | 9,915 | 7.92 |
| YDR135C | 530 | 4,658 | 13,777 | 2.96 | YML009C | 2353 | 362 | 3,581 | 9.89 |
| YDR139C | 529 | 413 | 1,544 | 3.74 | YML010W | 2374 | 3,276 | 6,475 | 1.98 |
| YDR140W | 718 | 802 | 1,153 | 1.44 | YML011C | 2352 | 667 | 970 | 1.45 |
| YDR143C | 528 | 1,964 | 2,793 | 1.42 | YML012W | 2375 | 810 | 16,285 | 20.59 |
| YDR144C | 527 | 2,021 | 10,063 | 4.98 | YML013W | 2376 | 1,979 | 3,188 | 1.63 |
| YDR148C | 526 | 1,724 | 5,510 | 3.20 | YML014W | 2377 | 956 | 1,476 | 1.54 |
| YDR151C | 525 | 1,113 | 1,727 | 1.55 | YML016C | 2351 | 2,471 | 3,256 | 1.32 |
| YDR152W | 719 | 866 | 2,187 | 2.53 | YML018C | 2350 | 1,436 | 6,782 | 4.72 |
| YDR155C | 17 | 610 | 207,650 | 347.53 | YML019W | 2378 | 1,326 | 9,959 | 7.51 |
| YDR156W | 720 | 544 | 3,752 | 6.90 | YML021C | 2349 | 1,080 | 2,648 | 2.45 |
| YDR158W | 721 | 1,244 | 54,700 | 43.97 | YML022W | 2379 | 813 | 22,784 | 28.02 |
| YDR161W | 722 | 1,282 | 3,522 | 2.75 | YML023C | 2348 | 1,923 | 2,713 | 1.41 |
| YDR165W | 723 | 1,613 | 4,343 | 2.69 | YML024W | 2380 | 552 | 27,774 | 76.70 |
| YDR167W | 724 | 725 | 2,664 | 3.67 | YML026C | 2347 | 644 | 36,565 | 83.20 |
| YDR168W | 725 | 1,649 | 3,002 | 1.82 | YML027W | 2381 | 1,615 | 1,685 | 1.04 |
| YDR170C | 524 | 6,476 | 11,736 | 1.81 | YML028W | 2382 | 730 | 115,479 | 198.18 |
| YDR172W | 726 | 2,273 | 14,673 | 6.46 | YML029W | 2383 | 3,020 | 4,295 | 1.49 |
| YDR174W | 727 | 932 | 11,097 | 12.00 | YML031W | 2384 | 2,034 | 8,537 | 4.20 |
| YDR177W | 728 | 795 | 2,763 | 3.47 | YML032C | 2346 | 1,752 | 2,012 | 1.15 |
| YDR178W | 729 | 904 | 3,870 | 4.28 | YML035C | 2345 | 2,594 | 6,356 | 2.45 |
| YDR182W | 730 | 1,600 | 3,294 | 2.06 | YML036W | 2385 | 819 | 860 | 1.05 |
| YDR186C | 523 | 2,778 | 3,276 | 1.18 | YML038C | 2344 | 1,622 | 2,308 | 1.42 |
| YDR188W | 731 | 1,855 | 13,465 | 7.27 | YML048W | 2386 | 1,440 | 15,637 | 10.94 |
| YDR189W | 732 | 2,135 | 5,740 | 2.69 | YML051W | 2387 | 1,465 | 6,727 | 4.59 |
| YDR190C | 522 | 1,479 | 5,546 | 3.75 | YML052W | 2388 | 1,029 | 14,529 | 14.12 |
| YDR194C | 521 | 2,151 | 2,507 | 1.16 | YML055W | 2389 | 612 | 678 | 1.11 |
| YDR196C | 520 | 829 | 1,275 | 1.54 | YML056C | 2343 | 1,935 | 68,173 | 40.28 |
| YDR204W | 733 | 1,210 | 3,882 | 3.21 | YML057W | 2390 | 1,927 | 5,105 | 2.65 |
| YDR205W | 734 | 2,284 | 2,747 | 1.20 | YML058W | 2391 | 585 | 19,694 | 33.84 |
| YDR206W | 735 | 3,023 | 6,297 | 2.08 | YML059C | 2342 | 5,260 | 7,049 | 1.34 |
| YDR208W | 736 | 2,492 | 3,441 | 1.38 | YML063W | 2392 | 861 | 55,910 | 85.31 |
| YDR210W | 737 | 446 | 2,896 | 6.54 | YML064C | 2341 | 972 | 1,097 | 1.13 |
| YDR211W | 738 | 2,362 | 4,280 | 1.81 | YML067C | 2340 | 1,087 | 5,968 | 5.49 |
| YDR212W | 739 | 1,807 | 16,769 | 9.28 | YML069W | 2393 | 1,802 | 3,899 | 2.16 |
| YDR214W | 740 | 1,250 | 8,446 | 6.76 | YML070W | 2394 | 1,881 | 10,525 | 5.60 |
| YDR221W | 741 | 2,314 | 2,813 | 1.22 | YML072C | 2339 | 4,931 | 18,859 | 3.82 |
| YDR222W | 742 | 1,471 | 2,367 | 1.61 | YML073C | 2338 | 609 | 72,969 | 138.27 |
| YDR224C | 519 | 536 | 9,896 | 18.92 | YML074C | 2337 | 1,361 | 5,006 | 3.70 |
| YDR225W | 743 | 617 | 18,191 | 36.45 | YML075C | 2336 | 3,391 | 10,945 | 3.23 |
| YDR226W | 744 | 842 | 38,362 | 45.56 | YML077W | 2395 | 608 | 1,170 | 1.92 |
| YDR231C | 518 | 791 | 1,762 | 2.23 | YML078W | 2396 | 699 | 6,720 | 9.67 |
| YDR232W | 745 | 2,052 | 20,637 | 10.06 | YML079W | 2397 | 696 | 2,586 | 3.72 |
| YDR233C | 517 | 991 | 23,134 | 23.36 | YML080W | 2398 | 1,493 | 1,946 | 1.30 |
| YDR234W | 746 | 2,451 | 9,838 | 4.01 | YML081C-A | 2335 | 364 | 3,706 | 10.18 |
| YDR236C | 516 | 761 | 2,814 | 3.70 | YML081W | 2399 | 3,927 | 4,645 | 1.18 |
| YDR237W | 747 | 1,142 | 2,302 | 2.01 | YML085C | 2334 | 1,524 | 12,446 | 8.22 |
| YDR238C | 515 | 3,230 | 16,507 | 5.11 | YML086C | 2333 | 2,055 | 10,967 | 5.34 |
| YDR245W | 748 | 1,383 | 6,929 | 5.01 | YML088W | 2400 | 2,190 | 2,624 | 1.20 |
| YDR248C | 514 | 717 | 786 | 1.10 | YML092C | 2332 | 854 | 9,017 | 10.56 |
| YDR249C | 513 | 1,241 | 1,781 | 1.43 | YML094W | 2401 | 588 | 1,005 | 1.71 |
| YDR251W | 749 | 2,907 | 3,712 | 1.28 | YML096W | 2402 | 1,693 | 1,778 | 1.05 |
| YDR257C | 512 | 1,556 | 1,875 | 1.20 | YML098W | 2403 | 739 | 1,524 | 2.06 |
| YDR260C | 511 | 696 | 1,262 | 1.81 | YML099C | 2331 | 2,760 | 3,340 | 1.21 |
| YDR261C | 510 | 1,848 | 5,484 | 2.97 | YML100W | 2404 | 3,462 | 8,248 | 2.38 |
| YDR262W | 750 | 1,055 | 1,847 | 1.75 | YML101C | 2330 | 511 | 1,995 | 3.90 |
| YDR264C | 509 | 2,591 | 21,773 | 8.41 | YML102W | 2405 | 1,570 | 2,885 | 1.84 |
| YDR265W | 751 | 1,297 | 1,496 | 1.15 | YML103C | 2329 | 5,056 | 7,591 | 1.50 |
| YDR266C | 508 | 2,235 | 3,467 | 1.55 | YML105C | 2328 | 931 | 4,034 | 4.33 |
| YDR267C | 507 | 1,179 | 2,059 | 1.75 | YML106W | 2406 | 1,050 | 25,170 | 23.97 |
| YDR268W | 752 | 1,324 | 1,369 | 1.03 | YML108W | 2407 | 581 | 675 | 1.16 |
| YDR272W | 753 | 921 | 2,160 | 2.35 | YML110C | 2327 | 1,215 | 11,723 | 9.65 |
| YDR276C | 506 | 418 | 10,471 | 25.05 | YML113W | 2408 | 946 | 1,249 | 1.34 |
| YDR280W | 754 | 1,177 | 1,888 | 1.60 | YML115C | 2326 | 1,684 | 5,622 | 3.34 |
| YDR281C | 505 | 466 | 746 | 1.60 | YML117W | 2409 | 3,405 | 4,115 | 1.21 |
| YDR284C | 504 | 1,200 | 8,176 | 6.81 | YML119W | 2410 | 1,249 | 1,280 | 1.02 |
| YDR286C | 503 | 469 | 549 | 1.17 | YML120C | 2325 | 1,933 | 2,441 | 1.26 |
| YDR288W | 755 | 1,057 | 1,411 | 1.33 | YML121W | 2411 | 1,250 | 2,045 | 1.64 |
| YDR292C | 502 | 2,045 | 5,334 | 2.61 | YML123C | 2324 | 1,910 | 169,170 | 88.57 |
| YDR293C | 501 | 4,253 | 9,031 | 2.12 | YML124C | 2323 | 1,470 | 9,836 | 6.78 |
| YDR294C | 500 | 1,839 | 7,655 | 4.16 | YML125C | 2322 | 1,011 | 16,221 | 16.04 |
| YDR296W | 756 | 812 | 1,283 | 1.58 | YML126C | 2321 | 1,476 | 23,263 | 15.76 |
| YDR297W | 757 | 1,274 | 11,942 | 9.37 | YML127W | 2412 | 2,775 | 6,158 | 2.22 |
| YDR298C | 499 | 928 | 11,839 | 12.76 | YML128C | 2320 | 1,813 | 2,294 | 1.26 |
| YDR300C | 498 | 1,537 | 4,013 | 2.61 | YML129C | 2319 | 393 | 740 | 1.88 |
| YDR302W | 758 | 861 | 1,084 | 1.26 | YML130C | 2318 | 1,830 | 4,537 | 2.48 |
| YDR304C | 497 | 767 | 39,818 | 52.49 | YML131W | 2413 | 1,321 | 2,733 | 2.07 |
| YDR307W | 759 | 2,198 | 3,126 | 1.42 | YMR002W | 2414 | 773 | 9,735 | 12.60 |
| YDR309C | 496 | 1,221 | 3,558 | 2.91 | YMR005W | 2415 | 1,301 | 2,044 | 1.57 |
| YDR319C | 495 | 1,015 | 1,144 | 1.13 | YMR006C | 2317 | 2,286 | 30,049 | 13.29 |
| YDR320C-A | 494 | 273 | 2,557 | 9.37 | YMR008C | 2316 | 2,447 | 4,498 | 1.86 |
| YDR321W | 760 | 1,263 | 25,583 | 20.26 | YMR010W | 2416 | 1,637 | 5,604 | 3.42 |
| YDR322C-A | 493 | 508 | 1,546 | 3.04 | YMR011W | 2417 | 1,786 | 13,078 | 7.49 |
| YDR322W | 761 | 1,104 | 1,173 | 1.06 | YMR012W | 2418 | 4,096 | 14,265 | 3.48 |
| YDR324C | 492 | 2,331 | 4,755 | 2.04 | YMR013C | 2315 | 1,844 | 2,050 | 1.11 |
| YDR326C | 491 | 4,648 | 6,735 | 1.45 | YMR015C | 2314 | 1,935 | 10,099 | 5.22 |
| YDR328C | 490 | 826 | 10,426 | 12.62 | YMR022W | 2419 | 618 | 4,290 | 6.94 |
| YDR329C | 489 | 1,430 | 2,565 | 1.79 | YMR024W | 2420 | 1,173 | 1,838 | 1.57 |
| YDR331W | 762 | 1,305 | 2,996 | 2.30 | YMR026C | 2313 | 1,363 | 1,786 | 1.31 |
| YDR333C | 488 | 2,399 | 4,293 | 1.79 | YMR027W | 2421 | 1,527 | 6,331 | 4.15 |
| YDR335W | 763 | 4,118 | 4,848 | 1.18 | YMR031C | 2312 | 2,696 | 2,954 | 1.10 |
| YDR337W | 764 | 963 | 1,405 | 1.46 | YMR033W | 2422 | 1,589 | 2,844 | 1.79 |
| YDR339C | 487 | 632 | 1,616 | 2.56 | YMR035W | 2423 | 657 | 1,738 | 2.64 |
| YDR341C | 486 | 2,021 | 19,301 | 9.55 | YMR038C | 2311 | 882 | 8,260 | 9.36 |
| YDR342C | 485 | 1,713 | 1,024 | 1.19 | YMR042W | 2424 | 665 | 2,100 | 3.16 |
| YDR345C | 484 | 1,924 | 27,982 | 18.36 | YMR047C | 2310 | 3,487 | 7,370 | 2.11 |
| YDR346C | 483 | 1,856 | 10,008 | 5.39 | YMR049C | 2309 | 2,493 | 5,114 | 2.05 |
| YDR347W | 765 | 1,167 | 3,125 | 2.68 | YMR054W | 2425 | 2,806 | 5,565 | 1.98 |
| YDR348C | 482 | 1,880 | 3,230 | 1.72 | YMR055C | 2308 | 1,115 | 1,524 | 1.37 |
| YDR349C | 481 | 1,977 | 8,173 | 4.13 | YMR058W | 2426 | 2,101 | 44,738 | 21.29 |
| YDR352W | 766 | 1,286 | 6,109 | 4.75 | YMR062C | 2307 | 1,411 | 3,186 | 2.26 |
| YDR353W | 767 | 1,067 | 36,137 | 34.19 | YMR067C | 2306 | 1,276 | 1,577 | 1.23 |
| YDR354W | 768 | 1,304 | 5,793 | 4.44 | YMR071C | 2305 | 597 | 5,033 | 8.44 |
| YDR357C | 480 | 482 | 752 | 1.56 | YMR072W | 2427 | 716 | 7,568 | 10.57 |
| YDR361C | 479 | 1,116 | 4,020 | 3.60 | YMR073C | 2304 | 1,401 | 1,798 | 1.28 |
| YDR363W-A | 769 | 369 | 1,076 | 2.92 | YMR074C | 2303 | 495 | 7,385 | 14.92 |
| YDR366C | 478 | 399 | 714 | 1.89 | YMR079W | 2428 | 1,276 | 8,649 | 6.78 |
| YDR367W | 770 | 785 | 6,629 | 8.44 | YMR080C | 2302 | 3,384 | 10,219 | 3.02 |
| YDR368W | 771 | 1,174 | 12,526 | 10.67 | YMR083W | 2429 | 1,289 | 21,631 | 17.03 |
| YDR370C | 477 | 1,329 | 1,930 | 1.45 | YMR088C | 2301 | 1,857 | 6,514 | 3.51 |
| YDR372C | 476 | 1,410 | 2,838 | 2.01 | YMR089C | 2300 | 2,776 | 3,267 | 1.18 |
| YDR373W | 772 | 728 | 2,423 | 3.33 | YMR091C | 2299 | 1,391 | 2,224 | 1.60 |
| YDR374W-A | 773 | 477 | 920 | 1.93 | YMR092C | 2298 | 1,988 | 14,173 | 7.13 |
| YDR375C | 475 | 1,417 | 2,092 | 1.48 | YMR093W | 2430 | 1,709 | 6,202 | 3.63 |
| YDR376W | 774 | 1,560 | 2,988 | 1.91 | YMR099C | 2297 | 985 | 10,326 | 10.48 |
| YDR377W | 775 | 787 | 7,272 | 9.27 | YMR105C | 2296 | 1,896 | 2,869 | 1.51 |
| YDR378C | 474 | 456 | 7,234 | 15.86 | YMR108W | 2431 | 2,458 | 23,048 | 9.38 |
| YDR380W | 776 | 2,177 | 4,527 | 2.08 | YMR109W | 2432 | 3,768 | 4,957 | 1.32 |
| YDR381W | 777 | 958 | 16,601 | 17.33 | YMR110C | 2295 | 1,763 | 4,033 | 2.29 |
| YDR382W | 18 | 546 | 170,919 | 338.65 | YMR112C | 2294 | 841 | 1,278 | 1.52 |
| YDR384C | 473 | 1,058 | 4,409 | 4.17 | YMR113W | 2433 | 1,372 | 1,431 | 1.04 |
| YDR385W | 778 | 2,710 | 11,711 | 30.49 | YMR116C | 2293 | 1,122 | 523,787 | 466.83 |
| YDR387C | 472 | 1,937 | 3,288 | 1.70 | YMR119W | 2434 | 2,096 | 3,499 | 1.67 |
| YDR388W | 779 | 1,615 | 8,472 | 5.25 | YMR120C | 2292 | 1,960 | 5,215 | 2.66 |
| YDR390C | 471 | 1,985 | 2,371 | 1.19 | YMR121C | 2291 | 747 | 5,592 | 10.84 |
| YDR391C | 470 | 788 | 991 | 1.26 | YMR122W-A | 2435 | 547 | 27,437 | 50.16 |
| YDR394W | 780 | 1,467 | 11,979 | 8.17 | YMR123W | 2436 | 443 | 3,682 | 8.31 |
| YDR395W | 781 | 3,034 | 11,099 | 3.66 | YMR125W | 2437 | 2,803 | 7,195 | 2.57 |
| YDR397C | 469 | 573 | 1,798 | 3.14 | YMR126C | 2290 | 1,194 | 1,738 | 1.45 |
| YDR398W | 782 | 2,186 | 5,574 | 2.55 | YMR127C | 2289 | 1,259 | 1,939 | 1.54 |
| YDR399W | 783 | 836 | 22,978 | 27.49 | YMR129W | 2438 | 4,180 | 8,729 | 2.09 |
| YDR400W | 784 | 1,380 | 3,446 | 2.50 | YMR130W | 2439 | 1,064 | 3,152 | 2.96 |
| YDR404C | 468 | 835 | 6,363 | 7.62 | YMR131C | 2288 | 1,675 | 4,683 | 4.27 |
| YDR407C | 467 | 3,911 | 4,117 | 1.05 | YMR139W | 2440 | 1,422 | 2,168 | 1.52 |
| YDR408C | 466 | 847 | 8,973 | 10.59 | YMR142C | 2287 | 689 | 70,671 | 133.78 |
| YDR410C | 465 | 755 | 4,094 | 5.42 | YMR143W | 2441 | 545 | 52,761 | 126.45 |
| YDR411C | 464 | 1,115 | 3,735 | 3.35 | YMR145C | 2286 | 1,782 | 11,453 | 6.43 |
| YDR414C | 463 | 1,388 | 3,506 | 2.53 | YMR146C | 2285 | 1,169 | 12,991 | 11.13 |
| YDR415C | 462 | 1,309 | 2,416 | 1.85 | YMR148W | 2442 | 862 | 1,659 | 1.97 |
| YDR418W | 785 | 600 | 52,616 | 108.91 | YMR149W | 2443 | 1,020 | 17,887 | 17.99 |
| YDR422C | 461 | 2,626 | 3,064 | 1.17 | YMR150C | 2284 | 668 | 1,467 | 2.23 |
| YDR424C | 460 | 332 | 982 | 2.96 | YMR152W | 2444 | 1,243 | 3,774 | 3.04 |
| YDR427W | 786 | 1,313 | 4,312 | 3.28 | YMR153W | 2445 | 1,618 | 3,120 | 1.93 |
| YDR429C | 459 | 921 | 6,660 | 7.23 | YMR157C | 2283 | 890 | 1,610 | 1.81 |
| YDR432W | 787 | 1,245 | 6,311 | 5.07 | YMR158C-A | 2282 | 222 | 634 | 2.93 |
| YDR434W | 788 | 1,819 | 6,693 | 3.68 | YMR158W | 2446 | 596 | 945 | 1.59 |
| YDR435C | 458 | 1,153 | 2,496 | 2.16 | YMR161W | 2447 | 976 | 1,773 | 1.82 |
| YDR441C | 457 | 861 | 1,884 | 2.19 | YMR171C | 2281 | 2,064 | 3,553 | 1.72 |
| YDR447C | 456 | 526 | 28,897 | 85.85 | YMR173W | 2448 | 1,405 | 1,919 | 1.96 |
| YDR450W | 789 | 583 | 46,325 | 121.52 | YMR178W | 2449 | 1,008 | 3,354 | 3.33 |
| YDR451C | 455 | 1,305 | 1,992 | 1.53 | YMR183C | 2280 | 1,043 | 4,569 | 4.38 |
| YDR452W | 790 | 2,215 | 6,017 | 2.72 | YMR184W | 2450 | 702 | 2,989 | 4.26 |
| YDR454C | 454 | 680 | 16,395 | 24.11 | YMR186W | 2451 | 2,234 | 131,814 | 72.43 |
| YDR456W | 791 | 1,975 | 7,720 | 3.91 | YMR189W | 2452 | 3,342 | 7,768 | 2.32 |
| YDR459C | 453 | 1,372 | 2,823 | 2.06 | YMR191W | 2453 | 1,311 | 4,526 | 3.46 |
| YDR461C-A | 452 | 405 | 491 | 1.23 | YMR194C-B | 2279 | 365 | 391 | 1.07 |
| YDR462W | 792 | 606 | 1,110 | 1.83 | YMR194W | 2454 | 402 | 11,622 | 35.54 |
| YDR463W | 793 | 1,765 | 2,255 | 1.28 | YMR195W | 2455 | 589 | 1,121 | 1.90 |
| YDR465C | 451 | 1,420 | 2,817 | 1.98 | YMR197C | 2278 | 847 | 1,539 | 1.82 |
| YDR469W | 794 | 620 | 642 | 1.04 | YMR199W | 2456 | 1,932 | 2,284 | 1.18 |
| YDR471W | 795 | 550 | 37,251 | 77.81 | YMR200W | 2457 | 1,224 | 4,071 | 3.33 |
| YDR472W | 796 | 1,360 | 2,252 | 1.69 | YMR202W | 2458 | 768 | 57,484 | 74.85 |
| YDR476C | 450 | 864 | 3,625 | 4.20 | YMR203W | 2459 | 1,440 | 14,564 | 10.11 |
| YDR477W | 797 | 2,285 | 4,128 | 1.81 | YMR205C | 2277 | 3,237 | 70,114 | 21.69 |
| YDR481C | 449 | 1,787 | 20,345 | 11.41 | YMR208W | 2460 | 1,440 | 3,873 | 2.69 |
| YDR482C | 448 | 688 | 972 | 1.41 | YMR209C | 2276 | 2,455 | 3,220 | 1.31 |
| YDR483W | 798 | 1,510 | 29,226 | 19.36 | YMR212C | 2275 | 2,569 | 7,755 | 3.02 |
| YDR486C | 447 | 842 | 949 | 1.13 | YMR214W | 2461 | 1,333 | 6,734 | 5.05 |
| YDR487C | 446 | 764 | 16,249 | 21.27 | YMR215W | 2462 | 1,724 | 18,343 | 10.64 |
| YDR489W | 799 | 885 | 1,600 | 1.81 | YMR216C | 2274 | 2,603 | 2,717 | 1.04 |
| YDR492W | 800 | 1,185 | 2,006 | 1.69 | YMR217W | 2463 | 1,704 | 81,170 | 47.63 |
| YDR493W | 801 | 560 | 1,454 | 2.60 | YMR220W | 2464 | 1,519 | 6,148 | 4.05 |
| YDR496C | 445 | 2,057 | 2,557 | 1.24 | YMR221C | 2273 | 1,626 | 5,348 | 3.29 |
| YDR497C | 444 | 1,916 | 24,928 | 13.02 | YMR222C | 2272 | 745 | 2,779 | 3.73 |
| YDR498C | 443 | 1,226 | 1,541 | 1.26 | YMR223W | 2465 | 1,899 | 2,082 | 1.10 |
| YDR500C | 442 | 342 | 24,757 | 86.47 | YMR225C | 2271 | 439 | 443 | 1.01 |
| YDR502C | 441 | 1,458 | 22,286 | 15.99 | YMR226C | 2270 | 872 | 7,525 | 8.63 |
| YDR503C | 440 | 1,006 | 2,018 | 2.01 | YMR229C | 2269 | 5,379 | 11,883 | 2.21 |
| YDR508C | 439 | 2,333 | 17,086 | 7.36 | YMR230W | 2466 | 444 | 29,974 | 84.99 |
| YDR510W | 802 | 508 | 9,261 | 18.23 | YMR235C | 2268 | 1,386 | 14,836 | 10.70 |
| YDR511W | 803 | 491 | 1,338 | 2.72 | YMR236W | 2467 | 607 | 4,191 | 6.90 |
| YDR512C | 438 | 598 | 1,219 | 2.04 | YMR237W | 2468 | 2,392 | 5,681 | 2.37 |
| YDR513W | 804 | 534 | 5,876 | 11.00 | YMR238W | 2469 | 1,596 | 6,640 | 4.16 |
| YDR516C | 437 | 1,694 | 7,095 | 4.19 | YMR241W | 2470 | 1,125 | 10,595 | 9.42 |
| YDR517W | 805 | 1,371 | 8,487 | 6.19 | YMR242C | 2267 | 690 | 22,978 | 49.56 |
| YDR518W | 806 | 1,624 | 7,189 | 4.43 | YMR243C | 2266 | 1,602 | 36,957 | 23.07 |
| YDR519W | 807 | 682 | 6,436 | 9.44 | YMR246W | 2471 | 2,486 | 23,299 | 9.40 |
| YDR524C-B | 19 | 361 | 168,412 | 469.49 | YMR250W | 2472 | 1,902 | 2,777 | 1.46 |
| YDR525W-A | 808 | 545 | 1,325 | 2.43 | YMR251W-A | 2473 | 412 | 20,078 | 49.02 |
| YDR529C | 436 | 600 | 4,264 | 7.11 | YMR252C | 2265 | 774 | 1,313 | 1.70 |
| YDR530C | 435 | 1,080 | 2,033 | 1.88 | YMR256C | 2264 | 350 | 903 | 2.58 |
| YDR531W | 809 | 1,230 | 5,442 | 4.42 | YMR258C | 2263 | 1,722 | 2,808 | 1.63 |
| YDR533C | 434 | 821 | 1,828 | 2.23 | YMR260C | 2262 | 616 | 6,087 | 9.88 |
| YDR538W | 810 | 940 | 1,762 | 1.87 | YMR261C | 2261 | 3,457 | 8,286 | 2.40 |
| YDR539W | 811 | 1,675 | 4,702 | 2.81 | YMR262W | 2474 | 1,074 | 2,079 | 1.94 |
| YDR541C | 433 | 1,345 | 2,098 | 1.56 | YMR264W | 2475 | 780 | 5,756 | 7.38 |
| YEL001C | 882 | 783 | 10,351 | 13.22 | YMR266W | 2476 | 3,104 | 9,580 | 3.09 |
| YEL002C | 881 | 1,467 | 20,301 | 13.84 | YMR267W | 2477 | 1,192 | 1,894 | 1.59 |
| YEL003W | 901 | 461 | 1,498 | 3.25 | YMR272C | 2260 | 1,340 | 8,869 | 6.62 |
| YEL006W | 902 | 1,195 | 2,821 | 2.36 | YMR274C | 2259 | 948 | 1,220 | 1.29 |
| YEL007W | 903 | 2,380 | 3,818 | 1.62 | YMR276W | 2478 | 1,252 | 20,322 | 16.23 |
| YEL009C | 880 | 1,489 | 75,457 | 50.68 | YMR277W | 2479 | 2,461 | 4,116 | 1.68 |
| YEL013W | 904 | 1,737 | 10,338 | 5.95 | YMR278W | 2480 | 2,023 | 2,183 | 1.08 |
| YEL015W | 905 | 1,735 | 3,145 | 1.81 | YMR281W | 2481 | 1,125 | 2,183 | 1.94 |
| YEL016C | 879 | 1,722 | 3,111 | 1.81 | YMR286W | 2482 | 391 | 428 | 1.09 |
| YEL017C-A | 878 | 609 | 25,238 | 42.56 | YMR290C | 2258 | 1,723 | 6,187 | 3.59 |
| YEL017W | 906 | 1,233 | 5,240 | 4.25 | YMR292W | 2483 | 559 | 1,804 | 3.23 |
| YEL020C | 877 | 1,683 | 2,157 | 1.28 | YMR295C | 2257 | 809 | 6,916 | 8.55 |
| YEL020W-A | 907 | 381 | 4,411 | 11.58 | YMR296C | 2256 | 1,821 | 15,468 | 8.49 |
| YEL021W | 908 | 804 | 2,496 | 3.10 | YMR297W | 2484 | 1,785 | 60,426 | 33.85 |
| YEL024W | 909 | 1,232 | 4,257 | 3.45 | YMR298W | 2485 | 592 | 4,325 | 7.30 |
| YEL025C | 876 | 3,845 | 3,991 | 1.04 | YMR300C | 2255 | 1,689 | 6,265 | 3.71 |
| YEL026W | 910 | 558 | 19,564 | 35.06 | YMR301C | 2254 | 2,247 | 2,497 | 1.11 |
| YEL027W | 911 | 788 | 48,154 | 61.11 | YMR303C | 2253 | 1,099 | 3,198 | 4.17 |
| YEL029C | 875 | 1,094 | 1,862 | 1.70 | YMR305C | 2252 | 1,461 | 46,887 | 32.21 |
| YEL031W | 912 | 3,759 | 38,016 | 10.11 | YMR307W | 2486 | 1,931 | 103,078 | 53.52 |
| YEL032W | 913 | 3,109 | 8,885 | 2.86 | YMR308C | 2251 | 3,429 | 18,499 | 5.39 |
| YEL034W | 914 | 693 | 83,184 | 141.72 | YMR309C | 2250 | 2,663 | 13,005 | 4.88 |
| YEL036C | 874 | 1,682 | 7,694 | 4.70 | YMR310C | 2249 | 1,043 | 2,180 | 2.09 |
| YEL037C | 873 | 1,492 | 8,841 | 5.92 | YMR312W | 2487 | 953 | 1,546 | 1.62 |
| YEL038W | 915 | 765 | 8,489 | 11.10 | YMR314W | 2488 | 921 | 11,316 | 12.29 |
| YEL040W | 916 | 1,538 | 34,336 | 22.38 | YMR315W | 2489 | 1,220 | 8,930 | 7.32 |
| YEL042W | 917 | 1,846 | 13,023 | 7.05 | YMR318C | 2248 | 1,331 | 30,660 | 23.03 |
| YEL043W | 918 | 2,948 | 3,577 | 1.21 | YMR319C | 2247 | 2,043 | 4,728 | 2.31 |
| YEL044W | 919 | 655 | 2,576 | 3.93 | YMR321C | 2246 | 318 | 309 | 2.39 |
| YEL046C | 872 | 1,728 | 53,744 | 31.11 | YNL002C | 2589 | 1,046 | 1,897 | 1.81 |
| YEL047C | 871 | 1,507 | 11,642 | 7.74 | YNL003C | 2588 | 1,060 | 1,922 | 1.81 |
| YEL048C | 870 | 1,031 | 3,060 | 2.97 | YNL005C | 2587 | 1,239 | 1,466 | 1.18 |
| YEL050C | 869 | 1,358 | 3,421 | 2.52 | YNL006W | 2600 | 1,120 | 3,161 | 2.82 |
| YEL051W | 920 | 922 | 15,700 | 17.03 | YNL007C | 2586 | 1,213 | 10,254 | 8.45 |
| YEL052W | 921 | 1,679 | 5,260 | 3.13 | YNL008C | 2585 | 2,010 | 2,054 | 1.02 |
| YEL054C | 868 | 714 | 44,224 | 73.96 | YNL010W | 2601 | 867 | 19,200 | 22.15 |
| YEL056W | 922 | 1,500 | 2,581 | 1.72 | YNL015W | 2602 | 363 | 2,355 | 6.49 |
| YEL058W | 923 | 1,782 | 19,798 | 11.11 | YNL016W | 2603 | 2,010 | 7,345 | 3.69 |
| YEL059C-A | 867 | 405 | 543 | 1.34 | YNL022C | 2584 | 1,552 | 2,369 | 1.53 |
| YEL060C | 866 | 2,370 | 4,284 | 1.81 | YNL023C | 2583 | 3,111 | 4,036 | 1.30 |
| YEL063C | 865 | 1,965 | 5,187 | 2.64 | YNL024C-A | 2582 | 533 | 4,368 | 8.19 |
| YEL065W | 924 | 2,098 | 9,996 | 4.76 | YNL026W | 2604 | 1,595 | 2,938 | 1.84 |
| YEL066W | 925 | 597 | 2,047 | 3.43 | YNL029C | 2581 | 1,608 | 1,661 | 1.03 |
| YEL071W | 926 | 1,641 | 17,645 | 10.76 | YNL030W | 2605 | 546 | 13,249 | 34.32 |
| YER001W | 927 | 2,751 | 7,591 | 2.76 | YNL031C | 2580 | 663 | 41,414 | 77.12 |
| YER003C | 864 | 1,375 | 23,509 | 17.10 | YNL035C | 2579 | 1,422 | 3,927 | 2.76 |
| YER004W | 928 | 1,092 | 8,779 | 8.04 | YNL036W | 2606 | 829 | 2,244 | 2.71 |
| YER005W | 929 | 1,984 | 5,430 | 2.74 | YNL037C | 2578 | 1,296 | 11,282 | 8.70 |
| YER006W | 930 | 1,648 | 6,410 | 3.89 | YNL38W | 2607 | 786 | 1,142 | 1.45 |
| YER007C-A | 863 | 741 | 1,742 | 2.35 | YNL040W | 2608 | 1,567 | 1,956 | 1.25 |
| YER009W | 931 | 559 | 40,083 | 71.88 | YNL044W | 2609 | 752 | 8,781 | 11.68 |
| YER010C | 862 | 876 | 3,448 | 3.94 | YNL045W | 2610 | 2,084 | 8,849 | 4.25 |
| YER011W | 932 | 985 | 4,130 | 4.85 | YNL046W | 2611 | 767 | 4,472 | 5.83 |
| YER012W | 933 | 776 | 3,075 | 3.96 | YNL048W | 2612 | 1,757 | 2,765 | 1.57 |
| YER014W | 934 | 1,761 | 2,096 | 1.19 | YNL049C | 2577 | 2,757 | 5,714 | 2.07 |
| YER017C | 861 | 2,423 | 3,635 | 1.50 | YNL052W | 2613 | 754 | 5,943 | 7.88 |
| YER018C | 860 | 865 | 2,300 | 2.66 | YNL053W | 2614 | 1,926 | 2,122 | 1.10 |
| YER019C-A | 859 | 347 | 14,305 | 41.22 | YNL055C | 2576 | 931 | 56,070 | 60.22 |
| YER019W | 935 | 1,634 | 6,550 | 4.01 | YNL056W | 2615 | 750 | 3,191 | 4.25 |
| YER020W | 936 | 1,632 | 2,219 | 1.36 | YNL058C | 2575 | 1,056 | 1,629 | 1.55 |
| YER021W | 937 | 1,700 | 13,139 | 7.73 | YNL061W | 2616 | 2,036 | 3,231 | 1.59 |
| YER023W | 938 | 958 | 16,821 | 17.56 | YNL062C | 2574 | 1,437 | 3,494 | 2.43 |
| YER025W | 939 | 1,891 | 22,741 | 12.03 | YNL063W | 2617 | 1,187 | 1,376 | 1.16 |
| YER026C | 858 | 1,003 | 13,025 | 12.99 | YNL064C | 2573 | 1,524 | 19,986 | 13.11 |
| YER027C | 857 | 1,382 | 3,231 | 2.34 | YNL066W | 2618 | 1,819 | 25,121 | 13.82 |
| YER031C | 856 | 812 | 7,891 | 9.72 | YNL067W | 2619 | 777 | 34,572 | 63.89 |
| YER034W | 940 | 799 | 797 | 1.02 | YNL069C | 2572 | 735 | 99,584 | 145.05 |
| YER035W | 941 | 783 | 798 | 1.04 | YNL070W | 2620 | 382 | 5,430 | 14.21 |
| YER036C | 855 | 1,940 | 31,912 | 16.45 | YNL071W | 2621 | 1,639 | 29,600 | 18.14 |
| YER043C | 854 | 1,560 | 62,895 | 40.32 | YNL072W | 2622 | 1,015 | 2,037 | 2.01 |
| YER044C | 853 | 619 | 4,010 | 6.48 | YNL073W | 2623 | 1,810 | 1,814 | 1.00 |
| YER048C | 852 | 1,344 | 2,292 | 1.70 | YNL074C | 2571 | 1,463 | 3,060 | 2.09 |
| YER048W-A | 942 | 686 | 4,116 | 6.00 | YNL075W | 2624 | 1,166 | 1,905 | 1.64 |
| YER049W | 943 | 2,119 | 10,730 | 5.06 | YNL078W | 2625 | 1,402 | 4,621 | 3.30 |
| YER050C | 851 | 534 | 841 | 1.57 | YNL079C | 2570 | 788 | 10,926 | 13.87 |
| YER052C | 850 | 1,754 | 8,959 | 5.12 | YNL080C | 2569 | 1,269 | 4,005 | 3.16 |
| YER055C | 849 | 1,093 | 23,911 | 21.88 | YNL081C | 2568 | 639 | 1,909 | 2.99 |
| YER056C | 848 | 1,778 | 22,682 | 13.82 | YNL084C | 2567 | 1,162 | 1,670 | 1.44 |
| YER056C-A | 847 | 443 | 14,359 | 45.00 | YNL085W | 2626 | 3,018 | 9,973 | 3.30 |
| YER057C | 846 | 479 | 9,977 | 20.83 | YNL087W | 2627 | 3,903 | 10,730 | 2.75 |
| YER060W | 944 | 1,854 | 4,010 | 2.19 | YNL090W | 2628 | 843 | 1,848 | 2.19 |
| YER060W-A | 945 | 1,593 | 1,739 | 1.20 | YNL094W | 2629 | 2,085 | 3,365 | 1.61 |
| YER062C | 845 | 1,098 | 8,305 | 7.65 | YNL096C | 2566 | 784 | 24,491 | 37.46 |
| YER063W | 946 | 871 | 2,765 | 3.17 | YNL099C | 2565 | 987 | 2,553 | 2.59 |
| YER064C | 844 | 1,843 | 4,250 | 2.31 | YNL100W | 2630 | 866 | 1,930 | 2.23 |
| YER070W | 947 | 2,911 | 44,006 | 15.16 | YNL101W | 2631 | 2,510 | 6,019 | 2.40 |
| YER072W | 948 | 576 | 19,044 | 33.06 | YNL104C | 2564 | 1,964 | 11,987 | 6.22 |
| YER073W | 949 | 1,872 | 6,120 | 3.27 | YNL108C | 2563 | 970 | 2,397 | 2.47 |
| YER074W | 950 | 540 | 32,518 | 120.58 | YNL111C | 2562 | 635 | 2,612 | 4.11 |
| YER074W-A | 951 | 347 | 1,908 | 5.50 | YNL112W | 2632 | 1,801 | 17,969 | 10.01 |
| YER078C | 843 | 1,659 | 1,665 | 1.00 | YNL113W | 2633 | 601 | 3,003 | 5.00 |
| YER080W | 952 | 1,966 | 2,099 | 1.07 | YNL115C | 2561 | 2,103 | 2,156 | 1.02 |
| YER081W | 953 | 1,790 | 2,507 | 1.50 | YNL116W | 2634 | 1,783 | 2,116 | 1.19 |
| YER082C | 842 | 1,808 | 2,002 | 1.11 | YNL118C | 2560 | 3,072 | 3,861 | 1.26 |
| YER083C | 841 | 902 | 4,277 | 4.74 | YNL121C | 2559 | 2,002 | 4,306 | 2.15 |
| YER086W | 954 | 1,935 | 21,985 | 11.36 | YNL122C | 2558 | 348 | 578 | 1.66 |
| YER087C-B | 840 | 470 | 7,106 | 15.12 | YNL123W | 2635 | 3,166 | 7,440 | 2.35 |
| YER088C | 839 | 2,593 | 3,624 | 1.40 | YNL125C | 2557 | 2,133 | 3,028 | 1.42 |
| YER089C | 838 | 1,754 | 6,532 | 3.72 | YNL130C | 2556 | 1,290 | 12,037 | 9.33 |
| YER090W | 955 | 1,680 | 8,147 | 4.85 | YNL131W | 2636 | 996 | 11,208 | 11.25 |
| YER091C | 837 | 2,558 | 24,401 | 9.54 | YNL132W | 2637 | 3,335 | 6,712 | 2.01 |
| YER092W | 956 | 378 | 1,505 | 3.98 | YNL134C | 2555 | 1,226 | 13,573 | 11.07 |
| YER094C | 836 | 962 | 15,654 | 16.27 | YNL135C | 2554 | 562 | 22,734 | 40.45 |
| YER095W | 957 | 1,486 | 5,539 | 3.73 | YNL137C | 2553 | 1,757 | 3,269 | 1.86 |
| YER099C | 835 | 1,281 | 2,590 | 2.02 | YNL138W | 2638 | 1,761 | 10,852 | 6.16 |
| YER100W | 958 | 1,112 | 2,045 | 1.84 | YNL138W-A | 2639 | 406 | 1,042 | 2.57 |
| YER102W | 959 | 1,036 | 15,809 | 28.76 | YNL141W | 2640 | 1,226 | 15,692 | 12.80 |
| YER105C | 834 | 4,434 | 7,475 | 1.69 | YNL147W | 2641 | 629 | 675 | 1.07 |
| YER107C | 833 | 1,388 | 4,535 | 3.27 | YNL149C | 2552 | 532 | 4,299 | 8.14 |
| YER110C | 832 | 3,459 | 34,111 | 9.86 | YNL151C | 2551 | 895 | 1,014 | 1.13 |
| YER113C | 831 | 2,121 | 3,413 | 1.61 | YNL153C | 2550 | 602 | 2,760 | 4.69 |
| YER117W | 960 | 510 | 33,519 | 102.23 | YNL154C | 2549 | 2,163 | 7,264 | 3.38 |
| YER118C | 830 | 1,455 | 4,180 | 2.87 | YNL155W | 2642 | 1,086 | 3,485 | 3.21 |
| YER119C | 829 | 1,575 | 2,859 | 1.81 | YNL156C | 2548 | 1,174 | 4,378 | 3.73 |
| YER120W | 961 | 861 | 19,958 | 23.23 | YNL157W | 2643 | 710 | 661 | 1.20 |
| YER122C | 828 | 1,686 | 5,204 | 3.09 | YNL158W | 2644 | 1,044 | 1,851 | 2.08 |
| YER124C | 827 | 2,060 | 10,002 | 4.85 | YNL159C | 2547 | 1,202 | 1,998 | 1.66 |
| YER125W | 962 | 3,315 | 13,912 | 4.20 | YNL160W | 2645 | 1,327 | 10,614 | 8.00 |
| YER126C | 826 | 935 | 2,569 | 2.75 | YNL161W | 2646 | 2,512 | 2,835 | 1.14 |
| YER127W | 963 | 1,182 | 1,201 | 1.02 | YNL162W | 2647 | 433 | 11,978 | 57.37 |
| YER131W | 964 | 867 | 14,680 | 19.14 | YNL168C | 2546 | 958 | 9,303 | 9.71 |
| YER133W | 965 | 1,228 | 4,483 | 3.65 | YNL169C | 2545 | 1,629 | 5,443 | 3.34 |
| YER134C | 825 | 794 | 1,596 | 2.01 | YNL173C | 2544 | 1,217 | 1,438 | 1.18 |
| YER136W | 966 | 1,762 | 7,730 | 4.39 | YNL175C | 2543 | 1,302 | 3,143 | 2.41 |
| YER141W | 967 | 2,711 | 4,108 | 1.79 | YNL177C | 2542 | 1,122 | 1,397 | 1.24 |
| YER143W | 968 | 1,502 | 2,362 | 1.57 | YNL178W | 2648 | 808 | 374,769 | 463.82 |
| YER145C | 824 | 1,327 | 16,160 | 12.18 | YNL182C | 2541 | 1,794 | 4,584 | 2.55 |
| YER146W | 969 | 367 | 7,138 | 19.45 | YNL185C | 2540 | 681 | 987 | 1.45 |
| YER147C | 823 | 1,968 | 2,678 | 1.36 | YNL186W | 2649 | 2,723 | 3,665 | 1.35 |
| YER148W | 970 | 913 | 4,971 | 5.44 | YNL189W | 2650 | 1,951 | 15,301 | 7.84 |
| YER149C | 822 | 1,586 | 2,560 | 1.61 | YNL190W | 2651 | 1,428 | 16,630 | 14.03 |
| YER150W | 971 | 633 | 840 | 1.33 | YNL191W | 2652 | 1,206 | 1,309 | 1.09 |
| YER152C | 821 | 1,364 | 9,996 | 7.33 | YNL192W | 2653 | 3,617 | 6,045 | 1.67 |
| YER153C | 820 | 765 | 878 | 1.15 | YNL197C | 2539 | 3,002 | 5,126 | 1.71 |
| YER154W | 972 | 1,297 | 2,712 | 2.09 | YNL200C | 2538 | 902 | 2,312 | 2.56 |
| YER155C | 819 | 6,694 | 10,240 | 1.53 | YNL207W | 2654 | 1,576 | 5,730 | 3.64 |
| YER156C | 818 | 1,121 | 9,582 | 8.55 | YNL208W | 2655 | 728 | 19,212 | 26.89 |
| YER157W | 973 | 2,406 | 2,735 | 1.14 | YNL209W | 2656 | 1,987 | 43,668 | 63.56 |
| YER159C | 817 | 718 | 894 | 1.24 | YNL216W | 2657 | 2,692 | 6,895 | 2.56 |
| YER163C | 816 | 753 | 3,531 | 4.69 | YNL217W | 2658 | 1,115 | 5,615 | 5.03 |
| YER165W | 974 | 2,019 | 29,564 | 14.65 | YNL219C | 2537 | 2,977 | 8,984 | 3.02 |
| YER166W | 975 | 4,798 | 6,168 | 1.29 | YNL220W | 2659 | 1,483 | 11,593 | 7.82 |
| YER168C | 815 | 1,723 | 2,664 | 1.55 | YNL221C | 2536 | 2,821 | 3,346 | 1.19 |
| YER174C | 814 | 820 | 4,594 | 5.60 | YNL222W | 2660 | 698 | 789 | 1.13 |
| YER177W | 976 | 1,021 | 62,595 | 63.70 | YNL231C | 2535 | 1,374 | 2,069 | 1.53 |
| YER178W | 977 | 1,415 | 53,201 | 37.60 | YNL232W | 2661 | 971 | 6,023 | 6.20 |
| YER183C | 813 | 708 | 3,425 | 4.84 | YNL238W | 2662 | 2,823 | 6,364 | 2.25 |
| YER186C | 812 | 1,101 | 4,008 | 3.64 | YNL239W | 2663 | 1,565 | 11,323 | 7.23 |
| YFL001W | 1011 | 1,370 | 1,421 | 1.04 | YNL240C | 2534 | 1,743 | 3,942 | 2.26 |
| YFL004W | 1012 | 2,707 | 10,187 | 3.76 | YNL241C | 2533 | 1,772 | 15,927 | 8.99 |
| YFL005W | 1013 | 1,028 | 11,565 | 11.25 | YNL243W | 2664 | 3,018 | 18,405 | 6.10 |
| YFL007W | 1014 | 6,623 | 9,496 | 1.43 | YNL244C | 2532 | 473 | 13,305 | 28.17 |
| YFL009W | 1015 | 2,606 | 3,670 | 1.44 | YNL246W | 2665 | 1,099 | 2,702 | 2.46 |
| YFL010C | 1007 | 790 | 8,449 | 10.79 | YNL247W | 2666 | 2,433 | 7,872 | 3.24 |
| YFL014W | 1016 | 543 | 1,008 | 1.86 | YNL248C | 2531 | 1,483 | 5,283 | 3.56 |
| YFL016C | 1006 | 1,631 | 4,940 | 3.03 | YNL251C | 2530 | 2,041 | 3,790 | 1.86 |
| YFL017C | 1005 | 681 | 1,434 | 2.11 | YNL255C | 2529 | 735 | 20,838 | 28.35 |
| YFL017W-A | 1017 | 337 | 677 | 2.01 | YNL256W | 2667 | 2,620 | 3,264 | 1.25 |
| YFL018C | 1004 | 1,652 | 12,406 | 7.51 | YNL259C | 2528 | 323 | 1,210 | 3.75 |
| YFL021W | 1018 | 1,679 | 1,868 | 1.11 | YNL261W | 2668 | 1,901 | 3,510 | 1.86 |
| YFL022C | 1003 | 1,666 | 21,248 | 12.75 | YNL263C | 2527 | 1,193 | 10,249 | 8.59 |
| YFL028C | 1002 | 953 | 2,534 | 2.66 | YNL264C | 2526 | 1,231 | 3,412 | 2.77 |
| YFL031W | 1019 | 1,201 | 64,554 | 53.75 | YNL265C | 2525 | 1,176 | 1,467 | 1.25 |
| YFL034C-A | 1001 | 460 | 5,199 | 11.36 | YNL268W | 2669 | 2,100 | 9,425 | 4.54 |
| YFL037W | 1020 | 1,666 | 30,461 | 18.29 | YNL280C | 2524 | 1,474 | 9,679 | 6.57 |
| YFL038C | 1000 | 768 | 9,137 | 11.90 | YNL281W | 2670 | 654 | 8,526 | 13.04 |
| YFL039C | 999 | 1,338 | 154,961 | 115.86 | YNL282W | 2671 | 588 | 1,020 | 1.73 |
| YFL041W | 1021 | 2,055 | 5,998 | 2.92 | YNL283C | 2523 | 1,619 | 6,387 | 3.94 |
| YFL044C | 998 | 1,118 | 3,271 | 2.93 | YNL284C | 2522 | 969 | 1,437 | 1.48 |
| YFL045C | 997 | 985 | 54,920 | 55.79 | YNL287W | 2672 | 3,234 | 18,302 | 5.66 |
| YFL046W | 1022 | 803 | 927 | 1.15 | YNL288W | 2673 | 1,402 | 5,551 | 3.96 |
| YFL047W | 1023 | 2,205 | 2,839 | 1.29 | YNL289W | 2674 | 1,069 | 1,395 | 1.30 |
| YFL048C | 996 | 1,448 | 15,802 | 10.91 | YNL290W | 2675 | 1,336 | 3,024 | 2.26 |
| YFR001W | 1024 | 734 | 917 | 1.25 | YNL291C | 2521 | 1,954 | 6,222 | 3.18 |
| YFR002W | 1025 | 2,619 | 3,481 | 1.33 | YNL292W | 2676 | 1,213 | 1,337 | 1.10 |
| YFR003C | 995 | 623 | 1,401 | 2.25 | YNL294C | 2520 | 1,910 | 3,856 | 2.02 |
| YFR004W | 1026 | 1,017 | 3,265 | 3.21 | YNL300W | 2677 | 659 | 4,686 | 7.11 |
| YFR005C | 994 | 1,573 | 2,758 | 1.75 | YNL301C | 2519 | 729 | 18,808 | 45.43 |
| YFR006W | 1027 | 1,879 | 6,792 | 3.61 | YNL302C | 2518 | 545 | 35,640 | 101.94 |
| YFR009W | 1028 | 2,391 | 10,825 | 4.54 | YNL305C | 2517 | 994 | 5,165 | 5.20 |
| YFR010W | 1029 | 1,675 | 2,955 | 1.76 | YNL306W | 2678 | 766 | 2,021 | 2.64 |
| YFR011C | 993 | 513 | 582 | 1.13 | YNL307C | 2516 | 1,617 | 20,848 | 12.90 |
| YFR014C | 992 | 1,737 | 1,927 | 1.11 | YNL310C | 2515 | 603 | 2,752 | 4.56 |
| YFR018C | 991 | 1,293 | 6,384 | 4.95 | YNL312W | 2679 | 928 | 3,550 | 3.82 |
| YFR021W | 1030 | 1,680 | 2,260 | 1.35 | YNL313C | 2514 | 2,782 | 4,223 | 1.52 |
| YFR024C-A | 990 | 1,533 | 10,318 | 6.74 | YNL315C | 2513 | 1,023 | 3,216 | 3.16 |
| YFR025C | 989 | 1,124 | 4,347 | 3.87 | YNL320W | 2680 | 1,019 | 2,427 | 2.38 |
| YFR028C | 988 | 1,749 | 4,390 | 2.51 | YNL321W | 2681 | 2,727 | 5,246 | 1.92 |
| YFR030W | 1031 | 3,210 | 3,792 | 1.18 | YNL322C | 2512 | 994 | 10,594 | 10.66 |
| YFR031C-A | 987 | 924 | 26,378 | 60.18 | YNL323W | 2682 | 1,441 | 2,782 | 1.93 |
| YFR032C-A | 20 | 588 | 100,484 | 198.09 | YNL326C | 2511 | 1,214 | 1,634 | 1.35 |
| YFR033C | 986 | 675 | 749 | 1.22 | YNL327W | 2683 | 3,278 | 22,775 | 7.14 |
| YFR034C | 985 | 1,317 | 2,459 | 1.95 | YNL329C | 2510 | 3,217 | 3,272 | 1.02 |
| YFR037C | 984 | 1,792 | 5,709 | 3.19 | YNL330C | 2509 | 1,389 | 4,022 | 2.90 |
| YFR039C | 983 | 1,692 | 3,454 | 2.04 | YNR001C | 2508 | 1,644 | 6,870 | 4.18 |
| YFR042W | 1032 | 748 | 3,388 | 4.53 | YNR003C | 2507 | 1,055 | 3,469 | 3.29 |
| YFR044C | 982 | 1,493 | 21,797 | 14.60 | YNR009W | 2684 | 1,052 | 1,168 | 1.11 |
| YFR045W | 1033 | 1,052 | 1,261 | 1.20 | YNR012W | 2685 | 1,680 | 2,248 | 1.34 |
| YFR047C | 981 | 971 | 4,411 | 4.54 | YNR013C | 2506 | 2,799 | 9,152 | 3.27 |
| YFR049W | 1034 | 652 | 932 | 1.43 | YNR015W | 2686 | 1,203 | 4,092 | 3.40 |
| YFR050C | 980 | 959 | 8,865 | 9.24 | YNR016C | 2505 | 7,327 | 57,034 | 7.78 |
| YFR051C | 979 | 1,734 | 15,035 | 8.67 | YNR017W | 2687 | 1,018 | 5,222 | 5.13 |
| YFR052W | 1035 | 1,066 | 4,090 | 3.84 | YNR018W | 2688 | 843 | 7,310 | 8.67 |
| YFR053C | 978 | 1,695 | 5,007 | 2.97 | YNR019W | 2689 | 2,046 | 5,199 | 2.56 |
| YFR055W | 1036 | 1,493 | 5,318 | 3.56 | YNR021W | 2690 | 1,422 | 16,839 | 11.84 |
| YGL001C | 1168 | 1,207 | 14,621 | 12.11 | YNR022C | 2504 | 529 | 1,736 | 3.28 |
| YGL002W | 1193 | 926 | 3,698 | 3.99 | YNR027W | 2691 | 1,235 | 2,763 | 2.24 |
| YGL003C | 1167 | 2,025 | 2,153 | 1.06 | YNR028W | 2692 | 1,019 | 2,991 | 2.93 |
| YGL006W | 1194 | 3,932 | 4,524 | 1.15 | YNR030W | 2693 | 1,875 | 3,579 | 1.91 |
| YGL008C | 1166 | 3,486 | 364,856 | 111.64 | YNR032C-A | 2503 | 329 | 739 | 2.25 |
| YGL009C | 1165 | 2,528 | 15,321 | 6.06 | YNR032W | 2694 | 1,236 | 3,490 | 2.82 |
| YGL010W | 1195 | 750 | 1,626 | 2.17 | YNR033W | 2695 | 2,456 | 4,723 | 1.92 |
| YGL011C | 1164 | 879 | 7,605 | 8.65 | YNR035C | 2502 | 1,096 | 12,610 | 11.50 |
| YGL012W | 1196 | 1,552 | 45,009 | 29.00 | YNR036C | 2501 | 609 | 7,452 | 12.32 |
| YGL014W | 1197 | 3,125 | 4,856 | 1.56 | YNR037C | 2500 | 383 | 2,360 | 6.16 |
| YGL016W | 1198 | 3,246 | 5,325 | 1.64 | YNR038W | 2696 | 2,052 | 2,912 | 1.42 |
| YGL017W | 1199 | 1,612 | 1,844 | 1.14 | YNR040W | 2697 | 855 | 1,208 | 1.41 |
| YGL019W | 1200 | 1,007 | 4,946 | 4.91 | YNR041C | 2499 | 1,165 | 3,502 | 3.01 |
| YGL020C | 1163 | 814 | 3,818 | 4.69 | YNR043W | 2698 | 1,348 | 14,561 | 10.80 |
| YGL021W | 1201 | 2,387 | 3,956 | 1.66 | YNR044W | 2699 | 2,275 | 4,089 | 1.86 |
| YGL022W | 1202 | 2,296 | 27,225 | 11.86 | YNR046W | 2700 | 560 | 6,300 | 11.25 |
| YGL023C | 1162 | 2,045 | 2,417 | 1.18 | YNR049C | 2498 | 785 | 984 | 1.25 |
| YGL025C | 1161 | 1,258 | 2,380 | 1.91 | YNR050C | 2497 | 1,535 | 15,310 | 9.97 |
| YGL026C | 1160 | 2,213 | 20,217 | 9.13 | YNR051C | 2496 | 2,274 | 2,557 | 1.12 |
| YGL027C | 1159 | 2,619 | 6,963 | 2.66 | YNR052C | 2495 | 1,431 | 6,776 | 4.82 |
| YGL028C | 1158 | 2,063 | 9,847 | 4.78 | YNR053C | 2494 | 1,582 | 7,878 | 4.98 |
| YGL030W | 21 | 464 | 186,050 | 400.97 | YNR055C | 2493 | 2,081 | 8,860 | 4.26 |
| YGL031C | 1157 | 1,064 | 32,200 | 35.64 | YNR061C | 2492 | 1,033 | 8,277 | 8.01 |
| YGL035C | 1156 | 1,856 | 2,507 | 1.36 | YNR067C | 2491 | 3,483 | 19,347 | 5.55 |
| YGL037C | 1155 | 822 | 15,209 | 18.50 | YNR074C | 2490 | 1,343 | 1,374 | 1.03 |
| YGL038C | 1154 | 1,661 | 3,601 | 2.17 | YOL001W | 2851 | 1,480 | 1,535 | 1.04 |
| YGL039W | 1203 | 1,255 | 6,213 | 4.95 | YOL002C | 2837 | 1,050 | 4,088 | 3.89 |
| YGL040C | 1153 | 1,290 | 14,603 | 11.32 | YOL003C | 2836 | 1,262 | 3,682 | 2.92 |
| YGL043W | 1204 | 1,184 | 4,970 | 4.20 | YOL004W | 2852 | 4,932 | 5,539 | 1.12 |
| YGL047W | 1205 | 670 | 945 | 1.41 | YOL005C | 2835 | 416 | 4,115 | 9.89 |
| YGL048C | 1152 | 1,308 | 12,327 | 9.43 | YOL007C | 2834 | 1,428 | 3,867 | 2.71 |
| YGL050W | 1206 | 822 | 1,221 | 1.49 | YOL008W | 2853 | 789 | 1,052 | 1.33 |
| YGL054C | 1151 | 502 | 7,191 | 14.32 | YOL010W | 2854 | 1,272 | 2,172 | 1.71 |
| YGL055W | 1207 | 1,852 | 36,054 | 19.47 | YOL011W | 2855 | 2,261 | 4,563 | 2.02 |
| YGL056C | 1150 | 1,748 | 2,313 | 1.32 | YOL012C | 2833 | 635 | 4,247 | 6.69 |
| YGL058W | 1208 | 800 | 4,838 | 6.11 | YOL013C | 2832 | 1,933 | 2,247 | 1.17 |
| YGL062W | 1209 | 4,007 | 6,870 | 1.82 | YOL013W-A | 2856 | 315 | 975 | 3.26 |
| YGL063W | 1210 | 1,113 | 1,467 | 1.32 | YOL014W | 2857 | 655 | 1,461 | 2.23 |
| YGL065C | 1149 | 1,512 | 1,667 | 1.10 | YOL020W | 2858 | 2,009 | 7,592 | 3.78 |
| YGL067W | 1211 | 1,298 | 2,781 | 2.14 | YOL021C | 2831 | 3,125 | 5,793 | 1.85 |
| YGL068W | 1212 | 743 | 8,333 | 11.21 | YOL022C | 2830 | 1,330 | 4,942 | 3.72 |
| YGL070C | 1148 | 698 | 1,558 | 2.23 | YOL026C | 2829 | 432 | 2,141 | 4.96 |
| YGL076C | 1147 | 847 | 48,212 | 123.09 | YOL027C | 2828 | 1,925 | 2,261 | 1.17 |
| YGL077C | 1146 | 1,811 | 9,002 | 4.97 | YOL030W | 2859 | 1,675 | 17,305 | 10.33 |
| YGL078C | 1145 | 1,818 | 3,882 | 2.13 | YOL031C | 2827 | 1,352 | 1,883 | 1.39 |
| YGL079W | 1213 | 851 | 1,084 | 1.27 | YOL036W | 2860 | 2,815 | 3,118 | 1.11 |
| YGL080W | 1214 | 711 | 1,623 | 2.28 | YOL038W | 2861 | 866 | 8,509 | 9.82 |
| YGL082W | 1215 | 1,381 | 2,669 | 1.93 | YOL039W | 2862 | 645 | 113,806 | 191.61 |
| YGL084C | 1144 | 1,832 | 7,160 | 3.91 | YOL040C | 2826 | 529 | 94,557 | 178.75 |
| YGL087C | 1143 | 545 | 874 | 1.60 | YOL041C | 2825 | 1,511 | 1,515 | 1.00 |
| YGL089C | 1142 | 363 | 34,800 | 97.10 | YOL042W | 2863 | 1,443 | 1,625 | 1.13 |
| YGL091C | 1141 | 1,175 | 1,863 | 1.59 | YOL048C | 2824 | 1,144 | 2,945 | 2.57 |
| YGL092W | 1216 | 4,051 | 5,081 | 1.25 | YOL049W | 2864 | 1,631 | 7,250 | 4.44 |
| YGL097W | 1217 | 1,589 | 6,861 | 4.32 | YOL052C | 2823 | 1,351 | 4,500 | 3.33 |
| YGL099W | 1218 | 2,006 | 2,100 | 1.05 | YOL052C-A | 2822 | 464 | 651 | 1.41 |
| YGL100W | 1219 | 1,162 | 5,006 | 4.31 | YOL056W | 2865 | 1,192 | 1,392 | 1.17 |
| YGL101W | 1220 | 1,003 | 2,831 | 2.82 | YOL057W | 2866 | 2,299 | 11,562 | 5.03 |
| YGL103W | 22 | 554 | 262,228 | 473.34 | YOL059W | 2867 | 1,801 | 12,170 | 6.77 |
| YGL105W | 1221 | 1,276 | 22,928 | 17.97 | YOL060C | 2821 | 2,478 | 3,543 | 1.43 |
| YGL106W | 1222 | 718 | 5,920 | 8.31 | YOL061W | 2868 | 1,685 | 22,296 | 13.24 |
| YGL111W | 1223 | 1,563 | 2,099 | 1.34 | YOL062C | 2820 | 1,731 | 6,691 | 3.86 |
| YGL112C | 1140 | 1,695 | 4,740 | 2.80 | YOL063C | 2819 | 2,940 | 2,985 | 1.01 |
| YGL114W | 1224 | 2,298 | 3,629 | 1.58 | YOL064C | 2818 | 1,164 | 6,752 | 5.80 |
| YGL115W | 1225 | 1,173 | 11,802 | 10.06 | YOL066C | 2817 | 1,845 | 2,132 | 1.16 |
| YGL120C | 1139 | 2,442 | 9,643 | 3.95 | YOL070C | 2816 | 1,660 | 1,703 | 1.02 |
| YGL122C | 1138 | 1,816 | 4,264 | 2.45 | YOL073C | 2815 | 1,048 | 4,110 | 3.92 |
| YGL123W | 23 | 952 | 442,048 | 464.54 | YOL077C | 2814 | 971 | 5,445 | 5.61 |
| YGL126W | 1226 | 1,435 | 13,827 | 9.68 | YOL077W-A | 2869 | 345 | 1,594 | 4.62 |
| YGL127C | 1137 | 492 | 2,055 | 4.19 | YOL082W | 2870 | 1,461 | 2,386 | 1.63 |
| YGL129C | 1136 | 1,564 | 2,073 | 1.32 | YOL086C | 2813 | 1,154 | 546,138 | 670.82 |
| YGL135W | 1227 | 813 | 36,219 | 165.42 | YOL086W-A | 2871 | 344 | 1,081 | 3.14 |
| YGL137W | 1228 | 2,880 | 16,454 | 5.71 | YOL088C | 2812 | 1,033 | 3,054 | 2.96 |
| YGL139W | 1229 | 2,819 | 4,735 | 1.69 | YOL092W | 2872 | 992 | 11,590 | 11.68 |
| YGL140C | 1135 | 3,923 | 4,496 | 1.15 | YOL094C | 2811 | 1,026 | 1,454 | 1.42 |
| YGL142C | 1134 | 2,008 | 4,254 | 2.12 | YOL097C | 2810 | 1,438 | 14,666 | 10.20 |
| YGL147C | 24 | 692 | 105,956 | 232.49 | YOL098C | 2809 | 3,434 | 10,761 | 3.13 |
| YGL148W | 1230 | 1,265 | 28,419 | 22.48 | YOL102C | 2808 | 834 | 879 | 1.05 |
| YGL155W | 1231 | 1,455 | 3,025 | 2.08 | YOL103W | 2873 | 2,183 | 4,694 | 2.15 |
| YGL157W | 1232 | 1,172 | 3,208 | 2.74 | YOL109W | 2874 | 537 | 56,663 | 105.52 |
| YGL160W | 1233 | 1,936 | 2,991 | 1.54 | YOL110W | 2875 | 898 | 2,373 | 2.64 |
| YGL161C | 1133 | 1,160 | 8,710 | 7.51 | YOL111C | 2807 | 805 | 2,432 | 3.02 |
| YGL166W | 1234 | 861 | 3,346 | 3.89 | YOL120C | 2806 | 651 | 42,695 | 128.72 |
| YGL167C | 1132 | 3,111 | 13,154 | 4.23 | YOL121C | 2805 | 537 | 23,809 | 69.68 |
| YGL169W | 1235 | 1,577 | 3,053 | 1.94 | YOL122C | 2804 | 2,013 | 5,608 | 2.79 |
| YGL172W | 1236 | 1,538 | 6,341 | 4.12 | YOL123W | 2876 | 2,091 | 8,102 | 3.87 |
| YGL173C | 1131 | 5,044 | 14,908 | 2.96 | YOL124C | 2803 | 1,390 | 2,370 | 1.70 |
| YGL181W | 1237 | 1,357 | 3,460 | 2.59 | YOL125W | 2877 | 1,514 | 2,344 | 1.55 |
| YGL186C | 1130 | 1,824 | 4,280 | 2.35 | YOL126C | 2802 | 1,514 | 1,905 | 1.26 |
| YGL187C | 1129 | 1,196 | 10,579 | 8.84 | YOL127W | 2878 | 523 | 151,867 | 291.08 |
| YGL189C | 1128 | 803 | 39,786 | 60.47 | YOL128C | 2801 | 1,330 | 1,353 | 1.02 |
| YGL191W | 1238 | 698 | 4,369 | 6.26 | YOL129W | 2879 | 734 | 6,145 | 8.37 |
| YGL193C | 1127 | 357 | 653 | 1.83 | YOL130W | 2880 | 3,091 | 7,205 | 2.34 |
| YGL194C-A | 1126 | 447 | 1,609 | 3.60 | YOL133W | 2881 | 653 | 4,262 | 6.53 |
| YGL195W | 1239 | 8,145 | 28,034 | 3.44 | YOL136C | 2800 | 1,707 | 2,394 | 1.40 |
| YGL196W | 1240 | 1,465 | 3,220 | 2.20 | YOL139C | 2799 | 797 | 30,133 | 37.83 |
| YGL198W | 1241 | 758 | 5,580 | 7.36 | YOL140W | 2882 | 1,338 | 2,653 | 1.98 |
| YGL200C | 1125 | 744 | 23,071 | 31.01 | YOL142W | 2883 | 797 | 1,881 | 2.36 |
| YGL202W | 1242 | 1,691 | 29,993 | 17.74 | YOL143C | 2798 | 608 | 13,439 | 22.10 |
| YGL206C | 1124 | 5,288 | 15,809 | 2.99 | YOL146W | 2884 | 670 | 1,659 | 2.48 |
| YGL207W | 1243 | 3,328 | 6,513 | 1.96 | YOL147C | 2797 | 911 | 3,460 | 3.80 |
| YGL210W | 1244 | 801 | 1,158 | 1.45 | YOL151W | 2885 | 1,118 | 6,215 | 5.56 |
| YGL211W | 1245 | 1,209 | 2,811 | 2.32 | YOL155C | 2796 | 2,904 | 3,330 | 1.24 |
| YGL213C | 1123 | 1,447 | 3,165 | 2.19 | YOL158C | 2795 | 1,971 | 9,229 | 4.68 |
| YGL215W | 1246 | 2,126 | 3,703 | 1.74 | YOL159C | 2794 | 575 | 842 | 1.46 |
| YGL219C | 1122 | 1,445 | 1,481 | 1.02 | YOR001W | 2886 | 2,302 | 3,269 | 1.42 |
| YGL220W | 1247 | 363 | 4,101 | 11.30 | YOR002W | 2887 | 1,754 | 13,061 | 7.45 |
| YGL221C | 1121 | 1,287 | 14,167 | 11.01 | YOR004W | 2888 | 958 | 2,414 | 2.52 |
| YGL223C | 1120 | 1,319 | 2,421 | 1.83 | YOR007C | 2793 | 1,154 | 22,861 | 19.82 |
| YGL224C | 1119 | 1,058 | 1,646 | 1.56 | YOR008C | 2792 | 1,631 | 4,729 | 2.90 |
| YGL225W | 1248 | 1,169 | 18,318 | 15.67 | YOR014W | 2889 | 2,593 | 4,369 | 1.68 |
| YGL226C-A | 1118 | 418 | 1,237 | 2.96 | YOR016C | 2791 | 832 | 4,060 | 4.88 |
| YGL228W | 1249 | 1,921 | 4,469 | 2.35 | YOR020C | 2790 | 424 | 14,235 | 33.57 |
| YGL231C | 1117 | 630 | 3,348 | 5.31 | YOR021C | 2789 | 795 | 5,357 | 6.74 |
| YGL234W | 1250 | 2,553 | 40,160 | 15.73 | YOR025W | 2890 | 1,536 | 1,832 | 1.19 |
| YGL236C | 1116 | 2,066 | 3,273 | 1.58 | YOR026W | 2891 | 1,066 | 1,520 | 1.43 |
| YGL238W | 1251 | 2,983 | 7,119 | 2.39 | YOR027W | 2892 | 2,045 | 13,611 | 6.65 |
| YGL242C | 1115 | 689 | 1,832 | 2.66 | YOR034C | 2788 | 2,351 | 3,759 | 1.60 |
| YGL245W | 1252 | 2,208 | 47,616 | 21.57 | YOR039W | 2893 | 1,131 | 4,118 | 3.64 |
| YGL246C | 1114 | 1,316 | 1,769 | 1.34 | YOR042W | 2894 | 1,287 | 2,857 | 2.22 |
| YGL248W | 1253 | 1,212 | 1,693 | 1.40 | YOR043W | 2895 | 1,673 | 3,438 | 2.05 |
| YGL252C | 1113 | 2,008 | 2,640 | 1.31 | YOR045W | 2896 | 379 | 14,119 | 37.25 |
| YGL253W | 1254 | 1,683 | 92,702 | 55.37 | YOR046C | 2787 | 1,534 | 5,572 | 3.63 |
| YGL254W | 1255 | 1,344 | 1,454 | 1.08 | YOR048C | 2786 | 3,121 | 7,586 | 2.43 |
| YGL255W | 1256 | 1,265 | 2,433 | 1.92 | YOR051C | 2785 | 1,437 | 5,077 | 3.53 |
| YGL256W | 1257 | 1,591 | 5,596 | 3.52 | YOR052C | 2784 | 814 | 3,493 | 4.29 |
| YGL257C | 1112 | 1,853 | 3,441 | 1.86 | YOR056C | 2783 | 1,442 | 2,024 | 1.40 |
| YGR001C | 1111 | 800 | 3,612 | 4.51 | YOR057W | 2897 | 1,526 | 1,873 | 1.23 |
| YGR004W | 1258 | 1,474 | 1,837 | 1.25 | YOR061W | 2898 | 1,359 | 2,034 | 1.50 |
| YGR007W | 1259 | 1,153 | 3,053 | 2.65 | YOR063W | 2899 | 1,308 | 523,283 | 400.17 |
| YGR008C | 1110 | 464 | 1,103 | 2.38 | YOR065W | 2900 | 1,547 | 2,619 | 1.70 |
| YGR010W | 1260 | 1,188 | 1,297 | 1.09 | YOR066W | 2901 | 2,134 | 2,681 | 1.26 |
| YGR012W | 1261 | 1,346 | 2,510 | 1.86 | YOR067C | 2782 | 1,807 | 6,037 | 3.34 |
| YGR014W | 1262 | 4,057 | 18,779 | 4.79 | YOR071C | 2781 | 1,797 | 2,700 | 1.56 |
| YGR017W | 1263 | 1,157 | 1,847 | 1.60 | YOR079C | 2780 | 1,107 | 2,191 | 1.98 |
| YGR019W | 1264 | 1,551 | 3,606 | 2.32 | YOR081C | 2779 | 2,362 | 2,474 | 1.05 |
| YGR020C | 1109 | 541 | 6,660 | 12.31 | YOR084W | 2902 | 1,323 | 2,365 | 1.79 |
| YGR024C | 1108 | 813 | 4,094 | 5.08 | YOR085W | 2903 | 1,383 | 8,768 | 6.34 |
| YGR026W | 1265 | 991 | 3,044 | 3.07 | YOR086C | 2778 | 3,858 | 10,066 | 2.61 |
| YGR027C | 1107 | 738 | 54,305 | 82.05 | YOR087W | 2904 | 2,128 | 4,754 | 2.23 |
| YGR028W | 1266 | 1,234 | 2,320 | 1.88 | YOR089C | 2777 | 699 | 1,171 | 1.68 |
| YGR029W | 1267 | 666 | 1,876 | 2.82 | YOR090C | 2776 | 2,006 | 3,224 | 1.61 |
| YGR031W | 1268 | 1,484 | 7,484 | 5.04 | YOR091W | 2905 | 1,166 | 2,429 | 2.08 |
| YGR033C | 1106 | 1,052 | 3,870 | 3.68 | YOR095C | 2775 | 995 | 6,931 | 6.97 |
| YGR034W | 1269 | 544 | 47,706 | 112.84 | YOR096W | 2906 | 695 | 135,254 | 240.10 |
| YGR036C | 1105 | 720 | 1,325 | 1.84 | YOR098C | 2774 | 3,362 | 5,477 | 1.63 |
| YGR037C | 1104 | 460 | 25,157 | 54.69 | YOR099W | 2907 | 1,339 | 30,353 | 22.68 |
| YGR038W | 1270 | 956 | 5,148 | 5.38 | YOR101W | 2908 | 1,227 | 3,953 | 3.22 |
| YGR040W | 1271 | 1,959 | 2,516 | 1.28 | YOR103C | 2773 | 503 | 3,696 | 7.35 |
| YGR044C | 1103 | 1,119 | 3,899 | 3.48 | YOR108W | 2909 | 1,998 | 19,590 | 9.98 |
| YGR046W | 1272 | 1,648 | 1,651 | 1.00 | YOR109W | 2910 | 3,487 | 5,034 | 1.44 |
| YGR048W | 1273 | 1,215 | 1,534 | 1.26 | YOR115C | 2772 | 937 | 1,952 | 2.08 |
| YGR054W | 1274 | 2,152 | 12,263 | 5.70 | YOR116C | 2771 | 4,727 | 11,284 | 2.39 |
| YGR055W | 1275 | 1,870 | 16,705 | 8.93 | YOR117W | 2911 | 1,505 | 6,379 | 4.24 |
| YGR060W | 1276 | 1,051 | 25,048 | 23.83 | YOR120W | 2912 | 1,125 | 1,229 | 1.09 |
| YGR061C | 1102 | 4,475 | 23,945 | 5.35 | YOR122C | 2770 | 582 | 39,934 | 68.61 |
| YGR062C | 1101 | 994 | 1,058 | 1.06 | YOR125C | 2769 | 867 | 2,324 | 2.68 |
| YGR063C | 1100 | 389 | 2,568 | 6.60 | YOR126C | 2768 | 834 | 1,371 | 1.64 |
| YGR065C | 1099 | 1,992 | 3,564 | 1.79 | YOR128C | 2767 | 1,787 | 4,705 | 2.63 |
| YGR074W | 1277 | 736 | 1,711 | 2.32 | YOR131C | 2766 | 855 | 2,632 | 3.08 |
| YGR077C | 1098 | 1,808 | 3,377 | 1.87 | YOR132W | 2913 | 1,758 | 2,420 | 1.38 |
| YGR078C | 1097 | 742 | 2,061 | 2.81 | YOR133W | 2914 | 2,777 | 9,220 | 20.15 |
| YGR079W | 1278 | 1,205 | 2,395 | 1.99 | YOR136W | 2915 | 1,266 | 22,323 | 17.64 |
| YGR080W | 1279 | 1,077 | 2,895 | 2.69 | YOR138C | 2765 | 2,100 | 3,417 | 1.63 |
| YGR081C | 1096 | 709 | 745 | 1.05 | TOR142W | 2916 | 1,385 | 10,913 | 7.88 |
| YGR082W | 1280 | 749 | 7,560 | 10.09 | YOR145C | 2764 | 895 | 3,862 | 4.31 |
| YGR084C | 1095 | 1,070 | 1,581 | 1.48 | YOR149C | 2763 | 1,627 | 3,222 | 1.98 |
| YGR085C | 1094 | 618 | 11,493 | 46.95 | YOR150W | 2917 | 605 | 1,308 | 2.16 |
| YGR086C | 1093 | 1,371 | 20,772 | 15.19 | YOR151C | 2762 | 3,971 | 11,638 | 2.93 |
| YGR090W | 1281 | 3,946 | 7,859 | 1.99 | YOR152C | 2761 | 1,137 | 1,661 | 1.46 |
| YGR094W | 1282 | 3,479 | 38,286 | 11.01 | YOR153W | 2918 | 4,783 | 143,333 | 30.01 |
| YGR095C | 1092 | 746 | 1,549 | 2.08 | YOR155C | 2760 | 1,605 | 2,056 | 1.28 |
| YGR096W | 1283 | 945 | 1,249 | 1.32 | YOR157C | 2759 | 857 | 6,905 | 8.06 |
| YGR099W | 1284 | 2,157 | 2,483 | 1.15 | YOR159C | 2758 | 285 | 423 | 1.48 |
| YGR101W | 1285 | 1,188 | 2,866 | 2.41 | YOR161C | 2757 | 1,620 | 4,876 | 3.01 |
| YGR102C | 1091 | 707 | 902 | 1.28 | YOR163W | 2919 | 680 | 4,057 | 5.97 |
| YGR103W | 1286 | 2,004 | 6,119 | 3.06 | YOR164C | 2756 | 1,074 | 9,038 | 8.41 |
| YGR105W | 1287 | 454 | 1,401 | 3.09 | YOR165W | 2920 | 2,503 | 3,929 | 1.57 |
| YGR106C | 1090 | 929 | 19,711 | 21.22 | YOR167C | 2755 | 318 | 94,668 | 334.25 |
| YGR108W | 1288 | 1,683 | 2,306 | 1.37 | YOR168W | 2921 | 2,531 | 25,388 | 10.03 |
| YGR111W | 1289 | 1,353 | 1,875 | 1.39 | YOR171C | 2754 | 2,105 | 3,418 | 1.62 |
| YGR116W | 1290 | 4,513 | 5,874 | 1.30 | YOR174W | 2922 | 1,196 | 1,222 | 1.02 |
| YGR117C | 1089 | 1,830 | 2,307 | 1.26 | YOR175C | 2753 | 2,077 | 12,636 | 6.08 |
| YGR118W | 1291 | 636 | 28,314 | 77.48 | YOR176W | 2923 | 1,414 | 8,027 | 5.68 |
| YGR119C | 1088 | 1,750 | 3,460 | 1.98 | YOR179C | 2752 | 816 | 895 | 1.10 |
| YGR122W | 1292 | 1,209 | 1,287 | 1.06 | YOR182C | 2751 | 249 | 18,722 | 109.15 |
| YGR123C | 1087 | 1,702 | 3,769 | 2.21 | YOR184W | 2924 | 1,286 | 5,469 | 6.69 |
| YGR124W | 1293 | 1,849 | 60,750 | 34.53 | YOR185C | 2750 | 1,044 | 1,277 | 1.23 |
| YGR125W | 1294 | 3,196 | 3,931 | 1.23 | YOR187W | 2925 | 1,509 | 25,787 | 17.09 |
| YGR128C | 1086 | 2,220 | 3,555 | 1.60 | YOR196C | 2749 | 1,406 | 3,059 | 2.17 |
| YGR132C | 1085 | 1,201 | 8,885 | 7.40 | YOR197W | 2926 | 1,369 | 10,857 | 7.93 |
| YGR133W | 1295 | 804 | 1,032 | 1.28 | YOR198C | 2748 | 1,613 | 7,942 | 4.92 |
| YGR135W | 1296 | 919 | 10,021 | 10.90 | YOR201C | 2747 | 1,324 | 3,103 | 2.34 |
| YGR136W | 1297 | 726 | 2,299 | 3.17 | YOR202W | 2927 | 755 | 3,514 | 4.65 |
| YGR141W | 1298 | 1,549 | 2,022 | 1.30 | YOR204W | 2928 | 2,558 | 31,268 | 12.23 |
| YGR143W | 1299 | 2,870 | 3,124 | 1.09 | YOR206W | 2929 | 2,229 | 4,715 | 2.11 |
| YGR147C | 1084 | 1,021 | 2,752 | 2.70 | YOR207C | 2746 | 3,638 | 6,161 | 1.69 |
| YGR148C | 1083 | 1,023 | 19,617 | 22.81 | YOR209C | 2745 | 1,523 | 6,853 | 4.50 |
| YGR149W | 1300 | 1,628 | 1,933 | 1.19 | YOR210W | 2930 | 538 | 3,237 | 6.02 |
| YGR152C | 1082 | 940 | 1,312 | 1.40 | YOR212W | 2931 | 1,560 | 6,267 | 4.02 |
| YGR155W | 1301 | 1,681 | 49,715 | 29.57 | YOR213C | 2744 | 1,167 | 1,669 | 1.43 |
| YGR157W | 1302 | 2,932 | 10,841 | 3.70 | YOR215C | 2743 | 727 | 1,029 | 1.41 |
| YGR158C | 1081 | 1,009 | 1,231 | 1.22 | YOR217W | 2932 | 2,889 | 5,616 | 1.94 |
| YGR159C | 1080 | 1,363 | 28,036 | 20.80 | YOR219C | 2742 | 3,206 | 3,287 | 1.03 |
| YGR161W-C | 1303 | 583 | 803 | 1.38 | YOR220W | 2933 | 997 | 1,067 | 1.07 |
| YGR162W | 1304 | 3,061 | 19,151 | 6.26 | YOR221C | 2741 | 1,296 | 1,380 | 1.06 |
| YGR165W | 1305 | 1,214 | 3,127 | 2.58 | YOR222W | 2934 | 1,434 | 5,487 | 3.83 |
| YGR167W | 1306 | 891 | 2,783 | 3.12 | YOR224C | 2740 | 533 | 18,427 | 34.57 |
| YGR172C | 1079 | 894 | 6,779 | 7.61 | YOR226C | 2739 | 651 | 1,902 | 2.92 |
| YGR173W | 1307 | 1,366 | 2,964 | 2.17 | YOR230W | 2935 | 1,541 | 28,943 | 18.79 |
| YGR175C | 1078 | 1,714 | 26,734 | 15.60 | YOR231W | 2936 | 1,805 | 2,084 | 1.15 |
| YGR177C | 1077 | 1,608 | 4,110 | 2.56 | YOR232W | 2937 | 943 | 5,458 | 5.79 |
| YGR178C | 1076 | 2,485 | 10,309 | 4.15 | YOR234C | 2738 | 416 | 82,011 | 222.63 |
| YGR180C | 1075 | 1,314 | 37,930 | 28.89 | YOR236W | 2938 | 700 | 1,498 | 2.14 |
| YGR181W | 1308 | 550 | 2,796 | 5.08 | YOR239W | 2939 | 2,140 | 3,631 | 1.70 |
| YGR183C | 1074 | 618 | 6,208 | 10.05 | YOR241W | 2940 | 1,714 | 4,048 | 2.36 |
| YGR185C | 1073 | 1,289 | 14,425 | 11.19 | YOR243C | 2737 | 2,193 | 5,291 | 2.41 |
| YGR189C | 1072 | 1,653 | 15,893 | 9.62 | YOR246C | 2736 | 1,119 | 10,900 | 9.74 |
| YGR191W | 1309 | 2,183 | 12,841 | 5.88 | YOR247W | 2941 | 633 | 24,202 | 38.23 |
| YGR192C | 25 | 1,129 | 248,847 | 730.28 | YOR251C | 2735 | 1,035 | 4,293 | 4.15 |
| YGR193C | 1071 | 1,409 | 3,424 | 2.43 | YOR253W | 2942 | 664 | 3,822 | 5.77 |
| YGR195W | 1310 | 923 | 2,996 | 3.25 | YOR254C | 2734 | 2,191 | 11,189 | 5.11 |
| YGR197C | 1070 | 1,733 | 1,789 | 1.03 | YOR259C | 2733 | 1,381 | 5,357 | 3.88 |
| YGR199W | 1311 | 2,280 | 5,019 | 2.20 | YOR260W | 2943 | 1,990 | 7,702 | 3.87 |
| YGR200C | 1069 | 2,473 | 6,330 | 2.56 | YOR261C | 2732 | 1,176 | 5,214 | 4.43 |
| YGR201C | 1068 | 792 | 2,009 | 2.54 | YOR264W | 2944 | 1,418 | 2,343 | 1.65 |
| YGR202C | 1067 | 1,518 | 2,313 | 1.52 | YOR265W | 2945 | 388 | 479 | 1.23 |
| YGR203W | 1312 | 962 | 1,385 | 1.44 | YOR270C | 2731 | 2,792 | 49,548 | 17.75 |
| YGR204W | 1313 | 2,962 | 33,970 | 11.47 | YOR271C | 2730 | 1,106 | 8,613 | 7.79 |
| YGR206W | 1314 | 428 | 658 | 1.54 | YOR272W | 2946 | 1,501 | 5,807 | 3.87 |
| YGR207C | 1066 | 897 | 1,289 | 1.44 | YOR273C | 2729 | 2,190 | 4,308 | 1.97 |
| YGR208W | 1315 | 1,022 | 5,091 | 4.98 | YOR276W | 2947 | 620 | 17,048 | 27.50 |
| YGR209C | 1065 | 413 | 55,338 | 134.48 | YOR278W | 2948 | 828 | 1,295 | 1.56 |
| YGR210C | 1064 | 1,429 | 8,002 | 5.60 | YOR280C | 2728 | 904 | 1,598 | 1.77 |
| YGR211W | 1316 | 1,719 | 12,032 | 7.00 | YOR283W | 2949 | 921 | 1,655 | 1.80 |
| YGR214W | 1317 | 840 | 77,456 | 112.72 | YOR285W | 2950 | 502 | 17,369 | 34.60 |
| YGR216C | 1063 | 1,945 | 2,497 | 1.28 | YOR286W | 2951 | 527 | 4,946 | 9.38 |
| YGR218W | 1318 | 3,743 | 9,741 | 2.60 | YOR288C | 2727 | 1,053 | 2,596 | 2.47 |
| YGR220C | 1062 | 922 | 2,687 | 2.91 | YOR291W | 2952 | 4,532 | 7,028 | 1.55 |
| YGR222W | 1319 | 1,000 | 1,045 | 1.05 | YOR292C | 2726 | 987 | 1,430 | 1.45 |
| YGR227W | 1320 | 1,724 | 2,718 | 1.58 | YOR293W | 2953 | 494 | 62,020 | 154.23 |
| YGR229C | 1061 | 1,947 | 4,249 | 2.19 | YOR294W | 2954 | 809 | 2,366 | 2.92 |
| YGR230W | 1321 | 645 | 785 | 1.22 | YOR297C | 2725 | 752 | 966 | 1.28 |
| YGR231C | 1060 | 1,156 | 6,194 | 5.36 | YOR298C-A | 2724 | 709 | 70,539 | 99.72 |
| YGR232W | 1322 | 768 | 2,078 | 2.71 | YOR299W | 2955 | 2,241 | 4,585 | 2.05 |
| YGR233C | 1059 | 3,671 | 4,855 | 1.32 | YOR301W | 2956 | 1,599 | 5,713 | 3.57 |
| YGR234W | 1323 | 1,491 | 49,695 | 33.33 | YOR302W | 2957 | 270 | 387 | 1.45 |
| YGR235C | 1058 | 838 | 2,497 | 2.98 | YOR303W | 2958 | 1,297 | 11,605 | 8.95 |
| YGR239C | 1057 | 1,092 | 1,113 | 1.02 | YOR306C | 2723 | 1,842 | 3,302 | 1.79 |
| YGR240C | 1056 | 3,393 | 62,236 | 18.37 | YOR307C | 2722 | 1,639 | 4,667 | 2.85 |
| YGR241C | 1055 | 1,856 | 1,909 | 1.03 | YOR310C | 2721 | 1,637 | 12,269 | 7.55 |
| YGR244C | 1054 | 1,474 | 5,948 | 4.04 | YOR311C | 2720 | 935 | 5,762 | 6.16 |
| YGR245C | 1053 | 2,480 | 2,558 | 1.03 | YOR312C | 2719 | 588 | 33,903 | 93.71 |
| YGR247W | 1324 | 873 | 1,575 | 1.82 | YOR315W | 2959 | 1,341 | 1,717 | 1.28 |
| YGR253C | 1052 | 868 | 5,137 | 5.92 | YOR316C | 2718 | 1,565 | 4,505 | 3.15 |
| YGR254W | 1325 | 1,505 | 32,998 | 50.65 | YOR317W | 2960 | 2,497 | 8,609 | 3.45 |
| YGR255C | 1051 | 1,730 | 2,673 | 1.54 | YOR319W | 2961 | 841 | 1,310 | 1.56 |
| YGR257C | 1050 | 1,226 | 3,094 | 2.52 | YOR320C | 2717 | 1,721 | 6,239 | 3.63 |
| YGR260W | 1326 | 1,718 | 17,260 | 10.05 | YOR321W | 2962 | 2,339 | 5,072 | 2.17 |
| YGR261C | 1049 | 2,533 | 5,459 | 2.15 | YOR322C | 2716 | 2,820 | 3,005 | 1.06 |
| YGR262C | 1048 | 893 | 3,735 | 4.18 | YOR323C | 2715 | 1,434 | 9,182 | 6.40 |
| YGR264C | 1047 | 2,487 | 25,381 | 10.20 | YOR326W | 2963 | 5,069 | 12,856 | 2.54 |
| YGR266W | 1327 | 2,403 | 3,111 | 1.29 | YOR327C | 2714 | 519 | 1,197 | 2.31 |
| YGR267C | 1046 | 1,007 | 4,956 | 4.94 | YOR332W | 2964 | 804 | 15,244 | 18.96 |
| YGR268C | 1045 | 749 | 1,028 | 1.38 | YOR335C | 2713 | 3,085 | 25,949 | 8.41 |
| YGR277C | 1044 | 990 | 1,941 | 1.96 | YOR336W | 2965 | 4,334 | 4,592 | 1.06 |
| YGR279C | 1043 | 1,242 | 60,884 | 49.21 | YOR337W | 2966 | 2,300 | 2,381 | 1.03 |
| YGR281W | 1328 | 4,816 | 5,601 | 1.16 | YOR340C | 2712 | 1,159 | 2,281 | 1.97 |
| YGR282C | 1042 | 1,141 | 48,901 | 42.88 | YOR341W | 2967 | 5,379 | 21,588 | 4.01 |
| YGR283C | 1041 | 1,102 | 1,237 | 1.12 | YOR342C | 2711 | 1,277 | 1,950 | 1.53 |
| YGR284C | 1040 | 1,137 | 17,055 | 15.00 | YOR347C | 2710 | 1,652 | 2,481 | 1.50 |
| YGR285C | 1039 | 1,361 | 64,781 | 47.60 | YOR354C | 2709 | 2,260 | 2,992 | 1.33 |
| YGR286C | 1038 | 1,533 | 2,990 | 1.95 | YOR355W | 2968 | 1,851 | 6,278 | 3.39 |
| YGR295C | 1037 | 1,146 | 1,189 | 1.96 | YOR356W | 2969 | 2,095 | 4,365 | 2.08 |
| YHL001W | 1401 | 563 | 15,143 | 46.51 | YOR357C | 2708 | 599 | 1,577 | 2.63 |
| YHL002W | 1402 | 1,415 | 1,591 | 1.13 | YOR359W | 2970 | 1,923 | 1,943 | 1.02 |
| YHL003C | 1395 | 1,567 | 14,313 | 9.14 | YOR360C | 2707 | 1,883 | 6,980 | 3.71 |
| YHL004W | 1403 | 1,368 | 2,856 | 2.09 | YOR361C | 2706 | 2,358 | 27,628 | 11.72 |
| YHL007C | 1394 | 2,939 | 3,467 | 1.18 | YOR362C | 2705 | 1,024 | 10,830 | 10.58 |
| YHL011C | 1393 | 1,178 | 14,250 | 12.10 | YOR367W | 2971 | 681 | 1,256 | 1.84 |
| YHL015W | 26 | 686 | 186,676 | 276.03 | YOR369C | 2704 | 586 | 422,461 | 720.92 |
| YHL017W | 1404 | 1,804 | 6,138 | 3.41 | YOR370C | 2703 | 2,279 | 6,763 | 2.97 |
| YHL020C | 1392 | 1,390 | 3,243 | 2.36 | YOR374W | 2972 | 1,825 | 3,941 | 2.16 |
| YHL021C | 1391 | 1,497 | 2,257 | 1.51 | YOR375C | 2702 | 1,503 | 10,587 | 7.05 |
| YHL025W | 1405 | 1,133 | 2,900 | 2.56 | YOR377W | 2973 | 1,894 | 2,014 | 1.06 |
| YHL027W | 1406 | 2,198 | 3,979 | 1.81 | YOR382W | 2974 | 598 | 1,626 | 2.72 |
| YHL031C | 1390 | 707 | 1,902 | 2.69 | YOR383C | 2701 | 781 | 4,580 | 6.07 |
| YHL032C | 1389 | 2,195 | 3,306 | 1.51 | YPL001W | 3095 | 1,187 | 1,596 | 1.34 |
| YHL033C | 1388 | 1,248 | 32,563 | 35.86 | YPL002C | 3081 | 885 | 1,246 | 1.41 |
| YHL034C | 1387 | 1,126 | 6,942 | 6.17 | YPL003W | 3096 | 1,438 | 1,597 | 1.11 |
| YHL039W | 1407 | 1,758 | 3,786 | 2.15 | YPL004C | 3080 | 1,244 | 16,510 | 13.33 |
| YHL040C | 1386 | 1,884 | 3,975 | 2.11 | YPL006W | 3097 | 3,629 | 4,466 | 1.23 |
| YHL044W | 1408 | 966 | 1,169 | 1.21 | YPL010W | 3098 | 785 | 14,028 | 17.87 |
| YHL047C | 1385 | 1,998 | 3,384 | 1.69 | YPL011C | 3079 | 1,188 | 1,263 | 1.06 |
| YHL048W | 1409 | 1,241 | 1,032 | 1.06 | YPL012W | 3099 | 3,913 | 6,214 | 1.59 |
| YHR001W | 1410 | 1,704 | 3,396 | 1.99 | YPL013C | 3078 | 528 | 761 | 1.44 |
| YHR001W-A | 1411 | 509 | 1,316 | 2.59 | YPL015C | 3077 | 1,421 | 7,466 | 5.25 |
| YHR003C | 1384 | 1,428 | 2,078 | 1.45 | YPL019C | 3076 | 2,689 | 38,296 | 14.24 |
| YHR005C | 1383 | 1,583 | 3,135 | 1.98 | YPL023C | 3075 | 2,223 | 4,869 | 2.19 |
| YHR005C-A | 1382 | 438 | 4,299 | 9.81 | YPL024W | 3100 | 960 | 1,454 | 1.53 |
| YHR007C | 1381 | 1,971 | 60,581 | 30.74 | YPL028W | 3101 | 1,333 | 61,420 | 46.08 |
| YHR008C | 1380 | 895 | 6,087 | 6.81 | YPL030W | 3102 | 1,838 | 2,562 | 1.39 |
| YHR009C | 1379 | 1,782 | 6,104 | 3.42 | YPL031C | 3074 | 1,005 | 4,467 | 4.44 |
| YHR010W | 1412 | 558 | 92,639 | 191.02 | YPL032C | 3073 | 2,772 | 4,161 | 1.50 |
| YHR012W | 1413 | 1,332 | 2,428 | 1.82 | YPL036W | 3103 | 3,216 | 7,042 | 2.35 |
| YHR013C | 1378 | 843 | 2,811 | 3.33 | YPL037C | 3072 | 535 | 62,711 | 117.22 |
| YHR016C | 1377 | 1,563 | 3,535 | 2.26 | YPL038W | 3104 | 660 | 1,257 | 1.90 |
| YHR017W | 1414 | 1,261 | 1,550 | 1.23 | YPL039W | 3105 | 1,117 | 1,122 | 1.02 |
| YHR018C | 1376 | 1,582 | 6,453 | 4.08 | YPL047W | 3106 | 720 | 3,641 | 5.06 |
| YHR019C | 1375 | 1,766 | 53,415 | 30.25 | YPL048W | 3107 | 1,463 | 21,622 | 14.95 |
| YHR020W | 1415 | 2,261 | 36,935 | 16.38 | YPL049C | 3071 | 1,628 | 3,555 | 2.20 |
| YHR021C | 27 | 360 | 74,988 | 217.24 | YPL050C | 3070 | 1,445 | 8,093 | 5.61 |
| YHR024C | 1374 | 1,713 | 3,152 | 1.84 | YPL053C | 3069 | 1,514 | 3,898 | 2.57 |
| YHR025W | 1416 | 1,182 | 26,973 | 22.82 | YPL057C | 3068 | 1,677 | 2,798 | 1.67 |
| YHR026W | 1417 | 893 | 26,241 | 29.38 | YPL058C | 3067 | 4,915 | 7,611 | 1.55 |
| YHR027C | 1373 | 3,262 | 15,667 | 4.80 | YPL059W | 3108 | 652 | 10,480 | 16.07 |
| YHR028C | 1372 | 2,547 | 8,300 | 3.26 | YPL061W | 3109 | 1,659 | 48,360 | 29.15 |
| YHR029C | 1371 | 885 | 2,127 | 2.40 | YPL063W | 3110 | 1,619 | 3,221 | 1.99 |
| YHR030C | 1370 | 1,661 | 3,158 | 1.93 | YPL066W | 3111 | 1,599 | 1,676 | 1.05 |
| YHR032W | 1418 | 1,746 | 7,368 | 4.22 | YPL067C | 3066 | 729 | 1,634 | 2.24 |
| YHR033W | 1419 | 1,322 | 2,654 | 2.01 | YPL075W | 3112 | 2,594 | 2,803 | 1.08 |
| YHR034C | 1369 | 1,181 | 1,538 | 1.30 | YPL078C | 3065 | 1,021 | 8,667 | 8.49 |
| YHR037W | 1420 | 1,884 | 3,222 | 1.71 | YPL079W | 3113 | 616 | 52,108 | 130.56 |
| YHR039C | 1368 | 2,097 | 15,623 | 7.45 | YPL081W | 3114 | 824 | 12,857 | 19.14 |
| YHR039C-A | 1367 | 415 | 3,879 | 9.35 | YPL083C | 3064 | 1,511 | 1,834 | 1.22 |
| YHR041C | 1366 | 855 | 4,830 | 5.65 | YPL086C | 3063 | 1,794 | 4,913 | 2.74 |
| YHR042W | 1421 | 2,234 | 24,781 | 11.09 | YPL087W | 3115 | 1,185 | 2,967 | 2.50 |
| YHR043C | 1365 | 894 | 1,459 | 1.82 | YPL090C | 3062 | 880 | 16,669 | 64.06 |
| YHR045W | 1422 | 1,846 | 3,564 | 1.93 | YPL091W | 3116 | 1,625 | 9,902 | 6.09 |
| YHR046C | 1364 | 1,104 | 1,306 | 1.19 | YPL092W | 3117 | 1,590 | 3,136 | 1.97 |
| YHR047C | 1363 | 2,743 | 12,127 | 4.42 | YPL093W | 3118 | 2,241 | 20,612 | 9.20 |
| YHR049W | 1423 | 892 | 9,587 | 10.75 | YPL094C | 3061 | 881 | 13,524 | 15.35 |
| YHR050W | 1424 | 1,696 | 6,539 | 3.85 | YPL095C | 3060 | 1,558 | 2,820 | 1.81 |
| YHR051W | 1425 | 951 | 5,434 | 5.71 | YPL096C-A | 3059 | 321 | 718 | 2.24 |
| YHR052W | 1426 | 1,291 | 4,657 | 3.61 | YPL098C | 3058 | 510 | 4,714 | 9.24 |
| YHR055C | 1362 | 367 | 1,007 | 4.02 | YPL099C | 3057 | 682 | 738 | 1.08 |
| YHR057C | 1361 | 708 | 2,708 | 3.82 | YPL101W | 3119 | 1,488 | 3,478 | 2.34 |
| YHR058C | 1360 | 978 | 1,388 | 1.42 | YPL105C | 3056 | 2,673 | 3,211 | 1.20 |
| YHR061C | 1359 | 1,382 | 1,489 | 1.08 | YPL106C | 3055 | 2,242 | 68,969 | 30.78 |
| YHR062C | 1358 | 993 | 2,307 | 2.32 | YPL111W | 3120 | 1,143 | 11,792 | 10.32 |
| YHR063C | 1357 | 1,214 | 5,690 | 4.69 | YPL112C | 3054 | 1,289 | 2,067 | 1.60 |
| YHR064C | 1356 | 1,720 | 119,941 | 69.75 | YPL116W | 3121 | 2,223 | 2,463 | 1.11 |
| YHR065C | 1355 | 1,622 | 2,780 | 1.71 | YPL117C | 3053 | 1,126 | 6,440 | 5.72 |
| YHR066W | 1427 | 1,510 | 1,693 | 1.16 | YPL118W | 3122 | 1,222 | 1,935 | 1.58 |
| YHR067W | 1428 | 1,199 | 1,544 | 1.33 | YPL126W | 3123 | 2,824 | 8,124 | 2.88 |
| YHR068W | 1429 | 1,378 | 22,116 | 16.48 | YPL127C | 3052 | 1,123 | 5,222 | 4.65 |
| YHR069C | 1354 | 1,203 | 5,698 | 4.74 | YPL128C | 3051 | 1,689 | 4,427 | 2.62 |
| YHR070W | 1430 | 2,079 | 6,384 | 3.07 | YPL129W | 3124 | 896 | 2,741 | 3.06 |
| YHR071W | 1431 | 808 | 1,279 | 1.61 | YPL131W | 3125 | 1,014 | 200,469 | 197.70 |
| YHR072W | 1432 | 2,373 | 10,309 | 4.34 | YPL132W | 3126 | 1,104 | 1,515 | 1.37 |
| YHR072W-A | 1433 | 396 | 35,255 | 89.03 | YPL133C | 3050 | 1,410 | 1,433 | 1.02 |
| YHR073W | 1434 | 3,217 | 5,652 | 1.76 | YPL134C | 3049 | 1,229 | 1,611 | 1.31 |
| YHR074W | 1435 | 2,282 | 15,949 | 6.99 | YPL135W | 3127 | 963 | 6,255 | 6.49 |
| YHR076W | 1436 | 1,176 | 2,494 | 2.12 | YPL137C | 3048 | 3,911 | 6,381 | 1.63 |
| YHR078W | 1437 | 1,857 | 3,173 | 1.71 | YPL143W | 3128 | 394 | 156,148 | 451.12 |
| YHR083W | 1438 | 1,086 | 4,949 | 4.56 | YPL144W | 3129 | 553 | 1,317 | 2.38 |
| YHR086W | 1439 | 2,108 | 3,164 | 1.50 | YPL145C | 3047 | 1,997 | 17,388 | 8.71 |
| YHR087W | 1440 | 589 | 649 | 1.10 | YPL148C | 3046 | 804 | 921 | 1.14 |
| YHR088W | 1441 | 1,013 | 1,282 | 1.27 | YPL149W | 3130 | 1,028 | 2,479 | 2.41 |
| YHR089C | 1353 | 841 | 8,375 | 10.00 | YPL154C | 3045 | 1,536 | 22,240 | 14.48 |
| YHR092C | 1352 | 1,921 | 2,667 | 1.56 | YPL160W | 3131 | 3,496 | 24,345 | 6.97 |
| YHR094C | 1351 | 1,853 | 17,194 | 10.38 | YPL162C | 3044 | 971 | 1,716 | 1.77 |
| YHR098C | 1350 | 2,987 | 14,273 | 4.78 | YPL163C | 3043 | 1,089 | 9,539 | 8.76 |
| YHR103W | 1442 | 2,677 | 5,542 | 2.07 | YPL169C | 3042 | 2,077 | 4,671 | 2.25 |
| YHR104W | 1443 | 1,212 | 3,947 | 3.26 | YPL170W | 3132 | 763 | 1,863 | 2.44 |
| YHR106W | 1444 | 1,332 | 1,812 | 1.37 | YPL172C | 3041 | 1,539 | 2,251 | 1.46 |
| YHR107C | 1349 | 1,441 | 2,590 | 1.80 | YPL176C | 3040 | 2,485 | 5,587 | 2.25 |
| YHR108W | 1445 | 1,958 | 13,535 | 6.91 | YPL177C | 3039 | 1,413 | 2,933 | 2.08 |
| YHR110W | 1446 | 763 | 5,512 | 7.22 | YPL178W | 3133 | 790 | 4,910 | 6.22 |
| YHR111W | 1447 | 1,323 | 2,267 | 1.71 | YPL179W | 3134 | 2,042 | 4,116 | 2.02 |
| YHR112C | 1348 | 1,378 | 2,533 | 1.84 | YPL183C | 3038 | 3,174 | 4,933 | 1.55 |
| YHR113W | 1448 | 1,586 | 6,084 | 3.84 | YPL183W-A | 3135 | 473 | 2,908 | 6.15 |
| YHR115C | 1347 | 1,503 | 5,076 | 3.40 | YPL184C | 3037 | 2,122 | 7,529 | 3.55 |
| YHR116W | 1449 | 722 | 921 | 1.28 | YPL187W | 3136 | 498 | 31,258 | 97.56 |
| YHR117W | 1450 | 1,920 | 6,399 | 3.33 | YPL189C-A | 3036 | 495 | 818 | 1.65 |
| YHR121W | 1451 | 731 | 2,780 | 3.80 | YPL198W | 3137 | 856 | 4,047 | 10.41 |
| YHR122W | 1452 | 897 | 2,946 | 3.28 | YPL199C | 3035 | 788 | 1,536 | 1.95 |
| YHR123W | 1453 | 1,337 | 7,487 | 5.60 | YPL203W | 3138 | 1,548 | 1,917 | 1.24 |
| YHR127W | 1454 | 930 | 971 | 1.04 | YPL204W | 3139 | 1,830 | 3,566 | 1.95 |
| YHR128W | 1455 | 932 | 15,668 | 16.81 | YPL206C | 3034 | 1,116 | 3,915 | 3.51 |
| YHR132C | 1346 | 1,479 | 12,039 | 8.14 | YPL207W | 3140 | 2,606 | 5,009 | 1.92 |
| YHR132W-A | 1456 | 745 | 1,216 | 1.64 | YPL208W | 3141 | 1,818 | 4,365 | 2.40 |
| YHR133C | 1345 | 1,047 | 10,331 | 9.87 | YPL210C | 3033 | 2,088 | 2,309 | 1.11 |
| YHR135C | 1344 | 1,810 | 4,697 | 2.63 | YPL211W | 3142 | 708 | 8,235 | 11.63 |
| YHR136C | 1343 | 613 | 3,339 | 5.45 | YPL212C | 3032 | 1,826 | 3,502 | 1.92 |
| YHR137W | 1457 | 1,869 | 8,085 | 4.33 | YPL214C | 3031 | 1,721 | 3,174 | 1.84 |
| YHR138C | 1342 | 478 | 831 | 1.74 | YPL215W | 3143 | 1,186 | 2,341 | 1.97 |
| YHR141C | 28 | 398 | 39,209 | 222.47 | YPL218W | 3144 | 689 | 29,209 | 42.39 |
| YHR142W | 1458 | 1,214 | 3,473 | 2.86 | YPL220W | 3145 | 826 | 17,129 | 73.84 |
| YHR143W | 1459 | 1,488 | 15,933 | 10.79 | YPL221W | 3146 | 2,576 | 10,052 | 3.90 |
| YHR143W-A | 1460 | 520 | 13,821 | 26.58 | YPL224C | 3030 | 1,795 | 2,841 | 1.58 |
| YHR144C | 1341 | 1,211 | 3,244 | 2.68 | YPL225W | 3147 | 601 | 2,614 | 4.36 |
| YHR146W | 1461 | 1,472 | 2,611 | 1.77 | YPL226W | 3148 | 3,905 | 26,982 | 6.92 |
| YHR147C | 1340 | 799 | 1,674 | 2.10 | YPL227C | 3029 | 1,197 | 2,046 | 1.71 |
| YHR148W | 1462 | 879 | 1,379 | 1.57 | YPL231W | 3149 | 5,830 | 89,342 | 15.32 |
| YHR149C | 1339 | 2,375 | 3,867 | 1.63 | YPL232W | 3150 | 1,131 | 3,407 | 3.01 |
| YHR151C | 1338 | 1,581 | 2,490 | 1.57 | YPL234C | 3028 | 663 | 16,983 | 25.61 |
| YHR161C | 1337 | 2,310 | 2,295 | 1.00 | YPL235W | 3151 | 1,575 | 5,096 | 3.24 |
| YHR162W | 1463 | 674 | 23,469 | 34.82 | YPL237W | 3152 | 967 | 11,947 | 12.35 |
| YHR163W | 1464 | 935 | 12,613 | 13.49 | YPL239W | 3153 | 878 | 2,645 | 3.01 |
| YHR167W | 1465 | 900 | 919 | 1.02 | YPL240C | 3027 | 2,264 | 15,893 | 8.66 |
| YHR169W | 1466 | 1,517 | 3,669 | 2.42 | YPL241C | 3026 | 875 | 1,685 | 2.05 |
| YHR170W | 1467 | 1,710 | 15,024 | 8.79 | YPL243W | 3154 | 1,995 | 7,146 | 3.58 |
| YHR174W | 29 | 1,555 | 342,805 | 496.14 | YPL244C | 3025 | 1,181 | 4,628 | 3.92 |
| YHR175W | 1468 | 774 | 3,369 | 4.57 | YPL245W | 3155 | 1,477 | 4,918 | 3.33 |
| YHR179W | 1469 | 1,355 | 27,667 | 20.42 | YPL246C | 3024 | 980 | 6,374 | 6.50 |
| YHR181W | 1470 | 835 | 6,353 | 7.61 | YPL247C | 3023 | 1,725 | 2,122 | 1.23 |
| YHR183W | 1471 | 1,669 | 43,343 | 26.04 | YPL249C-A | 3022 | 459 | 74,990 | 194.69 |
| YHR187W | 1472 | 1,076 | 1,915 | 1.78 | YPL250C | 3021 | 642 | 2,095 | 3.26 |
| YHR188C | 1336 | 1,922 | 16,854 | 8.77 | YPL252C | 3020 | 625 | 4,283 | 6.85 |
| YHR189W | 1473 | 573 | 696 | 1.21 | YPL254W | 3156 | 1,774 | 2,146 | 1.21 |
| YHR190W | 1474 | 1,485 | 15,335 | 10.33 | YPL256C | 3019 | 2,009 | 5,560 | 2.77 |
| YHR191C | 1335 | 624 | 837 | 1.34 | YPL260W | 3157 | 1,833 | 2,130 | 1.16 |
| YHR192W | 1475 | 941 | 1,450 | 1.54 | YPL262W | 3158 | 1,556 | 17,285 | 11.11 |
| YHR193C | 1334 | 587 | 36,803 | 62.70 | YPL263C | 3018 | 2,094 | 4,813 | 2.30 |
| YHR194W | 1476 | 1,827 | 2,222 | 1.22 | YPL265W | 3159 | 2,203 | 6,399 | 2.90 |
| YHR196W | 1477 | 1,831 | 2,780 | 1.52 | YPL266W | 3160 | 1,074 | 3,692 | 3.44 |
| YHR197W | 1478 | 2,405 | 3,250 | 1.35 | YPL270W | 3161 | 2,412 | 4,819 | 2.00 |
| YHR198C | 1333 | 966 | 1,838 | 1.90 | YPL271W | 3162 | 341 | 8,261 | 24.23 |
| YHR199C | 1332 | 977 | 5,334 | 5.46 | YPL273W | 3163 | 1,189 | 14,491 | 14.32 |
| YHR199C-A | 1331 | 222 | 449 | 2.02 | YPL274W | 3164 | 1,871 | 5,398 | 2.89 |
| YHR200W | 1479 | 906 | 7,587 | 8.37 | YPR004C | 3017 | 1,149 | 4,886 | 4.25 |
| YHR201C | 1330 | 1,342 | 2,305 | 1.72 | YPR009W | 3165 | 979 | 1,952 | 1.99 |
| YHR203C | 1329 | 919 | 24,101 | 71.17 | YPR010C | 3016 | 3,789 | 28,219 | 7.45 |
| YHR204W | 1480 | 2,720 | 6,541 | 2.42 | YPR010C-A | 3015 | 474 | 1,293 | 2.75 |
| YHR205W | 1481 | 2,655 | 5,137 | 1.94 | YPR011C | 3014 | 1,372 | 1,425 | 1.04 |
| YHR206W | 1482 | 2,467 | 4,042 | 1.64 | YPR016C | 3013 | 923 | 19,062 | 20.65 |
| YHR208W | 1483 | 1,361 | 16,040 | 11.79 | YPR017C | 3012 | 591 | 2,426 | 4.10 |
| YHR215W | 1484 | 1,516 | 2,522 | 5.18 | YPR019W | 3166 | 3,028 | 4,299 | 1.42 |
| YHR216W | 1485 | 1,948 | 7,565 | 4.40 | YPR020W | 3167 | 494 | 955 | 1.93 |
| YIL002W-A | 1546 | 295 | 1,121 | 3.80 | YPR023C | 3011 | 1,298 | 2,811 | 2.16 |
| YIL003W | 1547 | 1,003 | 1,967 | 1.96 | YPR024W | 3168 | 2,867 | 13,261 | 4.63 |
| YIL004C | 1537 | 589 | 802 | 1.36 | YPR028W | 3169 | 739 | 20,184 | 27.31 |
| YIL008W | 1548 | 480 | 700 | 1.46 | YPR029C | 3010 | 2,750 | 3,728 | 1.35 |
| YIL009C-A | 1536 | 704 | 2,682 | 3.81 | YPR033C | 3009 | 1,688 | 22,813 | 13.51 |
| YIL009W | 1549 | 2,292 | 6,272 | 2.74 | YPR034W | 3170 | 1,745 | 3,266 | 1.87 |
| YIL010W | 1550 | 856 | 1,194 | 1.39 | YPR035W | 3171 | 1,327 | 18,176 | 13.70 |
| YIL011W | 1551 | 955 | 1,216 | 1.28 | YPR036W | 3172 | 1,565 | 41,001 | 26.20 |
| YIL014W | 1552 | 2,046 | 2,804 | 1.37 | YPR036W-A | 3173 | 490 | 2,725 | 5.56 |
| YIL016W | 1553 | 524 | 1,613 | 3.08 | YPR037C | 3008 | 773 | 2,067 | 2.67 |
| YIL018W | 1554 | 880 | 53,220 | 139.74 | YPR041W | 3174 | 1,470 | 24,941 | 16.98 |
| YIL020C | 1535 | 938 | 1,308 | 1.39 | YPR043W | 3175 | 377 | 43,625 | 128.27 |
| YIL021W | 1555 | 1,154 | 2,579 | 2.23 | YPR048W | 3176 | 1,963 | 3,160 | 1.61 |
| YIL022W | 1556 | 1,632 | 5,298 | 3.25 | YPR051W | 3177 | 669 | 1,466 | 2.19 |
| YIL023C | 1534 | 1,353 | 7,686 | 5.68 | YPR052C | 3007 | 448 | 12,317 | 27.49 |
| YIL027C | 1533 | 549 | 1,932 | 3.52 | YPR056W | 3178 | 1,199 | 1,771 | 1.48 |
| YIL029C | 1532 | 889 | 2,275 | 2.62 | YPR058W | 3179 | 1,069 | 6,582 | 6.16 |
| YIL030C | 1531 | 4,038 | 11,576 | 2.87 | YPR060C | 3006 | 956 | 3,237 | 3.39 |
| YIL033C | 1530 | 1,454 | 6,695 | 4.60 | YPR062W | 3180 | 601 | 9,065 | 15.08 |
| YIL034C | 1529 | 931 | 5,621 | 6.04 | YPR063C | 3005 | 639 | 5,747 | 8.99 |
| YIL035C | 1528 | 1,319 | 1,391 | 1.05 | YPR067W | 3181 | 739 | 1,140 | 1.54 |
| YIL036W | 1557 | 1,926 | 2,025 | 1.05 | YPR069C | 3004 | 943 | 20,696 | 21.95 |
| YIL038C | 1527 | 2,867 | 2,937 | 1.02 | YPR071W | 3182 | 988 | 1,042 | 1.05 |
| YIL039W | 1558 | 1,563 | 13,910 | 8.90 | YPR072W | 3183 | 1,878 | 2,771 | 1.47 |
| YIL040W | 1559 | 501 | 1,443 | 2.88 | YPR073C | 3003 | 770 | 3,727 | 4.84 |
| YIL041W | 1560 | 1,091 | 16,115 | 14.77 | YPR074C | 3002 | 2,246 | 128,745 | 57.37 |
| YIL043C | 1526 | 951 | 47,612 | 50.06 | YPR075C | 3001 | 1,212 | 2,131 | 1.76 |
| YIL044C | 1525 | 1,031 | 1,689 | 1.64 | YPR079W | 3184 | 1,313 | 2,036 | 1.55 |
| YIL046W | 1561 | 2,116 | 3,134 | 1.48 | YPR080W | 3185 | 1,533 | 9,776 | 50.75 |
| YIL047C | 1524 | 2,709 | 12,310 | 4.54 | YPR082C | 3000 | 527 | 699 | 1.33 |
| YIL048W | 1562 | 3,912 | 3,957 | 1.01 | YPR086W | 3186 | 1,102 | 1,868 | 1.69 |
| YIL049W | 1563 | 838 | 909 | 1.08 | YPR088C | 2999 | 1,711 | 14,456 | 8.45 |
| YIL051C | 1523 | 565 | 29,278 | 51.82 | YPR091C | 2998 | 2,373 | 3,028 | 1.28 |
| YIL052C | 1522 | 426 | 31,620 | 104.79 | YPR098C | 2997 | 528 | 1,446 | 2.74 |
| YIL053W | 1564 | 931 | 47,658 | 51.94 | YPR100W | 3187 | 483 | 676 | 1.40 |
| YIL062C | 1521 | 550 | 8,079 | 14.69 | YPR102C | 2996 | 616 | 31,138 | 127.12 |
| YIL063C | 1520 | 1,030 | 1,268 | 1.23 | YPR103W | 3188 | 1,019 | 9,434 | 9.26 |
| YIL064W | 1565 | 855 | 2,307 | 2.70 | YPR105C | 2995 | 2,749 | 3,256 | 1.18 |
| YIL065C | 1519 | 572 | 1,580 | 2.76 | YPR106W | 3189 | 1,498 | 2,189 | 1.46 |
| YIL067C | 1518 | 2,074 | 2,384 | 1.15 | YPR108W | 3190 | 1,418 | 4,997 | 3.52 |
| YIL069C | 1517 | 637 | 13,521 | 36.95 | YPR109W | 3191 | 1,132 | 2,435 | 2.15 |
| YIL070C | 1516 | 937 | 1,406 | 1.50 | YPR110C | 2994 | 1,210 | 13,518 | 11.17 |
| YIL074C | 1515 | 1,551 | 8,135 | 5.43 | YPR113W | 3192 | 787 | 18,934 | 24.06 |
| YIL075C | 1514 | 2,966 | 13,949 | 4.70 | YPR114W | 3193 | 1,099 | 8,552 | 7.78 |
| YIL076W | 1566 | 1,067 | 8,612 | 8.07 | YPR118W | 3194 | 1,350 | 11,629 | 8.61 |
| YIL078W | 1567 | 2,318 | 53,427 | 23.05 | YPR119W | 3195 | 1,476 | 2,742 | 1.86 |
| YIL083C | 1513 | 1,284 | 3,530 | 2.75 | YPR124W | 3196 | 1,452 | 4,403 | 3.03 |
| YIL085C | 1512 | 1,668 | 2,684 | 2.16 | YPR125W | 3197 | 1,622 | 1,994 | 1.23 |
| YIL087C | 1511 | 520 | 739 | 1.42 | YPR127W | 3198 | 1,237 | 2,991 | 2.42 |
| YIL088C | 1510 | 1,591 | 6,713 | 4.22 | YPR128C | 2993 | 987 | 5,016 | 5.08 |
| YIL090W | 1568 | 1,916 | 8,609 | 4.49 | YPR129W | 3199 | 1,152 | 1,963 | 1.70 |
| YIL093C | 1509 | 1,040 | 1,461 | 1.42 | YPR131C | 2992 | 675 | 2,089 | 3.09 |
| YIL094C | 1508 | 1,207 | 16,661 | 13.80 | YPR132W | 3200 | 554 | 39,256 | 138.36 |
| YIL103W | 1569 | 1,379 | 1,468 | 1.06 | YPR133C | 2991 | 1,360 | 2,122 | 1.56 |
| YIL106W | 1570 | 1,131 | 1,404 | 1.24 | YPR133W-A | 3201 | 343 | 6,389 | 18.63 |
| YIL108W | 1571 | 2,313 | 3,930 | 1.70 | YPR137W | 3202 | 1,852 | 3,022 | 1.63 |
| YIL109C | 1507 | 2,966 | 32,200 | 10.86 | YPR138C | 2990 | 1,864 | 3,653 | 1.96 |
| YIL110W | 1572 | 1,739 | 3,938 | 2.26 | YPR139C | 2989 | 1,049 | 2,196 | 2.09 |
| YIL111W | 1573 | 512 | 2,598 | 5.07 | YPR140W | 3203 | 1,280 | 1,288 | 1.01 |
| YIL114C | 1506 | 931 | 3,949 | 4.24 | YPR144C | 2988 | 1,796 | 2,916 | 1.62 |
| YIL115C | 1505 | 4,577 | 6,509 | 1.43 | YPR145W | 3204 | 1,922 | 41,716 | 22.76 |
| YIL116W | 1574 | 1,274 | 5,460 | 4.30 | YPR147C | 2987 | 1,153 | 5,665 | 4.91 |
| YIL117C | 1504 | 1,136 | 1,893 | 1.67 | YPR148C | 2986 | 1,453 | 2,312 | 1.59 |
| YIL118W | 1575 | 958 | 4,598 | 4.80 | YPR149W | 3205 | 1,078 | 20,516 | 19.15 |
| YIL121W | 1576 | 1,833 | 4,386 | 2.39 | YPR154W | 3206 | 1,013 | 2,014 | 2.01 |
| YIL123W | 1577 | 2,250 | 25,936 | 11.57 | YPR156C | 2985 | 2,379 | 2,978 | 1.44 |
| YIL124W | 1578 | 1,044 | 9,776 | 9.36 | YPR159W | 3207 | 2,730 | 19,396 | 7.11 |
| YIL125W | 1579 | 3,258 | 4,936 | 1.51 | YPR161C | 2984 | 2,104 | 3,068 | 1.46 |
| YIL131C | 1503 | 1,660 | 3,306 | 1.99 | YPR163C | 2983 | 1,536 | 15,464 | 10.63 |
| YIL133C | 1502 | 717 | 34,708 | 51.85 | YPR165W | 3208 | 1,031 | 20,489 | 19.87 |
| YIL134W | 1580 | 1,152 | 1,525 | 1.32 | YPR166C | 2982 | 450 | 1,204 | 2.67 |
| YIL135C | 1501 | 1,517 | 1,774 | 1.17 | YPR167C | 2981 | 873 | 1,137 | 1.30 |
| YIL137C | 1500 | 2,937 | 5,946 | 2.02 | YPR169W | 3209 | 1,591 | 1,923 | 1.21 |
| YIL138C | 1499 | 829 | 2,408 | 3.05 | YPR170W-B | 3210 | 456 | 17,170 | 37.65 |
| YIL140W | 1581 | 2,472 | 5,115 | 2.07 | YPR172W | 3211 | 603 | 1,433 | 2.38 |
| YIL142W | 1582 | 1,638 | 13,196 | 8.06 | YPR173C | 2980 | 1,409 | 2,337 | 1.66 |
| YIL143C | 1498 | 2,704 | 3,634 | 1.34 | YPR176C | 2979 | 1,153 | 3,368 | 2.92 |
| YIL145C | 1497 | 1,305 | 9,751 | 7.47 | YPR181C | 2978 | 2,692 | 27,724 | 10.30 |
| YIL148W | 1583 | 467 | 27,593 | 107.35 | YPR182W | 3212 | 397 | 3,874 | 9.76 |
| YIL152W | 1584 | 708 | 784 | 1.12 | YPR183W | 3213 | 930 | 14,097 | 15.16 |
| YIL153W | 1585 | 1,242 | 3,248 | 2.62 | YPR187W | 3214 | 569 | 4,984 | 8.76 |
| YIL154C | 1496 | 1,112 | 3,434 | 3.09 | YPR188C | 2977 | 714 | 1,227 | 1.72 |
| YIL156W-B | 1586 | 314 | 2,676 | 13.08 | YPR190C | 2976 | 2,101 | 2,659 | 1.27 |
| YIL157C | 1495 | 832 | 2,967 | 3.57 | YPR191W | 3215 | 1,355 | 3,732 | 2.75 |
| YIL158W | 1587 | 744 | 1,084 | 1.46 | YPR198W | 3216 | 1,726 | 3,766 | 2.18 |
| YIL162W | 1588 | 1,834 | 5,264 | 2.87 | YPR199C | 2975 | 1,015 | 1,350 | 1.33 |
| YIL164C | 1494 | 600 | 651 | 1.08 | RDN18 | 2090-2091 | 1,800 | 2,894,527 | 1608.07 |
| YIR006C | 1493 | 4,593 | 10,092 | 2.23 | RDN25 | 2088-2089 | 3,396 | 7,814,920 | 2301.21 |
| YIR008C | 1492 | 1,286 | 2,675 | 2.08 | RDN37 | 2086-2087 | 5,354 | 11,423,085 | 2133.56 |
| YIR009W | 1589 | 563 | 632 | 1.12 | RDN5 | 2092-2097 | 121 | 176,933 | 1462.26 |
| YIR011C | 1491 | 1,063 | 1,363 | 1.28 | RDN58 | 2084-2085 | 158 | 706,763 | 4473.18 |
| YIR012W | 1590 | 2,480 | 11,028 | 4.45 | tA-UGC | 78, | 73 | 167 | 2.29 |
| | | | | | | 889, | | | |
| | | | | | | 1182, | | | |
| | | | | | | 2099, | | | |
| | | | | | | 2839 | | | |
| YIR015W | 1591 | 435 | 468 | 1.08 | tD-GUC | 229, | 72 | 481 | 6.68 |
| | | | | | | 623, | | | |
| | | | | | | 1183, | | | |
| | | | | | | 1175, | | | |
| | | | | | | 1540, | | | |
| | | | | | | 1539, | | | |
| | | | | | | 1692, | | | |
| | | | | | | 1693, | | | |
| | | | | | | 1687, | | | |
| | | | | | | 1694, | | | |
| | | | | | | 1871, | | | |
| | | | | | | 2122, | | | |
| | | | | | | 2123, | | | |
| | | | | | | 2370, | | | |
| | | | | | | 2592, | | | |
| | | | | | | 2847 | | | |
| YIR016W | 1592 | 925 | 2,283 | 2.47 | tE-CUC | 619, | 72 | 27,690 | 384.58 |
| | | | | | | 1542 | | | |
| YIR018W | 1593 | 904 | 1,374 | 1.52 | tE-UUC | 230, | 72 | 33,670 | 467.64 |
| | | | | | | 398, | | | |
| | | | | | | 896, | | | |
| | | | | | | 897, | | | |
| | | | | | | 888, | | | |
| | | | | | | 1184, | | | |
| | | | | | | 1174, | | | |
| | | | | | | 1185, | | | |
| | | | | | | 1543, | | | |
| | | | | | | 1686, | | | |
| | | | | | | 1870, | | | |
| | | | | | | 2124, | | | |
| | | | | | | 2371, | | | |
| | | | | | | 3085 | | | |
| YIR021W | 1594 | 1,136 | 3,024 | 2.66 | tF-GAA | 231, | 73 | 84 | 1.15 |
| | | | | | | 1010, | | | |
| | | | | | | 1173, | | | |
| | | | | | | 1398, | | | |
| | | | | | | 1397, | | | |
| | | | | | | 2372, | | | |
| | | | | | | 3084, | | | |
| | | | | | | 3083 | | | |
| YIR022W | 1595 | 606 | 5,542 | 9.14 | tI-AAU | 232, | 74 | 81 | 1.09 |
| | | | | | | 618, | | | |
| | | | | | | 887, | | | |
| | | | | | | 886, | | | |
| | | | | | | 1186, | | | |
| | | | | | | 1538, | | | |
| | | | | | | 1544, | | | |
| | | | | | | 2125, | | | |
| | | | | | | 2098, | | | |
| | | | | | | 2591, | | | |
| | | | | | | 2597, | | | |
| | | | | | | 3091, | | | |
| | | | | | | 3082 | | | |
| YIR026C | 1490 | 1,193 | 3,280 | 2.75 | tK-CUU | 397, | 73 | 116 | 1.59 |
| | | | | | | 624, | | | |
| | | | | | | 617, | | | |
| | | | | | | 885, | | | |
| | | | | | | 884, | | | |
| | | | | | | 1187 | | | |
| | | | | | | 1172, | | | |
| | | | | | | 1188, | | | |
| | | | | | | 1695, | | | |
| | | | | | | 1875, | | | |
| | | | | | | 2361, | | | |
| | | | | | | 3092 | | | |
| YIR034C | 1489 | 1,185 | 3,731 | 3.15 | tL-UAA | 234, | 84 | 196 | 2.33 |
| | | | | | | 235, | | | |
| | | | | | | 625, | | | |
| | | | | | | 1685, | | | |
| | | | | | | 1876, | | | |
| | | | | | | 2097, | | | |
| | | | | | | 2590 | | | |
| YIR035C | 1488 | 847 | 3,938 | 4.65 | tN-GUU | 401, | 74 | 105 | 1.42 |
| | | | | | | 1009, | | | |
| | | | | | | 1189, | | | |
| | | | | | | 1877, | | | |
| | | | | | | 2096, | | | |
| | | | | | | 2598, | | | |
| | | | | | | 2599, | | | |
| | | | | | | 2848, | | | |
| | | | | | | 2849, | | | |
| | | | | | | 3093 | | | |
| YIR036C | 1487 | 837 | 3,477 | 4.15 | tR-UCU | 236, | 72 | 134 | 1.86 |
| | | | | | | 626, | | | |
| | | | | | | 898, | | | |
| | | | | | | 1190, | | | |
| | | | | | | 1171, | | | |
| | | | | | | 1191, | | | |
| | | | | | | 1696, | | | |
| | | | | | | 1684, | | | |
| | | | | | | 1869, | | | |
| | | | | | | 2360, | | | |
| | | | | | | 2359 | | | |
| YIR037W | 1596 | 630 | 6,355 | 10.09 | tS-CGA | 402 | 82 | 479 | 5.84 |
| YIR038C | 1486 | 828 | 3,242 | 3.91 | tS-GCU | 1008, | 82 | 166 | 2.02 |
| | | | | | | 2850 | | | |
| YJL001W | 1698 | 803 | 10,864 | 13.53 | tS-UGA | 883, | 82 | 893 | 10.89 |
| | | | | | | 1545, | | | |
| | | | | | | 3094 | | | |
| YJL002C | 1683 | 1,552 | 30,243 | 19.49 | tV-AAC | 899, | 74 | 146 | 1.97 |
| | | | | | | 900, | | | |
| | | | | | | 1170, | | | |
| | | | | | | 1192, | | | |
| | | | | | | 1169, | | | |
| | | | | | | 1396, | | | |
| | | | | | | 1697, | | | |
| | | | | | | 1868, | | | |
| | | | | | | 1878, | | | |
| | | | | | | 2095, | | | |
| | | | | | | 2358, | | | |
| | | | | | | 2357, | | | |
| | | | | | | 2356, | | | |
| | | | | | | 2838 | | | |
| YJL004C | 1682 | 885 | 1,930 | 2.18 | SCR1 | 891 | 522 | 12,096 | 23.17 |
| YJL005W | 1699 | 6,450 | 6,627 | 1.03 | SRG1 | 895 | 551 | 1,009 | 1.83 |
| YJL008C | 1681 | 1,861 | 19,388 | 10.42 | TLC1 | 233 | 1,301 | 7,617 | 5.85 |
| YJL011C | 1680 | 657 | 789 | 1.20 | snR10 | 1178 | 245 | 2,929 | 11.95 |
| YJL012C | 1679 | 2,246 | 40,856 | 18.19 | snR11 | 2363 | 258 | 701 | 2.72 |
| YJL014W | 1700 | 1,759 | 10,055 | 5.72 | snR128 | 1691 | 126 | 5,511 | 43.74 |
| YJL016W | 1701 | 2,184 | 3,132 | 1.43 | snR13 | 622 | 124 | 1,107 | 8.93 |
| YJL020C | 1678 | 3,670 | 4,099 | 1.12 | snR161 | 226 | 161 | 179 | 1.11 |
| YJL024C | 1677 | 823 | 2,233 | 2.71 | snR17a | 2843 | 333 | 720 | 2.16 |
| YJL026W | 1702 | 1,379 | 23,105 | 16.76 | snR17b | 3089 | 332 | 643 | 1.94 |
| YJL034W | 1703 | 2,286 | 52,103 | 23.39 | snR18 | 77 | 102 | 1,545 | 15.15 |
| YJL035C | 1676 | 753 | 1,199 | 1.59 | snR190 | 1690 | 190 | 1,949 | 10.26 |
| YJL039C | 1675 | 5,248 | 6,542 | 1.25 | snR24 | 2362 | 89 | 459 | 5.16 |
| YJL041W | 1704 | 2,660 | 4,641 | 1.80 | snR30 | 2118 | 606 | 1,405 | 2.32 |
| YJL042W | 1705 | 4,398 | 4,869 | 1.11 | snR31 | 2842 | 225 | 1,406 | 6.25 |
| YJL044C | 1674 | 1,491 | 3,211 | 2.15 | snR32 | 1399 | 188 | 1,259 | 6.70 |
| YJL050W | 1706 | 3,454 | 6,593 | 1.91 | snR33 | 400 | 183 | 3,195 | 17.46 |
| YJL052W | 1707 | 1,205 | 4,182 | 6.25 | snR34 | 2119 | 203 | 1,482 | 7.30 |
| YJL053W | 1708 | 1,275 | 1,820 | 1.43 | snR37 | 1689 | 386 | 1,172 | 3.04 |
| YJL054W | 1709 | 1,648 | 3,124 | 1.90 | snR38 | 1872 | 95 | 259 | 2.73 |
| YJL055W | 1710 | 822 | 2,708 | 3.29 | snR39 | 1177 | 89 | 222 | 2.49 |
| YJL059W | 1711 | 1,418 | 1,990 | 1.40 | snR39B | 1176 | 96 | 539 | 5.61 |
| YJL060W | 1712 | 1,486 | 3,047 | 2.05 | snR4 | 892 | 186 | 1,421 | 7.64 |
| YJL061W | 1713 | 2,324 | 3,436 | 1.48 | snR40 | 2594 | 97 | 2,351 | 24.24 |
| YJL062W | 1714 | 2,610 | 6,399 | 2.45 | snR41 | 3088 | 95 | 403 | 4.24 |
| YJL062W-A | 1715 | 473 | 4,056 | 8.59 | snR43 | 399 | 209 | 1,063 | 5.09 |
| YJL063C | 1673 | 806 | 2,948 | 3.66 | snR44 | 2120 | 211 | 1,241 | 5.88 |
| YJL065C | 1672 | 602 | 2,930 | 4.88 | snR45 | 3090 | 172 | 916 | 5.33 |
| YJL066C | 1671 | 948 | 954 | 1.01 | snR46 | 1179 | 197 | 414 | 2.10 |
| YJL068C | 1670 | 986 | 5,831 | 5.91 | snR47 | 621 | 99 | 1,231 | 12.43 |
| YJL069C | 1669 | 1,875 | 3,315 | 1.77 | snR48 | 1180 | 113 | 1,424 | 12.60 |
| YJL072C | 1668 | 642 | 1,018 | 1.59 | snR49 | 2595 | 165 | 396 | 2.40 |
| YJL076W | 1716 | 3,659 | 6,838 | 1.87 | snR50 | 2844 | 90 | 632 | 7.02 |
| YJL078C | 1667 | 2,894 | 9,360 | 3.23 | snR51 | 3087 | 107 | 613 | 5.73 |
| YJL079C | 1666 | 1,249 | 5,906 | 4.74 | snR52 | 890 | 92 | 10,089 | 109.66 |
| YJL080C | 1665 | 3,934 | 24,390 | 6.20 | snR53 | 893 | 91 | 871 | 9.57 |
| YJL081C | 1664 | 1,623 | 3,688 | 2.27 | snR55 | 2103 | 98 | 257 | 2.62 |
| YJL091C | 1663 | 1,623 | 3,030 | 1.87 | snR56 | 228 | 88 | 1,556 | 17.68 |
| YJL093C | 1662 | 2,076 | 4,709 | 2.27 | snR57 | 2102 | 88 | 118 | 1.34 |
| YJL094C | 1661 | 2,803 | 3,761 | 1.34 | snR58 | 2841 | 96 | 2,235 | 23.28 |
| YJL096W | 1717 | 584 | 1,404 | 2.40 | snR60 | 1688 | 104 | 1,832 | 17.61 |
| YJL097W | 1718 | 754 | 8,081 | 10.72 | snR61 | 2101 | 90 | 164 | 1.82 |
| YJL101C | 1660 | 2,397 | 4,656 | 1.94 | snR63 | 620 | 255 | 1,061 | 4.16 |
| YJL104W | 1719 | 649 | 1,365 | 2.10 | snR64 | 1873 | 101 | 4,436 | 43.92 |
| YJL109C | 1659 | 5,398 | 14,092 | 2.61 | snR66 | 2596 | 86 | 433 | 5.03 |
| YJL111W | 1720 | 1,949 | 7,529 | 3.86 | snR67 | 894 | 82 | 698 | 8.51 |
| YJL117W | 1721 | 1,094 | 8,034 | 7.40 | snR68 | 1541 | 136 | 1,380 | 10.15 |
| YJL118W | 1722 | 829 | 1,109 | 1.35 | snR69 | 1874 | 101 | 1,362 | 13.49 |
| YJL121C | 1658 | 882 | 14,692 | 16.66 | snR70 | 3086 | 164 | 322 | 1.96 |
| YJL122W | 1723 | 714 | 2,165 | 3.03 | snR71 | 1400 | 90 | 423 | 4.70 |
| YJL124C | 1657 | 881 | 1,920 | 2.18 | snR72 | 2364 | 98 | 1,336 | 13.63 |
| YJL126W | 1724 | 924 | 1,154 | 1.25 | snR73 | 2365 | 106 | 1,181 | 11.14 |
| YJL127C-B | 1656 | 560 | 2,773 | 4.95 | snR75 | 2366 | 89 | 695 | 7.81 |
| YJL128C | 1655 | 2,222 | 4,821 | 2.17 | snR76 | 2367 | 109 | 215 | 1.97 |
| YJL129C | 1654 | 3,915 | 4,115 | 1.05 | snR77 | 2368 | 88 | 377 | 4.28 |
| YJL130C | 1653 | 7,346 | 30,212 | 4.11 | snR78 | 2369 | 87 | 395 | 4.54 |
| YJL133C-A | 1652 | 427 | 1,030 | 2.41 | snR79 | 2100 | 84 | 371 | 4.42 |
| YJL133W | 1725 | 1,248 | 2,126 | 1.70 | snR8 | 2845 | 190 | 714 | 3.76 |
| YJL134W | 1726 | 1,305 | 4,591 | 3.52 | snR81 | 2846 | 201 | 266 | 1.32 |
| YJL136C | 1651 | 405 | 25,144 | 75.40 | snR82 | 1181 | 268 | 677 | 2.53 |
| YJL137C | 1650 | 1,294 | 1,445 | 1.12 | snR9 | 2840 | 187 | 2,775 | 14.84 |
| YJL138C | 1649 | 1,442 | 10,348 | 24.12 | LSR1 | 227 | 1,175 | 2,194 | 1.87 |
| YJL140W | 1727 | 802 | 2,606 | 3.25 | snR19 | 2593 | 568 | 1,343 | 2.36 |
| | | | | | snR6 | 2121 | 112 | 691 | 6.17 |

Table 5: Provides a list of all RNA polynucleotides whose average nucleotide coverage is above 1.0 (methods). Columns show, for each gene, the annotated transcript length (in nucleotides), the total number of sequences mapping to that transcript and the computed load. While the poly-A purification process greatly reduces the overload of tRNAs and rRNAs in the total RNA, it is imperfect and non-polyadenylated transcripts are therefore recovered. Sequences of these transcripts are available through the NCBI web site (The Hypertext Transfer Protocol://world wide web (dot) ncbi (dot) nlm (dot) nih (dot) gov/). Note that several sequences representing different transcripts of rRNA or tRNA (each having a unique sequence identifier) are presented under a single GENE entry in the above Table. For example, SEQ ID NOs: 883, 1545 and 3094 are different transcripts of the gene entry tS-UGA, corresponding to the ts-UGA-E, ts-UGA-1 and ts-UGA-P, respectively, which are included in the “48677 Supplementary Data” file, which is being co-filed with the instant application.

The structural profiles of these transcripts, which include 3000 yeast coding transcripts, 14 tRNAs, 5 rRNAs, 58 snoRNAs and six other annotated non-coding genes was uncovered. In total, structural information for over 4.3 million transcribed bases was obtained, which is ˜100-fold more than all published RNA footprints to date.

The structural profile is provided in “Supplementary Data” file, in a text format. The information provided for each RNA polynucleotide includes “Designation” (the transcript name, e.g., “YLR110C”), “Sequence” (the nucleotide sequence of the RNA for which the pairability status was determined), “Length” (the length of the RNA polynucleotide for which the pairability status was determined (e.g., 507 for the first RNA transcript “YLR110C”), “SEQ ID NO:” (sequence identifier of the RNA polynucleotide for which the pairability status was determined), and “PARS score” (the log of the ratio between the number of reads obtained using RNase V1 and the number of reads obtained using RNase S1 for each of the nucleotides by order, separated by “;”). For example, for the first 11 nucleotides of the YLR110C RNA [CCAAGAAATTA (nucleotides 1-11 of SEQ ID NO:1)] the following data is presented: “YLR110C CCAAGAAATTA 507 1

2.92;1.96;1.34;2.04;0.86;1.24;1.77;2.36;2.93;−1.91;−1.86;”. This data indicates that the log ratio of the first nucleotide of YLR110C (i.e., “C” at position 1 of SEQ ID NO:1) is “2.92”, demonstrating that this nucleotide is in a “paired state”. Similarly, the log ratio of the second nucleotide of YLR110C (i.e., “C” at position 2 of SEQ ID NO:1) is “1.96”, demonstrating that this nucleotide is in a “paired state”. The log ratio of the tenth nucleotide of YLR110C (i.e.,“T” at position 10 of SEQ ID NO:1) is “−1.91”, demonstrating that this nucleotide is in an “unpaired state”.

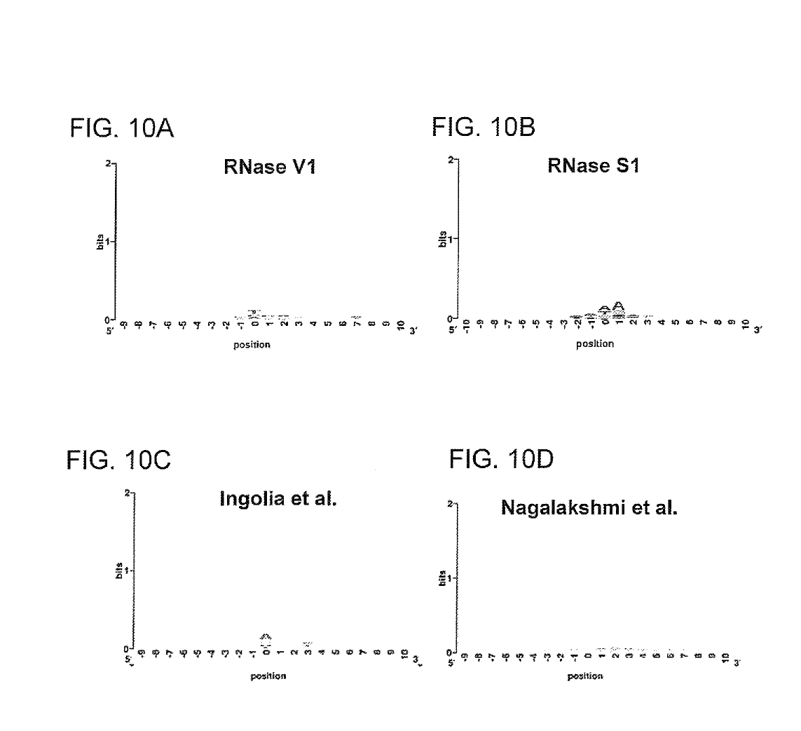

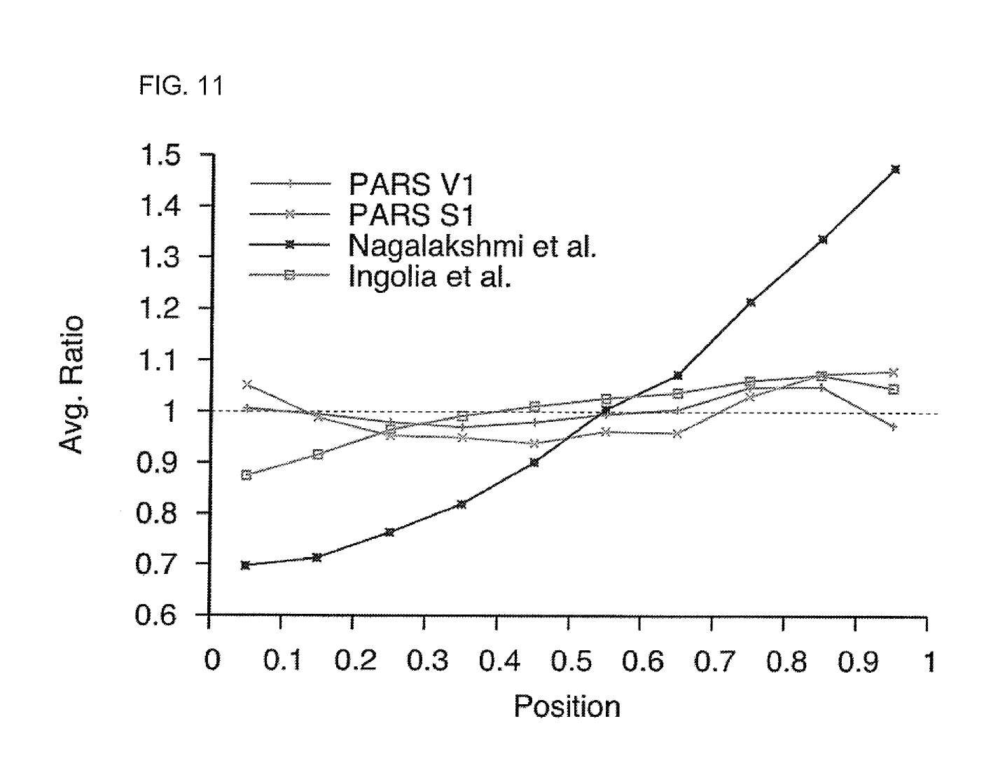

Several tests were used to determine whether there are biases in the method. First, to determine whether there is a bias towards RNA fragments with particular sequences, the nucleotide distribution over the first bases of the sequenced fragments were examined. The sequence composition at these bases did not show a strong sequence bias at the first base or around it, suggesting that RNase cleavage, adaptor ligation, and cDNA conversion do not introduce significant sequence biases (FIGS. 10A-D). Second, to test whether the obtained reads are uniform from both the 5′ and the 3′ end of the transcripts the present inventors plotted the number of reads as a function of position along each transcript and averaged the reads across all of the transcripts to obtain a global profile of 5′ to 3′ bias. Positions that exhibited the largest deviation from the mean coverage had 8% more reads than the mean coverage, suggesting that the protocol used by the present inventors has a relatively small bias towards particular regions along the transcript (FIG. 11). Third, to test whether the RNA fragments are cleaved and captured in proportion to their abundance in the initial pool the present inventors computed the average nucleotide coverage of each mRNA as the number of reads that map to that mRNA divided by the mRNA's length. A high correlation was found between mRNA coverage measurements across biological replicates (correlation>0.96, FIG. 9B), as well as among the measurements and previous sequencing-based approaches (Ingolia, N. T., 2009; Nagalakshmi, U. et al. 2008) that measured mRNA abundance in yeast (correlation=0.86 and 0.75 respectively, FIG. 9C). Fourth, to ensure that structure-specific signals are measured the present inventors also confirmed that signals generated by RNase V1 are highly distinct from those generated by RNase S1. Although as expected, V1 and S1 peaks are mostly distinct, some peaks overlap. Global inspection across all transcripts reveals that ˜18% of the V1 and S1 peaks are shared (data not shown). Because they are difficult to interpret, these joint peaks, as well as other background noise present in both experiments, are removed by the integrated PARS score.

### Example 3

### PARS Probes RNA Structures with High Accuracy

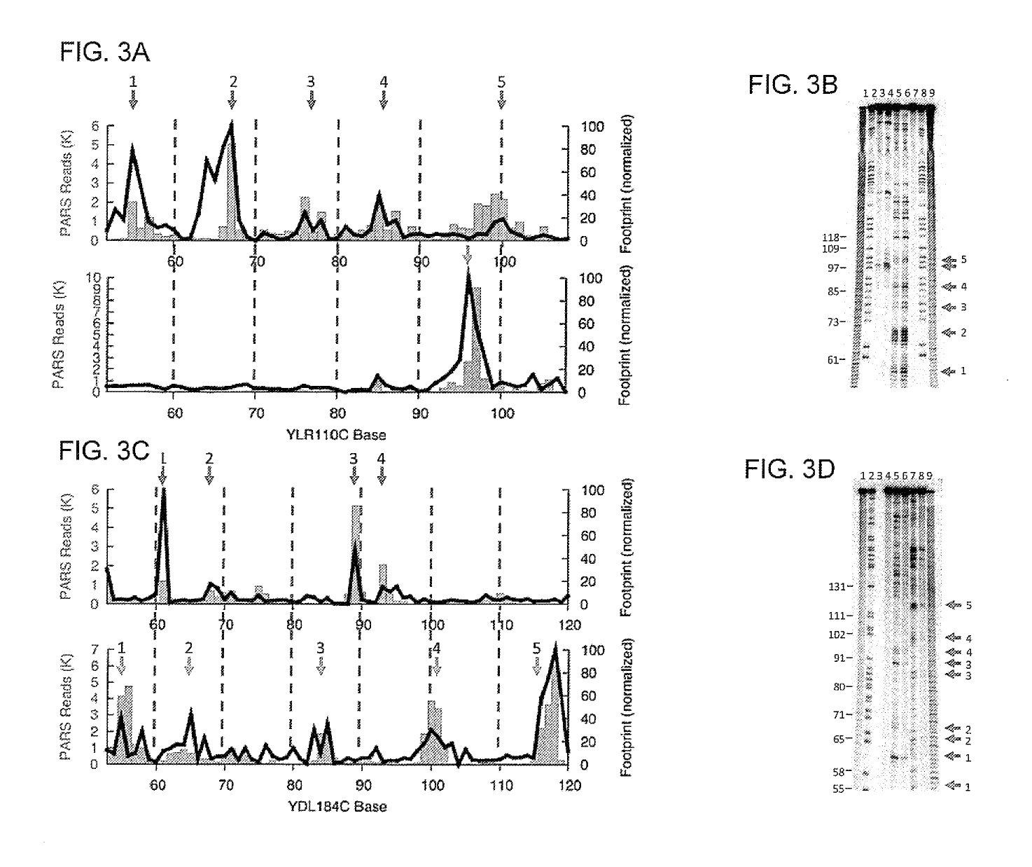

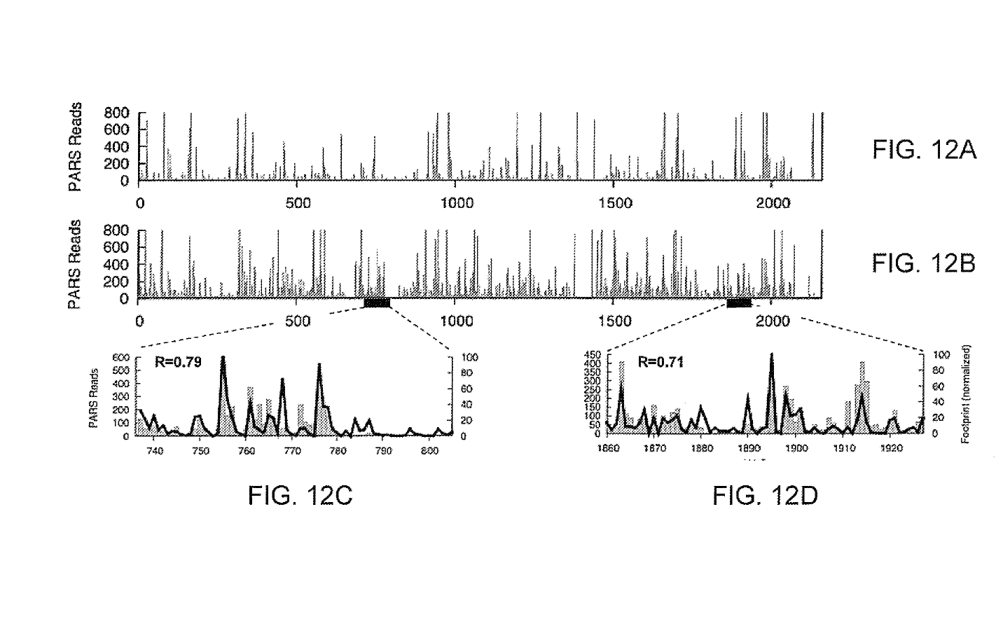

To test whether PARS accurately measures RNA structures, the present inventors confirmed that the signals obtained by the method of some embodiments of the invention are indeed similar to those obtained with traditional footprinting which was performed on a single RNA polynucleotide at a time. To this end, ten separate traditional footprinting experiments were conducted with either RNase V1 or S1, applied to two domains from the Tetrahymena ribozyme, and two domains from the human HOTAIR non-coding RNA, which were included in the samples (see above) and two domains of endogenous yeast mRNAs. The structure of the latter four were unknown and were first revealed by PARS. In all cases, high agreement was found between the PARS signals and traditional footprinting (correlations=0.63-0.97, FIGS. 3A-D; and FIGS. 12A-D, 13A-D, and 14A-D). Thus, nucleotides that are cleaved by RNase V1 or RNase S1 are accurately captured by PARS, and the relative intensities of such cleavage sites can be measured. Notably, due to length limitations of traditional footprinting, short domains were selected from each of the above transcripts, in vitro transcribed, and only then traditional footprinting was applied. Thus, traditional footprinting measures the structure of small RNA fragments that are excised from their larger encompassing RNA. This is not only laborious, but may also be inaccurate, since due to long-range interactions, the excised fragment may fold differently when taken out of context.

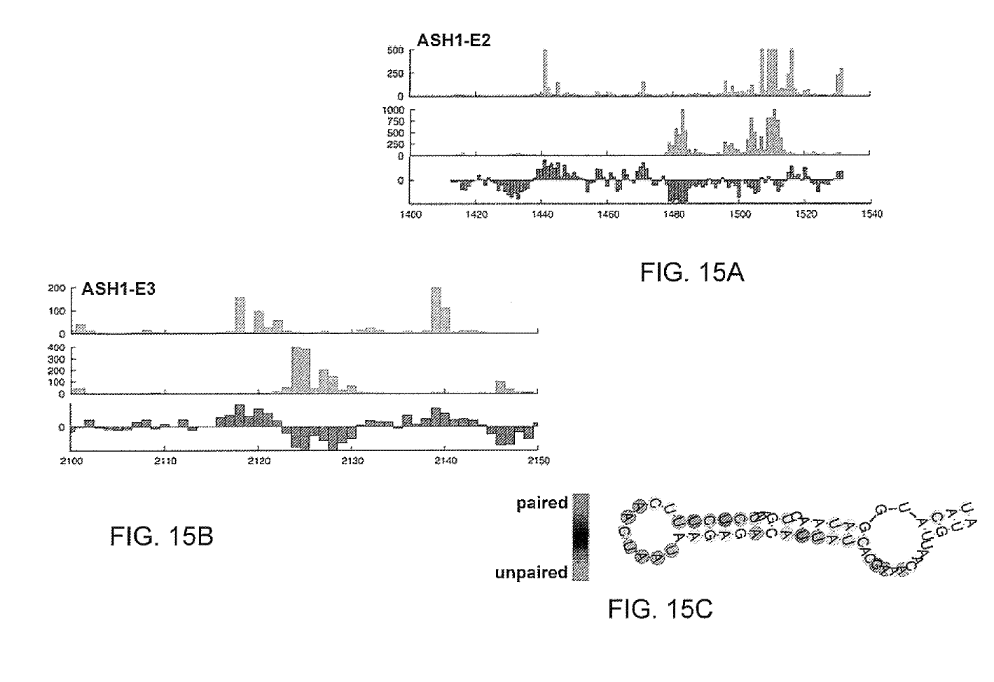

Finally, the PARS signals were compared to structures of yeast RNAs previously reported in the literature. Notably, PARS correctly reproduces the known secondary structure of three structured RNA domains of ASH1 [which are involved in mRNA localization at the bud tip (Chartrand, P., et al., 2002)] and of a structural element responsible for internal translation initiation in URE2 mRNA (Reineke, L. C., et al., 2008) (FIGS. 3A-D; FIGS. 15A-C). This result suggests that PARS is able to provide structural information of transcripts in their full-length context and endogenous abundance from within a complex RNA pool. Taken together, these results demonstrate that PARS recapitulates results obtained by low-throughput methods with high accuracy, and also has advantages over existing methods, stemming from its ability to probe structures of long RNAs.

### Example 4

### Comparison to RNA Folding Algorithms

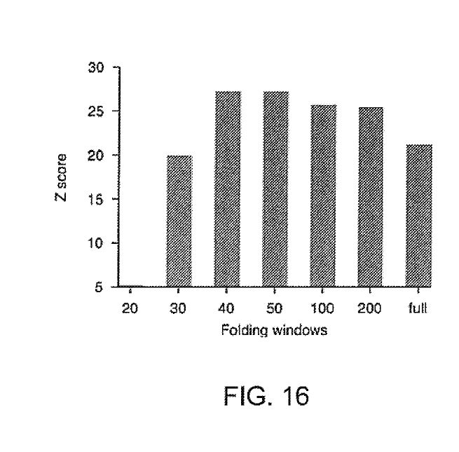

As the approach described herein provides genome-wide measurements of RNA structure, the present inventors sought to compare its results to algorithms that predict RNA structure. The Vienna package (Hofacker, I. L., et al., 2002) was used to fold the 3000 transcripts that were analyzed and a significant correspondence between these predictions and the PARS scores were found. The present inventors found that nucleotides with high double-stranded PARS score had a significantly higher average probability of being base paired according to Vienna and conversely, that nucleotides with high single-stranded PARS score (negative scores) were predicted by Vienna to have a significantly lower probability of being base paired. This result is highly significant, as can be seen when comparing to random samples of the same size (average of 0.577±0.006, p<10⁻²⁰⁰, FIG. 5A. Similar results were obtained from folding the yeast transcriptome in windows ranging from ˜40 nucleotides up to windows that cover the entire transcript (FIG. 16), suggesting that folding algorithms correctly capture local interactions but do not improve in accuracy when the entire transcript is made available. The present inventors suggest that genome-wide PARS data can be used to constrain folding algorithms and improve their accuracy, as has been previously shown for specific RNAs (Watts, J. M. et al. 2009; Mathews, D. H. et al. 2004). Overall, the significant correspondence between PARS and folding prediction provides further independent validation for the ability of PARS to provide genome-scale and high-quality measurements of RNA structure at single nucleotide resolution.

### Example 5

### Global Structural Properties of Yeast Transcripts

The present inventors used the structural measurements that were obtained for 3000 yeast transcripts to uncover global structural properties of yeast genes. First, examining the average PARS score across the coding regions and 5′ and 3′ untranslated regions (UTRs) of yeast transcripts, differences were found between the propensity for RNA structure across these regions, with coding regions exhibiting significantly more structure than 5′ and 3′ UTRs (p<10⁻³⁰ and p<10⁻⁵⁰ respectively, FIGS. 5B-C). Notably, the start and stop codons each exhibit local minima of PARS scores, indicating reduced tendency for double-stranded conformation and increased accessibility. These findings are in agreement with previous suggestions made on the basis of computational predictions for mouse and human genes (Shabalina, S. A., et al., 2006). The evolutionary conservation of this global organization of mRNA secondary structure suggests that it may have functional importance. The need to preserve regulatory interactions may have selected for decreased tendency to form secondary structures in UTRs, and in particular, in the start and stop codons (Shabalina, S. A., et al., 2006). Conversely, structured domains along coding regions may protect against ectopic translation initiation, or affect ribosome translocation and protein folding, as recently postulated (Watts, J. M. et al. 2009).

Second, aligning the coding regions of those 3000 genes and applying a discrete Fourier transform analysis to the average PARS signal, the present inventors detected a periodic structure signal across coding regions with a cycle of three nucleotides, such that on average, the first nucleotide of each codon is least structured and the second nucleotide is most structured. Notably, this periodic signal is only found in coding regions, and not in UTRs (FIGS. 5B-D). It is noted that triplet periodicity of the PARS signal is only detectable when averaging PARS signals over many genes and is less evident in mRNAs of individual genes. Thus, the periodic occurrence of RNA secondary structures cannot be used to set the proper phase of translation for individual mRNAs, and is more likely to be a consequence of the genetic code, codon usage and nucleotide distribution in yeast open reading frames.

### Example 6

### Local Structural Properties of Yeast Transcripts

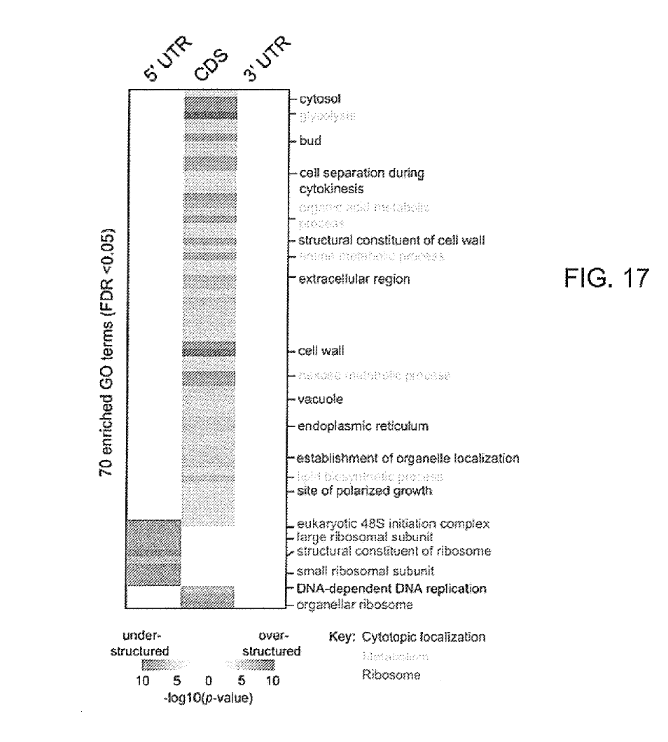

Having observed the pattern of RNA structure across yeast transcripts, the present inventors checked whether mRNAs of individual genes deviate from the canonical signature, and whether such deviations may be related to biological regulation. For each transcript, the present inventors ranked the overall PARS score of its 5′ UTR, CDS, and 3′ UTR, and used the Wilcoxon rank sum test to ask whether genes with shared biological functions or cytotopic localizations [REF GO] tend to have similar scores, which would correspond to similar degrees of secondary structures. A rich picture of biological coordination was found (FIG. 17) including increased RNA structure, especially with CDS and 3′ UTR, being significantly associated with cytotopic localization of the encoded proteins to distinct domains of the cell, such as the cell wall, the bud, cell division site, or the vacuole. The stronger association between RNA structure in CDS with cytotopic localization over that of UTRs was not anticipated and suggests that many RNA localization signals may reside in CDS. In addition, it was found that a decreased RNA structure is a feature of RNAs encoding many house-keeping enzymes, and that the mRNAs with the least secondary structure encode subunits of the ribosome. mRNAs encoding subunits of the same protein complex, such as the RENT complex, U2-splicesome, Smc5-Smc6 complex, and GINS complex, also tend to have the same pattern of RNA structures. These results suggest systematic organization of mRNA localization and function via specific patterns of RNA structure.

It has long been hypothesized that mRNA accessibility near the start codon affects protein translation (Kozak, M. et al., 2005). A recent work in E. coli suggested that the predicted degree of RNA folding near the translational start site explains much of the observed variation in translation efficiency of a reporter protein (Kudla, G., et al., 2009), and as shown in FIG. 5D, yeast start codon tends to lack RNA structure. The present inventors tested whether such a correlation between mRNA structure around the translation start site and translation efficiency exists in a eukaryotic organism and on a genome-wide scale. A correlation between the PARS scores in 40 by windows and ribosome density (Ingolia, N. T., 2009) was performed and used as a proxy for translation efficiency. A small but significant anti-correlation was found between translational efficiency and PARS scores around ten bases upstream of the translation start site (correlation=−0.1, p<10⁻⁴, FIG. 6A), which interestingly corresponds to the 5′ position of the first ribosome on yeast mRNAs (Ingolia, N. T., 2009). To study the structural patterns around the start codon in more detail, k-means clustering was applied to the structural profile of the ±40 by surrounding it. Three clusters were found with distinct structural profiles. Of particular interest are the genes found in cluster 4, as those exhibit significantly less structure in their 5′ UTR than in the beginning of their coding region. Notably, those genes also exhibit a higher ribosome density, providing further insight into the relationship between translational efficiency and the structural profile around translation starts sites (FIG. 6B). Overall, these results provide the first genome-wide experimental validation for the suggestion that mRNA secondary structure around the start codon may reduce translational efficiency (Kozak, M. et al., 2005), although the rather low correlation clearly implies that translational efficiency is determined by additional factors in vivo. The observed global difference in the extent of RNA structure between 5′ UTRs and coding regions suggests that lower RNA structure may be a feature of regulatory non-coding RNA sequences. Recently, RNA sequences encoding the signal sequence (termed the SSCR) of secretory proteins have been shown to function as an RNA element that promotes RNA nuclear export (Palazzo, A. F. et al., 2007) whereas the peptide encoded by SSCR directs the protein to the secretory pathway via the endoplasmic reticulum. The prevalence and structural basis of SSCR are not clear, and the present inventors wondered whether this dual function RNA/protein element, typically at the beginning of the coding sequence, would conform to the rule of lower RNA structure typical of UTRs. Indeed, the 5′ UTR region and first ˜40 coding nucleotides of transcripts predicted to encode a signal peptide have lower PARS signal, indicating increased single-stranded propensity, as compared to other transcripts (p<10⁻¹¹, FIG. 6D). Thus, these results raise the hypothesis that specific secondary RNA structure around gene starts may assist in the cytotopic localization of mRNAs and their resulting proteins. More generally, the present inventors suggest that PARS can be used to both generate and test hypotheses regarding signals of secondary structure that may characterize and have functional importance for classes of mRNAs.

### Example 7

### Determination of Pairability of RNA Molecules using Structure Sensitive Chemicals

Cells are subjected to binding with chemicals which specifically modify or bind to single stranded or double stranded RNA. Binding is performed in vivo or in vitro. Binding or covalent modification is performed for a certain amount of time, so that RNA nucleotides that are single-stranded are partially modified by the chemical (DMS—adenine and cytosine, or CMCT—uridine and some guanine).

For in-vivo structure probing: the chemical penetrates the cells and modifies the RNA in vivo. The RNA is then isolated from the cell. The proteins are removed from the RNA sample by conventional means. The RNA is subjected to RT-PCR to create cDNA. PCR falls off at modified sites, thus the first base of each DNA fragment represents a nucleotide that immediately follows a nucleotide that was in an “unpaired” conformation in the original RNA (in-vivo).

Adaptor ligation at the first base can be carried out to capture the first nucleotide.

For in-vitro structure probing: RNAs isolated from the cells are renatured in vitro and then subjected to partial modification by chemicals that recognize single/double stranded regions. After modification, the RNA ligated to adaptors and converted to cDNA.

The cDNA polynucleotides are subjected to deep sequencing-compatible library. Analysis of the outcome is similar to the analysis described in Examples 1-6 above. Each sequence fragment gives an “evidence point” about the sequence being in single/double-strand conformation, i.e., if the nucleotide immediately upstream of the first nucleotide in the sequenced fragment was in a single-strand conformation in the original RNA.

Analysis and Discussion

The invention according to some embodiments thereof provides PARS, the first high-throughput approach for experimentally measuring structural properties of RNAs at genome-scale. The present inventors show that PARS recovers structural properties with high accuracy and at a nucleotide resolution. Applying PARS to the entire transcriptome of yeast, the present inventors obtained structural information for over 3000 yeast transcripts and uncovered several global structural properties in them, including the propensity for more structure over coding regions compared to untranslated regions, a three-nucleotide periodic pattern of structure in coding regions, and a global anti-correlation between structure over translation start site and translational efficiency. While some of these findings have been hypothesized from computational predictions of RNA structure, the analysis provides the first large-scale and direct experimental validation for these hypotheses. These results reveal a systematic organization of secondary structure by RNA sequence, which can demarcate functional units of mRNAs.

PARS transforms the field of RNA structure probing into the realm of high-throughput, genome-wide analysis and should prove useful both in determining the structure of entire transcriptomes of other organisms as well as in systematically measuring the effects of diverse conditions on RNA structure. Applying PARS with other probes of RNA structure and dynamics should refine the precision and certainty of RNA structures. Probing RNA structure in the presence of different ligands, proteins, or in different physical or chemical conditions may provide further insights into how RNA structures control gene activity.

As a starting point, the present inventors implemented PARS with RNases, and it is likely that additional modification can improve the utility of PARS. More generally, many classical methods in molecular biology require precise mapping of ends of nucleic acids. The results presented herein provide the first experimental and computational frameworks to enable deep sequencing to precisely map ends of nucleic acid fragments in a complex pool, suggesting that many other powerful methods in structural and chemical biology can now be performed on a genomic scale.

It should be noted that simultaneous determination of the pairability (as used in the method of some embodiments of the invention) provides a significant advantage over the prior art methods [e.g., footprinting or SHAPE (e.g., Watts, J. M. et al. 2009] in which several sequence specific primers were designed along each RNA sequence in order to subject a single long RNA molecule (e.g., HIV) to deep sequencing, followed by repetitive sequencing runs (each begins from a distinct primer) in order to obtain information regarding the pairability state of each nucleotide. Thus, the prior art methods could not detect the pairability of a plurality of RNA polynucleotides simultaneously but instead are limited to analysis of a single RNA polynucleotide at a time. The prior art methods could not be used to detect a change in secondary structure of an RNA polynucleotide which is present in a mix of RNA polynucleotides such as in a cell.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Additional References are Cited in Text

1. Arava, Y. et al. Genome-wide analysis of mRNA translation profiles in Saccharomyces cerevisiae. in Proc Natl Acad Sci USA Vol. 100 3889-94 (2003).
2. Olivier, C. et al. Identification of a conserved RNA motif essential for She2p recognition and mRNA localization to the yeast bud. Mol Cell Biol 25, 4752-66 (2005).
3. Wang, Y. et al. Precision and functional specificity in mRNA decay. Proc Natl Acad Sci USA 99, 5860-5 (2002).
4. Takizawa, P. A., DeRisi, J. L., Wilhelm, J. E. & Vale, R. D. Plasma membrane compartmentalization in yeast by messenger RNA transport and a septin diffusion barrier. Science 290, 341-4 (2000).
5. Shepard, K. A. et al. Widespread cytoplasmic mRNA transport in yeast: identification of 22 bud-localized transcripts using DNA microarray analysis. Proc Natl Acad Sci USA 100, 11429-34 (2003).
6. Tucker, B. J. & Breaker, R. R. Riboswitches as versatile gene control elements. Curr Opin Struct Biol 15, 342-8 (2005).
7. Kato, J. & Niitsu, Y. Recent advance in molecular iron metabolism: translational disorders of ferritin. Int J Hematol 76, 208-12 (2002).
8. Chu, V. B. & Herschlag, D. Unwinding RNA's secrets: advances in the biology, physics, and modeling of complex RNAs. Curr Opin Struct Biol 18, 305-14 (2008).
9. Kertesz, M., Iovino, N., Unnerstall, U., Gaul, U. & Segal, E. The role of site accessibility in microRNA target recognition. in Nat Genet Vol. 39 1278-84 (2007).
10. Ingolia, N. T., Ghaemmaghami, S., Newman, J. R. S. & Weissman, J. S. Genome-Wide Analysis In Vivo of Translation with Nucleotide Resolution Using Ribosome Profiling. in Science 1168978v1 (2009).
11. Ameres, S. L., Martinez, J. & Schroeder, R. Molecular basis for target RNA recognition and cleavage by human RISC. Cell 130, 101-12 (2007).
12. Watts, J. M. et al. Architecture and secondary structure of an entire HIV-1 RNA genome. Nature 460, 711-6 (2009).
13. Bernstein, F. C. et al. The Protein Data Bank: a computer-based archival file for macromolecular structures. J Mol Biol 112, 535-42 (1977).
14. Brenowitz, M., Chance, M. R., Dhavan, G. & Takamoto, K. Probing the structural dynamics of nucleic acids by quantitative time-resolved and equilibrium hydroxyl radical “footprinting”. Curr Opin Struct Biol 12, 648-53 (2002).
15. Alkemar, G. & Nygard, O. Probing the secondary structure of expansion segment ES6 in 18S ribosomal RNA. Biochemistry 45, 8067-78 (2006).
16. Romaniuk, P. J., de Stevenson, I. L., Ehresmann, C., Romby, P. & Ehresmann, B. A comparison of the solution structures and conformational properties of the somatic and oocyte 5S rRNAs of Xenopus laevis. Nucleic Acids Res 16, 2295-312 (1988).
17. Deigan, K. E., Li, T. W., Mathews, D. H. & Weeks, K. M. Accurate SHAPE-directed RNA structure determination, in Proc Natl Acad Sci USA Vol. 106 97-102 (2009).
18. Das, R. et al. Structural inference of native and partially folded RNA by high-throughput contact mapping. Proc Natl Acad Sci USA 105, 4144-9 (2008).
19. Mitra, S., Shcherbakova, I. V., Altman, R. B., Brenowitz, M. & Laederach, A. High-throughput single-nucleotide structural mapping by capillary automated footprinting analysis. Nucleic Acids Res 36, e63 (2008).
20. Wilkinson, K. A. et al. High-throughput SHAPE analysis reveals structures in HIV-1 genomic RNA strongly conserved across distinct biological states. PLoS Biol 6, e96 (2008).
21. Zuker, M. Mfold web server for nucleic acid folding and hybridization prediction. Nucleic Acids Res 31, 3406-15 (2003).
22. Hofacker, I. L., Fekete, M. & Stadler, P. F. Secondary structure prediction for aligned RNA sequences. J Mol Biol 319, 1059-66 (2002).
23. Do, C. B., Woods, D. A. & Batzoglou, S. CONTRAfold: RNA secondary structure prediction without physics-based models. Bioinformatics 22, e90-8 (2006).
24. Mathews, D. H., Sabina, J., Zuker, M. & Turner, D. H. Expanded sequence dependence of thermodynamic parameters improves prediction of RNA secondary structure. J Mol Biol 288, 911-40 (1999).
25. Mathews, D. H. Revolutions in RNA secondary structure prediction. in J Mol Biol Vol. 359 526-32 (2006).
26. Rabani, M., Kertesz, M. & Segal, E. Computational prediction of RNA structural motifs involved in posttranscriptional regulatory processes, in Proc Natl Acad Sci USA Vol. 105 14885-90 (2008).
27. Dowell, R. D. & Eddy, S. R. Evaluation of several lightweight stochastic context-free grammars for RNA secondary structure prediction. BMC Bioinformatics 5, 71 (2004).
28. Doshi, K. J., Cannone, J. J., Cobaugh, C. W. & Gutell, R. R. Evaluation of the suitability of free-energy minimization using nearest-neighbor energy parameters for RNA secondary structure prediction. BMC Bioinformatics 5, 105 (2004).
29. Ziehler, W. A. & Engelke, D. R. Probing RNA structure with chemical reagents and enzymes. Curr Protoc Nucleic Acid Chem Chapter 6, Unit 61 (2001).
30. Rinn, J. L. et al. Functional demarcation of active and silent chromatin domains in human HOX loci by noncoding RNAs, in Cell Vol. 129 1311-23 (2007).
31. Nagalakshmi, U. et al. The transcriptional landscape of the yeast genome defined by RNA sequencing, in Science Vol. 320 1344-9 (2008).
32. Chartrand, P., Meng, X. H., Huttelmaier, S., Donato, D. & Singer, R. H. Asymmetric sorting of ashlp in yeast results from inhibition of translation by localization elements in the mRNA. Mol Cell 10, 1319-30 (2002).
33. Reineke, L. C., Komar, A. A., Caprara, M. G. & Merrick, W. C. A small stem loop element directs internal initiation of the URE2 internal ribosome entry site in Saccharomyces cerevisiae. J Biol Chem 283, 19011-25 (2008).
34. Mathews, D. H. et al. Incorporating chemical modification constraints into a dynamic programming algorithm for prediction of RNA secondary structure. Proc Natl Acad Sci USA 101, 7287-92 (2004).
35. Shabalina, S. A., Ogurtsov, A. Y. & Spiridonov, N. A. A periodic pattern of mRNA secondary structure created by the genetic code, in Nucleic Acids Res Vol. 34 2428-37 (2006).
36. Kozak, M. Regulation of translation via mRNA structure in prokaryotes and eukaryotes, in Gene Vol. 361 13-37 (2005).
37. Kudla, G., Murray, A. W., Tollervey, D. & Plotkin, J. B. Coding-sequence determinants of gene expression in Escherichia coli. in Science Vol. 324 255-8 (2009).
38. Palazzo, A. F. et al. The signal sequence coding region promotes nuclear export of mRNA. PLoS Biol 5, e322 (2007).
39. Cech, T. R., Damberger, S. H. & Gutell, R. R. Representation of the secondary and tertiary structure of group I introns. Nat Struct Biol 1, 273-80 (1994).
40. Hofacker L I., et al. Fast folding and comparison of RNA secondary structures. Monatshefte Fr. Chemie. 125:167-188, 1994;
41. Do C B., Woods D A., et al. CONTRAfold: RNA seconday structure prediction without physics-based models. Bioinfomatics 22:90-98, 2006.

### Supplementary Data Content

The following lists the content of the text file which is submitted herewith and filed with the application. File information is provided as: File name/byte size/date of creation/operating system/machine format.

Supplementary_Data/18,453,201 bytes/7 April 2010/PC/Notepad

### Text File Content

The following lists the content of the text file which is enclosed herewith and filed with the application. File information is provided as: File name/byte size/date of creation/operating system/machine format.

Sequence_Listing/6,374,281 bytes/19 April 2010/PC/Notepad

What is claimed is:

1. A method of predicting a pairability of nucleotides of a plurality of RNA polynucleotides, the method comprising:
(a) simultaneously determining a paired state or an unpaired state of nucleotides of the plurality of RNA polynucleotides; and
(b) corresponding said paired state or said unpaired state of said nucleotides to a database of nucleic acid sequences, said database comprises nucleic acid sequences representing the plurality of RNA polynucleotides,
thereby determining the pairability of nucleotides of the plurality of RNA polynucleotides.

2. A method of determining a secondary structure of a plurality of RNA polynucleotides, the method comprising:
(a) predicting the pairability of nucleotides of the plurality of RNA polynucleotides according to the method of claim 1; and
(b) determining the secondary structure of the plurality of RNA polynucleotides based on the predicted pairability of said nucleotides,
thereby determining the secondary structure of the plurality of the RNA polynucleotides.

3. A method of determining if a molecule is capable of modulating a secondary structure of at least one RNA polynucleotide of a plurality of RNA polynucleotides, the method comprising:
(a) contacting the plurality of RNA polynucleotides with the molecule; and
(b) comparing a secondary structure of the plurality of RNA polynucleotides following said contacting to a secondary structure of the plurality of RNA polynucleotides prior to said contacting,
wherein an alteration above a predetermined threshold in said secondary structure of an RNA polynucleotide following said contacting indicates that the molecule modulates the secondary structure of said RNA polynucleotide,
thereby determining if the molecule is capable of modulating the secondary structure of the at least one RNA polynucleotide of the plurality of molecules.

4. A method of determining if a molecule is capable of modulating a secondary structure of a plurality of RNA polynucleotides, the method comprising
(a) contacting the plurality of RNA polynucleotides with the molecule; and
(b) determining a secondary structure of the plurality of RNA polynucleotides according to the method of claim 2 following said contacting and comparing said secondary structure to a secondary structure of the same plurality of RNA polynucleotides prior to said contacting,
wherein an alteration above a predetermined threshold of said secondary structure following said contacting indicates that the molecule modulates the secondary structure of the RNA polynucleotides,
thereby determining if the molecule is capable of modulating the secondary structure of the plurality of RNA polynucleotides.

5. A method of screening for a marker associated with a pathology, the method comprising identifying at least one RNA polynucleotide having an altered secondary structure between cells associated with the pathology and cells devoid of the pathology, wherein an alteration above a predetermined threshold between said secondary structure of said at least one RNA polynucleotide in said cells associated with the pathology and said secondary structure of said at least one RNA polynucleotide in said cells devoid of the pathology indicates that said at least one RNA polynucleotide is associated with the pathology, thereby screening for a marker associated with the pathology.

6. The method of claim 1, wherein said determining said paired state or said unpaired state is effected using an RNA structure—dependent agent.

7. The method of claim 6, wherein said RNA structure—dependent agent is an RNase selected from the group consisting of:
(i) an RNase which specifically cleaves a phosphodiester bond of a paired RNA, and
(ii) an RNase which specifically cleaves a phosphodiester bond of an unpaired RNA.

8. The method of claim 7, wherein said RNase is an endonuclease.

9. The method of claim 6, wherein said RNA structure—dependent agent is a chemical selected from the group consisting of:
(i) a chemical which specifically binds to or modifies an unpaired RNA, and;
(ii) a chemical which specifically binds to or modifies a paired RNA.

10. The method of claim 9, wherein binding of said chemical to said RNA is effected covalently.

11. The method of claim 7, wherein said determining said paired state or said unpaired state of said nucleotides is effected by digesting the plurality of RNA polynucleotides with said RNase to thereby obtain digested RNA polynucleotides.

12. The method of claim 11, further comprising subjecting said digested RNA polynucleotide to reverse transcription to thereby obtain complementary DNA polynucleotides.

13. The method of claim 9, wherein said determining said paired state or said unpaired state of said nucleotides is effected by reverse transcription of said plurality of RNA polynucleotides following binding of said plurality of RNA polynucleotides with said chemical, to thereby obtain complementary DNA polynucleotides.

14. The method of claim 12, wherein said corresponding said paired state or said unpaired state of said nucleotides to said data base nucleic acid sequences is effected by comparing a nucleic acid sequence of said complementary DNA polynucleotides with said data base nucleic acid sequences.

15. The method of claim 14, further comprising computing an occurrence of a nucleotide of each of the plurality of RNA polynucleotides within said nucleic acid sequence of said complementary DNA polynucleotides.

16. The method of claim 14, wherein said nucleic acid sequence of said complementary DNA polynucleotides is determined using a sequencing apparatus selected from the group consisting SOLEXA™ (IIlumina), PYROSEQUENCING™ 454 (Roche Diagnostics Corporation), SOLiD™ (Life Technologies), and Helicos (Helicos BioSciences Corporation).

17. The method of claim 16, wherein determination of said nucleic acid sequence of said complementary DNA polynucleotides is effected for each of said complementary DNA polynucleotides.

18. The method of claim 15, wherein said computing said occurrence is performed on a nucleotide corresponding to a first nucleotide and/or a last nucleotide of each of said complementary DNA polynucleotides.

19. The method of claim 15, wherein a higher occurrence of said nucleotide within said complementary DNA polynucleotides obtained using said RNA structure—dependent agent which is specific to said paired RNA as compared to an expected occurrence of said nucleotide indicates that said nucleotide is in said paired state in the RNA polynucleotide prior to being treated with said RNA structure—dependent agent.

20. The method of claim 15, wherein a higher occurrence of said nucleotide within said complementary DNA polynucleotides obtained using said RNA structure—dependent agent which is specific to said unpaired RNA as compared to an expected occurrence of said nucleotide indicates that said nucleotide is in said unpaired state in the RNA polynucleotide prior to being treated with said RNA structure—dependent agent.

21. The method of claim 15, wherein a higher occurrence of said nucleotide within said complementary DNA polynucleotides obtained using said RNA structure—dependent agent which is specific to said paired RNA as compared to an occurrence of said nucleotide in said complementary DNA polynucleotides obtained using said RNA structure—dependent agent which is specific to said unpaired RNA indicates that said nucleotide is in said paired state in the RNA polynucleotide prior to being treated with said RNA structure—dependent agent, and vice versa.

22. The method of claim 15, wherein a higher occurrence of said nucleotide within said complementary DNA polynucleotides obtained using said RNA structure—dependent agent which is specific to said unpaired RNA as compared to an occurrence of said nucleotide in said complementary DNA polynucleotides obtained using said RNA structure—dependent agent which is specific to said paired RNA indicates that said nucleotide is in said unpaired state in the RNA polynucleotide prior to said being treated with said RNA structure—dependent agent, and vice versa.

23. The method of claim 1, further comprising removing proteins from the plurality of the RNA polynucleotides prior to said determining said paired state or said unpaired state of said nucleotides of the plurality of RNA polynucleotides.

24. The method of claim 1, further comprising denaturing the plurality of the RNA polynucleotides prior to said determining said paired state or said unpaired state of said nucleotides of the plurality of RNA polynucleotides.

25. The method of claim 24, further comprising subjecting the plurality of the RNA polynucleotides to conditions which allow folding of the RNA polynucleotides following said denaturing.

26. The method of claim 7, wherein said RNase which specifically cleaves said phosphodiester bond of said paired RNA is selected from the group consisting of RNase V1 and RNase R.

27. The method of claim 7, wherein said RNase which specifically which specifically cleaves said phosphodiester bond of said unpaired RNA is selected from the group consisting of RNase S1, RNase T1 and RNase A.

28. The method of claim 1, wherein the plurality of RNA polynucleotides are obtained from a cell of an organism.

29. The method of claim 3, wherein said secondary structure of the plurality of RNA polynucleotides is determined according to the method of claim 2.

30. The method of claim 1, wherein the pairability is determined for each of the nucleotides of at least two of the plurality of RNA polynucleotides.
